(19) United States
(12) Patent Application Publication
(10) Pub. No.: US 2022/0296845 A1
(43) Pub. Date: Sep. 22, 2022

(54) Systems and Methods for Nitric Oxide Generation and Delivery

(71) Applicant: Third Pole, Inc., Waltham, MA (US)

(72) Inventors: Nathaniel G. Jackson, Lexington, MA (US); Benjamin J. Apollonio, Lunenburg, MA (US); Gregory W. Hall, Belmont, MA (US)

(73) Assignee: Third Pole, Inc., Waltham, MA (US)

(21) Appl. No.: 17/693,279

(22) Filed: Mar. 11, 2022

Related U.S. Application Data
(60) Provisional application No. 63/159,981, filed on Mar. 11, 2021.
(60) Provisional application No. 63/194,145, filed on May 27, 2021.
(60) Provisional application No. 63/264,336, filed on Nov. 19, 2021.

(51) Int. Cl. A61M 16/12; A61M 16/20; A61M 16/00; C01B 21/20; H05H 1/46; B01D 53/14

(52) U.S. Cl. CPC A61M 16/12; A61M 16/208; A61M 16/0003; C01B 21/203; H05H 1/463; B01D 53/1456; B01D 53/1412; A61M 2202/0275; A61M 2205/3334; A61M 2016/0027; A61M 2205/3327; H05H 2245/10

(57) ABSTRACT

The present disclosure provides systems and methods for nitric oxide (NO) generation and/or delivery. In some aspects, a nitric oxide generation system comprises a plasma chamber configured to ionize a reactant gas including nitrogen and oxygen to form a product gas that includes NO, a scrubber downstream from the plasma chamber and having a volume at least partially containing NO₂ scrubbing material, and a flow controller downstream of the scrubber configured to control the flow of product gas from the scrubber to a delivery device. A pump is configured to convey product gas from the plasma chamber into the scrubber and is configured to pressurize the product gas in the scrubber when the flow controller is positioned to restrict the flow of product gas from the scrubber. The pressurized product gas accumulates within the scrubber and is at least partially scrubbed of NO₂ prior to passage through the flow controller.

### RELATED APPLICATIONS

This application claims the benefit of and priority to U.S. Provisional Application No. 63/159,981 filed Mar. 11, 2021, U.S. Provisional Application No. 63/194,145 filed May 27, 2021, and U.S. Provisional Application 63/264,336 filed Nov. 19, 2021, and the contents of each of these applications are hereby incorporated herein by reference in their entireties.

### FEDERALLY SPONSORED RESEARCH OR DEVELOPMENT

This invention was made with government support under Grant No. R44 TR001704, awarded by the National Institutes of Health (NIH). The government has certain rights in the invention.

### FIELD

The present disclosure relates to systems and methods for generating nitric oxide.

### BACKGROUND

Nitric oxide (NO) has been found to be useful in a number of ways for treatment of disease, particularly cardiac and respiratory ailments. Previous systems for producing NO and delivering the NO gas to a patient have a number of disadvantages. For example, tank-based systems require tanks of NO gas at a high concentration and are required to either purge oxidized NO with fresh NO when treatment is resumed or minimize exposure of delivery system NO gas to air between breaths. Synthesizing NO from NO₂ or N₂O₄ requires the handling of toxic chemicals. Prior electric generation systems involve generating plasma in the main flow of air to be delivered to patients or pumped through a delivery tube.

### SUMMARY

The present disclosure related to systems and methods for generating and/or delivering nitric oxide.

In some aspects, the present disclosure provides a nitric oxide generation system, comprising a plasma chamber configured to ionize a reactant gas including nitrogen and oxygen to form a product gas that includes nitric oxide (NO), a scrubber downstream from the plasma chamber and having a volume at least partially containing NO₂ scrubbing material, and a flow controller downstream of the scrubber, the flow controller configured to control the flow of product gas from the scrubber to a delivery device. A pump is configured to convey the product gas from the plasma chamber into the scrubber, the pump configured to pressurize the product gas in the scrubber when the flow controller is positioned to restrict the flow of product gas from the scrubber. The pressurized product gas accumulates within the scrubber and is at least partially scrubbed of NO₂ prior to passage from the scrubber through the flow controller.

In some embodiment, a reactant gas flow rate through the plasma chamber is continuous. In some embodiments, the reactant gas flow rate through the plasma chamber is a constant value. In some embodiments, a reactant gas flow rate through the plasma chamber is intermittent. In some embodiments, a pressure within the plasma chamber is at or below atmospheric pressure.

In some embodiments, the system can also include a pressure sensor to measure the pressure in the scrubber. In some embodiments, the system can also include a controller configured to regulate an amount of NO in the product gas by modulating a plasma in the plasma chamber, the controller utilizing a pressure measurement in the scrubber to determine a flow rate of the product gas out of the scrubber.

In some embodiments, the product gas is delivered intermittently. In some embodiments, a product gas delivery flow rate varies pulse to pulse. In some embodiments, a product gas delivery flow rate varies within a pulse. In some embodiments, a mass of the product gas in the scrubber is at least a mass of a single NO pulse.

In some embodiments, the volume between the scrubber and the flow controller is less than 5 ml. In some embodiments, the volume between the scrubber and the flow controller is less than 10 ml.

In some embodiments, the system includes a parallel flow path that includes a pressurized non-NOx containing gas. In some embodiments, the pressurized reactant gas is utilized to push an NO pulse to a patient and purge at least a portion of at least one of a pneumatic pathway within the system and the delivery device of NO and NO₂.

In some embodiments, the product gas is configured to accumulate such that an increase in an oxidation due to the pressure in the scrubber is more than offset by an improvement in scrubbing due to one or more of an increase in a residence time and the pressure in the scrubber.

In some embodiments, the system includes a controller configured to calculate an estimated amount of NO loss within the system due to at least one of oxidation of NO and interaction between the product gas and components of the system. In some embodiments, the controller is configured to control the plasma chamber to overproduce NO in anticipation of the estimated amount of NO loss calculated by the controller.

In some embodiments, a product gas flow rate entering the scrubber is different than from product gas flow rate exiting the scrubber. In some embodiments, a mass of gas between the pump and the flow controller, including the scrubber, is greater than a mass of a pulse of gas to be delivered to a delivery device.

A nitric oxide generation system is provided that comprises a plasma chamber configured to ionize a reactant gas including nitrogen and oxygen to form a product gas that includes nitric oxide (NO), a scrubber downstream having a volume at least partially containing NO₂ scrubbing material, and a flow controller downstream of the scrubber, the flow controller configured to control the flow of product gas from the scrubber to a delivery device. A pump is configured to push the product gas from the plasma chamber into the scrubber, the pump being configured to pressurize the product gas in the scrubber when the flow controller is positioned to restrict the flow of product gas from the scrubber. A controller is configured to regulate an amount of NO in the product gas by the plasma chamber, and the controller utilizes a pressure measurement in the scrubber to determine a mass flow rate of the product gas out of the scrubber. The pressurized product gas accumulates within the scrubber and is at least partially scrubbed of NO₂ prior to passage from the scrubber through the flow controller, and a mass of gas in the scrubber and pneumatic connections between the pump and the flow controller is greater than a mass of a pulse of gas to be delivered to a delivery device.

In some embodiments, a reactant gas flow rate through the plasma chamber is continuous. In some embodiments, the reactant gas flow rate through the plasma chamber is a constant value. In some embodiments, a reactant gas flow rate through the plasma chamber is intermittent. In some embodiments, a pressure within the plasma chamber is at or below atmospheric pressure.

In some embodiments, the system includes a pressure sensor to measure the pressure in the scrubber. In some embodiments, the system includes a controller configured to regulate the amount of NO in the product gas by modulating a plasma in the plasma chamber, the controller utilizing a pressure measurement in the scrubber to determine a flow rate of the product gas out of the scrubber.

In some embodiments, the product gas is delivered intermittently. In some embodiments, a product gas delivery flow rate varies pulse to pulse. In some embodiments, a product gas delivery flow rate varies within a pulse. In some embodiments, a mass of the product gas in the scrubber is at least a mass of a single NO pulse.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 illustrates an exemplary embodiment of a NO generation system;

FIG. 2 depicts an embodiment of a NO generation and delivery system;

FIG. 3 depicts an exemplary linear NO generation device architecture;

FIG. 4 depicts an embodiment of an NO generation device with a 3-way valve that can direct pumped gas into the device enclosure;

FIG. 5 depicts an embodiment of a linear NO generation device architecture with a pressurized reservoir;

FIG. 6 illustrates an embodiment of a NO generation and delivery system with a recirculation architecture;

FIG. 7 depicts an exemplary NO generation architecture with an internal recirculation loop;

FIG. 8 depicts an exemplary timing sequence for operating a NO generation system for pulsed NO delivery;

FIG. 9 depicts an exemplary architecture that enables flow to be established through a scrubber prior to breath detection.

FIG. 10 illustrates an embodiment of an NO generation device with a gas being circulated from a treatment controller to a delivery device and back to the controller;

FIG. 11A depicts an embodiment of a cannula with an intersection point at the base of the patient's neck;

FIG. 11B depicts an embodiment of a cannula with an intersection located at the patient's ear;

FIG. 11C depicts an embodiment of a cannula with an intersection located at the patient's nose;

FIG. 12 depicts an embodiment of a NO generation system that utilizes independent pumps for bypass and scrubber flow paths;

FIG. 13 presents an exemplary plot of NO2 concentration in gas exiting a soda lime scrubber at various pressures;

FIG. 14A depicts an exemplary embodiment of a bypass gas reservoir that is filled by a pump with a flow controller at an exit of the reservoir;

FIG. 14B depicts an exemplary embodiment of a bypass reservoir with a pressure relief valve;

FIG. 14C depicts an exemplary embodiment of a bypass reservoir with an actively controlled valve;

FIG. 15 depicts an embodiment of a pressurized scrubber architecture;

FIG. 16 depicts an exemplary embodiment of an architecture with a plasma chamber being located before the pump so that the pressure within the plasma chamber is constant and low;

FIG. 17 illustrates a graph of exemplary experimental NO production data from an electric NO generation device operating with 1.5 slpm of reactant gas flow and various plasma duty cycles;

FIG. 18A depicts an exemplary graph of performance of a system that is slow to terminate the NO pulse;

FIG. 18B depicts an exemplary graph of performance of a system that can terminate the NO pulse more rapidly;

FIG. 19 depicts an exemplary pressurized scrubber with a bypass design;

FIG. 20 depicts an exemplary bypass architecture with separate pumps for the bypass and scrubber pathways;

FIG. 21 depicts such an exemplary NO system with one or more pumps pressurizing an accumulator;

FIG. 22 depicts an embodiment of a pressurized scrubber/pressurized bypass design with a pneumatic flow path exiting the product gas scrubber;

FIG. 23 depicts a graph of an exemplary timing sequence for a pressurized scrubber/pressurized bypass system;

FIG. 24 depicts an exterior of an exemplary NO generation and delivery device;

FIG. 25 depicts an exemplary NO generation and delivery device with a GCC removed;

FIG. 26 depicts an exemplary NO generation and delivery device with the enclosure opened;

FIG. 27 depicts exemplary internal components of the NO device shown in FIG. 26;

FIG. 28 depicts an exemplary user interface for an ambulatory NO generation device;

FIG. 29 illustrates an exemplary graph of flow from the scrubber and flow from the bypass channel overlap;

FIG. 30 depicts a graph of performance of an exemplary pressurized scrubber/pressured purge system;

FIG. 31 illustrates an exemplary graph of an approach to spreading NO over a larger portion of a breath;

FIG. 32A depicts an embodiment of a NO generator where the NO and bypass paths intersect within the device;

FIG. 32B depicts an embodiment of a NO generator where flow through the bypass and NO channels remain independent within the NO generator and combine within a delivery device;

FIG. 32C depicts an embodiment of a NO generator where the NO and purge lines are independent in the controller and the NO line is scrubbed using a scrubber in a delivery device;

FIG. 33A depicts an exemplary graph showing a system that is slow to terminate delivery of NO to the patient.

FIG. 33B depicts an exemplary graph of a system that is slow to shut off the NO flow can be turned off earlier in the inspiratory event to prevent dosing the non-target part of the lung;

FIG. 33C depicts an exemplary graph of a system that can rapidly terminate the NO bolus;

FIG. 34 depicts a graph showing an exemplary approach to prolonging the NO pulse;

FIG. 35 depicts a graph of exemplary data from a NO pulsed device utilizing a pressurized scrubber, pressurized bypass architecture;

FIG. 36 depicts an exemplary graph showing a bolus of NO is released into the delivery device;

FIG. 37 depicts a graph of an example of a pulse delivery approach that varies the NO pulse flow rate in order to improve the consistency of NO concentration within the dosed portion of a tidal volume;

FIG. 38 depicts an exemplary graph of an embodiment where the cannula is primed with NO gas and purge gas flowing simultaneously;

FIG. 39A depicts an exemplary graph of an NO pulse being delivered as soon as possible within the inspiration;

FIG. 39B depicts an exemplary graph showing a delay occurring prior to delivery of the NO pulse through the delivery system;

FIG. 40 depicts an exemplary design of a NO generation system that cools a plasma chamber convectively with purge gas;

FIG. 41 depicts an embodiment of a NO generation system with a pressurized scrubber with pressurized bypass architecture that functions with a single pump;

FIG. 42 depicts an exemplary embodiment of a NO generation system whereby a single pump 532 runs continuously;

FIG. 43 depicts an exemplary embodiment of a push/pull architecture including an external recirculation loop with a shunt to create an internal recirculation loop;

FIG. 44 depicts an exemplary embodiment of a push/pull architecture that is open loop;

FIG. 45 depicts an exemplary embodiment of a pulsed NO delivery device;

FIG. 46 depicts an embodiment of an NO generation system with a pressured scrubber with pressurized bypass architecture;

FIG. 47 depicts an exemplary graph showing one minute of an exemplary treatment of a patient with a dosing rate of 6 mg/hr.;

FIG. 48A illustrates an exemplar graph of a target intra-lung concentration with actual lung concentration;

FIG. 48B depicts an exemplary graph of a patient breathing over time;

FIG. 48C shows an exemplary dosing scheme whereby a gas delivery system doses a current breath as if it was the prior breath;

FIG. 49 depicts an exemplary graph of a relationship between inspiratory duration and breath period;

FIG. 50 is an exemplary graph depicting the relationship between pulse duration and respiratory rate;

FIG. 51 depicts an exemplary graph showing the relative timing of NO and NO2 delivery from a pressured scrubber NO delivery system;

FIG. 52 depicts an exemplary embodiment of a tank-based NO delivery system with a purge feature;

FIGS. 53A, 53B, and 53C depict an example of pulse queueing with a system that queues a NO pulse within the delivery device based on a delay from the end of inspiration;

FIG. 54A depicts an embodiment of a delivery system filled with gas that does not contain NO;

FIG. 54B depicts an embodiment of a NO controller filling the cannula with NO containing gas;

FIG. 54C depicts an embodiment with a bolus of NO being delivered to the patient by pushing the NO gas through the delivery device with inert, non-NO containing gas;

FIG. 55 depicts an embodiment of a system that utilizes a compressed gas canister of purge gas 640 and a compressed gas cannister of NO gas;

FIG. 56 depicts a NO generation system that utilizes the purge gas flow through a heat exchanger to pull heat out of the product gas after it leaves the plasma chamber;

FIG. 57 illustrates an embodiment of a NO generation system that manages temperature by product gas cooling that utilizes purge gas;

FIG. 58 depicts an embodiment of a NO generation system that manages temperature within the product gas;

FIG. 59 presents an exemplary graph showing NO oxidation experimental data;

FIG. 60 depicts an exemplary embodiment of a NO generator with a removable cartridge that prepares reactant gas and scrubs and filters product gas;

FIG. 61 depicts an exemplary embodiment of a NO generation device with a pressurized scrubber and pressurized bypass architecture with independent gas inlets for each leg;

FIG. 62 depicts an exemplary disposable component that includes only the scrubber, filters and desiccant;

FIG. 63 depicts an exemplary embodiment of a cartridge design where the delivery device connects directly to the cartridge;

FIG. 64 depicts an exemplary embodiment of a cartridge with an elastomeric tube section between the scrubber and the delivery device connection;

FIG. 65 depicts an embodiment of a system and cartridge that uses of a needle and seat valve within the cartridge that is actuated by an actuator within the controller;

FIG. 66 depicts an exemplary embodiment of a cartridge with electrical connections to the controller and an electric valve to control flow exiting the scrubber;

FIGS. 67A and 67B depicts an exemplary embodiment of a cartridge where an endcap in the scrubber housing serves as a valve housing;

FIG. 68 depicts an embodiment of a cartridge where an actuator from the controller side can press on a diaphragm or flapper valve to control the flow of product gas exiting the scrubber;

FIG. 69 depicts an embodiment of a GCC that reduces an insertion force for a GCC;

FIGS. 70A and 70B depict an exemplary embodiment of a GCC for facilitating the installation of a GCC with multiple pneumatic connections;

FIG. 71 depicts an exemplary delivery device positioned on the head of a patient;

FIG. 72A depicts an embodiment of a long prong placement tool;

FIG. 72B depicts an embodiment of a long prong placement tool;

FIG. 73A depicts an exemplary cannula with three lumens between the controller and a junction point along the length of the tubing;

FIG. 73B depicts an exemplary embodiment of a delivery device for merging a NO lumen and a breath detect lumen;

FIGS. 74A and 74B illustrates a cross-sectional view of the dual-lumen cannula and a side cross-sectional view of the dual-lumen cannula;

FIG. 75A-75E depicts various mixing element designs within and/or affixed to the end of a gas delivery prong;

FIG. 76 depicts an exemplary embodiment of a nasal cannula that routes to the NO device first;

FIG. 77 depicts an exemplary embodiment of a delivery device that includes a proximal scrubber and/or particulate filter as part of a mask;

FIG. 78 depicts an exemplary delivery system that utilizes a NO2 scrubbing material splined filament;

FIG. 79 depicts a cross-sectional view of an exemplary multi-lumen NO and oxygen delivery device 870;

FIG. 80A depicts an exemplary high surface area delivery device with parallel slits;

FIG. 80B depicts a high surface area delivery device with multiple rings and spokes creating multiple lumens through the extrusion;

FIG. 80C depicts an embodiment of a high surface area delivery device for scrubbing with multiple equivalent lumens;

FIG. 81 depicts an exemplary embodiment of a delivery device with an oxygen delivery lumen in the center and multiple NO delivery lumens around the periphery;

FIG. 82 depicts an exemplary embodiment of a combination NO generator and humidification device;

FIG. 83 depicts an exemplary embodiment of a combination NO generator and humidifier;

FIGS. 84A, 84B, and 84C depict an exemplary embodiment of an EMG breath detection device;

FIG. 85 depicts an embodiment of a NO generator with an oxygen pass-through;

FIG. 86 depicts an exemplary embodiment of a NO generation system that uses an oxygen delivery lumen for breath detection;

FIG. 87 depicts an embodiment of a NO generator with oxygen through-flow;

FIG. 88A depicts an embodiment of a granular desiccant chamber that at least partially desiccates gas;

FIG. 88B depicts an embodiment of a desiccant chamber with solid, non-perforated baffles that force gas flow to pass through the desiccant material;

FIGS. 89A and 89B depict an embodiment of a gas conditioning cartridge (GCC);

FIG. 90 illustrates an exemplary embodiment of a cross-section of a gas conditioning cartridge;

FIG. 91 illustrates a cross section of an exemplary GCC in the region of the NO2 scrubber;

FIG. 92A illustrates an embodiments of additional scrubber sheet material being placed in the air gap;

FIG. 92B depicts an embodiment of a scrubber housing filled with scrubber material;

FIG. 92C depicts an exemplary scrubber chamber that has tapered or conical entry and/or exit geometry;

FIG. 92D depicts an exemplary scrubber chamber that utilizes granular scrubber material;

FIG. 93 depicts a horizontal cross section of an embodiment of a GCC;

FIG. 94 depicts a cross section of an exemplary embodiment of a GCC at the location of the scrubbed product gas and purge gas delivery path;

FIG. 95 depicts an exemplary gas delivery cannula that utilizes the cannula tubing as light pipes to send and receive optical information;

FIG. 96 depicts an exemplary connection of an optical measurement/gas delivery device with the gas source;

FIG. 97A depicts an exemplary embodiment of a NO generation device for use with concomitant oxygen delivery;

FIG. 97B depicts an exemplary embodiment of a NO delivery device that operates simultaneously with an oxygen delivery device;

FIG. 98A depicts an exemplary NO generator with a reactant gas preconditioning stage;

FIG. 98B depicts a NO generation device with a desiccant stage that dries reactant gas to extremely low humidity levels;

FIG. 99A depicts a NO generation system that blends a mixture of desiccated reactant gas and ambient gas to a target humidity level with a 3-way valve.

FIG. 99B depicts an exemplary embodiment of a device where all reactant gas flows through a desiccant stage prior to the plasma chamber;

FIG. 100 depicts an exemplary bypass architecture system that desiccates all of the reactant gas entering the plasma chamber;

FIG. 101 depicts an exemplary bypass architecture system with a fixed blending ratio for purge gas;

FIG. 102 an exemplary graph representing the dew point for gases of varying humidity as a function of pressure and humidity for a specific water content of gas;

FIG. 103 depicts an exemplary bypass architecture design with a variable blending stage at the inlet;

FIG. 104 depicts an exemplary look up table that a NO generation system and/or delivery system that operates at 10 psi max internal pressure can use to prevent condensation within the system;

FIG. 105A depicts an exemplary NO device connected to a patient end of the inspiratory limb;

FIG. 105B depicts an exemplary NO generation system operating independently of a concomitant therapy;

FIG. 106 depicts an exemplary ET tube for NO delivery;

FIG. 107 depicts an exemplary ET tube for NO delivery with a fast temperature sensor in the wall for breath detection;

FIG. 108A depicts an embodiment of a NO generation device connected to a ventilation circuit;

FIG. 108B depicts an embodiment of an NO generation device having a pressurized scrubber located at the patient Wye or ET fitting;

FIGS. 109A and 109B illustrates exemplary embodiments of NO generation systems that demonstrate that NO can be introduced at various locations within the inspiratory limb;

FIG. 110 depicts an exemplary NO injector design that interfaces with a patient Y-fitting and ventilator tubing;

FIG. 111 depicts an exemplary NO injection design that includes a gas sampling port;

FIG. 112 depicts an exemplary embodiment of an NO injection design where the NO is introduced through an NO lumen to the patient leg of the Wye fitting;

FIG. 113A depicts an embodiment of a dual-lumen inspiratory line with a dedicated lumen for NO delivery;

FIG. 113B depicts an embodiment of a dual lumen extrusion with one lumen flowing inspiratory gas and the other lumen delivering NO;

FIG. 114A depicts an exemplary graph showing flow rate and NO delivery over time using a NO system that delivers NO to an inspiratory limb continuously;

FIG. 114B depicts an exemplary graph showing flow rate and NO delivery over time where only the volume of inspiratory gas that is inhaled is dosed;

FIG. 114C depicts an exemplary graph showing flow rate and NO delivery over time in which NO is introduced to the first half of the breath;

FIG. 114D depicts an exemplary graph showing flow rate and NO delivery over time in which NO is delivered to the latter part of the inspired volume;

FIG. 115 depicts an embodiment of a NO generation and/or delivery device in use with a bag;

FIG. 116 depicts an embodiment of a NO generation device that utilizes a remote sensor located in the bag/mask assembly to detect an inspiratory event;

FIG. 117 depicts an embodiment of an NO device whereby the inspiratory gas flows through the NO device;

FIG. 118 depicts an embodiment of an NO device used with a manual resuscitation system;

FIG. 119 depicts an exemplary embodiment of a dual-lumen cannula with dual-lumen prongs and gas filtration;

FIG. 120A depicts an exemplary embodiment of an electrode array consisting of three pairs of parallel electrodes forming three gaps;

FIG. 120B depicts an exemplary embodiment of an electrode array with 5 electrodes forming 4 gaps; and

FIG. 120C depicts an exemplary embodiment of an electrode array with 5 electrodes forming 4 gaps.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure relates to systems and methods of nitric oxide (NO) delivery for use in various applications, for example, inside a hospital room, in an emergency room, in a doctor's office, in a clinic, and outside a hospital setting as a portable or ambulatory device. An NO generation and/or delivery system can take many forms, including but not limited to a device configured to work with an existing medical device that utilizes a product gas, a stand-alone (ambulatory) device, a module that can be integrated with an existing medical device, one or more types of cartridges that can perform various functions of the NO system, an inhaler, and an electronic NO tank. The NO generation system uses a reactant gas, including but not limited to ambient air, to produce a product gas that is enriched with NO.

An NO generation device can be used with or integrated into any device that can utilize NO, including but not limited to a ventilator, a resuscitation instrument, an anesthesia device, a defibrillator, a ventricular assist device (VAD), a Continuous Positive Airway Pressure (CPAP) machine, a Bilevel Positive Airway Pressure (BiPAP) machine, a non-invasive positive pressure ventilator (NIPPV), a nasal cannula application, a heated high-flow nasal cannula application, a nebulizer, an extracorporeal membrane oxygenation (ECMO), a cardio-pulmonary bypass system, an automated CPR system, an oxygen delivery system, an oxygen concentrator, an oxygen generation system, and an automated external defibrillator AED, MRI, and a patient monitor. In addition, the destination for nitric oxide produced can be any type of delivery device associated with any medical device, including but not limited to a nasal cannula, a manual ventilation device, a face mask, inhaler, scoop catheter, endotracheal (ET) tube, topical applicator, CPAP inspiratory limb, ventilator inspiratory limb, or other delivery circuit components. The NO generation capabilities can be integrated into any of these devices, or the devices can be used with a NO generation device as described herein. In some embodiments, the system is portable for use outside a hospital. In some embodiments, the system is used with an oxygen generator or an oxygen concentrator as concomitant therapy and to increase nitric oxide production.

Patients receiving NO therapy can either be dosed continuously or discontinuously. Continuous dosing is typically prescribed as a concentration of NO in the inspired gas (e.g., 20 ppm NO). In some embodiments, continuous dosing typically results in the delivering a consistent concentration of NO to the entire lung and airway. In some embodiments, discontinuous dosing can involve NO delivery for a subset of breaths (e.g., every other breath). Other embodiments of discontinuous dosing involve NO delivery to a portion of the inhaled volume (e.g., dosing the first ½ of a breath). Intermittent dosing is typically prescribed in terms of a mass of NO per unit time (e.g., 6 mg/hr). The dose varies with the clinical indication. Infections are typically treated with post-dilution NO concentrations of 150 ppm and higher (e.g., 1000 ppm) delivered to the entire breath, whereas hemodynamic/oxygenation benefits can be seen with post-dilution NO concentrations of 80 ppm or less delivered to a portion of the breath. Even patients receiving a post-dilution NO concentration of 1-2 ppm are increasing the tissue NO concentration within their lung by more than an order of magnitude.

Reactant gas for NO generation consists of nitrogen and oxygen-containing compounds (e.g. N₂, NO₂, N₂O, and O₂. It can be ambient air, but it can also be sourced from cylinders or other sources with either atmospheric or non-atmospheric ratios of N₂ to O₂. For example, NO delivery systems can source NO from gas tanks, solid sources, and liquid sources.

Any of these architectures can be coated or lined with NO₂-scrubbing material to clean gas as it travels through the pneumatic pathways. It will also be understood that a particle filter can be used downstream from a scavenger/scrubber in any of the architectures described herein.

It will also be understood that any pneumatic controls described herein can be directed by a microprocessor, FPGA or any other type of controller, for example, including a treatment controller as described below.

Many architectures depict 3-way valves, a means to direct flow in one direction or another. It should be understood that any equivalent means of directing gas flow is implied by these diagrams. For example, one or more binary valves or proportional valves could serve the same purpose. It will be understood that the use of “flow” in this document in the context of control and sensing includes “mass flow” and “volumetric flow” unless otherwise specified.

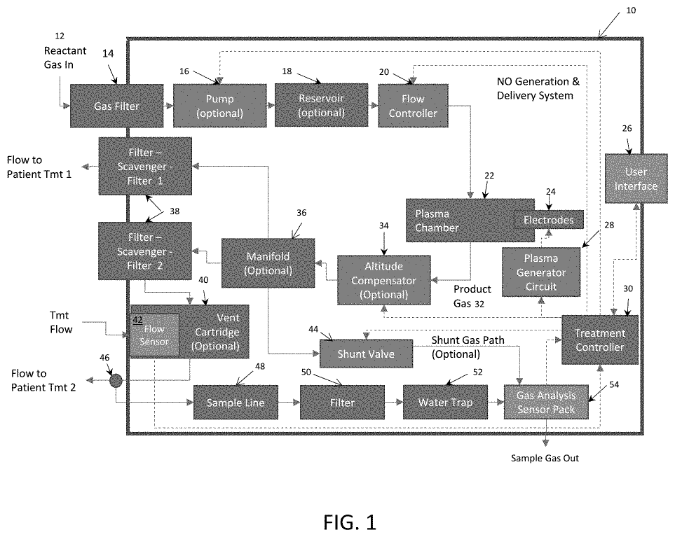

FIG. 1 illustrates an exemplary embodiment of a NO generation system 10 that includes components for reactant gas intake 12 and delivery to a plasma chamber 22. The plasma chamber 22 includes one or more electrodes 24 therein that are configured to produce, with the use of a high voltage circuit 28, a product gas 32 containing a desired amount of NO from the reactant gas. The system includes a controller 30 in electrical communication with the plasma generator 28 and the electrode(s) 24 that is configured to control the concentration of NO in the product gas 32 using one or more control parameters relating to conditions within the system and/or conditions relating to a separate device for delivering the product gas to a patient and/or conditions relating to the patient receiving the product gas and/or conditions relating to the reactant gas. In some embodiments, the plasma generator circuit is a high voltage circuit that generates a potential difference across an electrode gap. In some embodiments, the plasma generator circuit is a radio frequency (RF, e.g., microwave) power generator delivering RF power to one or more RF electrodes. In some embodiments, the RF power operates around 13.56 MHz with power in the 50-100 W range, however other frequencies and/or power ranges can be effective depending on electrode design, production targets and reactant gas conditions. In some embodiments, RF power operates around 2.45 GHz for improved coupling and excitation of N₂ molecules. The controller 30 is also in communication with a user interface 26 that allows a user to interact with the system, view information about the system and NO production, and control parameters related to NO production.

In some embodiments, the NO system pneumatic path includes a pump pushing air through a manifold 36. The manifold is configured with one or more valves; three-way valves, binary valves, check valves and/or proportional orifices. The treatment controller 30 controls the flow of the pump, the power in the plasma and the direction of the gas flow post-electrical discharge. By configuring valves, the treatment controller 30 can direct gas to the manual respiration pathway, the ventilator pathway or the gas sensor chamber for direct measurement of NO, NO₂ and O₂ levels in the product gas. In some embodiments, respiratory gas (i.e. treatment flow) is directed through a ventilator cartridge that measures the flow of the respiratory gas and merges the respiratory gas with NO product gas.

The output from the NO generation system in the form of the product gas 32 enriched with the NO produced in the plasma chamber 22 can either be directed to a respiratory or other device for delivery to a patient or can be directed to a plurality of components provided for self-test or calibration of the NO generation system. In some embodiments, the system collects gases to sample in two ways: 1) gases are collected from a patient inspiratory circuit near the patient and pass through a sample line 48, a filter 50, and a water trap 52, or 2) gases are shunted directly from the pneumatic circuit as they exit plasma chamber. In some embodiments, product gases are shunted with a shunt valve 44 to the gas sensors after being scrubbed but before dilution into a patient airstream. In some embodiments, product gases are collected from an inspiratory air stream near the device and/or within the device post-dilution. Within the gas analysis portion of the device, the product gas passes through one or more sensors to measure one or more of temperature, humidity, concentrations, pressure, and flow rate of various gasses therein.

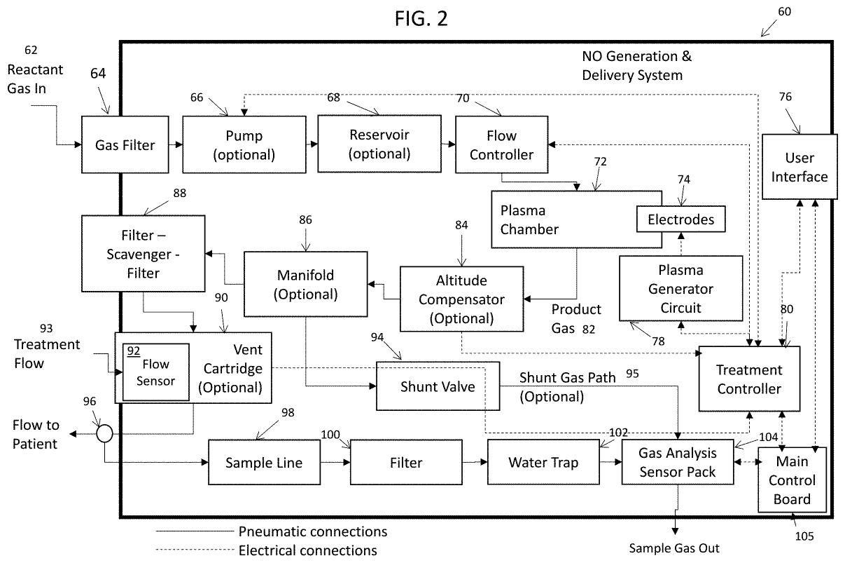

FIG. 2 depicts an embodiment of a NO generation and delivery system 60. Reactant gas 62 enters the system through a gas filter 64. A pump 66 is used to propel gas through the system. Whether or not a system includes a pump can depend on the pressure of the reactant gas supply. If reactant gas is pressurized, a pump may not be required. If reactant gas is at atmospheric pressure or passes through one or more flow-restrictive components, a pump or other means to move reactant gas through the system is required. A reservoir 68 after the pump attenuates rapid changes in pressure and/or flow from a pump. Coupled with a flow controller 70, the reservoir, when pressurized, can enable a system to provide flow rates to the plasma chamber 72 that are greater than the pump 66 flow rate. This enables the use of a smaller, lighter, quieter and more efficient pump. Electrodes 74 within the plasma chamber 72 are energized by a plasma generation circuit 78 that produces high voltage inputs based on desired treatment conditions received from a treatment controller 80. A user interface 76 receives desired treatment conditions (dose, treatment mode, etc.) from the user and communicates them to the main control board 105. The main control board 105 relays to the treatment controller 80 the target dose and monitors measured NO concentrations from the gas analysis sensor pack 104. The main control board 105 monitors the system for error conditions and generates alarms, as required. The reactant gas 62 is converted into product gas 82 when it passes through the plasma chamber 72 and is partially converted into nitric oxide and nitrogen dioxide. An altitude compensator 84, typically consisting of one or more valves (for example, proportional, binary, 3-way), is optionally used to provide a back-pressure within the plasma chamber 72 for additional controls in nitric oxide production. Product gases pass through a manifold 86, as needed, to reach a filter-scavenger-filter 88 assembly that removes nitrogen dioxide and/or particulate from the product gas. From the filter-scavenger-filter 88, product gas is introduced to a patient treatment flow directly, or indirectly through a vent cartridge 90. In some embodiments, the vent cartridge 90 includes a flow sensor 92 that measures the treatment flow 93. The treatment flow measurements from the flow sensor 92 serve as an input into the reactant gas flow controller 70 via the treatment controller 80. After product gas 82 is introduced to the treatment flow, it passes through inspiratory tubing. Near the patient, a fitting 96 is used to pull a fraction of inspired gas from the inspiratory flow, through a sample line 98, filter 100, water trap 102 and water-selective permeable membrane tubing (e.g., Nafion tubing) to prepare the gas sample and convey it to gas sensors 104. Sample gas exits the gas analysis sensor pack 104 to ambient air. In some embodiments, the system 60 can optionally direct gas through a shunt valve 94 and shunt gas path 95 directly to the gas sensor pack and out of the system. In some embodiments involving the shunt valve 94, the manifold 86 includes a valve (not shown) to block flow to the filter-scavenger-filter when the shunt valve 94 is open.

In some embodiments, systems and methods for portable and compact nitric oxide (NO) generation can be embedded into other therapeutic devices or used alone. The portable NO generation device allows NO to be generated and delivered to a patient in any location or setting as the device is small and lightweight enough to be mobile and used anywhere, including in a home of a patient or during travel. The size and portability of the ambulatory NO generation system allows a patient to use the system in a hospital or on-the-go outside a hospital and to have the benefit of NO delivery through a respiratory gas delivery device without having to be in a hospital, clinic or other medical setting. In some embodiments, an ambulatory NO generation system can be comprised of a controller and disposable cartridge. The cartridge can contain particle filters and/or scavengers for preparing the gas used for NO generation and/or for scrubbing and/or filtering output gases prior to patient inhalation. A memory device, magnetic strip, RFID, optically readable image (e.g. bar code) or other feature in/on the cartridge can provide cartridge information to the controller (e.g. scrubber type, such as loose media, packed media, or sheet material), scrubber chemistry (e.g. ingredients, ratios), scrubber dead volume, scrubber flow resistance, lot number, serial number, manufactured date, manufacturer identification code, expiration date, whether or not the cartridge has been used (binary), whether or not the cartridge has been installed (binary), date of first installation, etc. In some embodiments, a NO generation device can quantify the scrubber dead volume by pumping a known flow of gas into the scrubber with a known exit flow from the scrubber and analyzing the pressure levels within the scrubber. Typically, the scrubber outlet flow controller is closed so that the exit flow is zero for this operation. By monitoring the pressure increase within the scrubber resulting from the known inlet flow, the NO generation device can calculate the dead volume. This enables a NO generation device to distinguish between various sizes of scrubber.

In some embodiments, a NO generation device can identify the scrubber material form (e.g., sheet vs. granular), by knowing the dead volume and analyzing the pressure decay when the scrubber is depressurized. When dead volume is held constant, tighter-packed scrubber material results in slower pressure decay. In some embodiment, a NO generation device sets the output flow controller at a particular duty cycle and measures the time to depressurize the scrubber in order to determine the flow resistance of the scrubber. This enables a NO generation device to distinguish between various types of scrubber. For example, a packed soda lime scrubber has higher flow resistance and will release gas pressurized gas slower, resulting in a slower pressure decay when compared to a loosely filled granular scrubber or a sheet scrubber. A NO generation device can be compatible with various scrubber types. In some embodiments, a NO generation device characterizes one or more of the scrubber dead volume and flow restriction and automatically makes adjustments to the scrubber pressure and flow controller (degree of opening and timing) to deliver a target quantity of NO over a specific time period.

In some embodiments, the system can utilize an oxygen concentrator to increase nitric oxide production through higher reactant gas oxygen concentration, reduce the rate of NO₂ formation through lower oxygen levels in product gas and compliment oxygen generator activity as an independent device.

Architecture

Architecture of a NO generation device has a significant impact on the performance and physical characteristics of the device. Parameters influenced by architecture selection include but are not limited to NO/NO₂ ratio, acoustic noise, vibration, mass, size, power consumption, heat generation, battery life, battery rechargeability, power efficiency, control complexity, mechanical complexity, reliability and peak NO production. Some architectures support delivery of NO to the patient at constant/continuous flow while other architectures can only be used for pulsatile NO delivery.

Linear Architecture

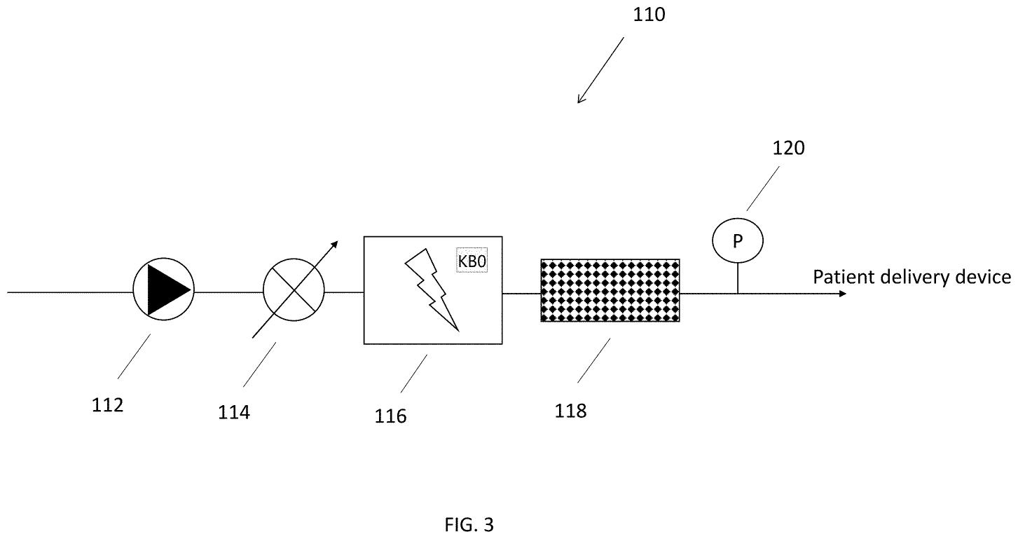

FIG. 3 depicts a linear NO generation device architecture 110 with a pump 112, a flow controller 114, a plasma chamber 116, and a scrubber 118 in series. A pressure sensor 120 at the end of the device is used to detect patient inspiration. The system can generate nitric oxide continuously or in a pulsed mode. In one mode, pump activity is continuous and plasma activity is intermittent. In some embodiments, breath detection is measured through an independent lumen to prevent interference between NO and/or air flow and breath detection measurements (e.g., pressure).

Various types of pumps can be utilized for this and the other architectures presented, including but not limited to diaphragm, screw, scroll, piezo, gear, piston, centrifugal and peristaltic. In some embodiments, the flow controller is a binary valve. In some embodiments, the flow controller is a proportional valve. In some embodiments, the flow controller is a mass flow controller. In some embodiments, a binary valve is utilized in a pulse-width modulated means to vary flow through the system. It will be understood that the pumps and controllers disclosed can be used with any NO system disclosed herein.

A linear system benefits from simplicity with very few components. This system can also be light weight.

In some embodiments, all NO generated goes to the patient.

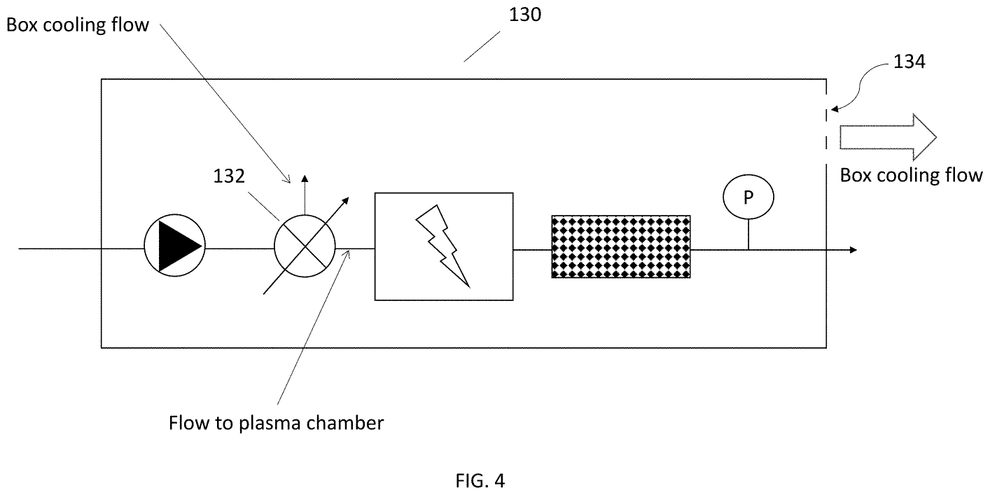

Starting and stopping a pump can be energy intensive. Hence, in some embodiments, a pump can run continuously. NO generation can be intermittent within the continuous flow of gas. It is also possible that only a portion of the generated NO is directed to the patient. In some embodiments, the pump runs more continuously. In some embodiments, when flow is not required to the patient, the pump flow is directed elsewhere. This flow can be used for a variety of purposes. In some embodiments, the pump flow is used to cool a device enclosure 130 of the nitric oxide generator. FIG. 4 depicts an embodiment where the proportional valve is a 3-way valve 132 that can direct pumped gas into the device enclosure 130. Cooling gas can exit the enclosure through a cooling flow exit 134, as shown in FIG. 4. In some embodiments, cooling gas contains NO. In some embodiments, the cooling gas does not contain NO. In embodiments where the cooling gas contains NO, gas can be optionally scrubbed of NO, NO2, and/or ozone prior to release into the enclosure for cooling, into the atmosphere, or both.

Linear Architecture with Pressurized Air Reservoir

FIG. 5 depicts an embodiment of a linear architecture 140 with a pressurized reservoir 142. A pump 144 fills the reservoir 140 with air between breaths to a target pressure, measured by a reservoir pressure sensor (Pr) 146. When a NO pulse is to be delivered, a flow controller 148 opens to release pressurized air through a plasma chamber 150 and scrubber 152 and on to the patient. This architecture enables faster NO pulse delivery due to the elevated pressure within the reservoir. In some embodiments, the flow controller is a proportional valve that varies the orifice size to achieve a more constant flow rate through the system as the pressure in the reservoir exponentially decays. Having a constant flow rate through the plasma chamber can facilitate the NO production controls within the plasma chamber. In some embodiments, a flow sensor (not shown) in the gas pathway provides feedback for the proportional valve position to ensure precise flow control through the plasma chamber. In some embodiments, the pump speed is selected so that it can run continuously to achieve the desired pressure within the reservoir and cumulative pulse flow. Continuous pump operation can conserve energy expenditure and reducing pump acoustic noise. In some embodiments, the pump runs intermittently, turning on when reservoir pressure is low and turning off when reservoir pressure reaches a threshold.

In some embodiments, the pressurized air reservoir system can purge the entire system between breaths to prevent NO₂ formation within the system. This can be accomplished, for example, by continuing to push gas through the NO generation system after the plasma is turned off and NO generation has ceased. In some embodiments, the reservoir is charged with sufficient gas to provide a NO pulse and purge volume as it depressurizes. Plasma is initially on as gas passes through the plasma chamber but is then turned off as the purge portion of the gas bolus passes through the system. Purging can apply to both the NO generation system and any delivery system between the NO generator and patient.

Linear architectures, as depicted in FIG. 4 and FIG. 5, are sensitive to the flow restriction of the scrubber component. By design, gas flow begins after breath detection, requiring flow through the scrubber to go from zero to a target level as quickly as possible. Restrictive scrubber materials and designs can result in delays of 100 ms or more between NO generation and pulse delivery. Low flow restriction scrubber designs (e.g., sheet material scrubbers, or granular scrubbers) can provide some reduction in pulse delivery time when a linear architecture is utilized.

Internal Recirculation Architecture

Some embodiments of the system can include an internal recirculation loop.

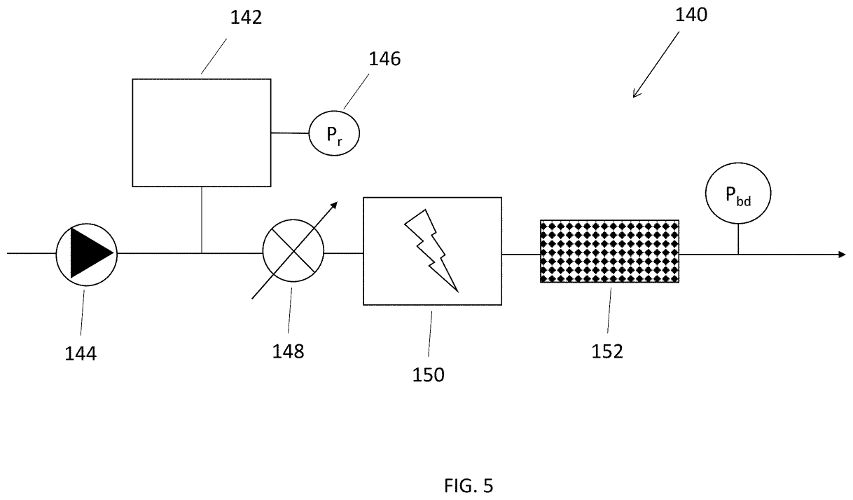

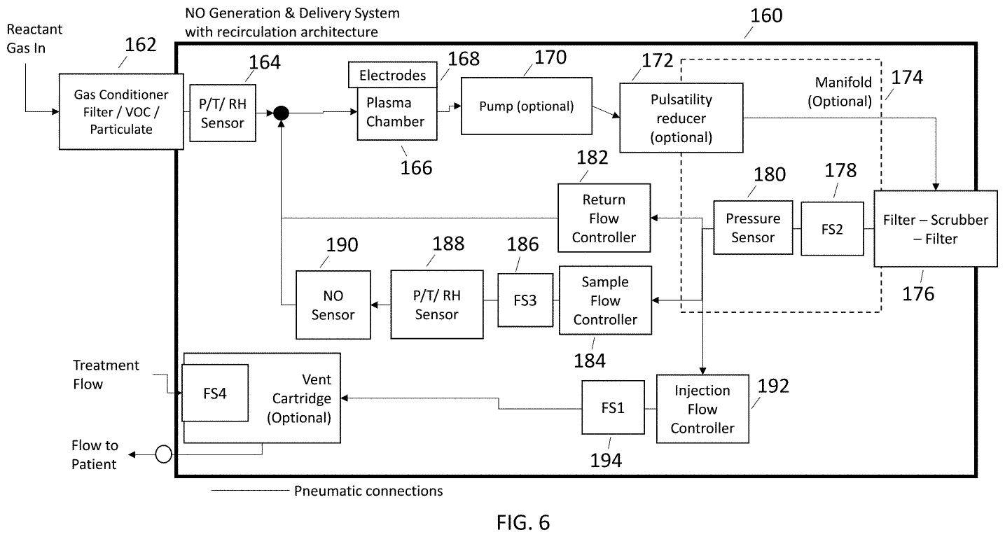

FIG. 6 illustrates an embodiment of a NO generation and delivery system 160 with a recirculation architecture that allows for a portion of product gas to be injected into an inspiratory stream and a portion of the product gas to be directed elsewhere. Reactant gas enters the system and passes through a gas conditioner 162 containing one or more of a particulate filter, VOC scrubber (e.g. activated charcoal), desiccant (e.g. molecular sieve, silica gel), and NO2 scrubber (e.g. soda lime). Gas flows through one or more sensors 164, including a pressure, temperature, and/or humidity sensor. In some embodiments, the gas conditioner is actively controlled based on feedback from the pressure, temperature and humidity sensor(s). For example, the degree of dehumidification in the gas conditioner is varied based on a humidity measurement. Pressure measurements in the reactant gas can be utilized to sense the presence or absence of plasma chamber activity as a safety measure. Gas flows to a plasma chamber 166 where high voltage is applied to electrodes 168 to generate nitric oxide product gas. Product gas passes through a pump 170 (optional) and on through an optional pulsatility reducer 172 to decrease fluctuations in the pressure and/or flow rate of the product gas. Various components of the system can be part of or attach to a manifold 174 to simplify pneumatic routing. After passing through the pulsatility reducer, product gas passes through a filter/scrubber/filter 176. The filter/scrubber/filter removes particulate and NO2 from the product gas. It should be noted that some scrubbers (e.g., one using sheet material) do not include one of the filters in some embodiments due to the lack of scrubber particulate generated. In some embodiments, the filter/scrubber/filter is user replaceable. From the filter/scrubber/filter, pressure and flow of the product gas is measured using a flow sensor 178 and a pressure sensor 180. Then, the product gas is divided into one to three separate flow paths. In one path, product gas flows through a return flow controller 182 and is directed back to before the plasma chamber. In another path, product gas flows through a sample flow controller 184, a flow sensor 186, one or more sensors 188 including a pressure sensor, temperature sensor, and /or humidity sensor, and a NO sensor 190. In another path, product gas flows through an injection flow controller 192 and a flow sensor 194 prior to being injected into a treatment flow of gas. Gas flowing through the return path merges with incoming reactant gas prior to entering the plasma chamber. In some embodiments, the plasma chamber is at or near atmospheric pressure. In some embodiments, the pressure within the plasma chamber is below atmospheric pressure, due to the flow restriction of the inlet filter/scrubber. Lower pressure within the chamber can reduce break-down voltage requirements and enable low levels of NO production. The return flow controller is modulated to maintain a constant pressure within the tubing upstream of the flow controllers while the sample flow controller maintains a target flow rate for the product gas NO sensor and the injection flow controller releases product gas at a target flow rate. In some embodiments, the target injection flow rate is proportional to the treatment flow. A constant pressure upstream of the injection flow controller improves flow control and dose accuracy. treatment controller (not shown) orchestrates the overall operation, interacting with system components and sensors to maintain the taken level of NO production. Also not shown are system components that are included in various embodiments, including a microphone, speaker, battery, charge circuitry, gas pressure sensor(s), gas humidity sensor(s), and gas temperature sensor(s).

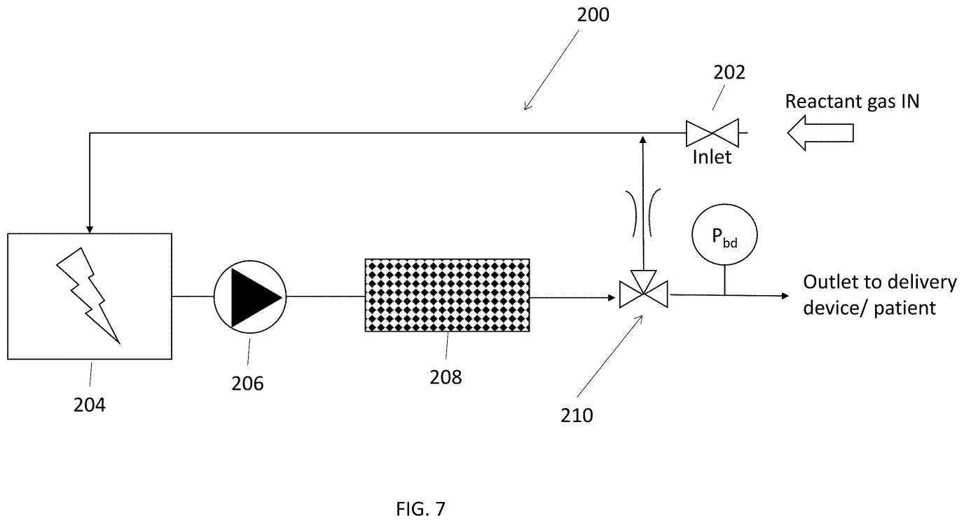

FIG. 7 depicts an exemplary nitric oxide generation architecture 200 with an internal recirculation loop. In some embodiments, NO is generated within the loop prior to breath detection. This approach can allow for faster pulse transit to the patient because the NO in the system already exists and flow through the scrubber has been established when a breath is detected. Hence NO delivery involves redirecting a flow of NO, rather than establishing a flow through a scrubber, thereby eliminating the delays associated with the flow resistance and volume of a scrubber. By generating and flowing NO in a loop, the ability to monitor and detect breaths within the patient delivery device is not affected.

Reactant gas, typically air, can enter the system through a valve, such as an inlet valve 202. Reactant gas can pass through a plasma chamber 204, a pump 206 and a scrubber 208 before reaching a recirculation valve 210, such as a 3-way valve. It should be understood that the three-way valve can be any combination of valves, such as binary valves and other flow controllers, that achieves the desired flow control. Air passively enters the system in proportion to the amount of product gas that has left the system through the 3-way valve.

When gas flows around the loop, the three-way valve can be configured to return gas to the loop through a flow restriction. In some embodiments, the flow restriction is a fixed value. In some embodiments, the flow restriction is variable. In one exemplary embodiment, the three-way valve is a binary valve, and a fixed flow restriction is selected to match the flow restriction of the patient delivery gas pathway. In another exemplary embodiment, the three-way valve is variable and provides a variable amount of gas flow on the return path. In some embodiments, the flow restriction is adjusted so that the load on the pump is continuous. By matching and/or manipulating flow restrictions of each leg of the gas pathway, the flow rate through the plasma chamber can remain constant as flow transitions from recirculation to patient delivery, thereby enabling improved control of NO generation without the need for reactant gas flow rate compensation. The flow restriction within the loop can be achieved with an orifice, for example.

When a NO generation and delivery system determines that it is time to deliver NO, the device controller transitions the pneumatic architecture from a recirculation state to a patient delivery state. This is done by adjusting the 3-way valve (or equivalent) to deliver product gas, rather than returning it to the device. As product gas leaves the system, there is an ongoing flow of product gas through the system. Where product gas once returned through the pneumatic pathway, now fresh reactant gas enters the loop to support continued gas flow through the system. This fresh reactant gas can be converted to additional NO as it passes through the plasma chamber in the event that a NO pulse is larger than the internal volume of the recirculation loop. When sufficient NO has been generated for the bolus, the device controller turns off the plasma within the plasma chamber while continuing to operate the pump. This additional pumped reactant gas is passed through the loop and patient delivery device to purge the system including the delivery device of NO, thereby mitigating against NO₂ formation between breaths. Once sufficient gas has been passed through the system, including delivery device with some margin, the controller turns off the pump and returns the 3-way valve to a recirculation setting. The controller continues to monitor the breath detection sensor for identifying the next respiratory event to dose.

It should be noted that when the device transitions from recirculation to open loop patient delivery, the volume of gas between the three-way valve and inlet stagnates. Any NO within this portion of the system will oxidize, forming some NO₂. While this NO₂ would eventually pass through the scrubber and be eliminated, some embodiments minimize NO loss by locating the air inlet in close proximity to the 3-way valve.

In some embodiments, the volume of the recirculation loop is equal to the volume of the NO pulse so that when a pulse is delivered, the entire volume of the recirculation loop is replaced with fresh gas. In this fixed pulse volume embodiment, the system varies the dose delivered to the patient by varying the concentration of NO within the recirculation loop. Concentration of NO within the loop can be varied by varying plasma parameters (frequency, duty cycle, AC waveform, energy, current, etc.) and or plasma duration (the amount of time that the plasma is ON prior to next inspiratory event). For example, electrical discharge frequency ranges from 1 to 1000 Hz, duty cycle varies from 0.005% to 100%, current varies from 10 to 1000 mA, and energy varies from 0.1 to 10,000 mJ.

Various factors can contribute to the flow rate through the recirculation loop architecture. In some embodiments, the flow rate through the recirculation loop is selected based on the plasma chamber design but can also be affected by the patient delivery device. For longer and/or higher volume patient delivery devices (for example, a cannula, scoop catheter, or other delivery structure) having a longer physical distance between the point of NO generation and the patient and/or larger diameter, a higher flow rate can be necessary to achieve acceptable transit time to the patient. Flow rates to the patient are typically limited due to the threshold of patient comfort. In some embodiments, the flow rate through the system is limited to 15 lpm to prevent patient discomfort. When NO and O₂ are delivered simultaneously, lower NO flow rates can be necessary to maintain patient comfort. In some embodiments, the flow rate is limited to 5 lpm.

The flow rate through a delivery device can vary within an individual NO pulse. In some embodiments, a two-flow rate approach is utilized where a rapid flow rate is utilized to prime a delivery device with NO followed by a slower flow rate of NO to deliver the NO and purge the delivery device. The rapid priming flow rates are on the order of 1 to 15 slpm while pulse delivery flow rates can range from 0.05 slpm to 15 slpm. In some embodiments, the NO flow rate during NO patient delivery (out the end of the delivery device) is varied throughout the duration of delivery. For example, in one embodiment, the flow rate of a NO pulse is delivered at a rate that is (or approximates) proportional with the inspiratory flow rate. In some embodiments, a controller (e.g., microprocessor) within a NO generation device varies the flow rate through the delivery device in various ways, depending on the system architecture. In some embodiments, the flow rate is varied by varying a pump speed. In some embodiments, the pressure within a reservoir is varied to vary a gas flow rate. In some embodiments, the degree of opening of one or more valves is varied to control a gas flow rate. In some instances, the controller varies the flow rate of NO directly, as it exits the system. In some embodiments, the controller varies the flow rate of purge gas directly which indirectly pushes out NO gas at a controlled rate.

Soda lime scrubbers are manufactured with water content (e.g., 15-20% by weight). Nitrogen dioxide is water soluble and neutralized by the highly alkaline hydroxides within the soda lime. As product gas passes through a soda lime scrubber, the water content within the soda lime can evaporate into the passing gas due to warmth and dryness of the product gas. When water content within the soda lime gets too low, NO₂ scrubbing can diminish, presenting a risk to the patient. In some embodiments, a humidity sensor measures the humidity of gas downstream of the scrubber. The location of measurement could be within a recirculation loop, a delivery device, or any other location between the NO₂ scrubber and patient. When the indicated humidity level downstream of the scrubber falls below a threshold, a NO generation system can prompt the user to replace the scrubber as low humidity indicates that the moisture content in the scrubber is nearing exhaustion or has been exhausted. It will be understood that a humidity sensor can be used with any embodiment of a NO system disclosed herein, including but not limited to linear architectures and recirculation architectures. In some embodiments, the humidity measurement is made within the NO₂ scrubber.

A recirculation architecture can also respond quickly to rapid breathing, owing to the fact that gas is already flowing through the scrubber, which typically presents a large flow restriction. For slower breathing, the same architecture can be used to deliver pulses as a linear architecture system by configuring the 3-way valve to the patient-delivery position. This approach can save power by enabling the system to operate at slower flow rates and pressures.

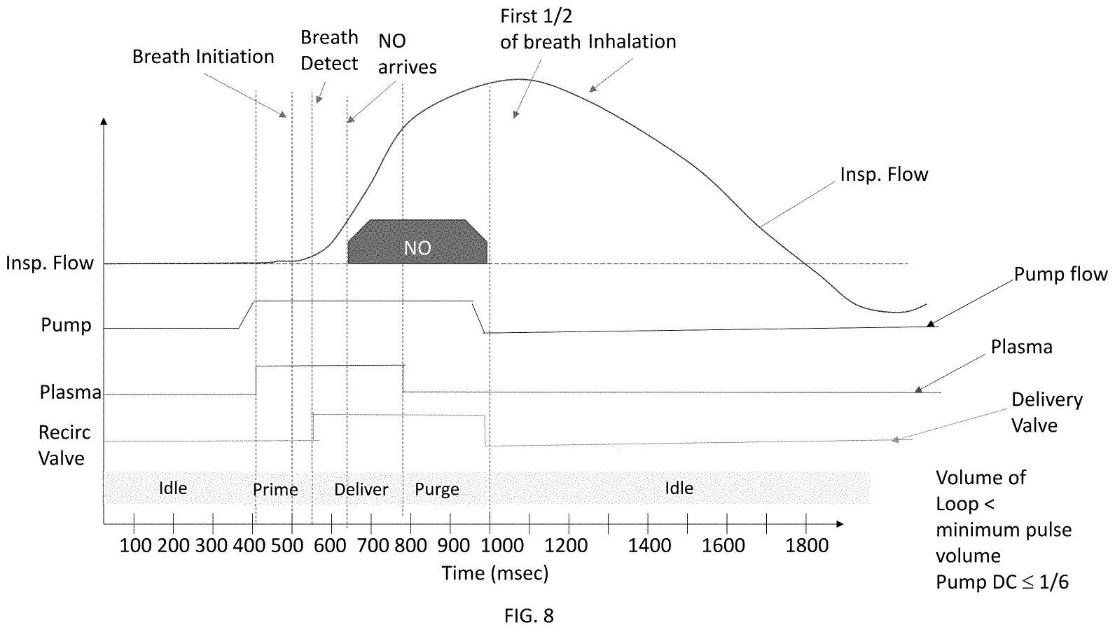

FIG. 8 depicts an exemplary timing sequence for operating a NO generation system for pulsed NO delivery. The pump and plasma are turned on first. The timing of pump and plasma can include a function of one or more of breath rate, prior inspiration onset timing, prior inspiratory peak flow rate timing, prior end of inspiration timing, prior end of expiration timing, completion of delivery device purge, and other factors. When breath detection occurs, the 3-way valve (recirculation valve) toggles to send NO down the delivery device (e.g., cannula). The inlet valve opens at the same time to permit make-up air to enter the system (not shown in the figure). NO travels down the delivery device, arriving at the patient's nose in ˜20-150 msec. Once the desired amount of NO has been generated, the plasma chamber turns off, but the pump continues until all of the NO has been delivered to the patient. Once all of the NO has been delivered to the patient, the cannula has been purged with air. In some systems, additional air is sent through the delivery system as a safety measure. Then, the pump turns off, the recirculation valve changes to closed-loop position and the inlet valve closes. The pulse delivery sub-system remains in an idle state until the time to prime the recirculation loop again. While this delivery system purging method is shown with a recirculation architecture, the approach of sending NO into a delivery system followed by a bolus of reactant gas to purge the delivery system can be achieved with many architecture designs, including with a pressurized scrubber/pressurized bypass approach or a linear architecture.

Flow Deflection Architecture

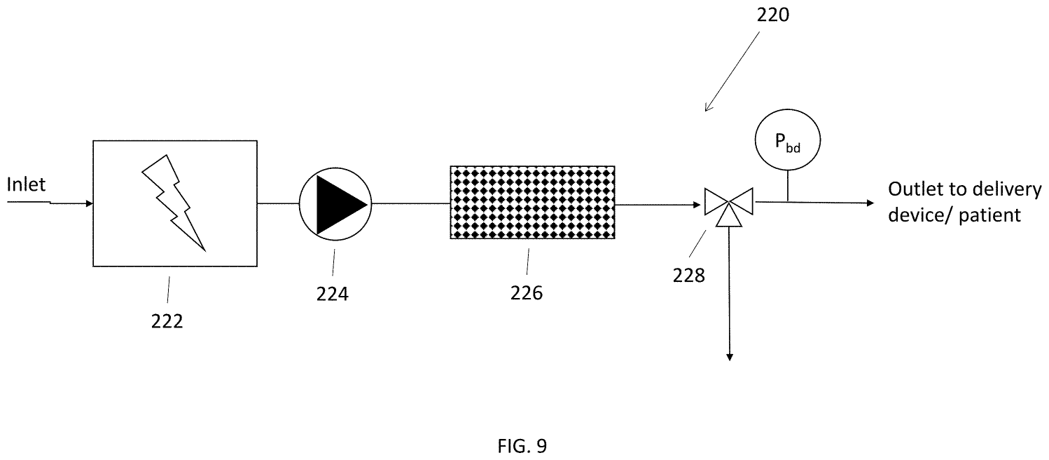

As mentioned above, establishing flow through a scrubber can take considerable time, up to hundreds of milliseconds. For example, in one embodiment, it takes 250 msec from the time an upstream pump is turned on for the flow rate downstream of a scrubber to increase from zero to 3 lpm. The amount of time that it takes to establish a target flow rate through a scrubber is related to the initial flow rate through the scrubber, upstream flow rate, upstream pressure, void space within the scrubber, scrubber geometry, and scrubber flow restriction. It can be possible that the time to establish scrubber flow can exceed the window available for NO pulse delivery. FIG. 9 depicts an exemplary architecture 220 that enables flow to be established through a scrubber prior to breath detection. Reactant gas flows through a plasma chamber 222, a pump 224, and a scrubber 226 and is directed by a 3-way valve 228 (or equivalent) which directs the gas back to the environment or into the device for cooling. Once pulse delivery is desired (for example, upon breath detection), the plasma chamber is turned on and the three-way valve is positioned to direct flow towards the patient. In some embodiments, the plasma is on at an earlier time and excess NO is released from the system, thereby eliminating delay from priming the scrubber which take upwards of 120 msec. After the desired quantity of NO plus any vented NO has been generated, the plasma chamber is turned off and flow continues down the delivery device to purge the delivery device of NO-containing bolus gas. After the delivery device is purged (typically based on time), the 3-way valve returns to directing reactant gas flow away from the patient. In some embodiments, flow directed away from the patient is scrubbed for NO and/or NO2 and/or filtered for particulates before release.

External Recirculation Architecture

Constant Concentration Circuit

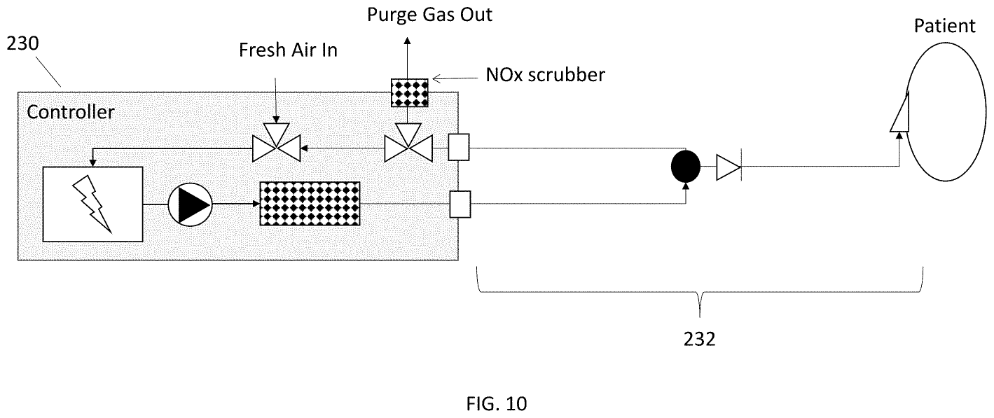

In some embodiments, NO gas is circulated from a treatment controller 230 to a delivery device 232 and back to the controller 230, as shown in FIG. 10. The gas delivery device includes a lumen for flow of NO-containing gas towards the patient and a separate lumen for flow of NO-containing gas away from a patient. The two lumens can join at a junction located near the patient. In some embodiments, the delivery system 232 (e.g., cannula or mask) is removably connected to the controller. In some embodiments, additional lumens may be utilized for oxygen delivery, breath detection, and redundant NO delivery. In this embodiment, the system includes a pneumatic pathway that can circulate a continuous stream of NO-containing gas to the patient and back. This allows for locating fresh, pressurized NO near the patient, thereby reducing the time between breath detection to NO arrival at the patient.

In some embodiments, the NO controller maintains a constant concentration of NO at the junction within the recirculation loop. Plasma activity is controlled to dose fresh reactant gas with NO as well as make NO to replace NO lost to oxidation and interaction with the scrubber. In some embodiments, a NO sensor is included in the recirculation loop to monitor NO concentration and serve as input into the plasma control. In the event that the NO concentration needs to increase, the plasma chamber begins producing more NO and it takes one or more cycles for the concentration within the loop to homogenize to the new concentration. In some embodiments, NO₂ is measured within the recirculation loop. When NO₂ levels reach above a threshold, a NO generation system can respond by prompting scrubber replacement, purging the loop and starting with fresh NO, and/or stopping treatment. In some embodiments, when a lower NO concentration is required, the system can vent some or all of the returning gas from the loop to atmosphere. In some embodiments, the vented gases pass through a NOx scrubber.

The flow rate within the recirculation loop can be the same as the patient flow rate or greatly exceed the patient flow rate. Faster flow rates allow for faster transit time from plasma chamber to patient for reduced NO oxidation and inhaled NO₂ levels. The pressure within the recirculation loop is greater than atmospheric to ensure that NO gas will travel from the loop to the patient when NO is delivered. This design can be operated in a pulsatile fashion or provide a continuous bleed of NO from the loop to the patient. When operated in a pulsatile fashion, the three-way valve permits fresh reactant gas to enter the loop while a NO pulse is exiting the loop. The 3-way valve can operate in non-binary means (like a proportional valve) as well to vary the flow rate of the pulse delivered to the patient. An optional check valve prevents retrograde flow into the recirculation loop from the patient end of the cannula. The check valve can also prevent loss of NO from the recirculation loop when pulse delivery is not occurring.

In some embodiments, an additional exhaust 3-way valve is utilized to release the contents of the recirculation loop. In some embodiments, released gas passes through a scrubber (e.g., NOx scrubber) prior to release to prevent environmental contamination which could harm the patient, caregiver and other creatures in the vicinity. The exhaust valve and inlet valve can be used in concert when the concentration within the recirculation loop needs to decrease. In this scenario, the contents within the recirculation loop can be mixed with a variable amount of fresh air sourced from the inlet valve as a portion of the circulating gas is released through the exhaust valve. At any time during treatment and at the end of treatment, the recirculation loop can be purged of NO and NO2 by fully opening the inlet and exhaust valves and operating the pump.

External Recirculation for Pulsed Delivery

In some embodiments, an external recirculation architecture is utilized with a push/pull method to generate pulsed NO and deliver it quickly to the patient. When it is time to deliver a NO pulse (typically after a breath detection trigger signal), the system turns on the pump and plasma chamber to send the pulse down the cannula to the patient. By utilizing a closed loop out and back through the cannula, the NO pulse can travel faster through the cannula to the intersection point. When the NO pulse reaches the intersection point, the three-way valve can begin sourcing fresh make-up gas to replace the volume of the NO pulse. This approach leaves the system void of any NO between breaths, reducing the potential for NO₂ formation.

Pulse generation and delivery with an external recirculation design provides a benefit of faster pulse delivery from pulling gas through the recirculation loop in addition to pushing the pulse through the cannula. Lower transit time can reduce NO oxidation occurring between the scrubber and the patient, thereby reducing inhaled NO₂ levels.

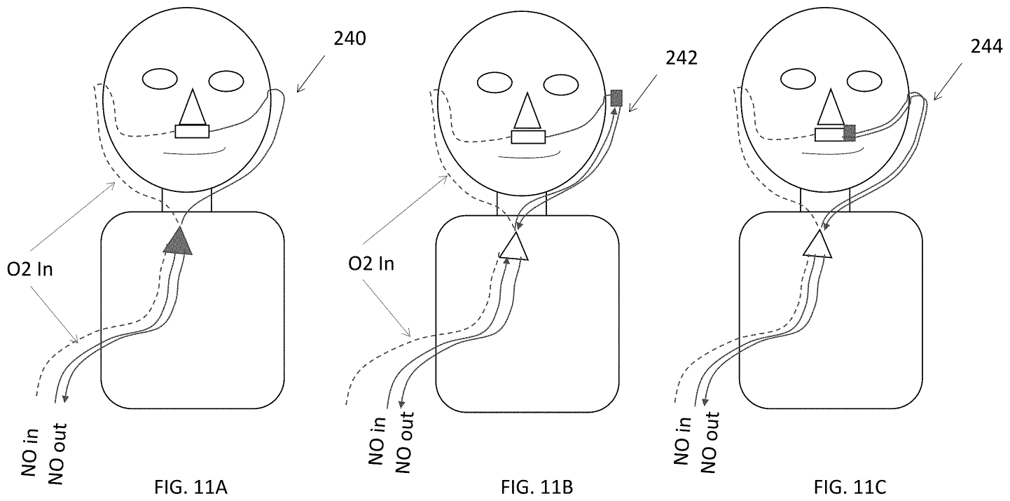

There can be a variety of locations in the cannula where the inbound NO path and the outbound NO path can intersect. In some embodiments, the intersection is a simple open tubing connection. In some embodiments, flow down one or more lumens is blocked by means of a valve. FIG. 11A depicts an embodiment of a cannula 240 with an intersection point at the base of the patient's neck. NO into the patient and NO out of (away from) the patient lumens are depicted as solid lines with directional arrows. The optional oxygen lumen is depicted as a dashed line. In some embodiments, a cannula 242 can include an intersection located at the patient's ear, as shown in the exemplary embodiment in FIG. 11B. In some embodiments, a cannula 244 can include an intersection located at the patient's nose, as shown in the exemplary embodiment shown in FIG. 11C. In some embodiments, a combination breath detection sensor and valve are located at either the base of the neck, the ear or the nose. The closer to the patient nose, the shorter the distance for NO to travel and faster the NO delivery.

In some embodiments, NO containing gas is scrubbed for NO₂ and filtered prior to leaving the delivery system (nasal prong, mask, ET tube, etc.). This is referred to as proximal scrubbing and will be described in more detail below. In some embodiments, the scrubber and filter are either within or part of a proximal length of tubing in the delivery system or separate components in series with the delivery system. The location of the proximal scrubber is similar to the intersection point locations depicted in FIGS. 11A, 11B, and 11C.

In some embodiments, the concentration of NO circulating within a recirculation loop is maintained by the controller to be constant and the dose is modulated based on the quantity of NO product gas delivered to the patient. In some embodiments, the quantity of gas delivered is controlled by one or more of the timing of the valve proximal to the patient, the flow rate of product gas and the concentration of the NO product gas. In some embodiments, the NO generation device controller works to maintain a constant NO concentration circulating within the external loop. In some embodiments, the mini-valve close to the patient does not require data/signal communication with the generation device because it is combined with a battery, processor and pressure sensor that can detect breath and control NO pulse timing independent of NO generator operation by adjusting valve/pulse timing. In some embodiments, make-up air to replace NO exiting the system is introduced to the recirculation loop by the mini-valve close to the patient concomitant with NO delivery. In some embodiments, make-up air is introduced to the loop within the NO generator. In some embodiments, make up air is introduced to the system when the pressure within the system drops due to loss of delivered gas. In some embodiments, the flow of make-up reactant gas (e.g., air) into a system is passive, where gas is flowing from a higher pressure (e.g., ambient pressure) to a lower pressure (e.g., vacuum). In some embodiments (not shown), make-up reactant gas is actively pumped into a NO generation system.

In some embodiments, the NO return lumen includes material to scrub the returning gas for NO and/or NO₂. The weight, cost and service life of the system components can be considered.

Pressurized Scrubber Architecture

As a patient's breath rate increases, the inspiratory time (ti) decreases and therefore the window of time for pulse delivery narrows. For example, a patient breathing at 40 breaths a minute with an inspiration:expiration ratio of 1:2 will have a 500 msec inspiratory event. When delivering to the first half of the inspiration (a 250 msec window), and after a roughly 50 msec delay for breath detection, there is only about 200 msec left to generate NO and completely deliver it through the cannula to the patient. It is beneficial to have NO already made and scrubbed prior to breath detection and at pressure so that pulse flow rates can be high with a reduced time for achieving the maximum flow rate, however NO oxidation increases with time and pressure.

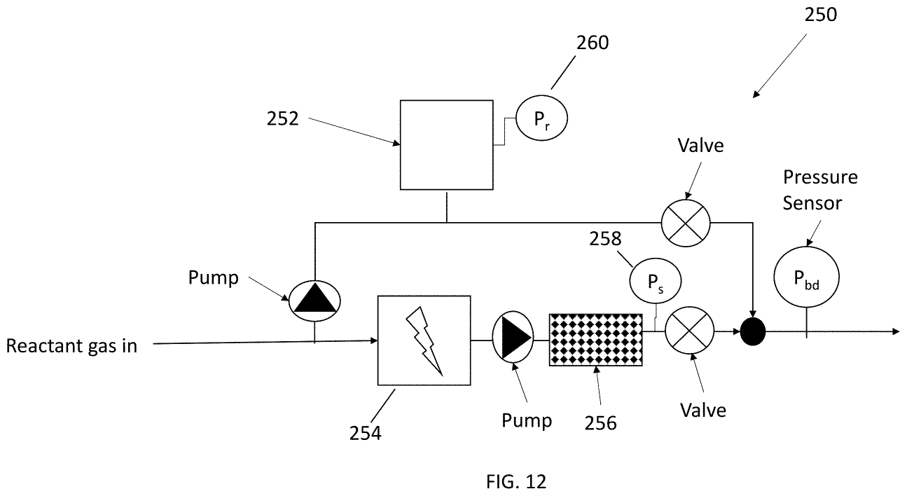

In some embodiments, NO is generated by a NO system 250, scrubber, and stored within a reservoir 252 prior to delivery to the patient, as shown in FIG. 12. NO in the presence of oxygen will oxidize, forming NO₂. Pressure within the reservoir increases the probability of collision between NO and O₂ molecules, leading to more opportunities for oxidation (i.e., NO2 formation) over a given period of time. Hence, the benefits of generating and pressurizing NO mixed with air for later use are not immediately apparent.

By briefly storing pressurizing NO, the scrubber does not have to be primed with NO for each pulse, which can take hundreds of milliseconds. This also allows for a decoupling of plasma reactant gas flow rate and flow rate through the delivery device to the patient. It decouples them in the sense that they do not have to be similar or equivalent. If the average flow rate is identical, the pump can be run continuously at a low level, reducing noise and vibration. If there is a mismatch in flow rates, either reservoir target pressure is achieved early, the system achieves a reservoir pressure just in time of delivery, or the system will not achieve target pressure in time before the next breath. Achieving target reservoir pressure early can be managed any number of ways including requiring the pump to be turned off for a period of time or slowing the pump speed, or releasing excess pressurized gas from the reservoir (e.g., through an NOX scrubber via a pressure relief valve). In some embodiments, a NO generation system utilizes a range of acceptable target reservoir pressures. This enables a system to operate at lower pressure when lower quantities of NO are required. This also reduces the chance that the system will not achieve the pressure needed for delivery.

The pressure within the reservoir (e.g., purge gas or product gas) operates within a range (e.g., 2 psi to 20 psi). In one example, a reservoir is at 10 psi when a breath is detected. A pulse of gas is delivered from the reservoir and the reservoir pressure decreases to a lower pressure (e.g., 8 psi). The pump, continuously operating, pushes more gas into the reservoir, thereby increasing the pressure. In one scenario, the patient breathes in rapid succession and the pressure only reaches 9.5 psi before it is time to deliver another pulse of gas. In this case, the reservoir pressure drops from 9.5 psi to 7.5 psi as the pulse is delivered. The pressure and reservoir volume are selected to provide adequate margin for changes in respiratory rate without excessive loss of pressure.

In the event that patient respiratory rate slows and a subsequent breath occurs later, continuous pump operation can result in pressure within the reservoir going higher than the target of 10 psi. In some embodiments, the NO generation system has margin on the top end of pressure as well. For example, one embodiment will operate at reservoir pressures between 8 psi and 12 psi, depending on the pump rate and patient respiratory rate. This range of operating pressure can be set by the controller based on one or more of the patient dose, patient respiratory rate, delivery system geometry, and other factors.

The controller ensures that the target number of moles of gas (e.g., NO) are delivered to a breath by adjusting the flow rate and duration of the gas pulse exiting the reservoir. The treatment controller can achieve a target flow rate exiting the reservoir by using the reservoir pressure as an input to the calculated flow controller setting. Rapid breath rate does not deplete the reservoir of pressure because smaller NO pulses are required when more breaths are dosed in a given amount of time. Thus, the mass flow rate of NO through the system remains at the target level (e.g., 6 mg/hr) but will be variably parsed across multiple breaths.

In some embodiments, the reservoir pressure at which the pulse is terminated is a function of respiratory rate, product gas flow, and reservoir volume but not starting pressure. In these embodiments, pulse volume will naturally fluctuate with breath rate and errors in the product flow rate or reservoir volume. However, since the mass flow rate of NO in the product gas through the system is constant and controlled to produce a target dose (mg/hr), any deviation in the pulse volume primarily affects the pulse concentration or the distribution of NO between breaths but not the average delivered dose. In some embodiments, the pulse termination pressure does not vary with respiratory rate and results in varying peak pressure. For example, a controller targeting a minimum pressure of 5 PSI may naturally achieve a peak pressure of 6 PSI at 40 breaths/minute and 10 PSI at 8 breaths/minute. One benefit of this approach is that it will never exceed the peak operating pressure of a system within the expected range of breath rates.

In some embodiments, the controller controls to a target peak reservoir pressure set point by discharging to a pressure that would result in recharging to the set point if the nominal reservoir volume were recharged at the nominal product gas flow rate for the current respiratory interval (e.g., instantaneous respiratory period, average respiratory period). In one example, a controller targeting 10 psi discharges to 9 psi at 40 breaths/minute and 5 PSI at 8 breaths/minute. If the product gas flow rate filling the reservoir in this example has a −10% error, then the reservoir will operate between 5 PSI and 9.5 PSI at 8 breaths/minute, delivering 90% of the nominal pulse volume at 111% of the nominal pulse concentration, thereby delivering 100% of the target dose. If the breath rate changes, the pulse delivered to one or more subsequent breaths will be either be too large or too small as the system acquires the new target reservoir pressure. Any errors in dosing of individual breaths average to zero over time as the breath rate varies around an average value. One benefit of this approach is that more pressure is available to prime the delivery device for each pulse.

Delivery of NO requires a minimum acceptable pressure to meet pulse delivery requirements. In some systems, the pressure within the gas reservoir is much higher than the minimum acceptable pressure (i.e., there is high margin for satisfying the pressure requirement). By operating with higher pressure, a pulsed NO generation system can continue to dose every breath in the presence of varying breath rates. For example, a system operating at the minimum acceptable pressure as a patient breaths 12 breaths per minute will require up to 5 seconds (60/12) to be at pressure for the next breath. A system operating at a higher pressure has pressurized product gas in reserve and can dose a subsequent breath immediately in the event that the respiration rate increases. A similar benefit can be had with the volume of gas available. The pressure within a larger volume decreases less when a given pulse of gas exits the reservoir. Thus, larger reservoirs of gas at pressure provide larger margin for addressing variations in breath rate by being affected less by pulses of gas exiting the reservoir. Some embodiments of a pressurized reservoir system have margin at the upper end of pressure as well to prevent over-pressurizing the reservoir or having to shut off the pump when the breath rate decreases.

Tighter NO generation control can be achieved when the flow rate and pressure within the plasma chamber are constant. Placing the pump after the plasma chamber separates the plasma chamber from the variable pressure within the reservoir. This approach also enables NO to be made over a greater amount of time, decreasing the size of pump required in some embodiments and allowing the electrodes to operate at lower production levels to prolong electrode longevity. Smaller pumps are lighter, quieter and draw less power (leading to longer battery life).

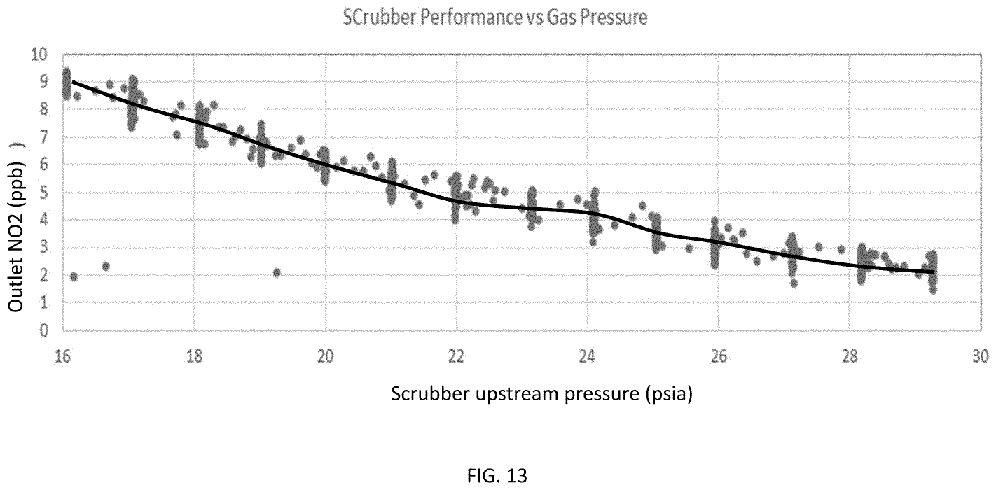

A further benefit is that this approach also enables pressurized NO to be released as a bolus at discrete time points in the inspiratory cycle. In some embodiments, the NO reservoir includes NO2-scrubbing material so that the product gas is continuously scrubbed as it waits for delivery. This can be advantageous because the NO-containing gas is exposed to scrubber material for longer time than if it simply flowed through a scrubber. Pressurizing the NO2 scrubber is also advantageous because some NO2 scrubbing materials (e.g., soda lime) are more effective at scrubbing NO2 at elevated pressure. FIG. 13 presents an exemplary plot of NO2 concentration in gas exiting a soda lime scrubber at various pressures to illustrate scrubber performance with respect to gas pressure. The data were collected by delivering a consistent mass flow of an NO2 containing gas through the scrubber. A mass flow controller upstream of the scrubber maintained the mass flow through the scrubber at 1.5 slpm while a needle valve downstream of the scrubber was adjusted to achieve varying levels of back-pressure within the scrubber. As can be seen in FIG. 13, an increase of pressure within the scrubber of roughly 1 atmosphere results in more than 3-times reduction in NO2 concentration in the effluent gas.

Pressurizing the NO2 scrubber and accumulating the product gas specifically enables a system to operate at a continuous, low NO production rate (determined by the dose) and reactant gas flow rate. It reduces the instantaneous power and flow requirements of the system, facilitating the use of smaller, lighter, quieter components such as pumps and transformer and simplifies the process control. It can also result in a more steady, more innocuous acoustic profile for the device. Production rate is matched to patient dose by accounting for NO absorption and oxidation, which occur between production and delivery to the patient. This approach is highly tolerant of deviations and errors in reactant gas flow, except for errors in calibration, owing to the homogenizing of gas within the reservoir over time. A pressurized NO reservoir approach can work with either purging the delivery device (e.g., cannula) between breaths or leaving the NO within the delivery device between breaths. Operating at a constant NO production rate and reactant gas flow results in a nominally constant NO concentration. The volume of the NO bolus entering the delivery device varies according to the breath rate, with faster breath rates having smaller pulses than slower breath rates for a given NO dose (e.g., 24 mg/hr).

In some embodiments of a pressurized NO device, the void space within a NO reservoir holds at least the amount of product gas for a single NO pulse. In various device embodiments, this void space can range from 10 ml to 5000 ml, depending on NO concentration, oxygen concentration in the product gas, degree of portability of the device, and product gas flow rate. NO concentration within a pressurized scrubber can vary from 0.1 ppm up to 10,000 ppm. Product gas flow rate into the reservoir/scrubber volume depends on the treatment requirements and can vary from 50 ml/min to 15 lpm. Additional volume pressurized with product gas reduces pulse delivery time by maintaining a higher reservoir pressure for the duration of the pulse (i.e., slower pressure decay within the reservoir as the pulse is delivered). In some embodiments, the reservoir is at least partially filled with NO2-scrubbing material. In some embodiments, to minimize the size and mass of the reservoir, a portion of the pressurized volume is located upstream of the scrubber material and is not filled with scrubber. Additional void space outside the scrubber material is preferably located upstream of the scrubber. This ensures that the entire NO pulse is scrubbed for a sufficient amount of time between breaths and that sufficient pressurized gas is available.

In some embodiments, the scrubber-filled portion of a NO reservoir holds at least the amount of product gas for a single NO pulse. This ensures that the entire product gas pulse has sufficient time to interact with the scrubber between breaths, resulting in lower NO2 levels than if a portion of the product gas passes through the scrubber at a high flow rate during the pulse. As an example, the moles of product gas compressed into 15 ml void space at 10 psi is the equivalent of 24.5 ml of product gas at ambient pressure, 20 C. This means that the 15 ml void space holds roughly two and half (2.5) 10 ml pulses. The void space includes dead volume before the scrubbing material, within the scrubbing material and after the scrubbing material but before the flow controller. Designs usually minimize the void space after the scrubber because the product gas in that volume is no longer being scrubbed. Typically, the volume within the scrubbing material holds at least the volume of product gas for the largest pulse that may be delivered. For example, the dead volume within the scrubber can be 6.15 ml and holds a single 10 ml pulse at 10 psi.

Gas exiting a pressurized reservoir must overcome flow resistance, the resistance coming from the flow controller, delivery device, and other gas flow path components. The flow controller at the exit of a reservoir, under the direction of the treatment controller, provides a variable resistance to flow. Variation in resistance is a means to modulate gas flow exiting the reservoir. For example, if the resistance is 10 kPa/slpm, the pulse volume is 40 sml and the pulse delivery duration is 400 msec, the pulse requires an average flow rate of 6 lpm (40 sml/400 msec). To achieve an average of 6 lpm flow through the system, the pressure is required to be an average of 60 kPa (6 slpm*10 kPa/slpm) and the average flow resistance is required to be 10 kPa/slpm. Hence, the pressure in the pressure in the reservoir begins at a pressure above 60 kPa and ends at a pressure below 60 kPa. The amount of dead volume under pressure determines the peak and minimum pressure during the gas pulse that will occur for a given pulse volume. The flow controller (e.g., prop valve) can be adjusted during the pulse to vary the flow resistance to achieve a controlled pulse flow profile. In some embodiments, a constant flow rate pulse flow profile is desired. In some embodiments, the pulse flow profile matches a typical inspiratory flow profile. In systems that continuously fill the reservoir (i.e., pump always on), the slope of the depressurization curve will be less (i.e., slower). The ability to adjust downstream resistance enables a system to achieve the same pulse profile with a variety of starting conditions. In some embodiments, the post-scrubber flow controller varies its effective orifice size during the NO pulse to account for pressure decay within the scrubber and achieve a target NO pulse flow rate. The target NO flow rate through the delivery device varies from tens of ml/min to tens of lpm, depending on one or more of the dose, treatment, NO concentration, delivery device, inspiratory flow, and other factors.

Some pressurized scrubber systems include a source of pressurized non-NO gas for pushing the NO pulse through the delivery system and purging the delivery system of NO. Pressure within a purge gas reservoir and the purge flow controller can be similarly manipulated to alter the flow rate of the NO pulse as the pressure in the reservoir decays and the NO pulse traverses and is expelled from the patient end of the delivery device.

Calculation of NO Oxidation and Loss

In some embodiments, a NO generation system calculates the quantity of NO that is expected to be lost between the plasma chamber and the patient and overproduces NO by an offsetting amount. NO can be lost to oxidation with O2 and also interaction (e.g., adsorption, absorption) with other materials in the gas pathway. These effects can be quantified with sufficient accuracy. In some systems, the amount of NO loss is modeled as a constant value. Given that respiratory rates vary and in turn NO residence time and average pressure varies, the estimate of NO loss is more accurate when it is calculated in real time. In some embodiments, NO oxidation is calculated using the following equation:

−d[NO]/dt=2*e\hat{ }(K/T)*[NO]\hat{ }2*[O2]

where [NO] and [O2] are nitric oxide and oxygen concentrations in moles/liter, t is time, T is temperature (Kelvin), and K is a constant.

In some embodiments, the oxidative losses are calculated based on the current pressure in the scrubber. Then, the amount of NO generated is adjusted accordingly. The distance from the plasma chamber to scrubber might suggest that there would be a phase shift in the NO flow concentration using this approach, however we have found the residence time to be adequate for NO diffusion to even out the concentration. This is fortunate since it would be difficult to predict the actual oxidative losses and resulting concentration, second-by-second, as an NO bolus traverses the scrubber.

Non-oxidation loss of NO is quantified with an equation based on characterization of the NO delivery system. In some embodiments, this equation takes into account one or more of gas temperature, gas pressure, gas water content, time, scrubber type, scrubber chemistry, scrubber geometry, scrubbed dead volume, non-scrubber dead volume, respiratory rate, NO concentration, NO2 concentration, reactant gas oxygen concentration, scrubber age, delivery system type, delivery system dimensions, delivery system material(s), and other factors. These equations are utilized by the treatment controller to quantify NO loss within the system and compensate for that loss with additional NO production in the plasma chamber. Some of the parameters in this calculation are fixed constants or relationships based on system characterization (e.g., scrubber volume). Other parameters are calculated based on sensor information, such as measured reactant gas humidity from a humidity sensor, measured reservoir pressure from a pressure sensor, reactant gas oxygen concentration from an oxygen sensor, etc. In some embodiments the equation is implemented using one or more look-up tables.

Reactant Gas Flow Rate

As a pressurized scrubber and/or reservoir increases in pressure, the pump load increases which can slow down the speed of the pump. With a constant voltage applied to the pump, the speed would slow down as the reservoir fills with pressure. This, in turn, can slow the flow rate of reactant gas through the plasma chamber and pressurized reservoir affecting NO production and/or NO loss due to oxidation.

In some embodiments, a flow sensor measures the flow of reactant gas and/or product gas and the controller can be configured to alter plasma parameters (e.g., frequency, duty cycle, dithering, current, and/or voltage) to compensate for differences in flow. This can be based on a mathematical function, look up table or other means. For example, the NO generation system can be characterized for NO generation across a range of reactant gas flow rates and production levels. In some embodiments, the controller receives a target dose level from a user/physician/pharmacist and calculates the pulse concentration and/or NO moles required for each breath as a function of dose level, breath rate, target subset of tidal volume to dose, humidity, temperature and other factors. In some embodiments, the concentration is calculated for every breath. In other embodiments, the concentration is updated every 2 or more breaths. The system then determines the plasma parameters (e.g., frequency, duration, AC waveshape, dithering, voltage) required to produce the target production level as a function of one or more of production level, reactant gas flow rate, pressure in the plasma chamber, humidity level in the reactant gas, oxygen level in the reactant gas, temperature of the reactant gas, scrubber type, delivery system type, scrubber age, expected NO loss, and electrode age. The determination of plasma parameters can be generated by mathematical equation, look-up table or other means.

In some embodiments, pump speed is varied based on a measurement of reactant gas flow rate in order to maintain a constant reactant gas flow rate.

In some embodiments, reactant gas flow rate is measured using a proxy for reactant gas flow, such as pump speed. In some embodiments, pump speed is measured via an encoder (e.g. optical) or tachometer. In some embodiments, pump speed is determined by observing the frequency of ripples in the motor current related to the changes in load from loading and unloading a pump diaphragm. In some embodiments, pump speed is determined based the commutation frequency of a brushless motor. In some embodiments, pump speed is determined based on vibrations of the pump as measured by an accelerometer or microphone. Vibrations are related to rotational speed of the pump motor and/or pump head. In some embodiments, pump speed is determined with an appropriate sensor based on ripples in flow or pressure related to operation of a pump diaphragm. In some embodiments, reactant gas flow rate within the plasma chamber is measured by the difference between atmospheric pressure and plasma chamber pressure, based on prior characterization of the system. In some embodiments, reactant gas flow rate is measured by proxy using the delta-pressure between two points within the system with a known pneumatic resistance between them.

Under the direction of the treatment controller, the pressure in the reservoir (e.g., bypass or scrubber) can vary over time, due to delivering differing volumes of product gas to the patient. In some embodiments, the pump speed is varied based on the measured reservoir pressure value, and a predetermined calibration of pump speed and pressure head vs, flow rate. It is possible to maintain a constant flow in this way by driving the pump harder (i.e., providing more current to the pump motor) as the pressure head increases. This also allows a check of the performance of both the pump and the pressure sensor against each other by comparing the expected rate of pressure change within the reservoir (dp/dt) given a set pump speed, against the measured dp/dt. Alternatively, some embodiments compare an expected mass flow rate and a calculated mass flow rate based on reservoir pressure rate of change (dp/dt) to ensure that the target flow rate is maintained. Using the ideal gas law (i.e. PV/RT=n), pressure, temperature and known dead volume, the amount of moles (n) can be calculated at two time points to determine the change in mass (i.e. mass-flow rate) between the time points. An additional feature of this approach is that it can be used to detect pneumatic leaks within the system and/or a blocked reactant gas intake.

In some embodiments, the pump has a tachometer (e.g., encoder). The controller monitors either the pump speed or the change in reservoir pressure per unit time (dp/dt) as an input to a closed-loop pump speed control. This solution is often used in combination with a flow rate sensor. Alternatively, the flow sensor can be used as the input to the feedback loop. Mismatches between pump speed, flow rate, and reservoir dp/dt can be utilized to detect component failures. In some embodiments, mass flow is either measured directly or calculated as a function of volumetric flow rate, temperature and pressure.

In some embodiments, the rate of pressure change within a fixed volume with respect to time can be used to derive the flow rate of a gas entering the pressure vessel. In some embodiments, the rate of pressure change in the scrubber reservoir is utilized to calculate the reactant gas flow rate and serve as an input to a pump speed controller. This same approach can be utilized to calculate the flow rate of product gas or purge gas exiting the system as a function of dp/dt of the respective reservoir.

In some embodiments, the controller modulates pump voltage to maintain a constant reactant gas flow as the reservoir and/or ambient pressure varies. In some embodiments, flow is regulated using closed loop control with one or more of the pump's speed and/or flow rate measurements identified above as feedback. In some embodiments, flow is regulated using a feed-forward model of pump flow as a function of one or more of speed, pressure, voltage, current, and power.

In some embodiments, the controller modulates pump voltage to maintain a constant pumping rate (e.g. rotational velocity, RPM) as pump load varies. Maintaining a constant pump speed results in a more innocuous noise and vibration profile for the device. In some embodiments, RPM is regulated using a closed loop control with one or more of the pump speed measurements identified above as feedback. In some embodiments RPM is regulated using an open loop feed-forward model of pump speed as a function one or more of speed, pressure, voltage, current, and power. In some embodiments, a combination of feed forward and closed loop controls are used to maximize accuracy and response time of the controller. A pressurized scrubber architecture can be operated with continuous NO production at a low-level, intermittent NO production at a high level, and/or a combination of the two to achieve a target NO dose to the patient. In some embodiments, constant, low production levels (e.g. <200 ppm·slpm) are achieved by varying the frequency of electrical discharges having a fixed duration. In some embodiments, constant low level NO production is achieved with electrical discharges at a fixed frequency but varying duty cycle. In some embodiments, NO is generated at an as-needed basis using a dithering approach. Although there can be advantages in noise, vibration, and power consumption when NO is generated continuously (i.e. a continuous series of electrical discharges), the NO that accumulates within the scrubber will age for a longer period of time. In some embodiments, NO is generated and pressurized within the scrubber reservoir as late as possible to minimize NO oxidation.

Patient breath rates can vary over time. A continuous NO generator will produce a target number of NO mg per unit time which will be distributed over a number of breaths. When there is a lengthy pause between breaths, a continuous NO generation system may exceed pressure limits within the NO reservoir and/or bypass reservoir. In some embodiments, the reservoir(s) include a pressure relief valve to release excess gas from the reservoir and maintain a target pressure. In some embodiments, the pressure relief valve is passive (e.g., pop off valve) while other embodiments utilize an actively controlled valve that is controlled based on the pressure within the reservoir (e.g., a backpressure regulator). Vented purge gas and/or NO-containing gas from the reservoir can be released directly into the ambient environment. In some embodiments, the vented NO-containing gas is scrubbed for NO and/or NO₂ prior to release (e.g., NOx scrubber).

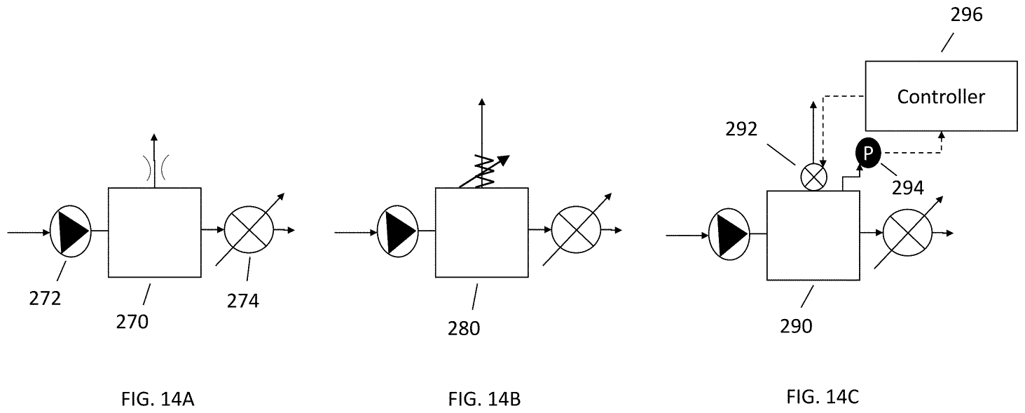

Continuous pump operation provides benefits in power consumption and noise generation. A challenge presents with the bypass reservoir (i.e., purge gas reservoir) because the time between breaths (breath period) can vary, making it difficult to select a single pump speed that will sufficiently fill the bypass reservoir before the next delivery device purge pulse during fast respiratory rates and prevent over pressurizing the reservoir between when respiratory rates are slow. FIG. 14A depicts an exemplary embodiment of a bypass gas reservoir 270 that is filled by a pump 272 with a flow controller 274 at an exit of the reservoir. A critical orifice at the top of the image continuously releases gas from the reservoir to prevent the reservoir from being over-pressurized. For example, the orifice is sized to release 140 ml/min at a pressure of 10 psi. FIG. 14B depicts an exemplary embodiment of a bypass reservoir 280 with a pressure relief valve. The cracking pressure of the pressure relief valve is set at or above the target purge gas pressure and below the pressure level that could damage the reservoir, flow controller, pump, or other components. FIG. 14C depicts an exemplary embodiment of a bypass reservoir 290 with an actively controlled valve 292 that is opened and closed using a controller 296 based on a measurement of the pressure in the reservoir from a pressure sensor 294. In some embodiments, a NO delivery system will prolong the purge gas pulse to decrease purge gas reservoir pressures to nominal levels after one or more long breath periods. In another embodiment (not shown), the pump speed is slowed as the pressure approaches the maximum pressure. This approach can prevent the pump from coming to a stop which mitigates against pump stalling, decreases perceived noise levels, and improves pump longevity.

In some embodiments, the NO generator can permit the reservoir to fill to a higher pressure during extended pauses between breaths. Then, when the next breath occurs, the system releases a longer-than-usual pulse to maintain the dosing run rate (e.g., mg/hr) and return reservoir pressures to a target level. This approach can allow for all produced NO to get delivered to the patient, albeit with some variance in pulse placement within the inspiratory volume. Some embodiments stop pump and plasma operation when a reservoir pressure reaches a specific threshold.

Scrubber/Sequestering Material

In some embodiments, NO₂ levels within pressurized NO are mitigated by filling the pressurized reservoir with NO₂ sequestering material. For example, the reservoir can be filled with soda lime granules so that the NO gas is scrubbed while it is stored. A scrubber can serve as a reservoir with NO₂ sequestering material. Scrubbers are filled with a NO₂ scrubbing material in the form of granules, sheets, tubes, coatings, co-extrusions, or other geometry with a balance of air. The volume of air space within the scrubber is referred to as the “void space.” The amount of void space within a scrubber is a function of the volume of the scrubbing material housing/enclosure and the packing ratio. The void space within a scrubber holds a NO bolus prior to delivery. The amount of residence time within the scrubber void space affects scrubbing efficiency, with longer residence time producing more opportunity for NO₂ removal. Depending on the rate of NO₂ removal produced by scrubber media, NO₂ may form at a faster rate due to NO oxidation than NO₂ removal, leading to a net increase in the concentration of NO₂ within the scrubber reservoir. Keeping in mind that residence time within the scrubber results in NO loss through oxidation as well as interaction with the soda lime, there is a balance to be achieved between NO concentration, pressure, and residence time within the pressurized scrubber. In one embodiment, this balance is optimized towards delivering the target NO dose with minimum NO₂ levels in the ejected gas. In some embodiments, this balance is optimized towards delivering the target dose while minimizing power draw of the device at the cost of higher NO2 levels.

In some embodiments, the size of a scrubber (for example, the scrubber media and the void space) is determined by the amount of scrubbing material required to last the service life of the scrubber. In some embodiments, the void space in a design is selected by the amount of pressurized gas required to deliver an NO pulse. In some embodiments, the size of the scrubber is determined by how much inherent water content is required for the scrubber to not dry out in extremely dry conditions over its service life. In some embodiments, the void space within the scrubber can hold NO product gas for multiple NO pulses (i.e., multiple breaths). This enables a generation system to flow continuously, utilizing smaller pumps. The additional residence time improves NO2 scrubbing of the product gas and reduces the magnitude of the pressure drop within the reservoir during delivery of a given NO pulse volume. These benefits must be balanced with the additional NO oxidation that occurs under pressure to determine the optimal system design, including but not limited to scrubber dead volume, product gas concentration, and product gas flow rate. Manufacturing variance can potentially introduce variance in void space and flow restriction between scrubbers. In some embodiments, a scrubber includes a memory device (e.g., EEPROM, RFID, etc.) that includes the scrubber void space, flow restriction, and other information obtained through individual scrubber characterization. This information can be read by the controller with wires or wirelessly and be utilized in selecting the pump flow rate profile, pump on-time, target scrubber pressure, pulse duration (valve timing), and NO generation settings (due to variance in void space). In some embodiments, a NO generation system measures the void space by introducing a known quantity of gas to the void space and measuring the resulting change in pressure.

The effective internal dead volume of a NO generation system includes all of the volume in pneumatic components between the pump and the valve downstream of the scrubber. This can include, but is not limited to, fittings, pneumatic pathway, dead volume in the pump, scrubber void space and dead volume on the scrubber-side of the valve.

It is important to minimize the post-scrubber dead volume (i.e., the volume between the scrubber media and downstream valve/flow controller) because gas within that space is high concentration, under pressure, and not getting scrubbed. Larger dead volume in this area results in longer transit time through this area and higher levels of NO₂ formation/NO loss between NO pulses. In some embodiments, the scrubber media is immediately adjacent to the output valve to minimize un-scrubbed volume between the scrubber media and flow controller (also between scrubber and patient). One characteristic of a pressurized scrubber/pressurized bypass architecture is that the residence time of any portion of the gas flowing through the system is similar. Slow respiratory rates result in larger volume NO pulses and faster respiratory rates result in smaller NO pulse volume for a given dose level. When pulse volumes are smaller, a larger portion of each pulse is comprised of gas from the post-scrubber dead volume. This results in pulse NO2 levels being higher during rapid respiration than during slow respiration. NO generation systems predict the amount of NO to be lost within the system and over-produce NO to compensate. In some embodiments, the NO production compensation is based on characterization of a representative system at various NO doses, residence times, and environmental conditions.

In some embodiments, the void space of the scrubber is designed as a function of one or more of: required pulse timing, target scrubber reservoir pressure, scrubber service life and acceptable inhaled NO₂ concentration. In some embodiments, a NO generation system can be utilized with a variety of scrubbers that differ in dead volume. In one exemplary embodiment, a NO generation system includes two sizes of scrubber to choose from. For example, the larger scrubber has twice the dead volume of the smaller scrubber and the system operates at twice the flow rate (i.e., reactant gas flow rate) to achieve a target pulse pressure. In this scenario, to achieve the same dose, the concentration of NO in the product gas in the larger scrubber can be roughly divided in two and the pulse volume doubled to achieve an equivalent dose as the small scrubber. Proportionally increasing dead volume and flow rate (in this exemplary case, doubling of both) maintains the same gas transit time through the system. Thus, when the NO concentration is held constant, a larger system can deliver a proportionally larger dose of NO with no additional NO oxidation penalty. The larger scrubber results in a longer scrubber service life because there is more scrubbing material present. In some embodiments, a NO generation system includes independent flow controllers that are sized for the dead volume and flow rates that are required for a scrubber. The controller selects between one or more scrubbers based on the patient treatment conditions and utilizes one or more corresponding flow controllers. To achieve a variety of flow rates through the NO generation system, some embodiments utilize more than one pump to achieve finer resolution of flow rate while retaining sufficient flow rate range.

Inhaled NO₂ concentration is a function of the NO₂ formed during NO production, the scrubber's ability to remove NO₂, and oxidation of NO into NO₂. The NO₂ formed during production is a function of electrode geometry, electrode temperature, electrode material, pulse grouping, pulse duration, duty cycle, voltage, current, reactant gas humidity, reactant gas temperature, and reactant gas pressure. The scrubber's ability to remove NO₂ is a function of scrubber surface area, media type, media quantity, water content (e.g., in the case of soda lime), product gas residence time, packing/void space, product gas pressure, extent the scrubber has been utilized, and flow rate. NO oxidation is a function of time, NO concentration, pressure, oxygen concentration and temperature. NO oxidation occurs within the pressurized part of the system and within the delivery system after NO pulse release from the flow controller. Time for oxidation occurring after release from the scrubber is based on the pulse flow rate, delivery system volume, and presence/absence of a purge pulse.

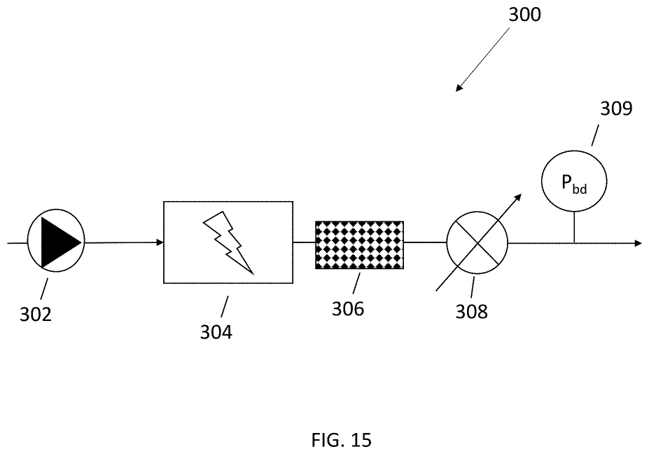

FIG. 15 depicts an embodiment of a pressurized scrubber architecture 300. A pump 302 sources reactant gas and passes it through a plasma chamber 304 where plasma activity converts nitrogen and oxygen into nitric oxide. The gas passes into a scrubber housing 306. A flow controller 308 downstream of the scrubber prevents flow of gas when closed, thereby causing product gas to accumulate within the scrubber. A breath detection sensor 309 detects a patient inspiration, and a controller (not shown) opens the flow controller to release a pulse of nitric oxide. In some embodiments, the flow controller is a proportional valve. This can be beneficial because the nitric oxide pulse flow rate can be controlled despite the varying pressure from the reservoir upstream. In some embodiments, the NO pulse flow rate through the delivery system is controlled to be a constant level (e.g., 5 slpm). In some embodiments, the flow controller consists of a binary valve. When a binary valve is used, the pulse flow will exponentially decay with reservoir pressure. In some embodiments, the pump and plasma chamber operate continuously to maintain consistent NO properties at the delivery point. Plasma operating point is automatically varied to account for changes in NO oxidation rate resulting from changes in pressure within the scrubber as breath rate varies. For example, if there is a long pause between breaths, a greater amount of NO will be lost to oxidation and the plasma activity (e.g., duty cycle) is increased. Continuous NO production can be advantageous because smaller (lighter) pumps can be utilized. Also, the continuous operation generates less noise and vibration than a large pump that runs intermittently.

In some embodiments, the pump shuts off when the scrubber reaches a target pressure based on a pressure measurement at the scrubber/plasma chamber. Depending on the pressure within the scrubber housing, flow restriction of the cannula and volume of the delivery device (e.g., cannula), the pulse transit times through a delivery device can be very fast (˜10 msec). In some embodiments, the NO pulse takes roughly 50 msec to travel from one end of the delivery device (e.g., cannula) to the other. It should be understood that the pulse travel time can vary but should be short enough to deliver the NO to patient based on the target window within the inspiratory event and the breath rate. For example, an exemplary fast breathing rate can be a 500 msec inspiratory event, so the travel time of the NO gas should be such that it can reach the patient in time for the NO to be inhaled during the target portion of the inspiratory event. The faster the pulse transit time, the longer the pulse can be before reaching the end of the pulse delivery window. For a given patient dose level and pulse flow rate, making pulses as long as possible enables lower NO concentration to be utilized, reducing inhaled NO₂ levels in turn. In some embodiments, longer pulses involve slow delivery flow rates of a higher concentration NO. Longer NO pulses dose a larger portion of the tidal volume, thereby treating more lung and/or airway tissue. This can be advantageous for improving patient oxygenation so long as the dosed tissue is still sufficiently functional in terms of gas exchange and able to increase oxygen transfer to the blood.

Still considering the device depicted in FIG. 15, as the scrubber pressurizes, the pressure within the plasma chamber increases. This changes the amount of reactant gas molecules between the electrodes, requiring higher voltage to break down (N₂ and O₂-containing gas is an electrical insulator). In some embodiments, the higher voltage requires more time to develop, thereby delaying electrical breakdown within the gap. The additional N2 and O2 molecules in the plasma chamber due to higher pressure also result in higher NO production rates once a plasma has been ignited for a given set of plasma parameters. The NO generation controller can compensate for the effects of reactant gas pressure on plasma generation in order produce a target NO production level (e.g., 200 ppm·slpm). In some embodiments, electrical discharge pulses are longer to compensate for the delayed onset, for example.

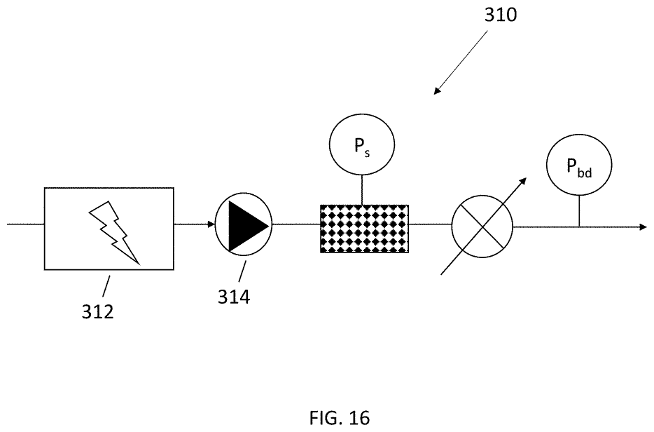

FIG. 16 depicts an exemplary embodiment of an architecture 310 with a plasma chamber 312 being located before the pump 314 so that the pressure within the plasma chamber is constant and low (near atmospheric). This can eliminate the need for pressure compensation due to scrubber filling, however compensation for differences in barometric pressure stemming from weather or elevation may still be required. In the event that barometric pressure significantly impacts NO production and compensation is required, a NO generation system can measure ambient air pressure and/or plasma chamber pressure and alter plasma parameters to achieve target production levels despite the change in pressure. For example, in some embodiments, the controller can measure that the plasma chamber is below atmospheric pressure and respond by increasing the duty cycle of the electrical discharges to prolong the plasma and produce a target amount of NO. The level of increase in duty cycle is determined by an equation, look-up table or similar approach based on characterization of the effects of varying plasma chamber pressure on NO production in a representative system. In some embodiments, a mass-flow sensor measures the mass flow of reactant gas entering the plasma chamber and the controller can compensate with plasma parameters, accordingly, to produce a target NO production level. In some embodiments, a controller can utilize a NO sensor downstream of the plasma chamber (not shown) for closed-loop control of NO production to control for effects of environmental conditions (humidity, pressure, temperature) on NO production. In cases where the flow rate of reactant/product gas is not known, a flow sensor is utilized in combination with the NO sensor to measure NO production (e.g., ppm·slpm).

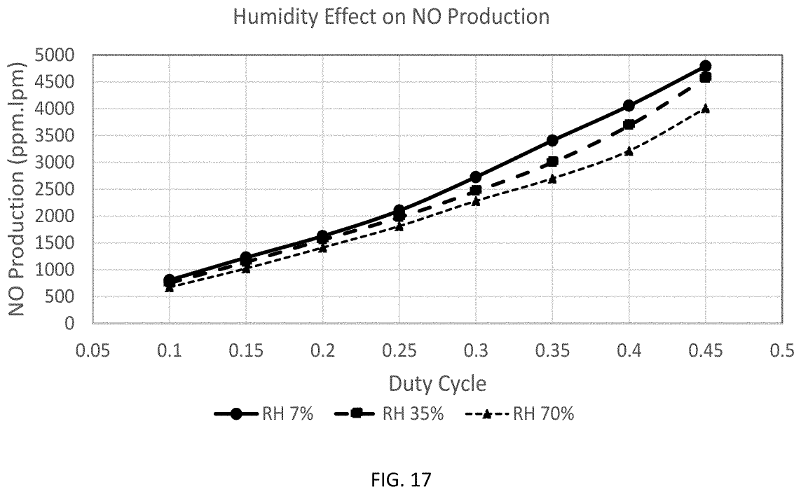

Reactant gas water content can affect NO production as well. FIG. 17 illustrates a graph of exemplary experimental NO production data from an electric NO generation device operating with 1.5 slpm of reactant gas flow and various plasma duty cycles. The system was calibrated with 7% RH reactant gas at 20° C. and swept through multiple duty cycles at various humidity levels. It can be seen that the effect of humidity on NO production are relatively small at low NO production levels. As the duty cycle increases (along with the temperature of the plasma chamber), the effects of humidity increase. A humidity compensation approach measures the humidity or water content in the reactant gas and adjusts the duty cycle to generate the target amount of NO. For systems that are calibrated with dry gas, this involves increasing the electrical discharge pulse sufficiently to make up for the otherwise lost production. If a system is to be used in an environment with a particular humidity, it can be beneficial to calibrate the unit with reactant gas at that humidity to improve accuracy and potentially eliminate the need for humidity compensation and/or reactant gas humidity management. In some embodiments, the humidity loss algorithm is stored as a look-up table. In some embodiments, humidity compensation factors are derived from an equation based on prior characterization data. In an example, a system that was calibrated with 0 g/m3 absolute humidity reactant gas is operated with 6 g/m3 absolute humidity reactant gas. An appropriate correction curve is calculated and applied to the characteristic production rate vs. duty cycle curve for the system's operating point (pressure, flow, temperature, etc), from which a duty cycle is selected that is 25% higher than the duty cycle would have been at 0 g/m3 absolute humidity. In some embodiments, NO production settings are based on closed loop feedback of the actual NO output of the system thereby decreasing the need for compensation specifically for humidity.

In some embodiments, after a NO pulse is released, the delivery system continues to retain nitric oxide-containing gas within it. In some embodiments, the delivery system includes scrubbing material to remove any NO₂ that forms within the delivery system between breaths. In some embodiments, the plasma chamber is turned off and the entire scrubber and delivery system are purged with reactant gas (e.g., air) between breaths.

A pressurized scrubber architecture can produce rapid NO pulse delivery to the patient. This is owing to the existence of scrubbed, pressurized NO on tap that can be released into the cannula within milliseconds of breath detection and pressurized bypass gas on tap to push the NO pulse completely through the delivery device. This approach can deliver a NO pulse to the patient within, for example, 10-20 msec, depending on the geometry of the delivery device. Factors that contribute to NO pulse delivery timing are scrubber pressure, scrubber void space, delivery device length, delivery system cross-sectional area, delivery system dead volume, delivery system flow restriction, and/or the presence/absence of filters.

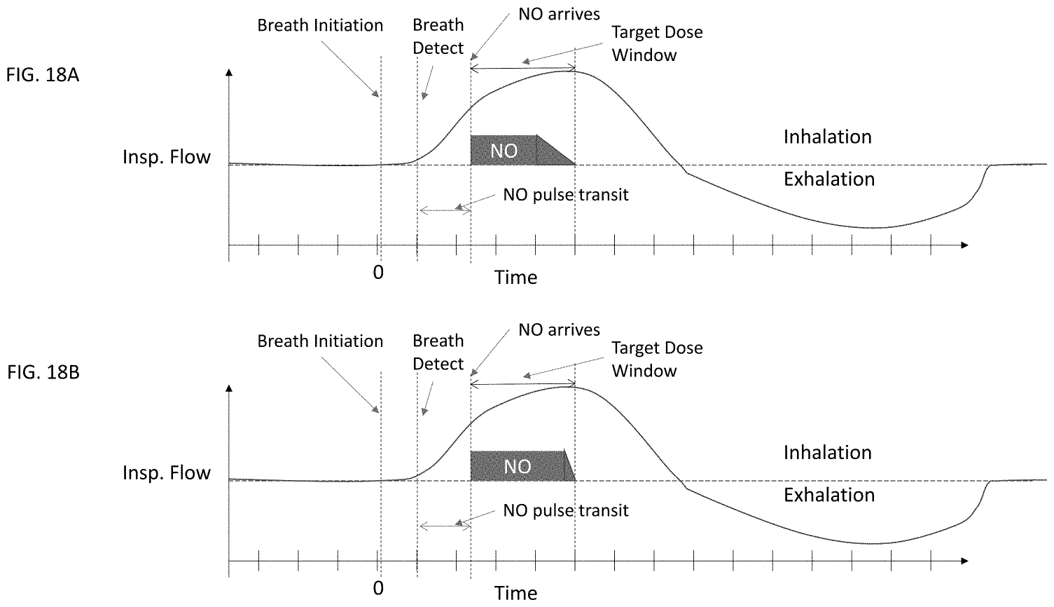

Abrupt termination of the trailing edge of a NO pulse is important for accurate dosing of particular regions of the lung. In embodiments with NO within the delivery device at all times, forward flow of NO can be arrested, however NO will bleed into the patient between breaths unless a flow control device exists near the patient. A NO delivery device that includes a valve proximal to the patient to arrest or redirect NO flow can provide a clean termination to a NO delivery pulse. Alternatively, a purge flow of non-NO gas can push the NO pulse to the patient and cleanly terminate a NO pulse. Abrupt termination of a NO pulse enables a NO delivery system to deliver NO up to a particular point of the inspiratory event without delivering NO beyond that time point. For example, a system that is programmed to deliver NO to the first half of a 500 msec inspiratory event (i.e., a 125 ms pulse) but requires 40 ms to terminate the NO pulse, must actually deliver an 85 msec pulse so that the trailing edge of the pulse is complete before the 125 msec time limit. Contrastingly, a system that can terminate the NO pulse in 5 msec and deliver NO consistently up until 120 msec into the inspiratory event before the pulse is terminated. Given that inspiratory flow rates are pulsatile, themselves, with maximal flow rate occurring mid-breath, systems that have to terminate the NO pulse earlier due to slow pulse termination can result in underdosing the middle portion of the breath where the inspiratory flow rates are maximal. FIG. 18A depicts exemplary performance of a system that is slow to terminate the NO pulse. In order prevent NO delivery to an undesired portion of the inspiratory volume, the system begins termination of the NO pulse early. FIG. 18B depicts exemplary performance of a system that can terminate the NO pulse more rapidly and thus deliver at a target NO delivery rate for a longer period of time while still only delivering NO within the delivery window.

The dose delivered to the patient can be altered by varying one or more of pump flow rate, plasma duty cycle, reservoir peak pressure, reservoir concentration, pulse duration, pulse flow rate, and scrubber void space. In some embodiments, NO product gas is diluted after production. This can decrease the concentration, decreasing the NO oxidation rate. In some embodiments, a scrubber reservoir is filled with a mix of NO-containing product gas and unaltered reactant gas. The unaltered reactant gas can be sourced through the plasma chamber with plasma off or from another flow path within the system. Dilution of product gas can enable a NO generation system to deliver product gas concentrations that would otherwise not be achievable due to the low-end production limitations.

Pressurized Scrubber with Bypass, Single Pump

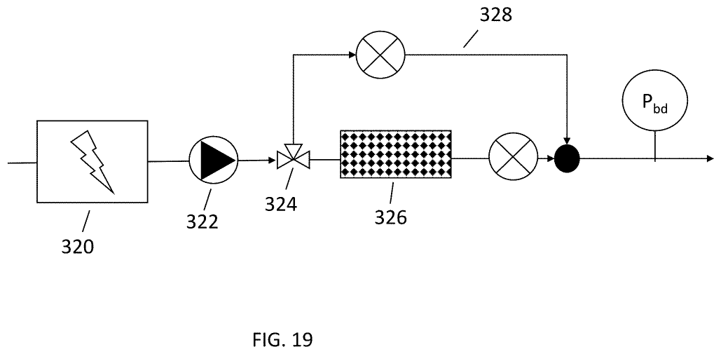

In some embodiments, a pressurized scrubber is utilized to provide a charge of NO for the NO pulse. A secondary flow path for non-product gas is utilized to push the NO pulse through the cannula and purge the cannula with non-NO-containing gas between breaths. This can enable purging of the cannula between breaths to minimize aging of NO without having to purge the scrubber. Purging the scrubber takes time and gas volume which can affect the NO dose and range of breath rates that can be supported by a NO generation and/or delivery system. FIG. 19 depicts an exemplary pressurized scrubber with a bypass design. In this embodiment, reactant gas passes through a plasma chamber 320 and into a pump 322. A three-way valve 324 (or equivalent) directs flow either to a scrubber 326 or a bypass channel 328. When reactant gas passes to the scrubber, the plasma chamber is ON. When reactant gas passes to the bypass channel, the plasma chamber is OFF. In some embodiments, the plasma chamber turns off an amount of time prior to changing the state of the 3-way valve to provide sufficient time to clear the plasma chamber of NO and deliver all NO to the scrubber path.

When a breath is detected, the valve downstream of the scrubber opens to release a pulse of NO. In some embodiments, the end of the NO pulse is controlled by closing the valve downstream of the scrubber and opening the bypass valve. Flow through the bypass channel is provided by the pump. Bypass flow pushes the NO bolus down the cannula to the patient. After the NO has cleared the cannula, the pump can resume filling of the scrubber with NO. In some embodiments, the pump runs continuously. In some embodiments, the pump pauses operation at a point in the cycle between scrubber filling and bypass flow. In some embodiments, the pump pauses operation after bypass flow and before scrubber filling. Pump operation depends on pump size and flow rates, where smaller pumps are expected to be on more of the time. Small to medium sized pumps (3 lpm or less) are advantageous because they draw less power and produce less acoustic noise and vibration. In some embodiments, larger pumps (>3 lpm) are required to produce sufficient flow for the bypass flow to deliver the NO pulse on time.

Pressurized Scrubber with Bypass, Dual Pump

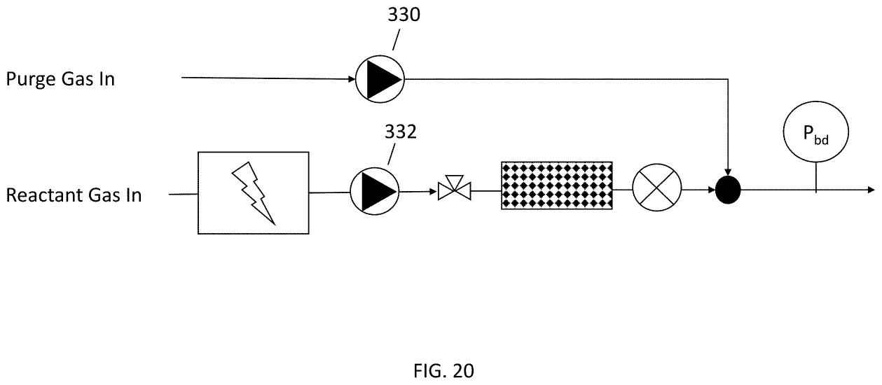

FIG. 20 depicts an exemplary bypass architecture with separate pumps 330, 332 for the bypass and scrubber pathways. This design can allow the pumps to be sized and controlled independently for optimum performance. A valve upstream and a valve downstream of the scrubber enable the system to maintain pressure within the scrubber without static pressure acting on a stopped pump during off-periods of the pump. This architecture can simplify the control system with a simple feedback loop that pressurizes the scrubber and triggers the scrubber downstream valves to open in response to the breath detect and timing/pressures. The upper pump provides a flow of gas to push the NO pulse down the delivery system at the end of NO pulse release from the scrubber. In some embodiments, the product gas pathway flows at a constant rate while the bypass flow is intermittent, only operating when bypass flow is needed to deliver a NO pulse to the patient.

In some embodiments, reactant gas and purge gas are the same. For example, both reactant gas and purge gas can be atmospheric air. In some embodiments, the reactant gas and purge gas are different. For example, the humidity level may differ between the two gases. In some embodiments, the reactant gas humidity is controlled to improve control of NO generation, for example. In some embodiment, reactant gas is dried completely or nearly completely for predictable NO production and/or prevention of water condensation within the system. In some embodiments, purge gas is dried completely. In some embodiments, purge gas is dried to a level that it will not condense within the system. Any additional drying of purge gas comes at the cost of additional mass of desiccant and/or energy expenditure to dry gas. In some embodiments, the chemical make-up of the reactant gas and the bypass/purge gas is different. For example, the nitrogen/oxygen ratio in the reactant gas could be 50/50 to enhance NO production and the purge gas nitrogen/oxygen ratio could be the same as atmospheric air. In some embodiments, the purge gas has a high level of N₂ to decrease the rate of oxidation of the NO pulse once in the delivery system. Utilizing reactant gas and purge gas with different chemistries can improve NO production and decrease NO loss to oxidation. In some embodiments, oxygen concentration technology is utilized to create a reactant gas with N₂ to O₂ ratios near stoichiometric (50/50) and a purge gas that is high in N₂.

Purging of the delivery device (e.g., cannula) involves displacing NO-containing gas within the delivery device with non-NO containing gas. In order to purge the entire delivery device, the purge bolus of gas must be larger in volume than the delivery device internal volume at the same pressure. In some embodiments, as shown in FIG. 12, bypass gas is accumulated in a purge gas reservoir 252. Flow exiting the purge gas reservoir is typically controlled by a binary valve, proportional valve, or other type of flow controller. The flow controller is activated to initiate the purging process. In some embodiments, the flow is active for a set amount of time which is sufficient for gas at the flow rate dictated by the pneumatic design to complete purging of the delivery system. In some embodiments, the flow is actively ceased (e.g., closing a valve) when the reservoir pressure reaches a target minimum value. This pressure-based approach can account for variance in delivery device flow restriction due to kink, tortuosity, manufacturing variance and other factors. These same approaches of pulse flow control apply to the NO bolus.

Air Compressor Architecture

In some embodiments, flow paths in a bypass architecture source gas from a common reservoir in the system. FIG. 21 depicts such an exemplary system 340 with one or more pumps pressurizing an accumulator. In some embodiments, the pump flow rate is set so that the pump can run continuously, matching the demand for gas from the two flow paths. Running a pump continuously provides advantages in sound levels, total pump mass and power draw. Sound levels are improved when the pump operates continuously. Total pump mass can be reduced because a single pump is providing the required gas flow to the two channels, rather than two independent pumps. Similarly, a single pump, when sized appropriately, can require less power than two separate pumps.

In some embodiments, flow through the plasma chamber is controlled by a flow controller. In some embodiments, the flow is controlled to be at a constant flow rate for NO generation. At the end of NO generation, additional air flow equivalent to the dead volume of the plasma chamber and flow path to the flow controller is flowed into the scrubber to prevent NO from entering the bypass gas stream. Bypass flow is controlled by a separate flow controller, driven by the pressure from the accumulator.

Pressurized Scrubber, Pressurized Bypass (PSPB) Architecture: Dual Pump

FIG. 12 depicts an embodiment of a NO generation system that utilizes independent pumps for bypass and scrubber flow paths. Product gas from a plasma chamber 254 is collected in a scrubber 256 and bypass gas is collected in a reservoir 252, each with respective pressure sensors 258, 260 (Pₛ and Pᵣ, respectively). In some embodiments, pumps run continuously. In some embodiments, pumps run on an as-needed basis, based on respective gas flow requirements and/or respective reservoir pressures. Breath detection is detected by a sensor in in the delivery system (P_{bd}).

The scrubber is filled with NO between breaths. When breath detection occurs, a flow controller downstream of the scrubber initiates the flow of NO gas. The flow controller can be comprised of multiple types of flow controller device, including but not limited to one or more binary valves, a mass-flow controller, or one or more proportional valves, depending on the flow resolution and flow range required. The NO pulse shape (i.e., duration and flow profile) is related to but not limited to NO scrubber concentration, valve timing, flow restriction within the scrubber, scrubber pressure, void space within the scrubber, and pump flow rate. The trailing edge of a NO pulse is shaped by the bypass flow, which is related to bypass reservoir pressure, delivery system dead volume and bypass pump flow rate. The NO pulse can result in partial or complete depressurization of the scrubber, depending on the control scheme and treatment parameters. The use of a pressurized bypass enables the system to push the NO pulse faster through the cannula, creating more of a square NO pulse shape. Being able to terminate pulse with a sharper trailing edge enables pulses to maintain a NO mass flow rate (concentration*flow rate) for a longer period of time without dosing at undesirable times (later in the breath or even after inspiration has ceased), permitting lower NO concentrations with broader treatment of the lung for a given patient dose.

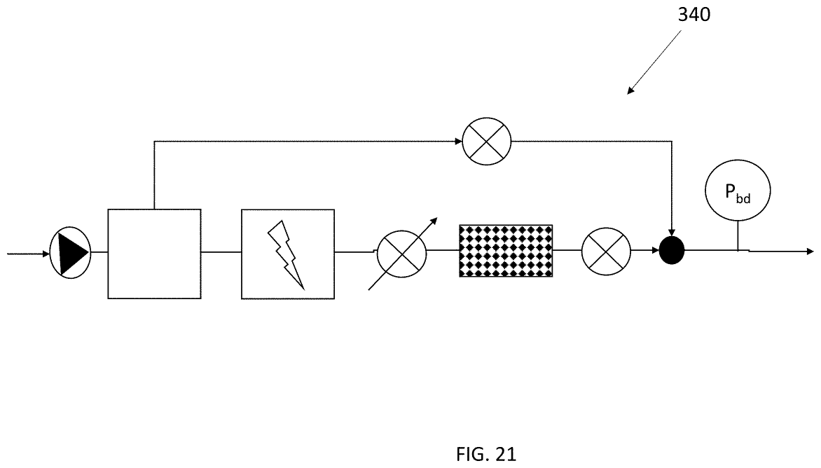

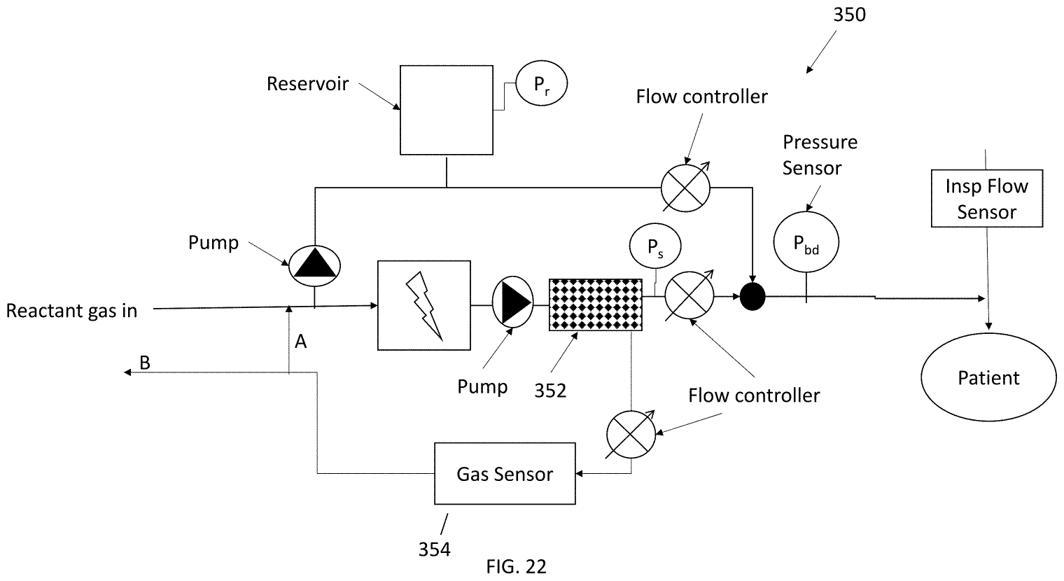

FIG. 22 depicts an embodiment of a pressurized scrubber/pressurized bypass design 350 with a pneumatic flow path exiting the product gas scrubber 352. The flow path leads to one or more gas sensors 354 that are used to analyze the product gas. Flow rate through the sensor flow path is either passively (e.g., critical orifice) or actively controlled (e.g., flow controller). In some embodiments, a sensor that measures NO concentration is utilized. In some embodiments, the flow rate is measured for compensation in the NO measurement. The NO measurement can serve as feedback to the NO production control algorithm to compensate for variation in NO oxidation, reactant gas properties, scrubber aging, NO loss, and other effects within the system. In some embodiments, the sampled gas is introduced to the incoming reactant gas flow after sampling (flow path A in FIG. 22). In some embodiments, the sampled gas is returned to the atmosphere (flow path B in FIG. 22). In some embodiments, the sampled gas is scrubbed for one or more of NO and NO2 prior to introduction to the atmosphere.

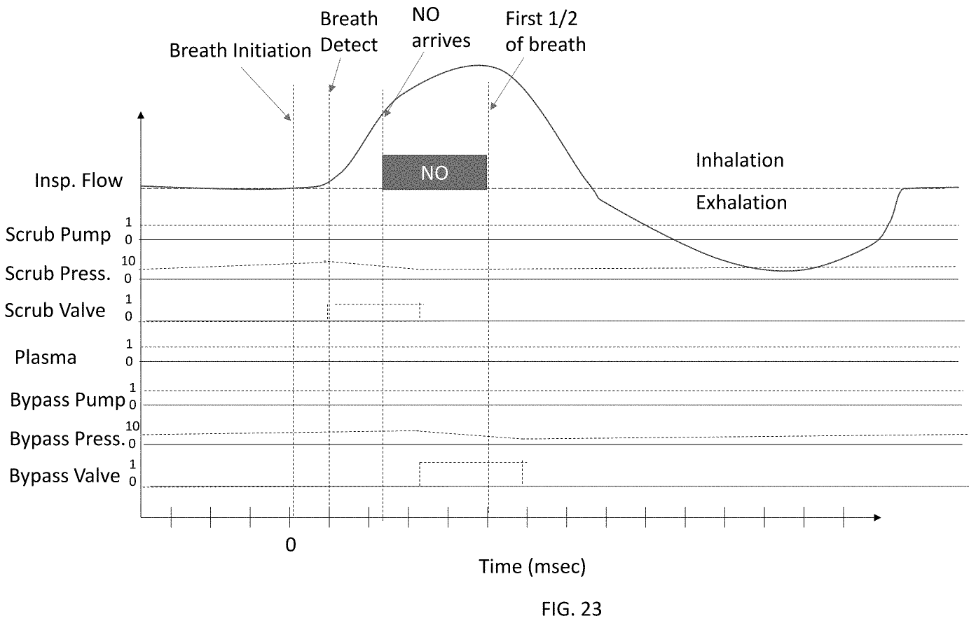

FIG. 23 depicts a graph of an exemplary timing sequence for a pressurized scrubber/pressurized bypass system. From top to bottom, there are several parameters plotted. The top curve depicts the inspiratory flow rate with inspiration being a positive value and exhalation negative. A dark rectangle depicts the timing and relative flow rate of the NO pulse delivery to the patient (proximal end of delivery system) as compared to the inspiratory flow. The next curve down depicts the product gas pump which pulls reactant gas through the plasma chamber. In the embodiment shown, the product gas pump (labeled “scrub pump”) operates continuously at a constant level. In other embodiments, the product gas pump operates intermittently. For example, the product gas pump can operate until the scrubber reservoir pressure reaches a threshold and then stops. In some embodiments, gas pump speed is controlled with a proportional integral derivative (PID) controller that targets a specific pressure. In some embodiments, a gas pump slows down its pumping speed as it approaches a target pressure without fully stopping. This can help the pump restart while pumping against a resistance.

The next curve down shows the scrubber pressure. The scrubber fills with NO over time. The pressure within the scrubber decreases when the release valve is open, as depicted in the next curve down. In the embodiment shown, the pressure within the scrubber does not reduce to zero gauge pressure before the end of the pulse. In other embodiments, the pressure reaches zero at the end of each pulse. The pressure at the end of the pulse relates to the volume of the scrubber/reservoir, the pressure of the scrubber/reservoir, the duration of the pulse, and the flow restriction of the delivery device.

Plasma activity is continuous in the depicted embodiment. This is not to say that the plasma itself is continuous. Depending on the level of NO generation required, plasma is pulsed at specific frequency and/or duty cycle to produce a target level of nitric oxide. In general, the plasma is typically active when there is gas flow through the plasma chamber. It should be noted that the target level of nitric oxide production is greater than the target patient dose in order to account for expected oxidation and loss within the system.

The next curve down depicts the bypass gas pump which is on continuously. In some embodiments (not shown), the pump is on long enough to pressurize the bypass reservoir and then turns off. Pressure within the bypass reservoir is depicted in the next curve where pressure increases to a target level (e.g., 10 psi). When the bypass valve opens, gas exits the bypass gas reservoir to push NO within the delivery system to the patient. Pressure within the bypass reservoir decreases as bypass gas exits the reservoir at a faster rate than the pump provides. Flow out of the bypass gas reservoir ceases when the bypass gas valve closes. This is typically done after sufficient time that the delivery device has been purged of NO-containing gas plus some safety margin (as shown). In practice, the valve closing time may also be determined based on pressure within the bypass reservoir reaching a specific minimum pressure or pressure delta. This helps ensure that a specific volume of gas has exited the fixed volume reservoir to displace the volume of gas within the delivery system and helps overcome variance in purge time that may occur due to variance in flow restriction of the delivery system that can occur due to flexing, kinking, manufacturing variance and other factors.

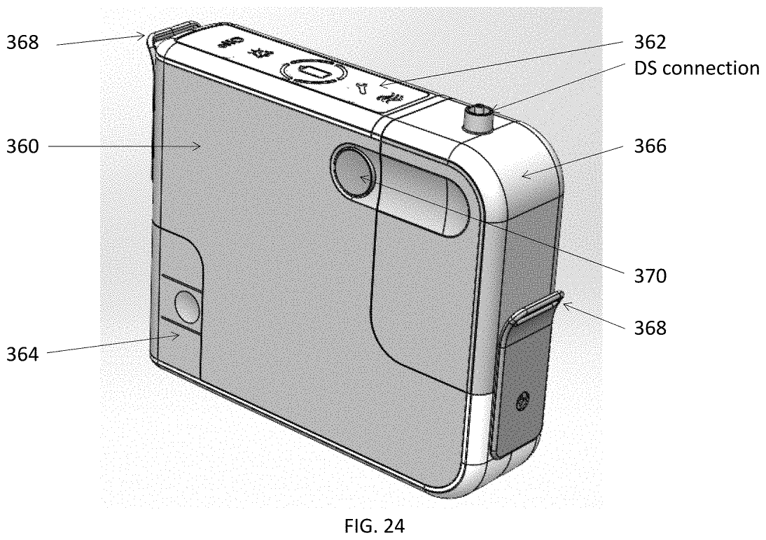

FIG. 24 depicts an exterior of an exemplary NO generation and delivery device 360. The device is housed in an enclosure that can withstand fluid ingress, drop impact and shields for electromagnetic interference. The enclosure features a user interface 362, a removable battery 364, a removable gas conditioning cartridge (GCC) 366, shoulder strap attachment points 368 and a release button 370 for removing the GCC.

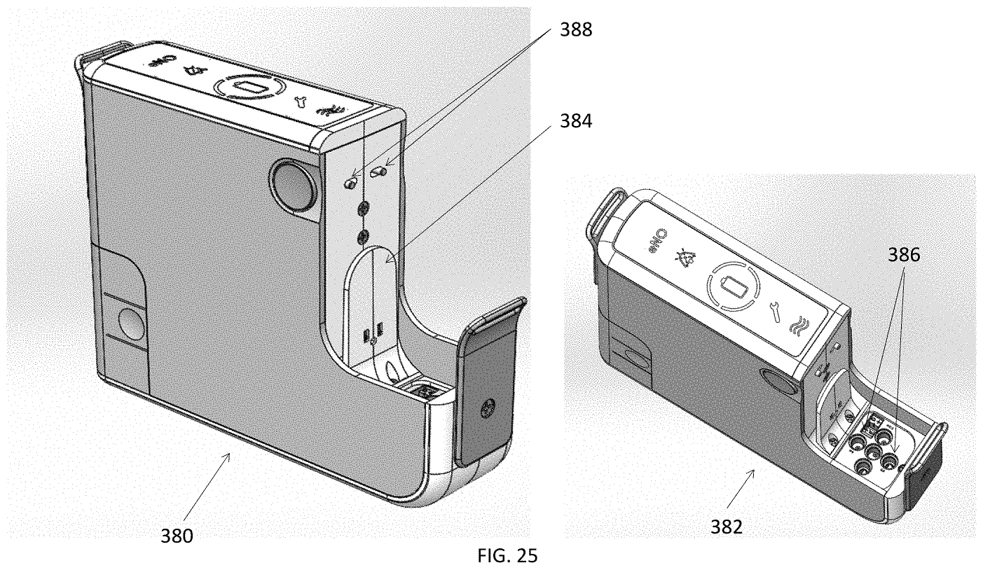

FIG. 25 depicts an exemplary NO generation and delivery device 380 with a GCC 382 removed. The GCC registers with the device geometry by sliding on an alignment feature 384, like a duck tail groove. Pneumatic connections 386 are registered as the GCC is installed. When fully seated, retention features 388 lock the GCC in place. This exemplary design includes five pneumatic connections to the GCC, two for conditioned (e.g., desiccated, filtered, VOC scrubbed) reactant gas to enter the NO device (one to the plasma chamber and one to the bypass pump), product gas out to scrubber, product gas return from scrubber, and NO/purge gas passage to the delivery device. In some embodiments (not shown), conditioned reactant gas exits the GCC through one pneumatic fitting and bifurcates into plasma chamber and bypass pump pathways from there.

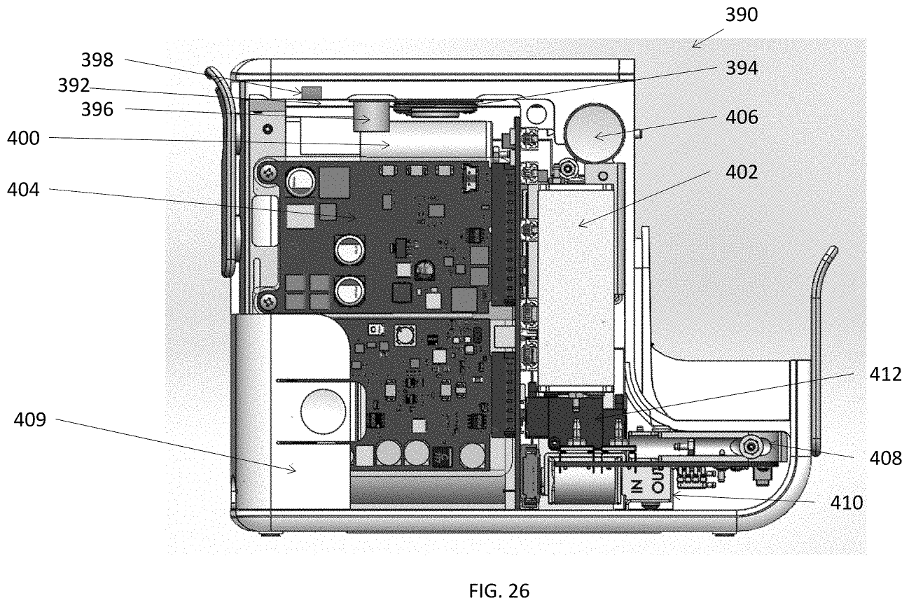

FIG. 26 depicts an exemplary NO generation and delivery device 390 with the enclosure opened. A user interface board 392 is mounted the top of the device, beneath the user interface. The user interface board interfaces with a speaker 394 for annunciating alarms, a piezo buzzer 396 for sounding alarms in the event of power system failure, one or more indicator LEDS, and a microphone 398 for receiving user inputs. In some embodiments, the user interface board includes a dedicated processor for driving the user interface and interacting with the treatment controller. This particular design utilizes a pressurized bypass flow to purge the delivery system between NO pulses. The necessary bypass reservoir volume is achieved with two reservoirs 400, 402, that are in fluid communication with each other. A control board 404 (e.g. user interface board, or treatment control board) includes circuitry for NO generation control, pump control, one or more sensors, sensor signal conditioning, alarm handling, power management, data acquisition, wireless communication (e.g., antenna), memory for data storage, and one or more processing units for running device software. A cartridge release button 406 can be used for releasing the GCC. A manifold 408 can provide pneumatic pathways that route reactant gas, product gas and bypass gas through the system. Proportional valves 410 are utilized to shape the NO gas and bypass gas flow, as required for accurate and timely patient dosing. Pressure sensors 412 are utilized to monitor plasma chamber pressure, scrubber pressure, bypass reservoir pressure, and delivery device pressure (for breath detection and/or kink detection).

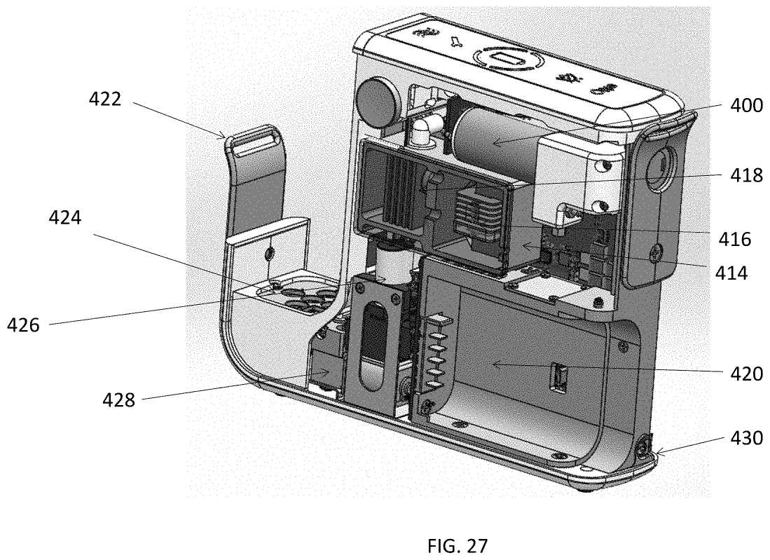

FIG. 27 depicts internal components of the device 390 on the opposite side of the system depicted in FIG. 26. The top of the device features the user interface. Below the bypass reservoir 400 is the high voltage (HV) housing 414, enclosing the high voltage transformer 416 and a plasma chamber 418. In some embodiments, the high voltage housing is potted with silicone, epoxy or similar materials to prevent electrical creepage and breakdown outside of the plasma chamber. In some embodiments, the high voltage circuit operates at a resonant frequency. Some systems are able to measure the resonant frequency. Shifts in the resonance frequency can be indicative of a high voltage component failure (e.g., transformer). A battery bay 420 receives a replaceable, rechargeable battery 409 (shown in FIG. 26). The system includes power circuitry to charge the battery when connected to external power through the power jack 430. The system also includes an internal battery (not shown) that keeps the system on during battery exchange and alarms while the main battery is removed. Also included are a shoulder strap loop 422, pneumatic connections for the GCC 424, pumps 426, and proportional valves 428. In some embodiments, the device enclosure is made from one or more of metal, polymer, and metal-coated polymer.

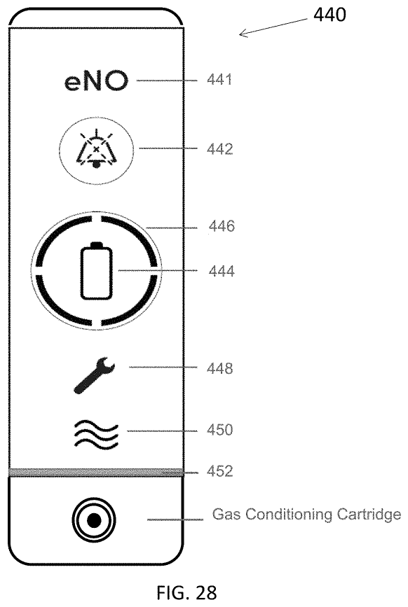

FIG. 28 depicts an exemplary user interface 440 for an ambulatory NO generation device. Each of the features depicted are backlight with lights. For example, a therapy indicator 441, such as an “eNO” symbol, is illuminated when NO therapy is active. An alarm silence button 442 temporarily cancels the alarm sound (e.g., 2 minutes). A battery status indicator 444 indicates whether or not a battery is installed (solid green when battery is installed and blinking red when battery has been removed). A segmented ring acts as a charge indicator 446 to indicate the level of charge by illuminating 1 to 4 segments. In some embodiments, the light moves from one segment to another around the circle to indicate charging is taking place. A service indicator 448 indicates that service is required when illuminated. A breath detect indicator 450 illuminates when a breath is detected. A cartridge status indicator 452 depicts the status of the gas conditioning cartridge. In some embodiments, the cartridge status indicator illuminates green, yellow or red to indicate OK, warning, or failure, respectively. In some embodiments, the shell of the GCC is illuminated by the cartridge status indicator light, like a light pipe to provide additional illuminated surfaces.

FIG. 23 shows the NO gas and purge gas flowing sequentially. In this application, the concentration of the pulse is the concentration within the scrubber minus any NO loss that occurs during pulse transit through the delivery system. In some embodiments (not shown) the concentration of NO within the scrubber is measured by a sensor. In some embodiments, the concentration of NO within the scrubber is derived as a function of reactant gas properties (O2/N2 ratio, humidity, pressure, temperature), plasma settings (frequency, duration, gap, flow rate, dithering), aging properties of the product gas (duration, pressure, temperature, scrubber NO loss, scrubber age, NO oxidation losses). In some embodiments, the concentration within a NO pulse can be varied by blending bypass and NO gas flows as the pulse is generated. NO pulses are generally pushed in the end with 100% bypass flow so that the delivery device only contains non-NO gas between breaths to prevent NO₂ formation.

This architecture allows for a higher instantaneous flow to be generated to prime the delivery device. This provides benefits in reducing NO delivery time, the ability to dose earlier in the breath and lower NO oxidation due to shorter transport time. In some embodiments, the rapid priming flow is achieved by opening the flow controller more during priming. In other embodiments, priming is achieved when NO and purge gas flow simultaneously. Rapid priming of the delivery device can be beneficial because it shortens the time between breath detection and NO delivery, enabling earlier portions of the inspiratory event to be dosed. As the delivery device is primed, the contents of the delivery device (typically purge gas, or air) are pushed to the patient and replaced with either full concentration or diluted product gas.

A further feature of a pressurized scrubber/pressurized bypass architecture is that it can enable both pumps to be run continuously when the flow into the system (reactant gas) and average flow out (NO+air boluses) are balanced. This architecture is also not sensitive to the dead volume of the plasma chamber and scrubber as they relate to dose volume since the amount of NO delivered to the patient is solely a function of concentration and amount released from the scrubber.

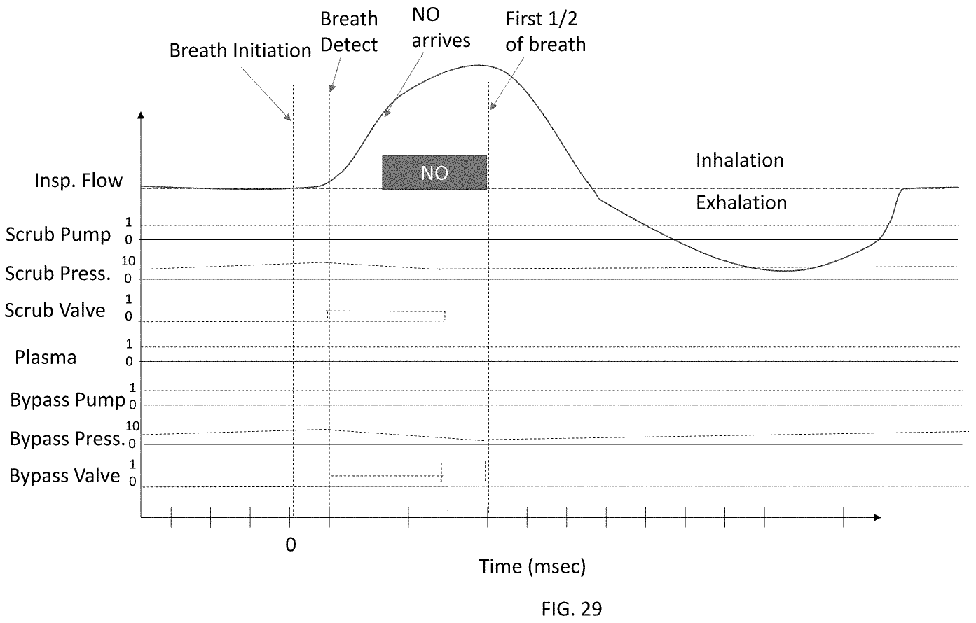

In an exemplary embodiment, depicted in FIG. 29, flow from the scrubber and flow from the bypass channel overlap (i.e., occur simultaneously) for a period of time. For example, FIG. 29 shows an exemplary graph of performance relating to the pressurized scrubber/pressurized bypass architecture of FIG. 12. When the two flows overlap, the sum of their flows is kept below safety and patient comfort thresholds. In some embodiments, the sum of the flows equals 5 lpm. Overlapping the flows provides the following benefits: 1) The finite NO charge within the pressurized scrubber channel is spread out across a longer period of time, dosing a larger portion of the inspired volume, 2) Transit time of NO remains minimal because the flow rate remains high. This helps minimize NO oxidation and NO2 formation, 3) NO2 formation is also minimized due to dilution of the NO-containing gas earlier (i.e., within or near the NO generator instead of after traveling to the patient through the delivery device). In some embodiments, the concentration within a NO pulse can be varied by blending bypass and NO gas flows as the pulse is generated. NO pulses are generally pushed in the end with 100% bypass flow so that the cannula only contains non-NO gas between breaths to prevent NO₂ formation.

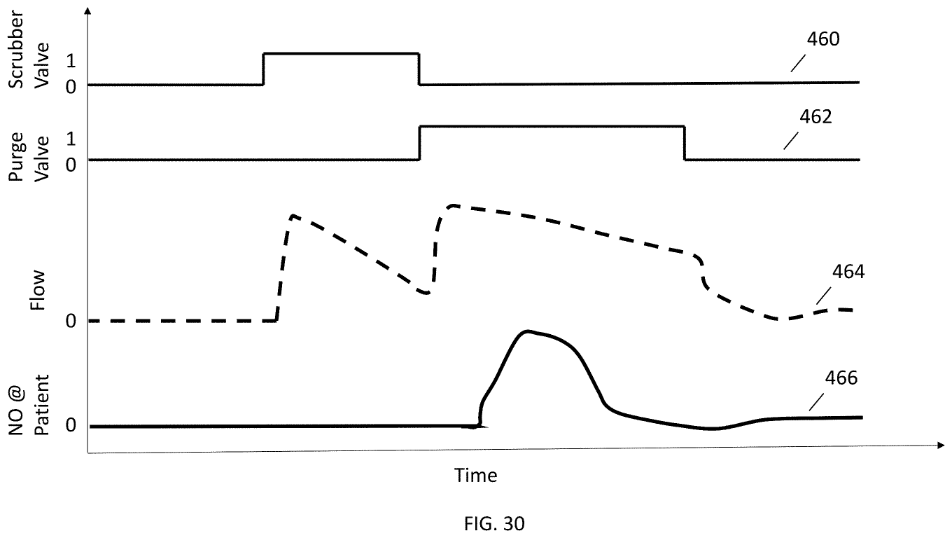

Flow exiting the pressurized scrubber and pressurized reservoir can be controlled with a proportional valve, mass flow controller, needle valve, or other flow control device at the exit of each gas chamber. When a binary valve is utilized, the pressure can decay and flow rate can decay exponentially over time. FIG. 30 depicts a graph of performance of an exemplary pressurized scrubber/pressured purge system. Curve 460 represents the scrubber valve opening (open=1) and closing to introduce NO to the delivery device. Curve 462 represents the purge gas reservoir valve opening to push the NO to the end of the delivery device. Curve 464 depicts the flow within the delivery device. It can be seen that as the pressure decays within the respective reservoirs, the flow through the delivery device decays. Curve 466 depicts the NO pulse arrival at the patient. Additional flow of purge gas after the NO pulse ensures that the delivery device has been cleared of NO and NO2 between breaths.

When a proportional valve is utilized, the flow rate exiting a scrubber can be controlled to a specific flow rate over time. In one exemplary embodiment, the NO delivery system is designed to deliver gas of varying concentration at a specific flow rate. It achieves this by blending product gas from the scrubber and purge gas in the correct amounts to achieve a specific concentration of NO at a specific flow rate. In some embodiments, for example, flow exiting the pressurized scrubber and bypass reservoir are both half of a 5 lpm target flow rate (e.g., 2.5 lpm and 2.5 lpm, respectively). Once the target amount of NO has been delivered to the delivery device, the NO-containing gas flow ceases and the bypass gas flow continues to push the NO-containing gas to the patient. In some embodiments, the bypass gas flow continues at a less than target flow rate, (e.g. the half or target flow rate). In some embodiments, the bypass gas flow rate increases to the target gas flow after NO delivery to the delivery device ceases. This approach provides for pushing the last portion of the NO pulse through the delivery device as quickly as possible to minimize transit time and related NO oxidation. In some embodiments, the flow rate of the gas being controlled is measured by a flow sensor (not shown). In some embodiments, the flow rate of gas is calculated from the rate of change in the respective reservoir pressure (i.e., scrubber reservoir, purge reservoir).

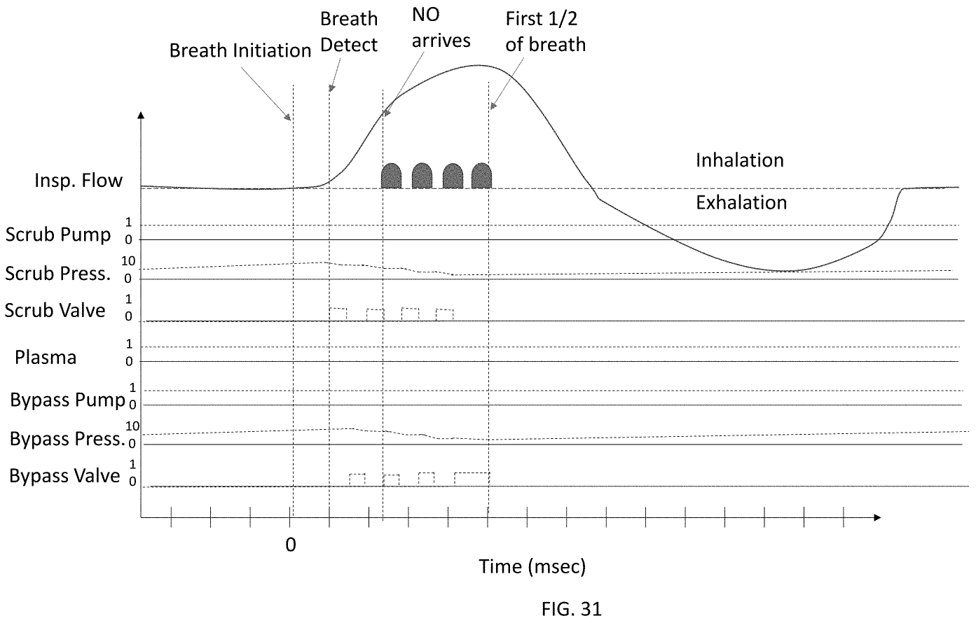

Another approach to spreading NO over a larger portion of a breath is to alternate between NO gas flow and bypass flow multiple times within a patient breath, as depicted in the exemplary graph shown in FIG. 31. Once breath is detected, a bolus of NO product gas is released from the scrubber. This is followed by a bolus of bypass gas. The objective is to spread NO delivery across a longer portion of the inspired volume with the expectation that NO boluses will diffuse into the purge gas during transit and within the patient. The bolus sizes are shown to be the same in the figure, however they could also be different. In the event that the purge gas boluses are less than the volume of the delivery device, the final purge gas bolus is larger because it will push NO all the way out of the delivery device.

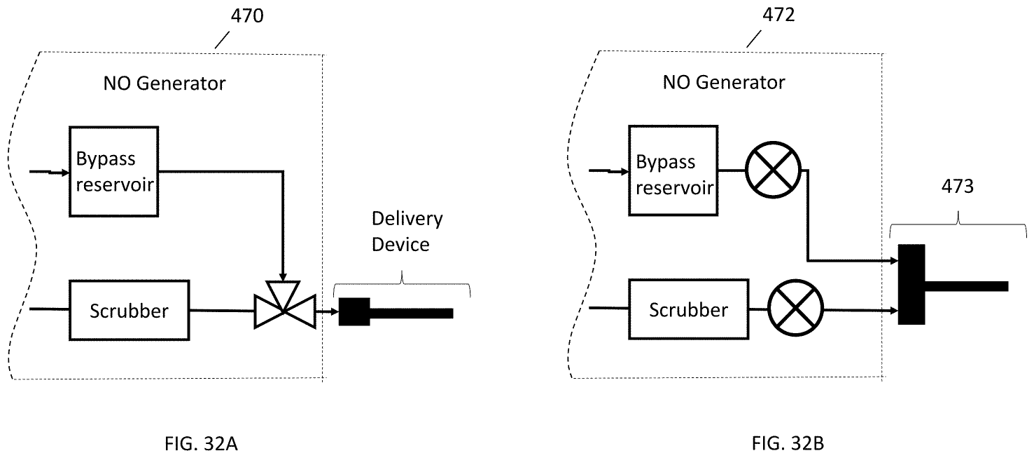

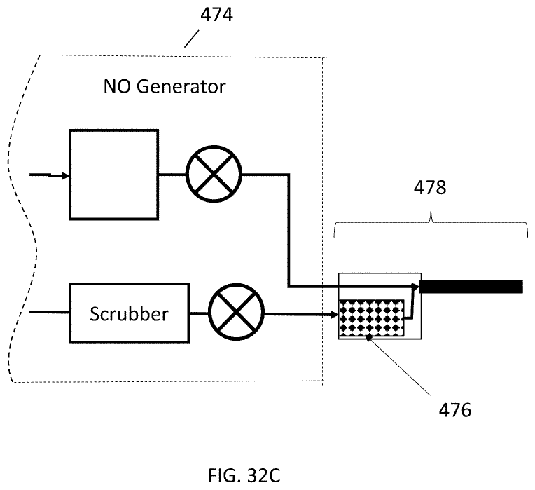

The point of mixing of the bypass and NO gas can be within either the NO generator or the delivery device, as shown in FIGS. 32A and 32B. FIG. 32A depicts an embodiment of a NO generator 470 where the NO and bypass paths intersect within the device. This embodiment utilizes a 3-way valve which provides the benefit of fewer valves. In some embodiments, the 3-way valve is a proportional valve while other embodiments, each path is binary (open/shut). FIG. 32B depicts an embodiment of a NO generator 472 where flow through the bypass and NO channels remain independent within the NO generator and combine within a delivery device 473. FIG. 32C depicts an embodiment of a NO generator 474 where the NO and purge lines are independent in the controller and the NO line is scrubbed using a scrubber 476 in a delivery device 478.

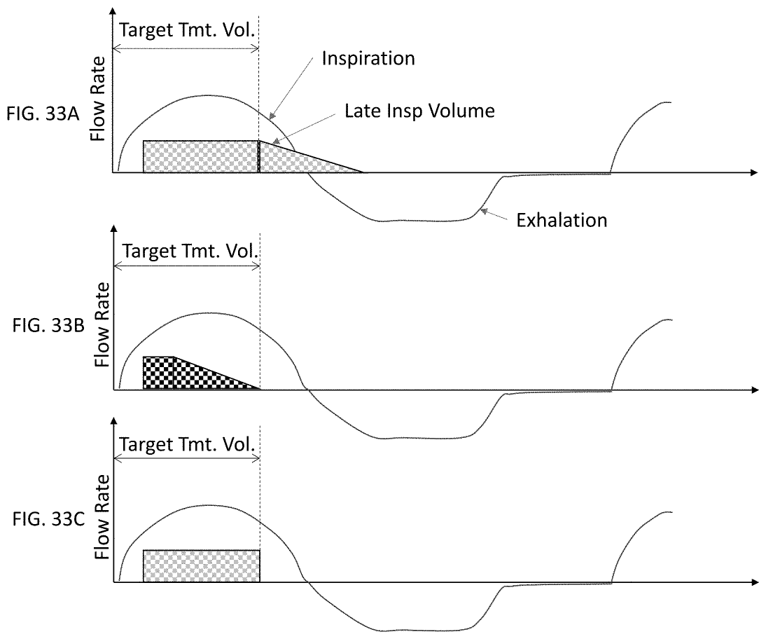

FIGS. 33A, 33B, and 33C depict exemplary NO pulse profiles for the same inspiratory flow pattern. Each of the NO pulses are delivered at the same flow rate, as indicated on the y-axis. For these exemplary scenarios, it is desirable to deliver NO only during the first two thirds (66%) of the inspiratory volume. For the purpose of this illustration, each of the scenarios depicted in FIGS. 33A, 33B, and 33C have the same breath detection and NO transit time through the delivery system. FIG. 33A depicts a system that is slow to terminate delivery of NO to the patient. This is representative of the performance of the linear system depicted in FIG. 3 where once the NO is shut off, reactant gas travels through the scrubber and delivery device to the patient to purge the system, taking hundreds of milliseconds. Because of the long amount of time for the NO to pass through the system, NO continues to reach the patient at a decreasing concentration for an amount of time after NO generation stops. In some scenarios, the NO delivery timing extends beyond the target timing window and doses later in the breath, potentially dosing unhealthy portions of the lung in some patients.

FIG. 33B depicts how a system that is slow to shut off the NO flow can be turned off earlier in the inspiratory event to prevent dosing the non-target (i.e., unhealthy) part of the lung. The shaded area of NO is smaller in this figure, which is representative of the NO molecules within the pulse being delivered as a function of time. In order to deliver the same number of moles to the patient as in FIG. 33A, the concentration of NO must be higher. This higher concentration is depicted by the darker shading of the NO pulse. The higher concentration increases the rate of NO oxidation to NO₂ which affects power consumed in generating NO (due to higher NO loss), battery life, scrubber life and, in some cases, inhaled NO levels. In order to not extend NO delivery beyond the target window within the inspiration, NO delivery is declining as the inspiratory flow rate is increasing. This can result in ineffective concentrations of NO in the latter portions of the inspiratory window in some instances.

FIG. 33C depicts a system that can rapidly terminate the NO bolus, such as a pressurized scrubber/pressurized bypass system. The NO pulse is quickly terminated by shutting off the valve at the exit of the scrubber and the pressurized bypass gas pushes that trailing pulse edge through the delivery device to the patient rapidly. This approach enables a NO system to deliver NO at target concentrations for a larger portion of the target inspiratory window. It also enables an NO system to deliver lower product gas concentrations because a larger volume of NO can be delivered, as depicted by the rectangle of NO in FIG. 33C vs. the trapezoidal pulse shape of FIG. 33B. As mentioned above, lower concentrations of NO are beneficial with respect to NO oxidation and inhaled NO2 levels.

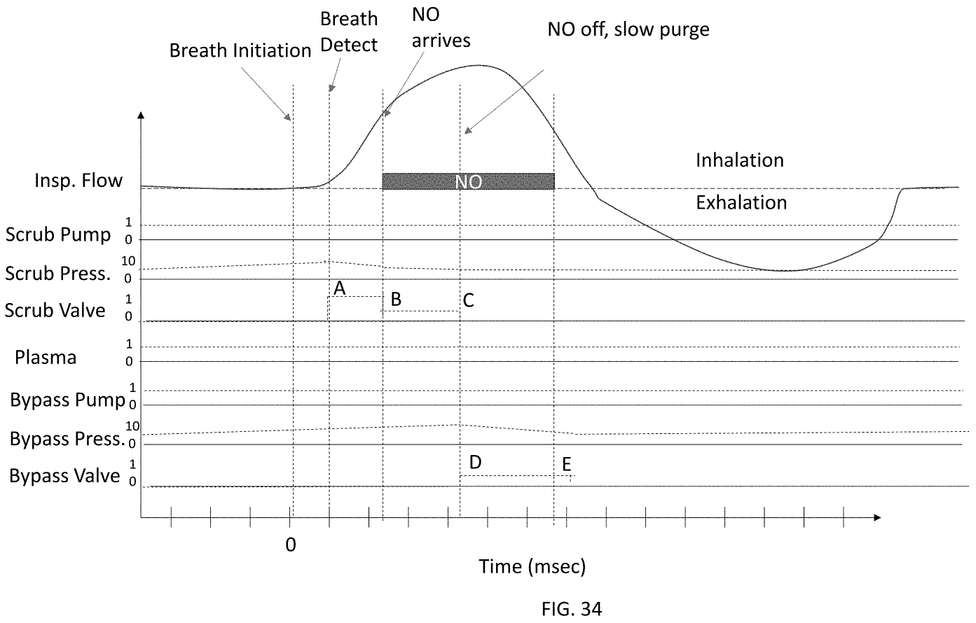

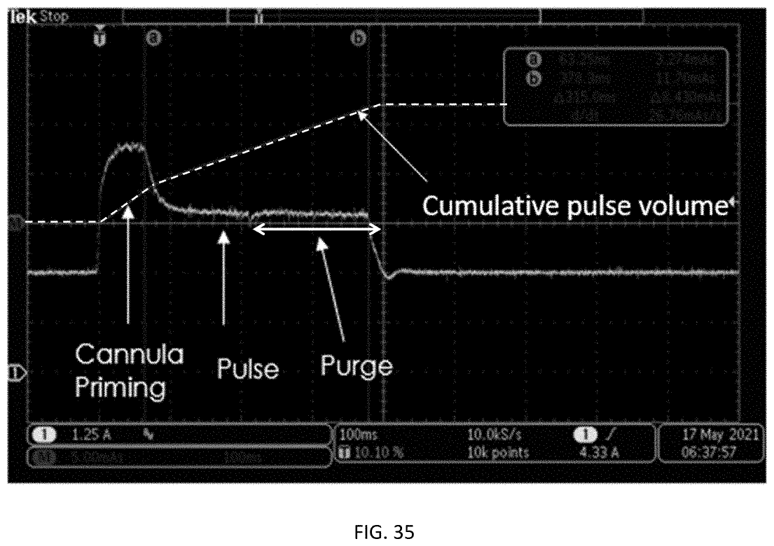

FIG. 34 depicts a graph showing another exemplary approach to prolonging the NO pulse. Upon breath detection, the flow controller at the exit of the NO path releases an initial high flow rate to prime the cannula and get the leading edge of the NO bolus to the proximal (patient) end of the delivery device (point A). After a volume roughly equivalent to the volume of the delivery device has been introduced to the delivery device, the flow controller partially closes to slow the flow rate of NO through the delivery device and to the patient (point B). This slower flow rate effectively stretches out the duration of the delivered pulse of NO to the patient. The trailing edge of the NO pulse is managed by closing off the NO flow controller completely (point C) and flowing bypass/purge gas through the delivery system at the same flow rate (point D). In doing so, the NO gas is pushed through the delivery device at a constant rate until it has exited the proximal end of the delivery device entirely at which point the purge gas flow is turned off In some embodiments, the purge gas is left on for a short amount of time longer to ensure that the delivery device is purged (point E). This approach is applicable to pressurized scrubber/pressurized bypass architecture, however any other architecture that can provide these pulse profiles could be applicable. FIG. 35 presents actual exemplary data from a NO pulsed device utilizing a pressurized scrubber, pressurized bypass architecture. The solid line represents gas flow through a delivery cannula. The dashed line represents cumulative gas flow into the cannula (NO and purge gas combined). Upon breath detection, NO gas is released from the scrubber at a fast flow rate until the cannula is primed. Then, the NO flow rate slows as can be seen by the decrease in the solid line and change in slope in the dashed line. Midway through pulse delivery, the target amount of NO has been delivered to the delivery system. Purge gas begins to flow from the bypass reservoir. A ripple in the flow data indicates the transition from one flow source to the other. Bypass gas pushes the NO gas through the remainder of the cannula and then stops. This particular example was generated with a target NO pulse length of 400 msec and a patient respiratory rate of 20 bpm.

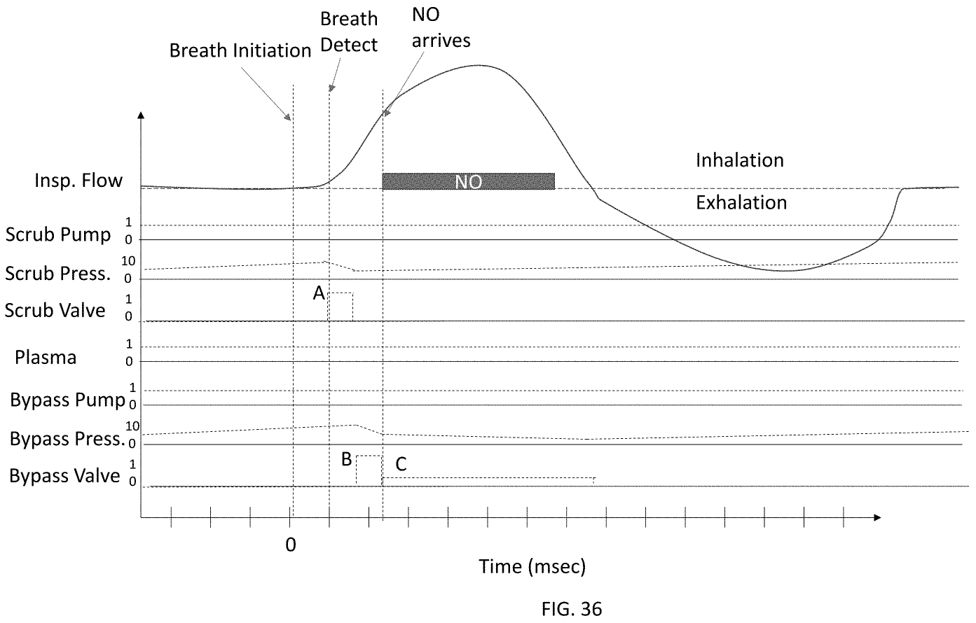

In some applications, such as with a high respiratory rate, the volume of the NO pulse may be less than the volume of the delivery device. As shown in FIG. 36, a bolus of NO is released into the delivery device (point A). Any delay between breath detection and NO release, be it intentional or inherent to the system, is not depicted in FIG. 36. While the volume of the bolus as it relates to the volume of the delivery device can vary, the volume of the released bolus is only ½ of the delivery device volume in this example. The system releases purge gas at a fast rate (point B) to propel the NO bolus to the proximal end of the delivery device but not out of the delivery device. Then, the purge gas flow rate is slowed (point C) to meter the NO out of the end of the delivery system at a slow flow rate to introduce NO over a large portion of the inspiration. The NO pulse depicted in FIGS. 34 and 36 dose a large portion of the breath. This approach doses a larger portion of the lung, which can be advantageous in healthy patients that are seeking performance enhancement in low ambient oxygen conditions, for example.

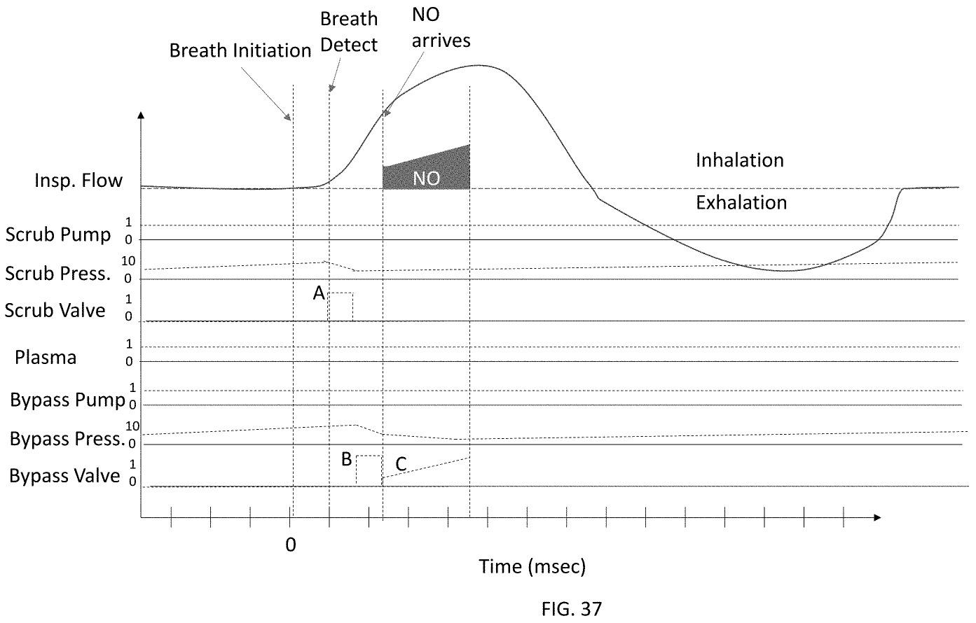

A patient inspiration has an initiation point, a ramp in inspiratory flow rate, a maximum inspiratory flow rate, and a decrease in inspiratory flow rate back to zero. The shape of the inspiratory flow curve has been modeled as the positive portion of a sine wave, a rectangle, or a trapezoid in some embodiments. When constant concentration NO is introduced to a varying inspiratory flow rate, the concentration of NO in the inspiration will vary. FIG. 37 depicts an example of a pulse delivery approach that varies the NO pulse flow rate in order to improve the consistency of NO concentration within the dosed portion of a tidal volume. NO is introduced to the delivery device at point A. NO is pushed through the delivery device with purge gas at point B. The flow rate of NO exiting the delivery device is varied at Point C.

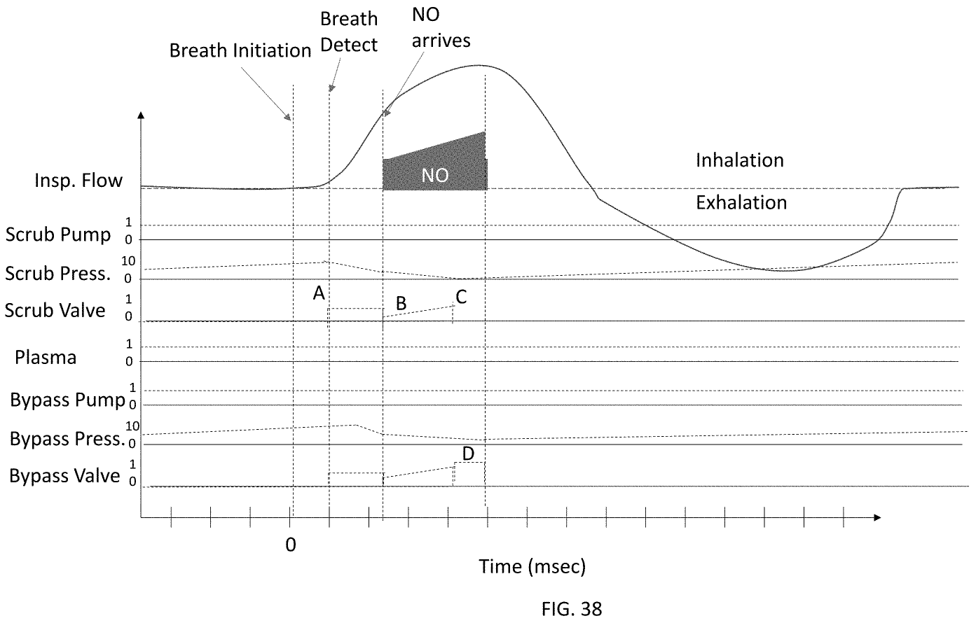

FIG. 38 depicts an exemplary embodiment where the cannula is primed with NO gas and purge gas flowing simultaneously (point A). This has the effect of diluting the NO to a target concentration and minimizing the time required to prime the delivery device due to a higher flow rate. Both dilution and reduced transit times provide a benefit in reducing NO oxidation, thereby reducing inhaled NO2. In the depicted embodiment, once the delivery device is primed, the treatment controller sets NO gas and purge gas flow into the delivery device at a particular dilution level (50/50 in the example) and varies the flow during the breath (point B) in approximate proportion to the inspiratory flow rate. The treatment controller achieves target product gas and bypass gas flow rates by varying respective flow controller settings. Proportional NO flow with inspiratory flow provides a more consistent concentration NO within the target zones of the lung and airway. After the target amount of NO has been introduced to the distal end of the delivery device (in moles), the NO gas is shut off (point C) and the purge gas pushes the trailing edge of the pulse to the patient (point D). The purge gas flow rate is higher at the end of the NO pulse than at the beginning because it is the only gas flowing. This approach of delivering variable amounts of purge and NO gas can be utilized to dynamically vary the concentration and/or moles of NO delivered throughout the duration of the inspiratory event. In some embodiments, a NO controller utilizes sensor information (e.g., diaphragm EMG, or inspiratory sounds from a microphone) as a proxy for direct measurement of inspiratory flow. The treatment controller utilizes the inspiratory flow measurement information as an input for ramping the pulse flow rate during inhalation in real time.

When NO and bypass flows occur simultaneously, it can be necessary to control the flow rate of one or both of the flows to ensure that the combined flow rate does not exceed a flow rate threshold (e.g., patient comfort threshold). It is also important that the flow from one reservoir does not inhibit flow from another reservoir. In some embodiments, flow controllers are located at the exit of each reservoir (bypass and scrubber) to ensure proper flow. In some embodiments, one-way valves are utilized to ensure that flow from one path does not flow into the other channel. Examples of one-way valves are check valves, duck-bill valves, ball and socket valves, mass flow controllers, etc.

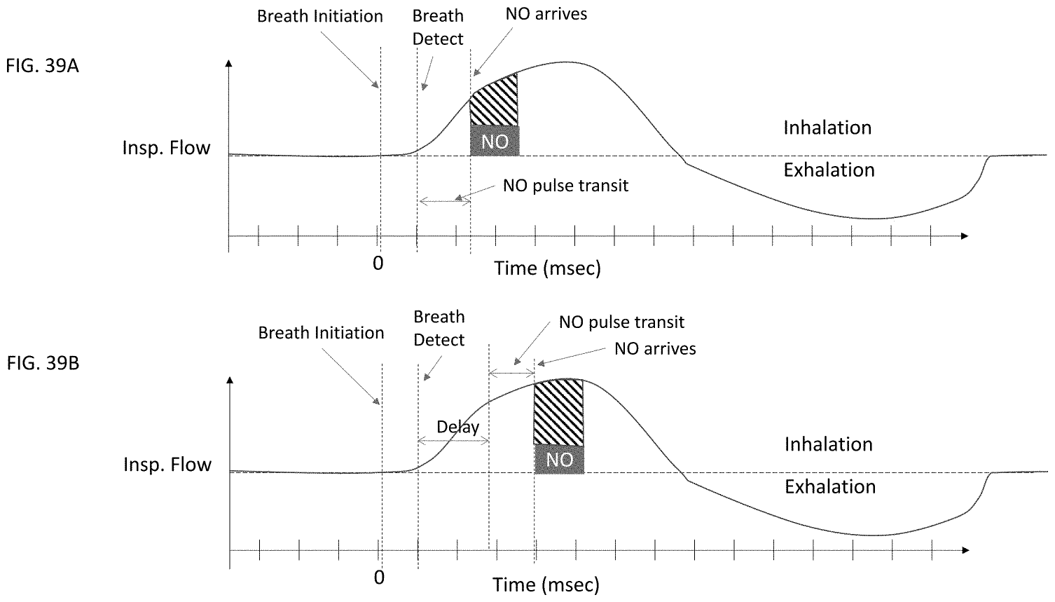

Another approach utilized to place a NO pulse within a target subset of an inspired volume is to delay the NO pulse delivery. How early a NO pulse arrives at the patient is typically limited by the time it takes to detect breath, release the NO and/or deliver NO along the length of the delivery system. In some embodiments, it is desirable to further delay the introduction of NO to the patient. This delay can be designed to place the NO within a particular location in the lung and/or to treat a larger subset of the inspired volume. In some embodiments, a delayed pulse overlaps with the higher velocity portion of the breath that corresponds with more elastic, healthy lung. FIGS. 39A and 39B depict graphs of exemplary scenarios with the same inspiratory profile but different pulse timing. FIG. 39A depicts the NO pulse being delivered as soon as possible within the inspiration, immediately after the breath detection and transit time. FIG. 39B depicts a delay occurring prior to delivery of the NO pulse through the delivery system. Although it is possible to send the NO pulse partially through the delivery system prior to applying the timing delay, this involves NO aging for a longer time within the delivery system and can result in higher NO₂ levels. The delay in FIG. 39B results in delivering the NO during the point of peak inspiratory flow rate. By dosing the peak flow rate, a larger volume of inspired gas is mixed with NO during inhalation. The dosed portion of the inspired volume is represented by the NO pulse plus the shaded region of the pulse. This can be beneficial because it exposes a larger volume and more surface area of the lung to NO for increased pulmonary vasculature relaxation and oxygen uptake. Delays can range from a few milliseconds to hundreds to thousands of milliseconds, depending on the breath trigger point (inspiration start, inspiration end, exhalation end), patient response, inspiratory flow profile, NO pulse duration, NO pulse transit time, breath detect duration, breath rate and other factors). This concept of pulse delay is applicable to all types of NO delivery systems (tank, electric, and chemical).

In some embodiments, the magnitude of the pulse delay is dynamic. In some embodiments, the pulse delay is tied to respiratory rate. In some embodiments, respiratory rate is measured over a series of n prior breaths. In some embodiments, the pulse duration is a function of the respiratory rate as measured by the NO delivery system, where shorter durations are used for faster respiratory rates and longer durations are used with slower respiratory rates. The relationship between pulse delivery delay and respiratory rate can be captured in a look-up table or equation and utilized by the NO delivery system controller to determine the delay duration for each NO pulse delivered.

In one exemplary embodiment, the objective of a NO delivery system is to deliver a 100 msec pulse of NO to the middle of the breath. This type of approach can be utilized to dose a specific region of the lung and/or airway. This exemplary system requires 50 msec on average to detect breath and another 20 msec to deliver NO from the device to the patient. Thus, a 70 msec delay is associated with every pulse delivered. For fast breath rates such as 40 breaths per minute, the inspiratory duration is 0.5 second and the midpoint occurs 125 msec into the breath. Given that it takes 70 msec to deliver the NO pulse, the system does not add additional delay so that pulse delivery begins at 70 msec and lasts until 170 msec, thereby straddling the midpoint of inspiration at approximately 125 msec. As respiratory rate slows, inspiratory events are typically longer. For example, when respiratory rate is 12 breaths per minute, the inspiratory event can last more than a second. Delivery of the NO as quickly as possible with a delay of 70 msec would place the entire NO pulse earlier than the mid-breath target. Thus, at slower respiratory rates, the NO pulse in this scenario is delayed to begin at an appropriate time that enables the pulse to straddle the mid-point of the breath. In an example where the breath lasts 1 second, the midpoint of the breath is 500 msec. The target beginning of NO delivery is 450 msec into the breath. Accounting for delivery time (20sec) and breath detection time (50 msec), the NO pulse should be released after a delay of 380 msec. In this example, for respiratory rates ranging from 12 to 40 breaths per minute, the delay would be calculated as follows: D=−26.5*RR+1061, where D=the delay in milliseconds and RR=the respiratory rate in breaths per minute. For respiratory rates exceeding or less than the stated range of respiratory rate, there would be no delay or a 380 msec delay, respectively.

The flow rate for the NO pulse can be measured directly with a flow sensor or by comparing pressure before and after a flow restriction. In some embodiments, the scrubber pressure and reservoir pressure are compared with the pressure downstream of the flow controllers (a form of flow restriction), where the NO and bypass gases merge. The downstream pressure sensor is typically in addition to the breath detection sensor since the breath detection sensor typically has a lower range and will saturate (i.e., rail) during the NO pulse event. In some embodiments, the pressure before and after the scrubber is compared to determine a flow rate through the scrubber. The difference between the up and downstream pressures, combined with the flow controller setting (e.g., orifice size) can be used to determine the flow rate through the flow controller. In some embodiments, the pressure within the scrubber/reservoir and flow controller setting are used to approximate the flow into the cannula. In many cases, the flow restriction through the delivery device is considered a constant but may vary between different delivery devices. In some embodiments, the NO device controller can sense the type of delivery device and adjust the pressure delta to gas flow rate relationship accordingly. In some embodiments, the rate of change of pressure in a known volume (e.g., scrubber or bypass reservoir) can be used to determine a gas flow rate. NO flow rate is necessary for priming a delivery device and/or positioning a NO pulse in a particular location within a delivery device. The flow rate is multiplied by time to calculate a volume. This volume is compared with the known delivery device volume to understand how much of the delivery device volume has been displaced by the NO pulse and/or where the leading edge of the NO pulse is located within the delivery device. When an NO pulse is small with respect to the volume of a delivery device and the NO pulse is to be positioned at the proximal (i.e., patient) end of the delivery device, a known volume of purge gas can be used to push the NO volume a known distance along the delivery device using the same approach.

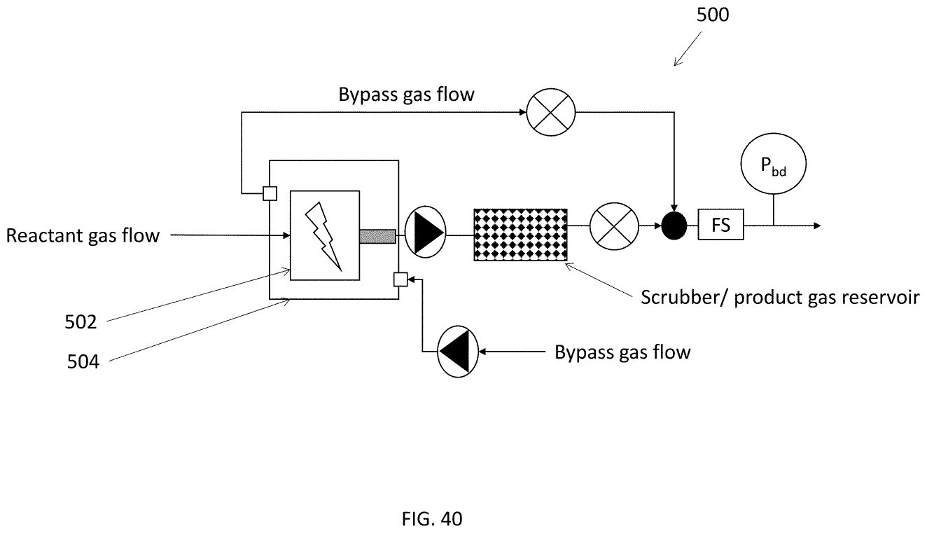

FIG. 40 depicts an exemplary design of a NO generation system 500 that cools a plasma chamber convectively with purge gas. As shown in FIG. 40, the plasma chamber 502 is at least partially covered by a purge gas flow conduit. In some embodiments, cooling fins on the plasma chamber increase thermal transfer. In some embodiments, the outlet port of the plasma chamber is metallic to increase heat transport. In some embodiments, the direction of purge gas flow is opposite that of product gas so that the coolest temperature purge gas can be used for maximizing heat exchange. In some embodiments, the flow path of the purge gas has an electrically conductive layer or is entirely constructed from electrically conductive material so that it can serve as a Faraday cage to shield other parts of the system and/or external devices and Users from electromagnetic interference. In some embodiments, the volume around the plasma chamber filled with purge gas serves as a pressurized reservoir 504 for the purge gas. In some embodiments (not shown), purge gas flows over the plasma chamber prior to passing through the pump and getting pressurized.

Pressurized Scrubber, Pressurized Bypass Architecture: Single Pump

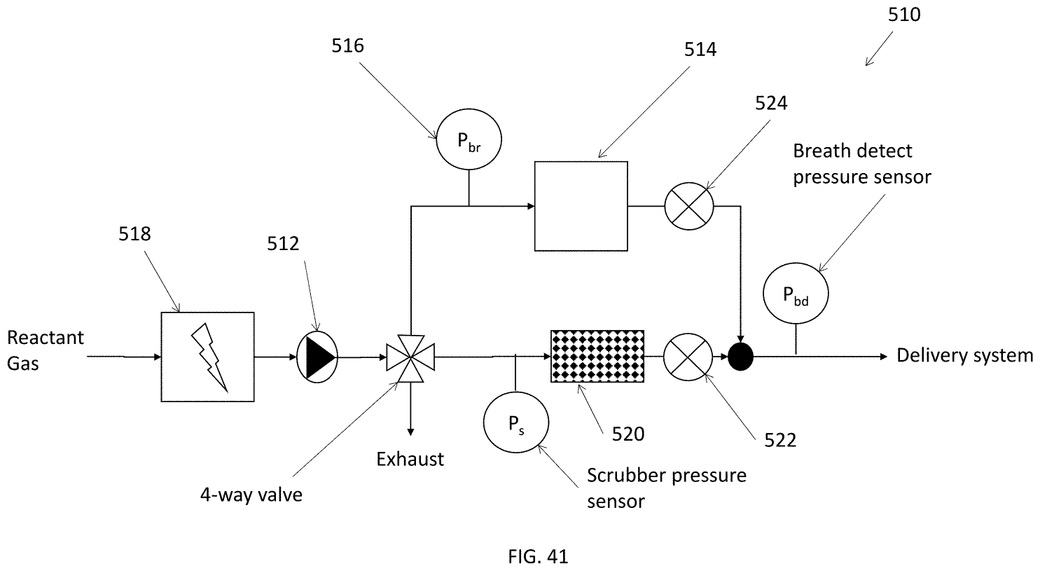

FIG. 41 depicts an embodiment of a NO generation system 510 with a pressurized scrubber with pressurized bypass architecture that functions with a single pump. This embodiment provides potential weight and power savings. In some embodiments, the pump 512 fills a bypass reservoir 514 first to a target pressure according to the reservoir pressure measured by a bypass reservoir pressure sensor 516, Pbr. Filling the bypass reservoir first can be done because the reactant gas that fills the reservoir will not change over time, as compared to the nitric oxide containing gas which will oxidize over time, forming NO₂. Then, the plasma chamber 518 is turned on and gas flows to the scrubber 520 to fill it with NO. Prior to ceasing flow to the scrubber the plasma chamber is turned off and remaining gas within the chamber and dead volume between chamber and scrubber upstream valve is also delivered to the scrubber, leaving no NO in the plasma chamber. In some embodiments, the pump turns off after the reservoir and scrubber have been pressurized.

In some embodiments (as shown in FIG. 41) the pump continues running, directing gas out of the pneumatic system (labeled “exhaust” in the figure). In some embodiments, this released gas is used to cool the enclosure of the NO generation device in some embodiments. When breath detection occurs, the downstream scrubber valve 522 opens to release the scrubber pressure to deliver NO. Then, the downstream scrubber valve 522 closes, and the downstream bypass valve 524 opens to release bypass gas to push the NO pulse down the delivery system to the patient. Then, the bypass downstream valve is closed so that the bypass reservoir can be refilled with gas. The downstream valves can be various kinds of flow control devices, including but not limited to binary valves, proportional valves, or mass flow controllers.

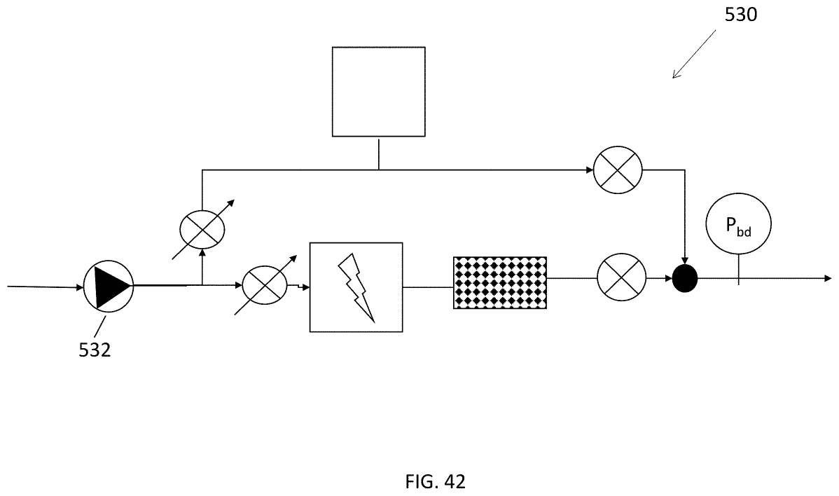

FIG. 42 depicts an exemplary embodiment of a NO generation system 530 whereby a single pump 532 runs continuously, providing the bypass pathway and the NO generation pathway simultaneously. This approach differs from the approach described in FIG. 41, where the pump runs continuously but provides gas to the two flow paths sequentially. Flow controllers on each leg ensure that the ratio of flow to each leg is in accordance with desired proportions. This embodiment provides an advantage in that a single pump is used instead of two, decreasing mass, size and noise. In some embodiments, the flow controllers are simply fixed orifices.

Push/Pull Architecture

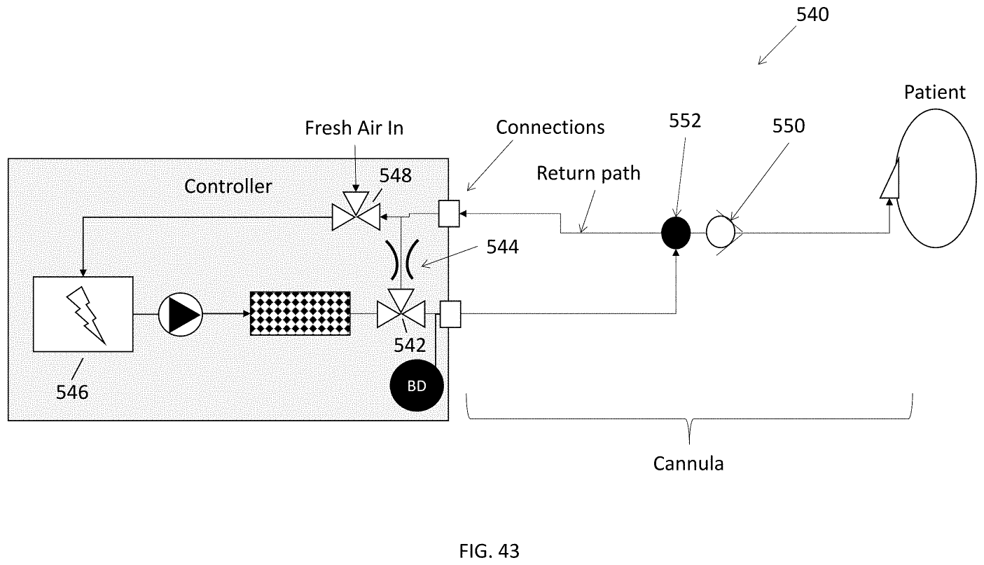

FIG. 43 depicts an exemplary embodiment of a push/pull architecture 540 consisting of an external recirculation loop with a shunt to create an internal recirculation loop. A push/pull architecture is designed to deliver the NO pulse faster through the delivery system by decreasing the pressure downstream of the NO pulse as it is pushed towards the patient. In one mode of operation, NO is generated within the internal recirculation loop prior to NO delivery to the patient. An optional flow restriction 544 (e.g., critical orifice) above the 3-way valve 542 can be utilized to match the flow restriction with the delivery device to assist in maintaining a constant flow rate through the plasma chamber 546 when the system changes from recirculation to NO delivery. At the time of NO pulse delivery (typically after breath detection), NO flow changes from the internal shunt flow to the external loop flow down the delivery device (the “push”). This approach can allow for the following: 1) A flow of fresh, pressurized NO is already established at the time of breath detection, eliminating delays associated with establishing a flow through a scrubber, and 2) Return flow within the cannula helps pull the NO pulse down the cannula in addition to pushing the pulse for faster transit times (the “pull”). When the NO pulse reaches the intersection point within the delivery device, return flow can be halted by toggling the three-way valve 548 to permit fresh air into the system, forcing the NO flow to travel towards the patient and make-up gas to enter the system. In some embodiments, the 3-way valve and/or fresh air source is part of the delivery device. The timing of the NO pulse arrival at the intersection can be based on characterization of the system and is a function of the gas flow rate and pathway volume. This characterization is typically unique to a specific NO generator/delivery system combination. In some embodiments, an NO controller is programmed with timing characteristics of the system. A check valve 550 at the patient end of the system prevents the return line pulling in air from the patient end.

In some embodiments, a typical operating sequence for the push/pull architecture can be as follows: Step 1) Generate NO in the internal recirculation loop; Step 2) Upon breath detect, push the NO to the patient while pulling gas back through a return lumen in the cannula; and Step 3) When the NO pulse is getting close to the intersection of outbound and return lumens, change the source gas to be coming from an outside source, thereby blocking the pulled gas flow. In some embodiments, the timing of step 3 is based on an understanding of the gas flow rate and volume of the delivery device between NO generation device and intersection point. For example, the NO controller can mark time from the point that NO begins traveling down the delivery device. In some embodiments, the amount of time that it takes for the NO pulse to reach the return point is calculated as the volume of delivery system between the 3-way valve 542 (point A) and the return point 552 (Point B) divided by the product gas flow rate. In some embodiments, the amount of time from the valve 542 to the return point 552 is characterized for the system at different product gas flow rates. In some embodiments, the system only operates at a single product gas flow rate and the transit time is known for each type of delivery device based on system characterization.

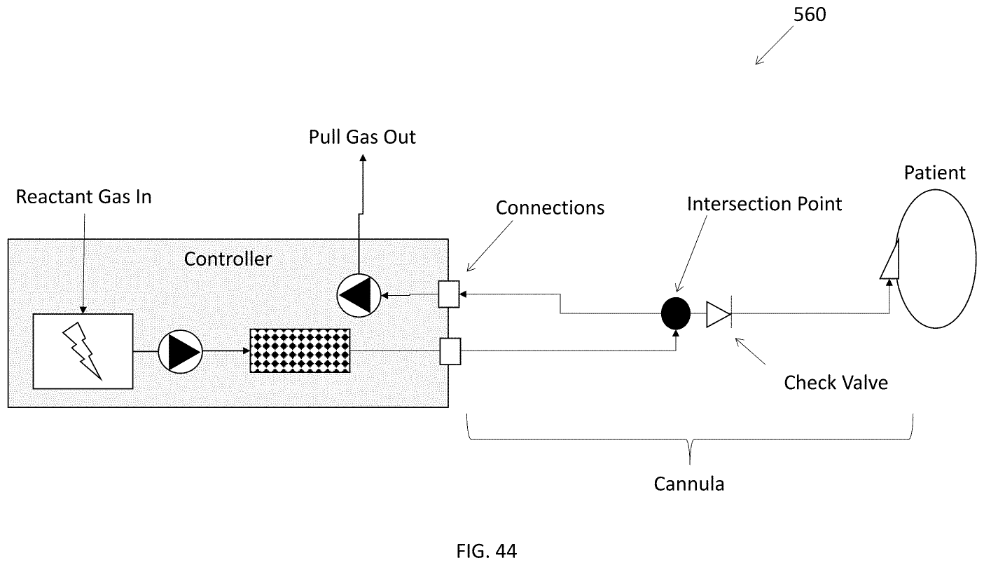

FIG. 44 depicts an exemplary embodiment of a push/pull architecture 560 that is open loop. A second pump is utilized to pull the pulse along the delivery device. Returning gas from the delivery device can be vented to atmosphere because it does not contain NO/NO₂. When the NO pulse reaches the intersection point at the proximal (patient) end of the cannula, the return flow pump is arrested to make NO flow go to the patient. A check valve or one-way valve on the patient side of the intersection point prevents the pull pump from drawing air from the patient end during the pulse pulling step. In some embodiments, the valve downstream of the intersection point is a pressure-relief valve that opens under a particular pressure.

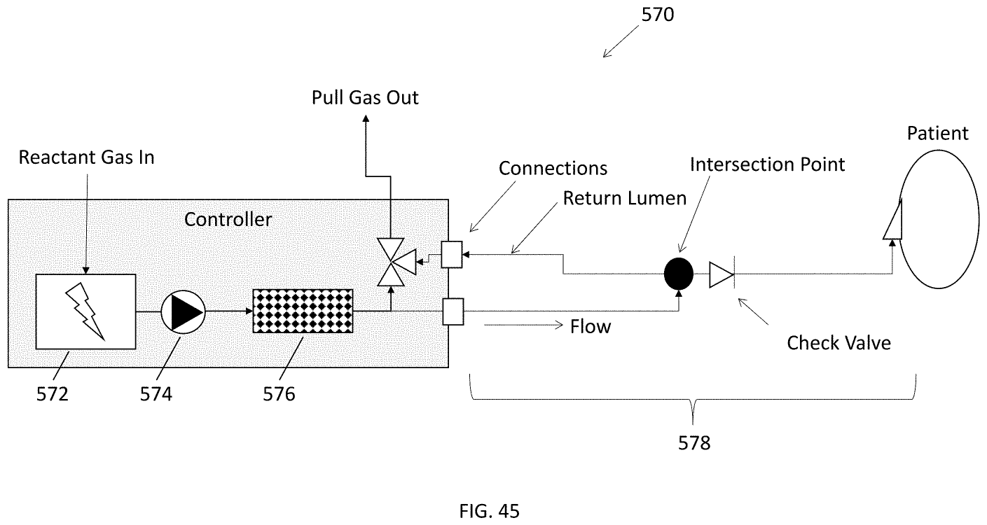

FIG. 45 depicts an exemplary embodiment of a pulsed NO delivery device 570 including a plasma chamber 572, a pump 574, a scrubber 576 and a cannula 578 with out and back flow lumens. In this embodiment, NO can be generated as a pulse and sent towards the patient prior to breath detection. NO is generated and sent into the delivery device towards the patient. Gas flows down the delivery device to the intersection point and back towards the controller. Air formerly within the delivery device is expelled out a 3-way valve 580. Once breath is detected, the 3-way valve redirects flow so that both lumens are connected to the pump and flow towards the patient. In some embodiments, a pressurized scrubber and/or reservoir is used to aid in propelling the gas. This embodiment allows for the NO pulse is be closer to the patient at the time of breath detection but is not aging for the entire duration between breaths. In some cases, breath is detected before the NO pulse reaches the intersection point. In some cases, the NO pulse passes the intersection point and is returning to the controller prior to breath detection. Both scenarios (and any in between) are acceptable because both NO lumens of the cannula are emptied with flow towards the patient to deliver the NO to the patient.

In some embodiments, as the NO is delivered to the patient, the ratio of flows in the delivery device lumens could be varied based on the location of the NO and how much flow is needed to get all the NO in each lumen into the patient. For example, if all of the NO pulse had passed the intersection point at the time of breath detection, a greater amount of flow could be delivered through the return lumen to hasten delivery of the NO to the patient. The actual ratio of flow through the lumens would be based on an understanding of the delivery device length, volume and flow rate to know where the NO pulse is located within the delivery device. In some embodiments, the ratio of flow rates between outbound lumens to the patient is controlled by flow controllers in each lumen (not shown).

Pressurized Bypass with Separate Desiccant Chambers

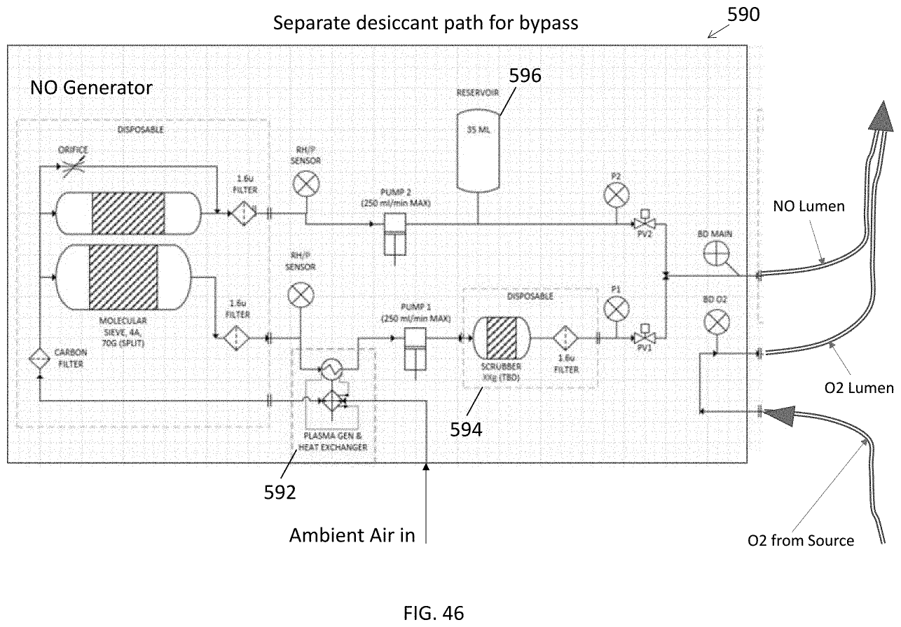

FIG. 46 depicts an embodiment of an NO generation system 590 with a pressured scrubber with pressurized bypass architecture. Reactant gas (e.g., ambient air) enters the system and passes over a heat exchanger 592 that cools the plasma chamber and/or product gas. The air flow passes through a scrubber 594 (e.g., one or more of activated carbon, potassium permanganate, molecular sieve) to remove contaminants (e.g., VOCs) and then separates into 3 separate flow paths: an undesiccated bypass flow path, a desiccated bypass flow path, and a desiccated reactant gas flow path. A fixed orifice on the undesiccated bypass flow balances the flow through the bypass desiccant chamber to provide a mixture of gas that will not produce water condensate within the system at the elevated pressures. After the two bypass gas flows merge, they are filtered and pass through a pump that will pressurize a reservoir 596. Gas flow out of the reservoir is controlled by a flow controller downstream of the reservoir (e.g., valve). A pressure sensor in fluid communication with the bypass reservoir provides pressure measurements to the system controller. The pressure within the bypass reservoir is monitored and can be altered, based on the treatment dose, breath rate, delivery device and other factors. In some embodiments, the pump fills the reservoir to a specific pressure and then stops filling the reservoir. After the reservoir pressure is partially or totally released, the pump turns on again to generate pressure within the reservoir. In some embodiments, the pump speed is modulated to a level that matches the required output flow of NO gas so that the pump can run continuously.

Continuing with FIG. 46, air also passes through a desiccant chamber designed to remove water from the incoming air. In some embodiments, all of the water is removed from the air. In some embodiments, the amount removed is sufficient to prevent condensation within the system but not complete removal of water. Gas then passes through a particle filter and a relative humidity sensor measures the humidity. In some embodiments, the relative humidity is utilized to detect whether or not the desiccant is functioning as required. In the event that the humidity is not at target, the system can alert the user to replace the desiccant and/or move to a drier environment. In some embodiments, the humidity measurement is used as an input to the NO generation controller. Humidity can affect NO production as much as 40%. When the humidity of the reactant gas is known, a NO generator can alter the plasma parameters to make up for lost production due to humidity. Reactant gas then passes through the plasma chamber where it is heated from plasma within the plasma chamber and N₂ and O₂ molecules are separated into monatomic species (i.e., ionizing the reactant gas). A portion of the ions reform into NO and some NO₂ with many ions forming N₂ and O₂ again. This product gas exits the plasma chamber and is optionally quenched (cooled) at the outlet.

This NO+air, also known as product gas, passes through a pump and collects within a reservoir filled with NO₂-scrubbing material. One benefit of this design is that the product gas is in contact with scrubber material for a long time (seconds in some cases), resulting in greater levels of scrubbing than simply passing through a scrubber. An additional benefit of pressurizing the product gas is that some scrubber materials (e.g., soda lime) remove more NO2 from a gas stream at elevated pressure. When breath is detected, a flow controller downstream of the scrubber opens to release pressurized product gas from the chamber. A pressure sensor in fluid communication with the scrubber provides the system with pressure measurements that can be used for one or more of controlling the filling of the scrubber, generating alarms if the scrubber does not fill or over-fills, estimating the flow rate out of the scrubber based on the pressure decay, estimating the flow rate out of the scrubber based on a comparison with another pressure measurement downstream of a known flow restriction within the system, and for determining when to stop flow out of the reservoir. After the target quantity of NO has been released from the scrubber, the NO flow controller closes, and the bypass flow controller opens. The bypass flow pushes the NO through the delivery device and ensures that the delivery device is filled with air between breaths.

FIG. 46 also shows flow from an oxygen source that is removably connected to the NO generator. The oxygen flow passes through the NO generator and one or more measurements are made: pressure, flow rate for example. The oxygen flow exits the NO generator in the vicinity of the NO exit point to facilitate connection of a dual-lumen delivery device that carries the NO and oxygen flow to the patient. One or more pressure sensors in fluid communication with the NO and/or oxygen connections sense pressure fluctuations within the cannula for input into a breath detection method. These same pressure sensors can be used to detect a kink or other type of partial or complete obstruction of the delivery device when flow through the delivery device is impeded. These pressure sensors can also be used to detect the absence or incomplete connection of a delivery device based on a lower than expected back flow restriction during pulse delivery as indicated by lower pressure levels required to push the pulse and/or faster pressure decay within the reservoir when the pulse is delivered. Delivery device connections are generally designed (e.g., keyed) to prevent reverse connection of the NO and oxygen lumens. In some designs, the NO delivery device can detect a reverse connection due to the reduction in back pressure when a NO pulse is sent through an oxygen lumen than a NO lumen. In the event that the delivery device is missing or installed incorrectly, a NO delivery system can alert the user of the issue with an alarm.

The rate of decay (or lack thereof) within either a pressurized scrubber or a pressurized purge gas reservoir can be utilized to detect an obstruction or kink in the delivery device. A pressure sensor in fluid communication with a reservoir can also be used to quantify the flow rate of gas entering and/or exiting the reservoir by characterizing the rate of change of pressure over time with flow rate.

Dose Control

In some embodiments, the dose of NO administered to a patient is essentially the number of moles of NO molecules delivered to the patient per unit time. The NO is delivered in pulses that are synchronized with the patient inspirations. Pulsing the NO has multiple benefits including but not limited to power savings, targeting specific parts of the lung, minimizing environmental contamination of NO/NO₂, and prolonging the service life of various components including the scrubber and electrodes. The amount of NO within each pulse is controlled by varying the NO concentration within the product gas, the level of dilution of the product gas (if any), the product gas flow rate and/or the NO pulse duration. These pulse features are controlled indirectly by the controller via control of the pump(s) flow rate, reservoir pressure(s), NO pulse timing, bypass pulse timing, and/or plasma activity (frequency, duty cycle).

In some embodiments, prior to NO generation and/or NO delivery, the controller monitors the timing of a series of breaths and calculates a mean breath rate. The controller then calculates and/or looks up a NO mass per breath based on the target patient dose and mean respiratory rate. After determining the starting point for dose per breath, the controller starts dosing according to the breath rate. The breath rate is tracked with a moving average and dose per breath is altered over time to maintain the target dosing run rate. In some embodiments, the breath rate is calculated as a weighted average with more weight applied to more recent breaths. One or more of the concentration of NO, moles of NO, reservoir pressure, flow rate, NO pulse duration, and purge pressure can be varied with each pulse to maintain a target dose rate.

In some embodiments, a NO generation system determines a target NO production rate (e.g., ppm·lpm or μl/min) based on the prescribed dose (e.g., 6 mg/hr). The controller sets the plasma activity (e.g., duty cycle) based on measurements of the system (e.g., temperature, pressure, humidity), the reactant gas flow rate and a look-up table for production in ppm-lpm. Variations in the low flow rates don't impact production rate substantially, hence some embodiments of a NO generation system do not compensate for variation in concentration within the product gas based on flow rate. These systems only consider the variability in NO loss due to changes in transit time through the system resulting from variation in breath rate. Overall, in these systems, the breath rate doesn't actually affect the NO production setting, the system just uses the breath period (the reciprocal of breath rate) to determine how much NO to release.

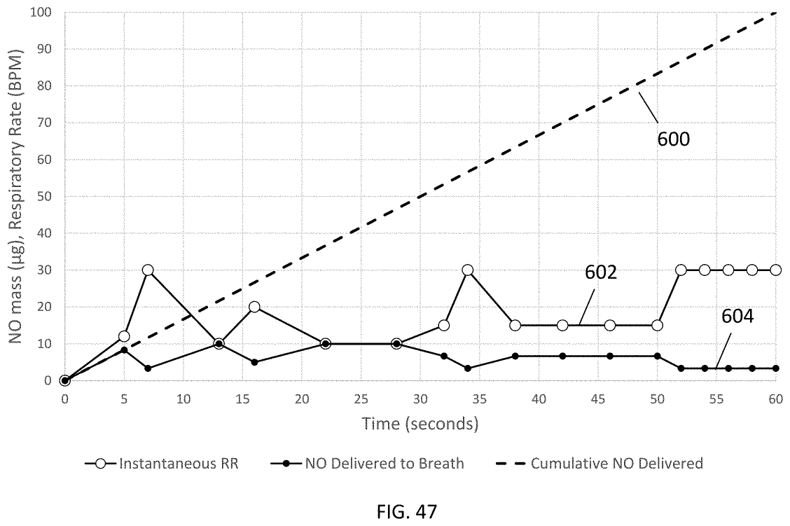

FIG. 47 depicts an exemplary graph showing one minute of an exemplary treatment of a patient with a dosing rate of 6 mg/hr (i.e. 100 ug/minute). The X axis is time spanning 1 minute. The Y axis depicts NO mass delivered in micrograms and respiratory rate in breaths per minute (BPM). The cumulative dose (dashed line 600) linearly increases over time as pulses of NO are delivered so that 100 ug of NO have been delivered in 60 seconds. The instantaneous respiratory rate (line 602) is calculated with each breath based on one or more recent breath periods. In this example, the instantaneous respiratory rate is based on the duration of a single breath and varies from 10 to 30 breaths per minute. The amount of NO delivered (line 604) is inversely related to the instantaneous respiratory rate. In other words, the moles of NO delivered to a particular breath is proportional to the amount of time since the previous breath. This variation in NO delivery per breath accommodates the variation in breath period to maintain an accurate dosing rate. In a constant concentration NO system, NO mass delivered is varied by varying the flow rate and duration of the NO pulse (i.e., the pulse volume). In some embodiments, longer breath periods are associated with longer NO pulses, for example.

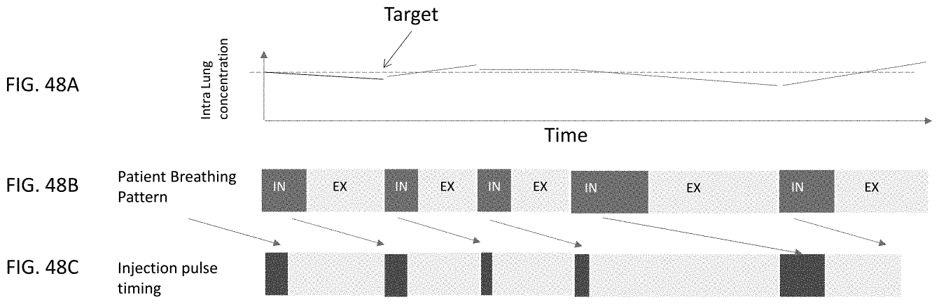

FIGS. 48A, 48B, and 48C depict graphs showing an embodiment of a pulsed gas (e.g., NO) dosing strategy that targets a particular intra-lung concentration. FIG. 48A illustrates the target intra-lung concentration (straight, dashed horizontal line) with actual lung concentration (shorter, sloping lines). FIG. 48B depicts the patient breathing over time. Dark regions represent inhalation and light regions represent exhalation and any pause between breaths. It should be noted that the duration of inspirations varies from breath to breath. A gas delivery device does not know when a current inspiratory event will end. Nor does it know when the next inspiratory event will occur. The device can determine the breath period and inspiration duration for one or more prior breaths and may use this information to predict injection pulse timing on a subsequent inspiratory event. In some embodiments, data on prior breaths are stored in device memory. FIG. 48C shows a dosing scheme whereby a gas delivery system doses a current breath as if it was the prior breath. Dose is delivered during the darker regions. Higher levels of dosing for a particular breath can be achieved through one or more of gas concentration, gas flow rate and pulse duration. In one embodiment, the medical gas concentration and flow rate are constant and the only variable is gas pulse duration. Arrows from the inspiratory event of one breath to the gas delivery timing of the subsequent breath highlight that the system dose activity is one breath behind the patient. In the depicted example, the second and third inspiratory events have similar duration. Thus, the NO dose for the 3^{rd} breath is an accurate prediction of the amount of NO required. This accuracy is reflected in the horizontal region in the intra-lung concentration plot during the third breath period. In another embodiment, a gas delivery system determines the dose (i.e., one or more of concentration, duration, moles of NO) for a current breath as a function of the breath period and/or inspiration duration of one or more prior breaths. In this way, the system is always one breath behind in dosing but can maintain the intra-lung NO concentration within an acceptable level. In other embodiments, a gas delivery device consistently delivers a corresponding dose two or more breaths behind a particular inspiration.

In some instances, a breath is not detected. Making up for missed breaths can be done by lengthening one or more subsequent pulses and/or increasing the concentration of one or more subsequent pulses. These increases in pulse NO content are limited by the volume of the reservoir and the NO production capability of the device.

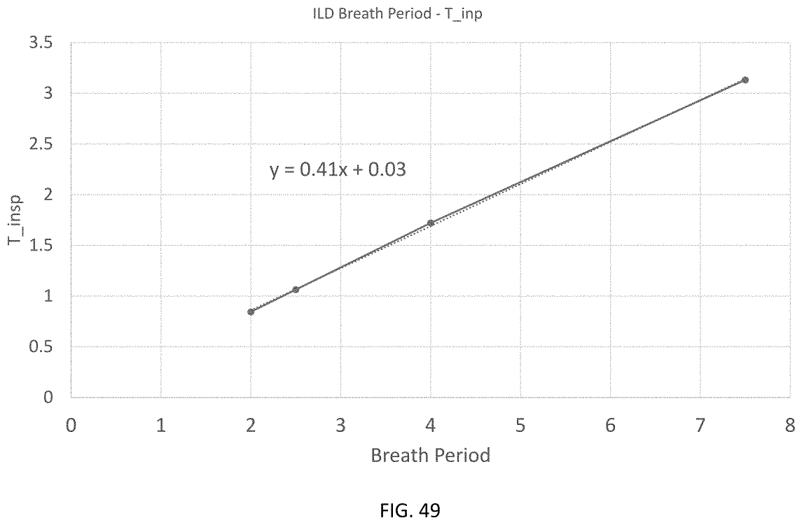

The duration of inspiration is a fraction of the breath period. Observation of multiple patient breath profiles and patients has revealed that the inspiration duration for a given breath period (or breath rate) is very repeatable over the range of breath rates for a given patient. FIG. 49 depicts an exemplary relationship between inspiratory duration (y axis) and breath period (x axis). In some embodiments, the relationship is linear across breath durations, with the inspiratory duration being 41% of the breath period. More complex regressions of this relationship could be used, but a linear approximation is usually sufficient for a given patient. In treating a specific patient, a drug delivery device can predict the optimal period and amount to dose the patient. The optimal period varies between patients and by disease type and state.

In some embodiments, the relationship between inspiration duration and breath period can be determined for a given patient in the clinic prior to sending a patient home with the device. In some embodiments, the device utilizes the sensed timing of inspiration, beginning of exhalation and end of exhalation over a range of breaths to build a relationship between inspiration duration and breath duration for a specific patient. This relationship is then stored in system memory and utilized during treatment. In some embodiments, the relationship is periodically updated with new data.

In some embodiments, the system calculates a NO pulse width based on the expected inspiratory duration, as predicted by the breath rate. In some embodiments, the NO delivery system utilizes a mathematical relation and/or model between the breath period and inhalation period. The NO pulse width is a function of the inhalation period. In one specific embodiment, the NO pulse width is a linear function of breath rate. In some embodiments, the parameters (e.g., coefficients, exponents, etc.) of the relation/model are updated each sequential breath. This enables a NO delivery system to adjust to the patient breathing patterns as the patient respirations change due to the patient environment, patient activity, delivery device interface with patient (e.g., nasal prong insertion level), and patient condition. In some embodiments, at the beginning of treatment, the system performs the calculations above for one or more breaths before actual delivery of NO begins.

Pulsed NO, or any therapeutic gas, can be delivered during the entire inspiratory event or a portion of the inspiration. In some embodiments, depending on the patient condition, it can be advantageous to dose an initial portion of a breath and not dose a latter portion of a breath. This is because some health conditions cause the unhealthiest regions of the lung to be filled with air last. Dosing unhealthy regions of the lung with NO can promote blood flow in a region that is not as effective in oxygen uptake and worsening intrapulmonary shunt in some instances. In some embodiments, a NO generation system delivers the NO pulse within the first ⅓ of the inspiration event (time and/or volume). In some embodiments, the NO pulse is delivered within the first ½ of the inspiration, for example. In some embodiments, the NO or therapeutic gas pulse can be spread out over a longer period of time (pulse stretching) at a slower flow rate to overlap with more of the inspiratory event and deliver the NO or therapeutic gas into more parts of the lungs. In some embodiments, the same pulse duration can be selected to correspond to each inspiratory breath, for example 250 msec, to dose an entire breath.

Delivering a NO pulse early in the breath can provide for improving ventilation/perfusion (V/Q) matching to optimize oxygenation in patients with particular lung disease conditions. Other patients (e.g., those with pulmonary infection) may benefit from oxygen and/or NO delivery across a larger portion of inspiration, dosing later-recruited regions of the lung, airway and respiratory tract. A problem arises in that it is challenging to know for an individual patient how much of the inspiratory volume is well ventilated lung and how much is poorly ventilated. If lung regions with V/Q ratios>1 are vasodilated and the V/Q ratio moves towards a value of 1.0, that is beneficial. However, if V/Q is reduced below 1.0, or low V/Q regions are further dilated, then the patient's oxygenation will worsen.

Gas that is inhaled towards the end of inspiration travels into the patient anatomical dead space (a volume of approximately 150 ml in an adult), but not into the alveolar volume. An exemplary patient could have a tidal volume of 500 mls and an anatomical dead space of 150 mls, representing 30% of the tidal volume. In practice, the anatomical dead space represents 20% to 40% of the tidal volume, depending on the patient size and the tidal volume. One method of estimating anatomical dead space is to calculate it as 1 ml per lb of ideal body weight. The residence time within the anatomical dead space is a few seconds at most depending on respiratory rate and NO uptake within the anatomical dead space is incomplete. It follows that a significant proportion of NO that is dosed into the anatomical dead space and any NO₂ that is formed will be exhaled into the environment. Over time, environmental NO will oxidize into NO₂ which can accumulate if there is insufficient environmental air exchange in the environment e.g. a nonventilated space. In one embodiment, the anatomical dead space is intentionally not dosed with NO to mitigate against exhalation of NO and NO₂ to the environment. In one embodiment, a latter portion of the inspiratory volume is intentionally not dosed with NO to prevent dosing of the anatomical dead space and loss of NO to the environment.

In some embodiments, an oxygen generation/delivery system and/or a NO generation/delivery system can personalize the duration, volume, dose, flow rate profile, concentration and timing of the delivered gas pulse for each patient. In some embodiments, patient SpO₂ and/or methemoglobin measurements are used to quantify the effects of various NO pulse parameters and can serve as feedback to the dose controller. In some embodiments, this characterization is done in real time as the patient wears and utilizes the device. In other embodiments, this characterization of a patient is done periodically when the patient is at rest with quiet regular breathing. The system monitors the patient and then sets the personalization parameters within the NO generation/delivery device accordingly and those parameter settings are utilized for the remainder of the treatment period (days, weeks, months, years) or until the next patient characterization test. In some embodiments, the device varies the NO dose while monitoring SpO2 to determine the NO dose levels to be utilized. In some embodiments, the NO dose level is selected associated with the highest SpO2 level. In other embodiments, the NO dose level is selected as the minimum NO dose that achieves a specific increase in SpO2.

In some embodiments, the device settings are configured in a clinic with the patient. The device either automatically or through manual control sweeps through pulse personalization parameters (e.g., NO moles, durations and delays) to achieve an optimal SpO₂ value. In some embodiments, the sequence is as follows 1) Deliver the shortest pulse as early as possible, 2) Step-wise, increase the duration of the pulse until the maximum is reached, 3) Step-wise, delay the maximum length pulse. The system maintains the performance of the system at each step for a set amount of time to confirm compatibility with the patient (e.g., 1 minute), as measured by SpO₂ and MetHg levels. If at any point, the SPO2 begins to decline, the procedure is stopped and the device is set at a prior setting that represents the longest pulse that the patient was compatible with. Longer pulses ensure that the NO concentration is as low as possible, thereby reducing NO oxidation into NO₂. Longer pulses also dose the largest portion of the breath, thereby affecting more lung tissue than short pulses. In some embodiments, short pulses are administered with step-wise increases in delay to determine how late into the breath NO can be administered without a deleterious effect, thus establishing the pulse delivery window. Once the trailing edge of the dose window has been established, the NO delivery device delivers pulses that begin as early as possible in the breath and terminate at or just prior to trailing edge of the dose with the pulse concentration and delivery flow rate selected to deliver the prescribed amount of NO over time.

In some embodiments, a NO pulse is defined by a duration, a flow rate, and a concentration. The volume of a pulse is equal to the mathematical product of the average flow and pulse duration. This volume of gas delivers a finite number of NO molecules over a finite duration of time with a specific transit time. In some embodiments, pulses and purge gases are delivered at a maximal combined rate that patients can tolerate to minimize transit time and NO oxidation to NO₂. When the NO pulse flow rate and duration are kept constant, the only variable left for achieving a target dose setting is NO concentration. NO pulse concentration can be varied by either making NO product gas with less concentration or diluting NO product gas with another gas. Dilution of a NO pulse can be done continuously or intermittently in a pattern, such as an alternating pattern (e.g., NO, Purge, NO, Purge, etc.)

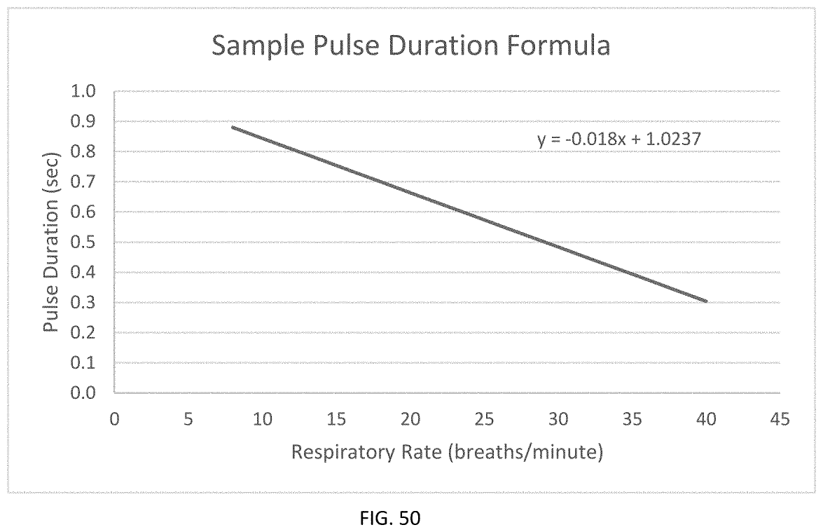

For some patient applications, like lung diseases (e.g., ILD, COPD), it is preferable to dose lung that still participates in gas exchange. The termination of the NO pulse should be before inspiration into the sickest lung that does not participate in gas exchange and the anatomical dead space (no additional clinical benefit) and/or the beginning of reductions in patient SpO₂ based on dosing the sickest lung (adverse clinical impact). In some embodiments, dosing a range of 30-60% of the inhaled volume is found to be effective in improving V/Q matching for sick lung tissue while avoiding dosing of the sickest lung. In some embodiments, the target NO pulse duration is calculated by the treatment controller as a function of one or more of respiratory rate, and inspiratory duration. For example, FIG. 50 is an exemplary graph depicting the relationship between pulse duration and respiratory rate. This information is stored in either an equation or a look-up table within the NO controller memory and processed by the NO controller software. In some embodiments, the relationship between NO pulse duration and respiratory rate is patient-specific (i.e., determined in the clinic with a patient based on their condition characterization of the patient's response to NO, and characterization of their respiratory patterns (e.g., measurement of their tidal volume as a function of respiratory rate)).

In some embodiments, the duration of the medical gas pulse is based on an assessment of patient breathing patterns. A moving average of prior inspiratory event durations is used to characterize the current patient breathing pattern and apply medical gas (NO, oxygen or other) to a target range of the inspiratory volume. The duration of inspiration is determined as the difference in time between beginning of inspiration and end of inspiration. In one embodiment, the beginning and end of inspiration events are detected as zero-crossing points of the inspiratory pressure signal. In one embodiment a medical gas delivery device detects the beginning of inhalation and end of inhalation for each breath in order to calculate an inhalation duration and then maintains a moving average of inhalation durations. Then, the device controller determines the medical gas pulse delay and pulse duration in order to optimally dose a portion of the inspiratory volume. In some embodiments, the amount of inspiratory volume dosed is 30-60% of the inhaled volume.

In some embodiments, pressure measurements within the delivery device are correlated with inspiratory flow measurements to customize NO delivery treatment for a specific patient and delivery system. In some embodiments, nasal and mouth breathing are characterized separately. In other embodiments, this characterization is done for varying levels of nasal cannula insertion. By calibrating delivery device pressure as a proxy for inspiratory flow rate, a NO delivery system can estimate inspiratory flow rate and integrate that flow rate into an inspiratory volume. In some embodiments, the NO pulse volume, flow rate, delay and concentration are customized based on the inspiratory flow rate, respiratory rate and tidal volume to improve dose accuracy and placement within the target zone within the respiratory tract.

In some embodiments, a NO delivery device delivers a flow of gas from the pressurized scrubber and the bypass flow simultaneously, enabling the system to spread out the NO pulse over a longer period of time. This approach can be advantageous because it is capable of delivering NO to the patient over a larger duration of the inspiratory event, thereby dosing a larger portion of the inspired volume of gas. By flowing bypass gas at the same time as NO-containing gas, the NO gas can be diluted early, and transit times are minimized, both contributing to minimizing NO oxidation and inspired NO₂ levels. This same approach can apply to chemical and tank-based NO delivery systems as well. In an embodiment shown in FIG. 29, a NO delivery system maintains a target NO pulse flow rate (e.g., 5 lpm) by delivering a combination of NO gas and dilution flow (bypass gas in the subject example above), the respective proportions of which are selected to dose a particular subset of the inspiratory volume. In some embodiments, the ratio of NO gas flow rate to inspiratory gas flow rate is a constant ratio. In some embodiments, after introducing the entire amount of NO for a NO pulse to the delivery system, the NO delivery flow controller closes, and the purge gas flow controller opens further to maintain a constant flow rate within the delivery system, as shown in FIG. 29.

In some embodiments, NO is introduced into the delivery system as multiple discrete pulses during a single inspiratory event to spread out the NO dosing over a larger portion of the breath. In one embodiment, discrete NO pulses are merged with a dilution and/or bypass flow through the delivery system that can be continuous throughout the inspiratory event (FIG. 29). In some embodiments, the bypass flow is pulsed, out of phase with the NO pulses to create a NO pulse train within the delivery system (FIG. 31).

FIG. 35 depicts a pulse elongation approach whereby the medical gas (NO in this case) is flowed at a fast rate to prime the delivery device. The flow of NO gas from the scrubber ends when the target volume of NO of known concentration (NO mass) has been expelled from the scrubber. Then, the flow rate of the medical gas is slowed to stretch the timing of the pulse to the target duration (400 msec in this case). Purge gas is flowed at the same flow rate for continuous medical gas delivery until the medical gas within the delivery system has all be delivered. The dashed line depicts the cumulative volume of medical gas delivered to the patient during one gas pulse while the solid line depicts the gas flow rate within the delivery system. As the delivery device is primed, gas flow rates are higher and the slope of the cumulative volume is steeper. As medical gas begins to exit the delivery system and enter the patient, the rate of flow within the system slows, as shown by the decrease in the solid line value and decrease in slope in the dashed line. As the system transitions to purge gas, the flow rate and cumulative volume slope continue at their previous values. Once all of the medical gas has been delivered, the flow through the delivery system reduces to zero.

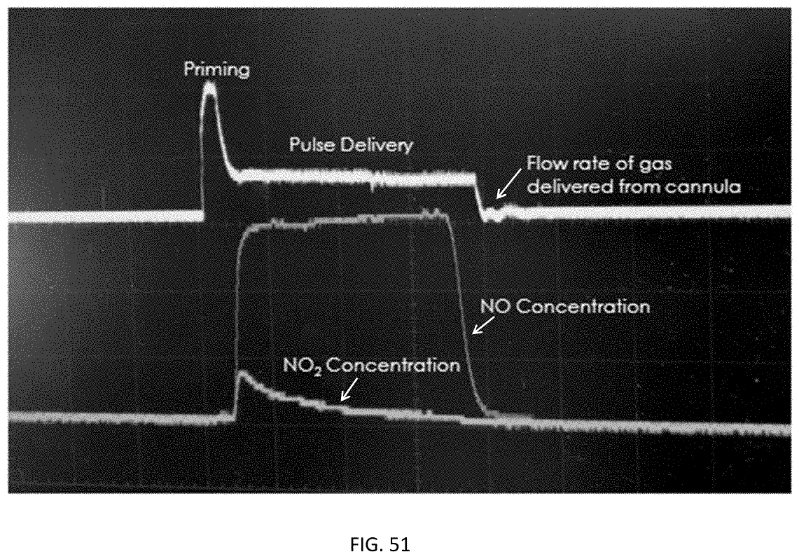

FIG. 51 depicts an exemplary graph showing the relative timing of NO and NO2 delivery from a pressured scrubber NO delivery system. The upper curve presents flow through a delivery device beginning with a fast flow rate to prime the delivery device with NO, followed by a slower flow rate that delivers a target quantity of NO (e.g., moles) over a target time period. This priming step typically takes 50 to 100 msec. The middle curve represents NO concentration detected at the patient. It can be seen that NO delivery out the proximal end of the delivery device begins after the priming pulse. NO2 concentration at the patient is represented by the lowest curve. It should be noted that the NO and NO2 curves are not on the same scale. The NO2 scale is 10× greater on the plot so that it can be seen. The NO2 level begins at a higher level and ends at a lower level. This is because the first gas to arrive at that the patient was located between the scrubber and product gas flow controller between breaths and was not scrubbed since the prior breath. As gas flow continues, gas is delivered directly from the scrubber through the delivery device to the patient with lower NO2 levels.

Operating Characteristics

Delivery System Purge

Tank-based NO delivery systems typically maintain a column of NO within the delivery device and deliver NO to the patient in a first in/first out fashion. Two issues stem from this approach. First, NO within the delivery system can oxidize as it waits to be delivered. This is addressed in tank-based systems by delivering NO in a balance of nitrogen so that there is no oxygen to oxididize with. Second, NO within the delivery system can leak out the proximal end (i.e., the patient end) of the delivery system due to its pressure and a strong diffusion gradient since NO is not normally present in the patient environment. To address these issues, some embodiments purge the cannula of NO between breaths with non-NO gas. Purging consists of displacing all or nearly all of the nitric oxide-containing gas within the delivery device with a more inert gas. The frequency of purging can vary. In some embodiments, purging is done after every pulse of NO is delivered to the patient. This prevents NO+air from aging within the delivery device (e.g., cannula) which results in elevating NO₂ levels, decreasing NO concentration, and more dose delivery uncertainty. Other embodiments, purge the delivery system intermittently, based on breath count, oxidation time of the gas in the delivery system, NO concentration and other parameters. Delivery device purging eliminates the risk of NO leaking out the end of the delivery device between breaths which causes environmental contamination and dose uncertainty. In some embodiments, the purge gas consists of one or more of air, nitrogen, oxygen and/or reactant gas. The process of delivery device purging, as with most if not all pneumatic activity, is directed by the treatment controller. The treatment controller regulates the pump operation pre-purge gas reservoir to ensure adequate pressure within the purge gas reservoir. Then, at the appropriate time, the treatment controller controls the flow controller downstream of the purge reservoir to release purge gas. In some embodiments, a specific volume of purge gas is released as indicated by a specific change in pressure in a known volume. In some embodiments, purge gas is maintained at or above a minimum pressure and released for a specific amount of time. In some embodiments, the flow of purge gas is measured by the treatment controller with a purge gas flow sensor and integrated over time to track the volume of gas delivered.

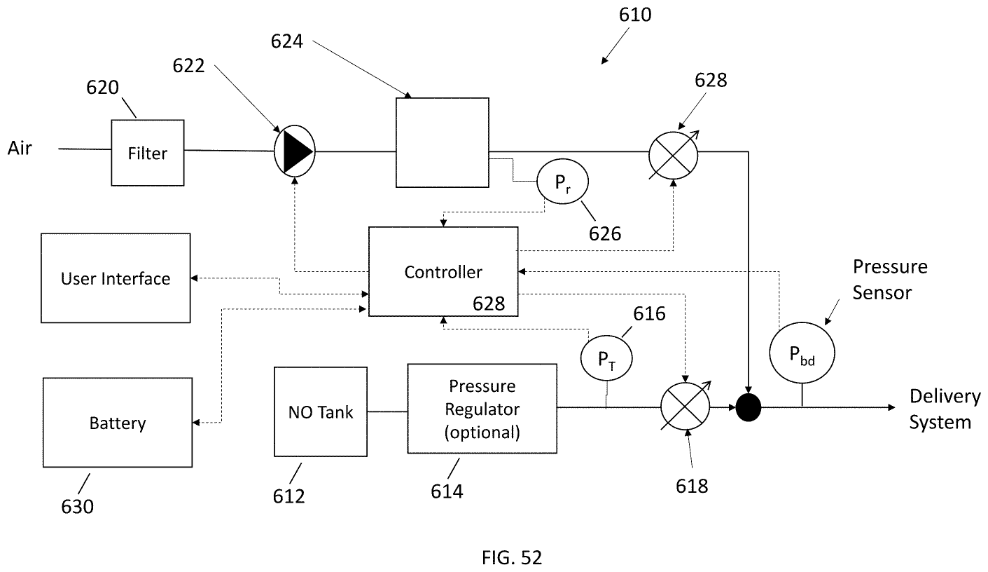

FIG. 52 depicts an exemplary embodiment of a tank-based NO delivery system 610 with a purge feature. High pressure, high concentration NO is stored in a tank/cylinder 612. The pressure is optionally reduced with a pressure regulator 614. The pressure level of the NO gas is measured using a pressure sensor 616 (Pₜ) as an input to a flow controller 618. In some embodiments, the flow controller is simply a binary valve. In other embodiments, the flow controller is a mass flow controller that utilizes the upstream pressure and known gas mixture to deliver a known mass flow rate of NO.

Continuing with FIG. 52, air is drawn into the system through a filter 620 by a pump 622. In some embodiments, the air filter is 20 micron. Air accumulates in a reservoir 624 where the internal pressure (Pᵣ) is measured by a pressure sensor 626. A flow controller 628 downstream of the reservoir is utilized to purge the delivery device with air. Operation of the system is managed by a controller 628 (e.g., microcontroller, FPGA, Arduino) that collects sensor information and inputs from a user and controls the pump and flow controllers. The entire system is powered by a battery 630. In some embodiments, the battery is replaceable and/or rechargeable.

Pulse Queuing

In some embodiments, a pulse is queued within the delivery device prior to the next breath. Pulse queuing involves positioning a volume of NO-containing gas within a delivery device prior to the injection time. This is done to decrease the amount of time that NO is aging within the delivery device and to decrease the transit time for the leading edge of the NO volume to reach the patient. The amount of volume queued within the delivery device can vary from very small (a few ml) to larger (tens of ml) to the internal volume of the delivery device, depending on the volume of NO gas planned to delivery.

Pulse queuing involves introducing a known volume of NO to the delivery device that is approximately equal to or less than the internal volume of the delivery device. Queuing is typically started prior to breath detection but may not be complete before breath detection. In some embodiments, the NO pulse volume to be delivered to the patient is greater than the internal volume of the delivery device. In this case, the queued NO volume is equal to or slightly less than the internal volume of the delivery device and effectively primes the delivery device with NO. In other cases, the NO pulse to be delivered has a much smaller volume than the internal volume of the delivery device. In this case, the NO pulse volume is equal to queued NO volume. The queued NO volume is introduced to the delivery device and then pushed down to the patient end of the delivery device using a non-NO-containing gas, such as air or nitrogen. In either case, the NO pulse is generated as late as possible to minimize NO₂ formation while being sufficiently early to permit breath detection (in cases where NO pulse queuing could interfere with breath detection) and complete delivery to the breath.

The NO controller can determine the time to queue the NO pulse in a myriad of ways. In some embodiments, pulse queuing begins coincident with a respiratory event (e.g., end of inhalation, end of expiration). In some embodiments, a time delay is added to the time of a respiratory event (e.g., end of inhalation+1 second) in determining the time to initiate pulse queing. In some embodiments, the time delay applied is a function of the patient breath rate (e.g., beginning of exhalation+20% of the moving average of breath period). This is accomplished by recording the time of two or more respiratory events in a series of respirations. For example, in one embodiment, the treatment controller records the time for every inspiration event detected. In another embodiment, the treatment controller records the time for two exhalation events 5 breaths apart. Then, then treatment controller determines an average respiratory period. This is done by calculated the mean respiratory period when each respiratory event is recorded. It is calculated as the duration of time divided by the number of respiratory events when only and beginning and end time are recorded. Once a breath period is determined, it is multiplied by a fraction to determine the delay. In some embodiments, the fraction is a fixed number programmed into the system. In other embodiments, the fraction is a function of the breath period. In some embodiments, the fraction is determined by the treatment device based on a characterization of the patient breathing patterns (e.g., typical Inspiration to expiration ratio across a range of respiratory rates).

After determining the correct time to que an NO pulse in a delivery device, a treatment controller queues the pulse in various ways, depending on the pneumatic architecture of the system. In a linear architecture, for example, the treatment controller directs the pump to generate flow and the plasma chamber to generate NO. NO is generated for the correct amount of time to generate a target number of NO molecules, including excess in anticipation for losses in the system. The system then turns off the plasma and continues operating the pump to push the NO bolus to the queued location in the delivery device. In another embodiment with a pressurized scrubber/pressurized bypass architecture, the treatment controller releases a target amount of NO from the scrubber and an appropriate amount of purge gas to que the NO pulse within the delivery system by controlling the NO and bypass gas flow controllers.

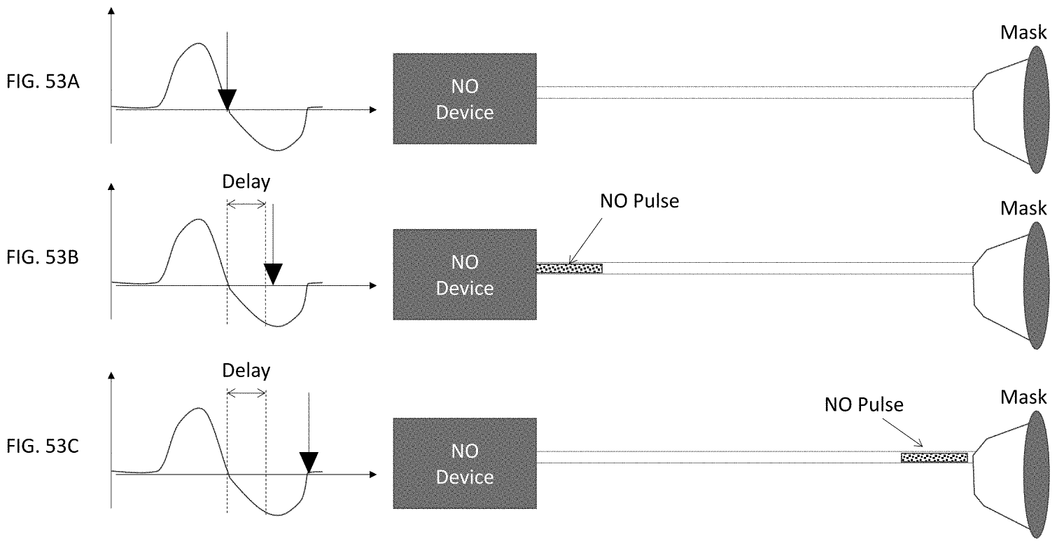

FIGS. 53A, 53B, and 53C depict an example of pulse queueing with a system that queues a NO pulse within the delivery device based on a delay from the end of inspiration. The NO pulse in this example is smaller in volume than the delivery device. A downward arrow on the inspiratory waveform plot shows the time point depicted, which is at the end of inhalation with the delivery device is devoid of NO. FIG. 53B shows that after an intentional delay with respect to the end of inspiration, the pulse is introduced to the delivery system. The time point of FIG. 53B is just after the delay so that NO has started to enter the delivery system. NO is pushed into the delivery system until the target number of moles of NO have been introduced to the delivery system plus any additional NO required to make up for expected losses (e.g., leak, oxidation, etc.). The pulse is then pushed to the proximal end of the delivery system with non-NO gas (e.g., bypass gas, reactant gas, air, nitrogen, oxygen, inert gas (e.g., argon). FIG. 53C shows the NO pulse after it has been pushed to the end of the delivery device. The pulse will remain in this location until the pulse is delivered, which is typically after a breath detection event. Delivery of the pulse involves pushing the NO pulse with additional gas (typically non-NO gas). In some embodiments, a new NO pulse is introduced to the delivery system as an old pulse is delivered out of the delivery system. In other embodiments, multiple NO pulses may be stacked in a delivery system, separated by purge gas. This is most applicable to very long delivery systems where the transit through the delivery system would take longer than the breath period.

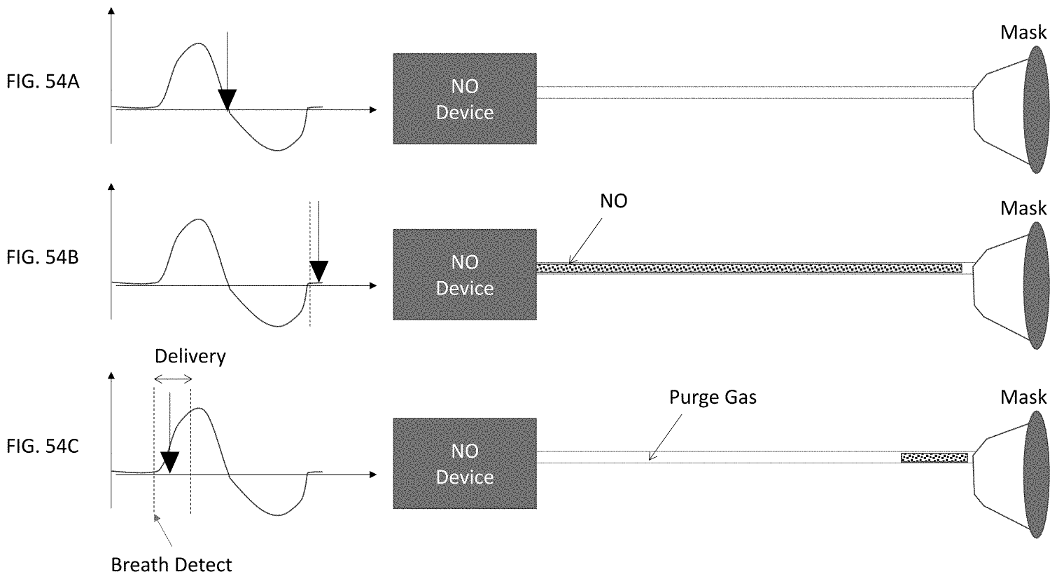

FIGS. 54A and 54B depict pulse queuing examples where the NO pulse to be delivered has greater volume than the delivery device. In this example, the pulse is queued at the end of expiration, however it could be queued at other time points within the respiratory cycle. FIG. 54A depicts the delivery system filled with gas that does not contain NO. FIG. 54B depicts the NO controller filling the cannula with NO containing gas. In some embodiments, the NO containing gas is product gas. In some embodiments, it is diluted product gas. In some embodiments, it is NO-containing gas from a tank. Once the leading edge of the NO containing gas reaches the proximal (patient) end of the delivery device, the NO controller stops the flow of NO, having effectively primed the delivery device. FIG. 54C depicts the bolus of NO being delivered to the patient by pushing the NO gas through the delivery device with inert, non-NO containing gas. After the NO pulse is completely delivered, the system returns to the status depicted in FIG. 54A.

In some embodiments, the delivery device is lined with a scrubbing material so that the pulse is scrubbed as it waits to be delivered.

In some embodiments, turbulence-inducing features within the delivery device promote mixing and improve overall scrubbing of the product gas. In some embodiments, a static mixer is used. In other environments, scrubber material is formed in the shape of a static mixer.

Controller Design

Internal Chassis

In some embodiments, a NO generation device includes an internal manifold made from an elastomeric material. This approach can leverage the material properties of an elastomeric material to mold in undercuts and cavities. For example, a reservoir volume can be molded into an elastomeric manifold without the use of fittings which add weight and potential leak points. An elastomeric manifold can also add shock-absorption and vibration-tolerance to a system. In some embodiments, sensors are over-molded with the elastomeric material to provide gas-tight seals and simplify mounting. In some embodiments, silicone is used as an elastomeric material, owing to its significant shock absorption properties and NO compatibility.

Pump Selection

Gas propulsion through a NO generation system is typically generated with a pump. Any type of pump can be used, such as diaphragm, screw, scroll, piezo, gear, piston, centrifugal and peristaltic. Each type of pump has their plusses and minuses related to energy consumption, acoustic noise, mechanical vibration, flow pulsatility, mass, pressure generation, ability to rapidly change speeds, flow rate range, etc. In an ambulatory application, sound generation and vibration are key contributors to the user experience. In some embodiments, a pump with piezoelectric actuation is utilized for its ability to silently provide a non-pulsatile flow of gas (reactant or product) through the system.

Diaphragm pumps generate pulsatile flow. The pulsatile flow results in pressure fluctuations within the plasma chamber. NO production in a plasma is affected by reactant gas pressure. In some embodiments, plasma activity is controlled to occur at one or more particular pressure levels in the reactant gas pressure cycle to improve NO production accuracy. In some embodiments, reactant gas pressure is continuously monitored and plasma activity is continuously adjusted for the reactant gas pressure value in real time.

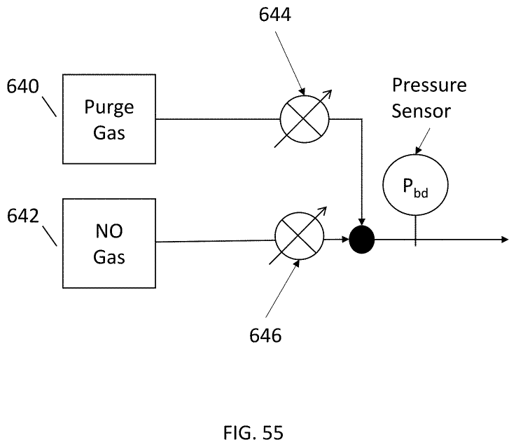

It should also be noted that many of the architectures presented herein can utilize compressed gas to function. For example, FIG. 55 depicts an embodiment of a system that utilizes a compressed gas canister of purge gas 640 and a compressed gas cannister of NO gas 642. A controller (not shown) interfaces with flow controllers 644, 646 that control the flow exiting the gas canisters. This approach enables a NO delivery system to deliver a range of concentrations of NO gas by blending the NO gas with the purge gas. The gas can be delivered at any point within the breath for any duration. After the target number of moles of NO have been introduced to the delivery system for each breath, the purge gas pushes the NO through the delivery system, eliminating the potential for NO to oxides or leak into the environment between breaths. Purge gas can be one or more of oxygen, air, nitrogen, or other gases that do not contain NO.

Chamber Cooling

Generation of NO with electricity can result in heating of the plasma chamber. Unchecked, this heat can accumulate and cause damage to the NO generator, and/or burn a patient or clinician handling the device. Thus, it is important to cool the plasma chamber in some applications. In one embodiment, cooling is done with forced air from the environment using a fan, or equivalent. In other embodiments, internal gas flow (reactant gas, purge gas, and/or product gas) is used to convectively cool the plasma chamber. Heating these gases has the added benefit of decreasing the propensity for condensation when humidity is present.

FIG. 40 depicts an exemplary design that cools a plasma chamber convectively with purge gas. As shown in FIG. 40, the plasma chamber is at least partially covered by a purge gas flow conduit. In some embodiments, cooling fins on the plasma chamber increase thermal transfer. In some embodiments, the outlet port of the plasma chamber is metallic to increase heat transport. In some embodiments, the direction of purge gas flow is opposite that of product gas for cooling efficiency. In some embodiments, the flow path of the purge gas has an electrically conductive layer or is entirely constructed from electrically conductive material so that it can serve as a Faraday cage to shield other parts of the system and/or external devices and Users from electromagnetic interference. In some embodiments, the volume around the plasma chamber filled with purge gas serves as the pressurized reservoir for the purge gas. In some embodiments (not shown), purge gas flows over the plasma chamber prior to passing through the pump and getting pressurized.

Another feature of FIG. 40 is that it utilizes a flow sensor to measure the flow rate of gas delivered to the delivery device. In this embodiment, the flow sensor measures the flow rate of product gas, purge gas and the combination thereof. Other embodiments have flow sensors located in the product gas line and bypass line to independently measure the flow of the product gas and purge gas, respectively. The treatment controller can then sum the product gas and purge gas flows to know the total flow to the patient. The gas flow measurements can be utilized by the treatment controller as feedback to the gas flow controllers to control the flow rate of the respective gases. The flow sensor can also serve as input to an alarm if flow is not occurring when it is expected, as would happen when there is a kink or obstruction of the delivery device.

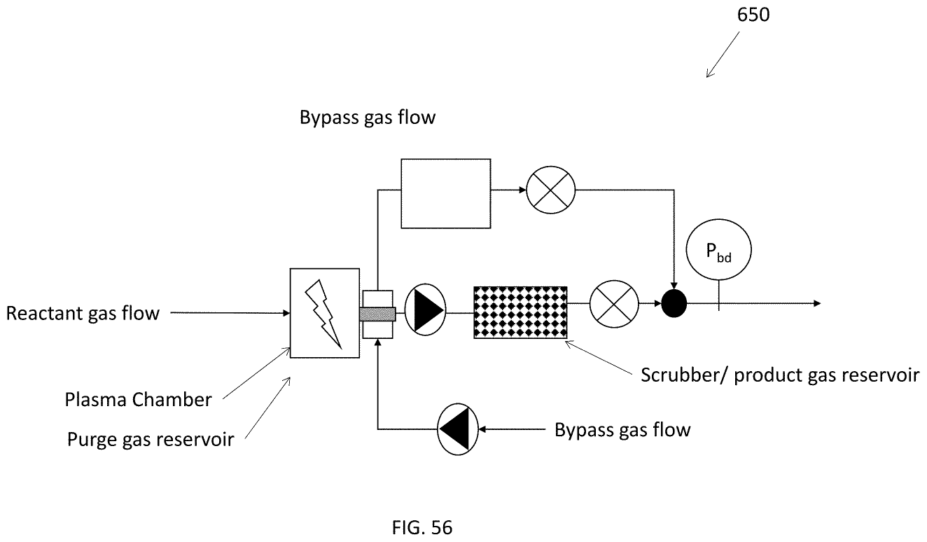

FIG. 56 depicts a NO generation system 650 that utilizes the purge gas flow through a heat exchanger to pull heat out of the product gas after it leaves the plasma chamber. This can provide benefits in pump longevity by flowing cooling gas through the pump. A further benefit is that cooler product gas flowing through a soda lime scrubber will remove less moisture from the soda lime, thereby prolonging the scrubber service life.

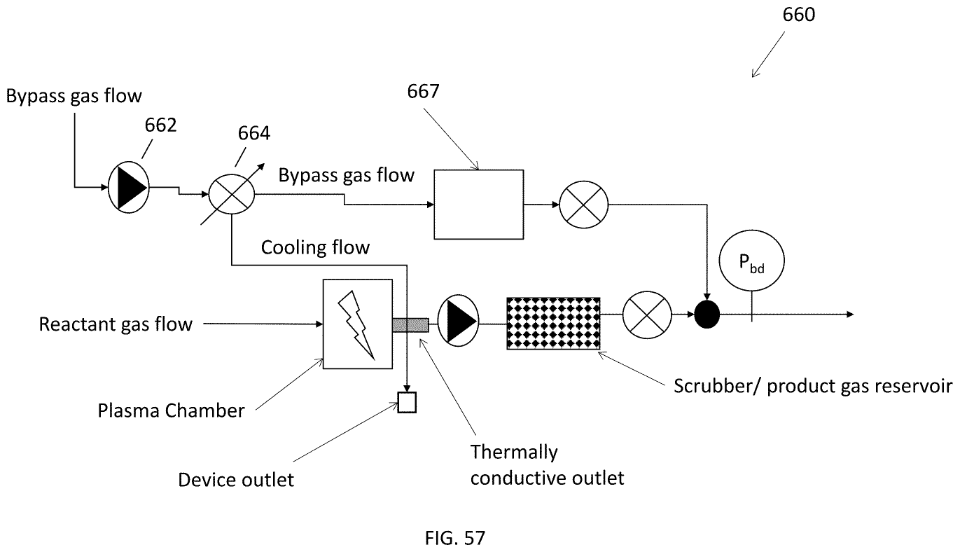

FIG. 57 illustrates an embodiment of a NO generation system 660 that manages temperature by product gas cooling that utilizes purge gas. A pump 662 draws purge gas into the system where it flows into a flow diverter valve 664. In some embodiments, the diverter valve delivers a fixed ratio of flow and in other embodiments, the ratio is variable. One portion of the flow goes to the bypass reservoir 667, where it accumulates until the next purge gas pulse. The other portion of flow travels through a heat exchanger to remove heat from the product gas.

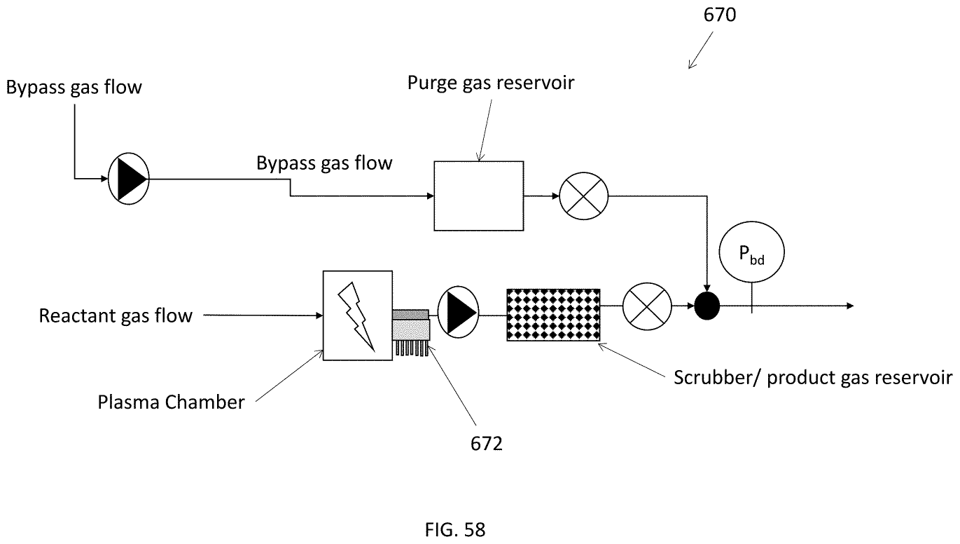

FIG. 58 depicts an embodiment of a NO generation system 670 that manages temperature within the product gas. Product gas flows through a heat exchanger 672 with cooling fins as it exits the plasma chamber. The cooling fins transfer heat to the surrounding air. In some embodiments, the surrounding air is convectively flowed over the heat exchanger for increased thermal transfer.

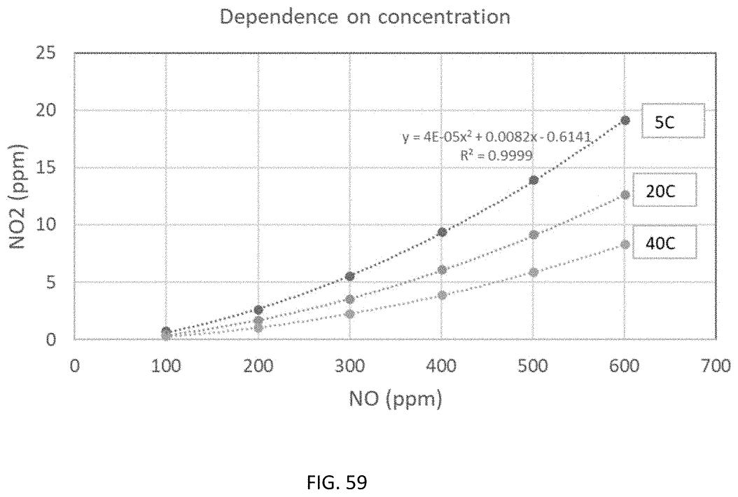

Thermal management within an NO generation device is important to maintain the longevity of internal components of the system. Temperatures within the system must be kept below the operating limits of the internal components. In some embodiments, however, the internal temperatures are kept as high as the internal components can tolerate while still satisfying their service life requirements. This is because NO oxidation rate decreases with temperature. Hence, a NO generation system can retain more NO in the product gas when the product gas is kept hot. FIG. 59 presents an exemplary graph showing NO oxidation experimental data. A canister of 1600 ppm NO gas was diluted with house compressed air and oxygen to 21% O2 (atmospheric level) and various NO concentrations on the X axis. The gas mixture was maintained at atmospheric pressure and one of three temperatures, 5° C., 20° C. or 40° C. NO2 levels were measured after the same amount of residence time for each data point (roughly 2 seconds). When the gas temperature increased from 5° C. to 40° C., the product gas NO2 level decreased 50% for all NO concentrations.

In some embodiments, the product gas temperature is measured with a temperature sensor. In some embodiments, the product gas temperature is managed to be at the highest level that is compatible with product-gas contacting components. For example, when NO production levels are low and product gas temperatures are relatively low, there is no active cooling of product gas. Contrastingly, when NO production levels are high and product gas temperature is high, some embodiments of an NO generation system cool the product gas to prevent thermal damage of system components.

In some embodiments, a NO generation system is designed to preserve NO in the product gas by keeping the product gas hot. Various passive approaches exist for maintaining thermal energy in the product gas including insulating the conduits that convey product gas, utilizing non-thermally conductive materials for product gas plumbing (e.g., Teflon). Various passive approaches exist for heating the product gas, including routing product gas conduits near other system components that are inherently hot (e.g., plasma chamber, pump). There are also many means to actively heat product gas, including resistive heaters, thermoelectric heaters, combustion heaters, exothermic chemical reaction heaters, etc. NO systems with oxygen in the product gas especially benefit from product gas heating after the scrubber since any NO₂ formed downstream of the scrubber could be inhaled by the patient. Heat transfer to the product gas and/or product gas conduit can be accomplished via convection, conduction, and radiation. In one embodiment, one or more heat sources (e.g., gas pump, plasma chamber) are attached in a way to conduct heat to a heat sink, the heat sink including pathways for product gas flow. In some embodiments, the heat sink is a gas manifold. In some embodiments, the heat sink is a gas reservoir. In some embodiments, a gas conditioning cartridge (GCC) includes a electrically resistive heater to elevate the temperature of product gas within the scrubber and pneumatic pathways within the GCC. In this kind of embodiment, electrical connections between the GCC and NO generation device provide the necessary electricity to power the heater. In another embodiment, the GCC includes a thermally conducting surface that aligns with a hot surface of the NO generation device when installed. The hot surface on the NO generation device can be passively heated (e.g. pump and plasma chamber heat) or actively heated. Heat is transferred to the GCC through thermal contact. In some embodiments, thermal contact is enhanced with thermal paste. In some embodiments, a NO device is actively cooled with cooling fans and the warm exhaust of the cooling system is routed toward and/or through the GCC to elevate the temperature within the GCC.

Disposables

It should be noted that the term “disposables” here applies to removable components of the NO delivery system and includes components that are rechargeable, reusable, and semi-disposable. A portable NO generation device includes a reusable controller containing the necessary pumps, valves, battery, sensors, pneumatic pathways, reservoirs, high voltage circuitry, control circuitry, software, electrodes, plasma chamber, user interface, power interface and more. The system also includes components that are removeable and/or disposable. For example, the NO₂ scrubber will have a finite life and require replacement or refurbishing from time to time. Similarly, the delivery device will require replacement, particularly if the delivery device includes a scrubbing capability. Humidity management materials (e.g., desiccant) will also have a service life. In some embodiments, the delivery device, desiccant and scrubber are permanently housed with each other to form one assembly. This offers benefits in usability with fewer use steps required. In some embodiments, the cannula, desiccant and scrubber cartridge are replaced independently. This can offer advantages in operating cost if each of the disposable elements have different service lives. In some embodiments, the replacement schedule for disposable components is selected to either be every 24 hours (daily) or every week (7 days) or every month (31 days) to make it easier for users to abide by the replacement schedule.

Scrubber Cartridge Design

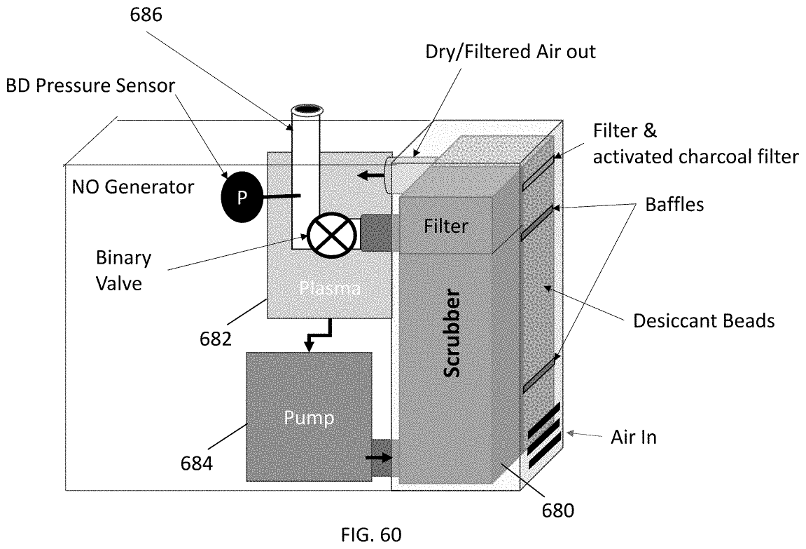

FIG. 60 depicts an exemplary embodiment of a NO generator with a removable cartridge that prepares reactant gas and scrubs and filters product gas. Air enters the cartridge 680 and travels along a tortuous path through desiccant material to remove humidity. In cases where an expansive desiccant is utilized (e.g., silica gel), ample expansion volume is provided to accommodate the desiccant in its hydrated state. As the air exits the desiccant stage, it passes through a VOC scrubber and through a particulate filter to remove particles from the ambient air, desiccant and scrubber. In some embodiments, the particulate filter removes particles greater than 20 microns in diameter. In some embodiments, the VOC filter is located prior to the desiccant material. In some embodiments, desiccant and VOC scrubber materials are dispersed within a common chamber. In some embodiments, both the VOC scrubber material and desiccant materials are granular. In some embodiments, the VOC scrubbing material consists of a sheet of pure activated carbon and may have additional additives to remove specific contaminants (e.g., ammonia). The gas then passes through a pneumatic connection into the controller. In some embodiments, the controller includes a VOC sensor (e.g., photo ionization sensor (PID)) to detect VOC levels in the reactant gas. When VOC levels are elevated above acceptable thresholds, this can be indicative of one or more of levels of VOC in the environment exceeding the capacity of the VOC scrubber, VOC scrubber exhaustion, and/or VOC scrubber incompatibility with specific VOCs. When excess VOCs are detected in the reactant gas, a NO generation device can generate an alarm, prompting the user to replace their VOC scrubber and/or move to a different environment.

On the controller side, the reactant gas (e.g., preconditioned air) enters the plasma chamber 682 where plasma converts a fraction of the N₂ and O₂ molecules into NO and a smaller portion into NO₂ and ozone. Ozone rapidly reacts with NO, producing more NO₂. This product gas passes through a pump 684 and back into the cartridge 680 through a pneumatic connection. The product gas passes through a scrubber (e.g., soda lime) to remove NO₂ from the gas stream and through a filter to remove particulates. In some embodiments, the particulate filter removes particles greater than 0.2 microns. The product gas passes back into the controller side where a flow controller (e.g., valve) controls the flow of product gas. When the valve is closed, product gas accumulates in the volume between the pump and the valve which is predominantly filled with scrubber material. When the valve opens, pressurized, scrubbed product gas within the scrubber is released and travels through the valve and out of the controller into a patient delivery device (e.g., cannula). A pressure sensor in the controller near the delivery device connection is utilized to detect pressure fluctuations within the delivery device that are indicative of respiratory events (e.g., onset of inhalation).

FIG. 60 depicts a delivery device connection 686 (for example, an output barb) on the controller. In some embodiments, the output barb is located on the cartridge instead. Connection of the delivery device to the cartridge can be advantageous in designs where the delivery device is replaced at a similar frequency to the scrubber/desiccant cartridge, such as when the delivery device includes scrubbing material as well (e.g., scrubbing cannula). This decreases complexity and use steps for device users. It also enables the manufacturer to establish the delivery device to cartridge pneumatic connection with greater durability and reliability than a user-established connection.

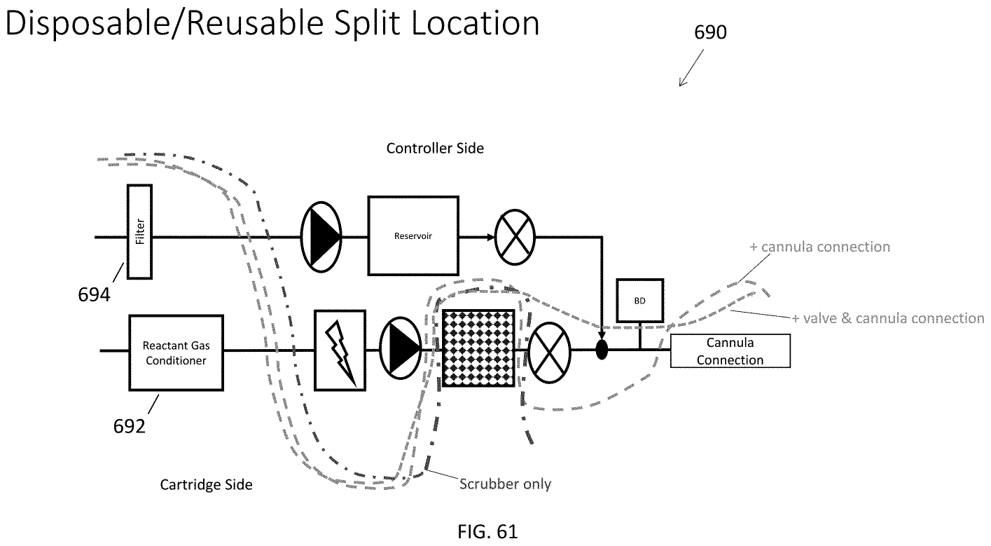

FIG. 61 depicts an exemplary embodiment of a NO generation device 690 with a pressurized scrubber and pressurized bypass architecture with independent gas inlets for each leg. Reactant gas passes through a conditioner 692 that does one or more of particle filtration, VOC scrubbing, and water removal. Purge gas simply passes through a particle filter 694. Dashed and dotted lines depict various ways to split the system into reusable and disposable components. All of the embodiments shown depict the reactant gas conditioning to be part of the disposable. If the inlet particle filters are sufficiently sized, they could be part of the controller as well. Each of the embodiments also involve the desiccant material being reusable or replaceable. In some embodiments, desiccant can be removed, dried, and reused.

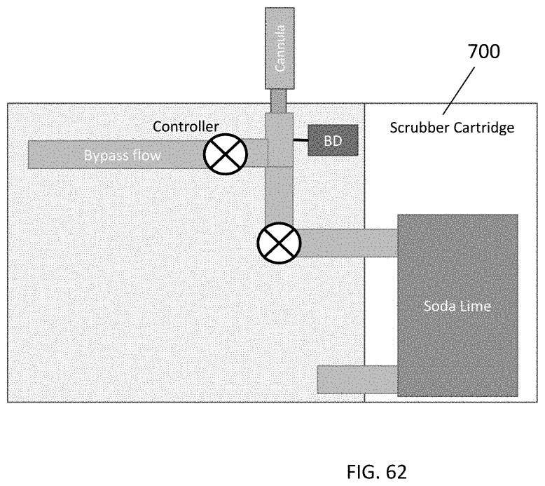

In some embodiments, as shown in FIG. 62, an exemplary disposable component 700 (cartridge) includes only the scrubber, filters and desiccant. This allows for the use of two pneumatic connections such that there is lower leak potential, lower insertion force to insert the disposable component, and less mass to the disposable component. This also allows for the life of the delivery device, such as a cannula, and the life of the scrubber to be independent. For example, these can be instances in which the cannula lasts longer than the scrubber, or the cannula can be a non-scrubbing cannula. If the cannula does also scrub the gas, there can be an independent way to track the usage and replacement of the cannula. This embodiment is similar to the design in FIG. 60 with the exception that desiccant is not included.

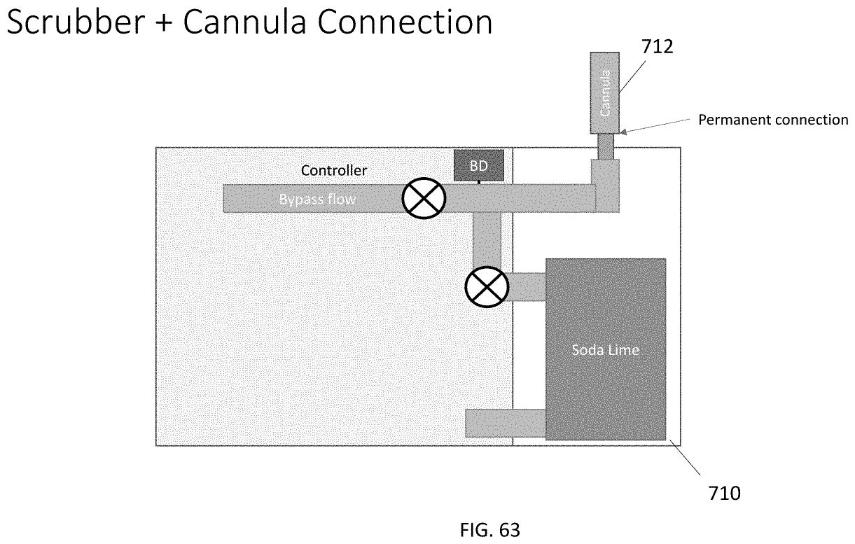

FIG. 63 depicts an exemplary embodiment of a cartridge design 710 where the delivery device 712 connects directly to the cartridge. This can be used when the delivery device is required to be replaced at a similar frequency as the cartridge. In some embodiments, the delivery device is permanently affixed to the cartridge. Permanently joining the delivery device and cartridge also enables a system to detect replacement of both components by only detecting replacement of the cartridge.

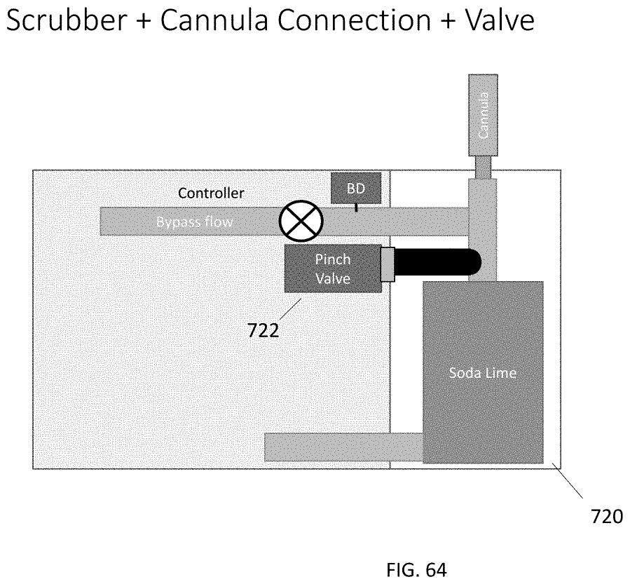

FIG. 64 depicts an exemplary embodiment of a cartridge 720 with an elastomeric tube section between the scrubber and the delivery device connection. A linear actuator 722 on the controller side pinches the tubing to block flow exiting the scrubber, enabling the system to pressurize the scrubber. This approach can minimize dead volume between scrubber and valve, thereby decreasing the amount of NO2 formed in the product gas between breaths. The post-scrubber, pre-flow controller volume is an important feature of any NO generator, particularly for pressurized scrubber designs where the elevated pressure and longer residence time can result in unacceptable NO2 levels. Multiple approaches to minimizing the post-scrubber dead, pre-flow controller volume are presented here-in. In general, this volume should be a small portion of the overall NO pulse. In some embodiments, this volume is less than 1 ml. In some embodiments, this volume is less than 2 ml. When the post-scrubber, pre-flow controller volume is >2 ml, NO2 levels can approach unacceptable levels, particularly at high NO concentrations where NO oxidation is more rapid and/or high respiratory rates where the volume of post-scrubber, pre flow controller product gas makes up a larger portion of the delivered NO pulse.

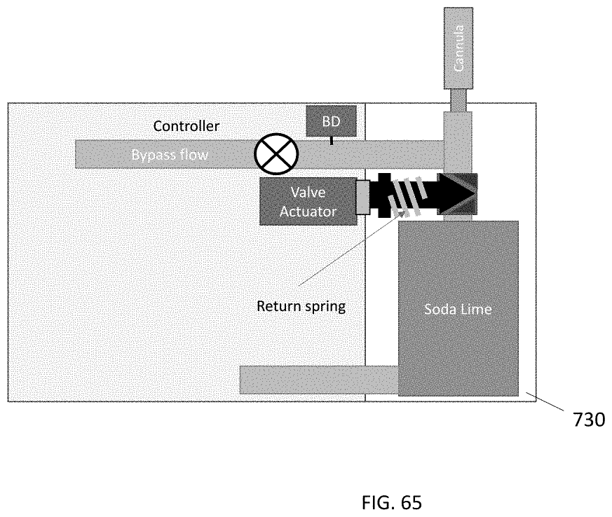

The system embodiment and cartridge 730 shown in FIG. 65 is similar to the embodiment of the cartridge shown in FIG. 64 except for use of a needle and seat valve within the cartridge that is actuated by an actuator within the controller. Return actuation of the needle and seat valve can be driven by the actuator or passively returned using a spring. The actuator can be linear (e.g., linear motor, piezo actuator, solenoid, pneumatic piston, rotation of a screw, etc.) or rotational (e.g., motor, peristaltic valve).

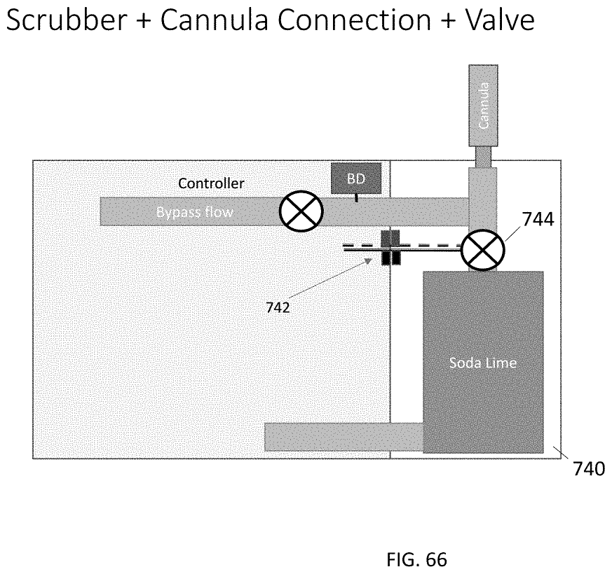

FIG. 66 depicts an exemplary embodiment of a cartridge 740 with electrical connections 742 to the controller and an electric valve 744 to control flow exiting the scrubber. This embodiment minimizes the non-scrubbed volume between the scrubber and the NO valve, thereby minimizing the volume of post-scrubber, pre-flow controller product gas in the NO pulse, reducing inhaled NO₂ levels. For applications where the delivery device connects to the disposable cartridge, there are further benefits of reduced pathway length to the delivery device when the valve is placed in the scrubber cartridge. Electrical connections to the cartridge can be accomplished with brushes, pogo pins, an electrical connector and other means.

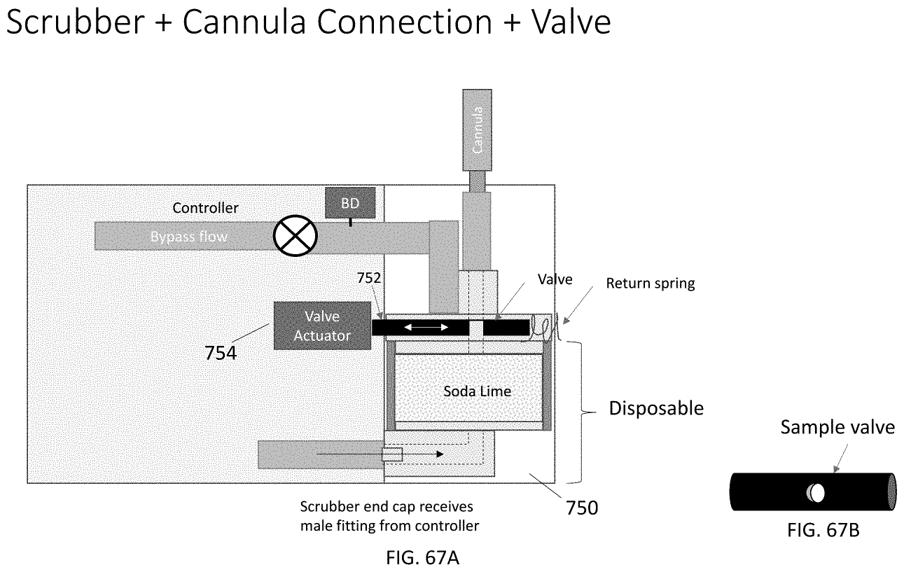

FIGS. 67A and 67B depicts an exemplary embodiment of a cartridge 750 where an endcap in the scrubber housing serves as a valve housing. A pin 752 is actuated using a value actuator 754 from the controller side to open and close the valve. This design further reduces the post-scrubber, pre-flow controller dead volume downstream of the scrubber and parts count. In some embodiments, the pin is solid and moves completely in and out of the flow path. In some embodiments, the pin has a hole in it, as shown in FIG. 67B, like the valve in a trumpet, that aligns with the flow path when the valve opens. In some embodiments, the pin is rotated to open the valve, like a French horn valve. In some embodiments, the pin is moved in both directions by a solenoid. In some embodiments, the pin moves one direction by a solenoid and the opposite direction by a spring force. In some embodiments, the system is designed so that the valve is energized to release NO and does not require power when the valve is closed to conserve energy. A solenoid valve can also open a valve faster than a spring, resulting in quicker NO delivery to the patient.

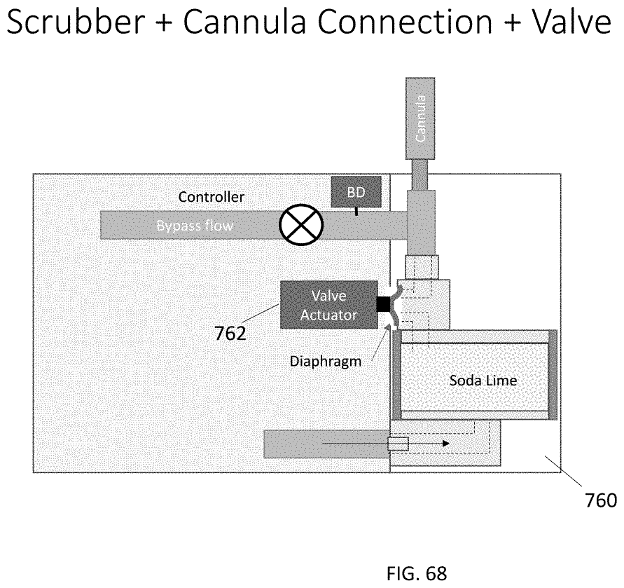

FIG. 68 depicts an embodiment of a cartridge 760 where an actuator 762 from the controller side can press on a diaphragm or flapper valve to control the flow of product gas exiting the scrubber. This design allows for low GCC installation force, a low-cost disposable and low chance of leakage since the diaphragm seal is established and tested during GCC manufacturing. The cannula connects directly to the GCC. In some embodiments, the cannula is replaced at the same frequency as the GCC and the connection between the two components is permanent. This decreases the potential for a partial connection established by the user.

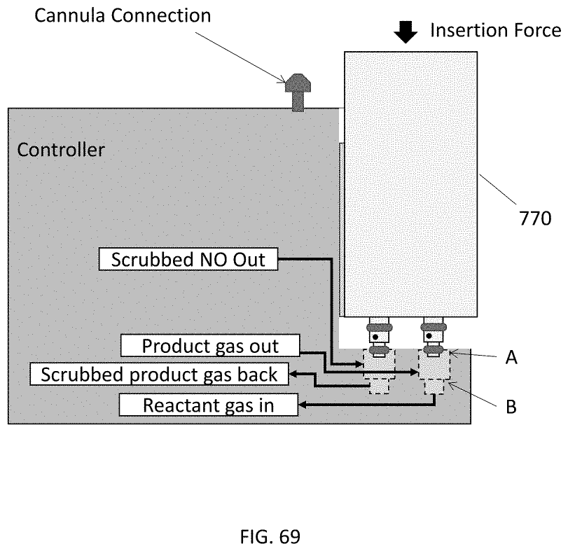

Combining all of the expendable components of a NO generation unit into a single disposable provides benefits in usability by requiring the user to manage fewer tasks and service intervals. Gas conditioning cartridges that provide multiple process steps require multiple pneumatic connections to be established when they are installed. Each pneumatic connection requires force to connect and when they all are engaged simultaneously, considerable force can be required to insert a gas conditioning cartridge. FIG. 69 presents an embodiment of a GCC 770 that reduces insertion force for a GCC. This design utilizes two co-axial pneumatic fittings with staggered engagement. The upper O-rings are larger diameter than the lower O-rings in the depicted design, however that is not a requirement. As the user inserts the device, the upper O-rings contact the controller first at point A. The user overcomes the force to insert two O-rings and continue sliding the GCC into place, overcoming dynamic friction from the upper two O-rings. Then, the lower two O-rings engage the controller at point B. This approach decreases the amount of force required at one time, facilitating GCC installation. In another embodiment, not shown, the O-rings engagement is sequentially staggered so that only one O-ring engages at a time to smooth out the force profile as the GCC is inserted.

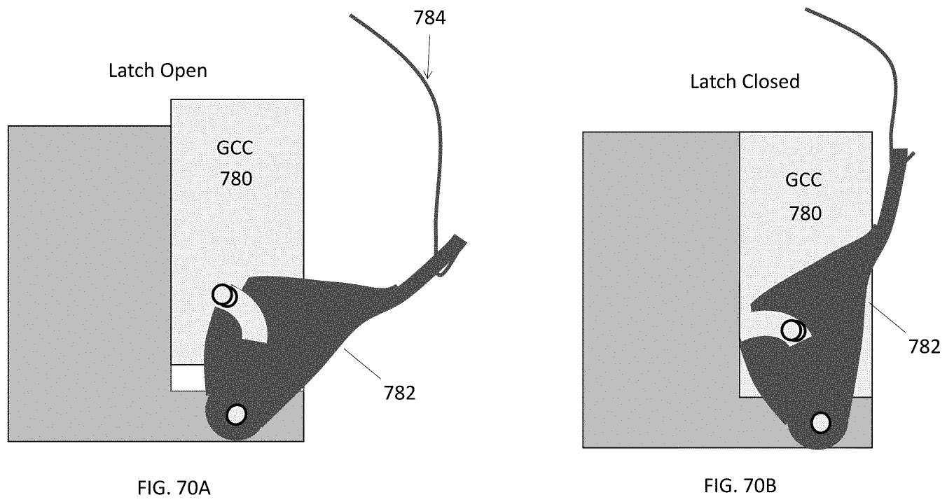

FIGS. 70A and 70B depict another exemplary embodiment of a GCC 780 for facilitating the installation of a GCC with multiple pneumatic connections. A latch 782 is used to engage the GCC pneumatic connections and hold it in place. The latch handle provides mechanical advantage so that less force is applied over a longer throw. In some embodiments, the latch is connected to a shoulder strap 784, as shown. As the latch is moved to the closed position, a curved groove or slot engages one or more pins on the GCC and draw the GCC into the fully-seated position.

Cannula Design

Dimensions

A nasal cannula serves as a conduit for communicating gas flow from the controller to the patient. When the NO generation device is worn on the patient's body, in some embodiments, a length of 4-feet is found to be a functional length. When a patient wants to separate from the NO generation device, for example when the NO device is in a shopping cart and the patient wants to reach the shelves, a 7-foot cannula length functions well. In general, cannula lengths of 1.5 to 10 feet have been contemplated. Cannula length is proportional to cannula dead volume. As dead volume increases, there is more volume to displace to deliver a NO pulse and pulse timing can take longer. Decreasing the diameter of a cannula is one way to maintain an acceptable dead volume while providing acceptable length. There are limits to cannula internal diameter as well, however, as smaller diameters increase the flow restriction of a cannula requiring greater amounts of pressure to deliver nitric oxide pulses in time. Higher levels of pressure can result in faster NO oxidation within the NO generation device, requiring additional NO to be made. This additional NO formation comes at the expense of additional electrical energy, requiring a larger/heavier battery. Optimization of these interconnected features results in cannula cross-sectional area equivalent to a circular cross-section with a 1 to 3 mm internal diameter. In some embodiments, an optimal internal diameter for the NO delivery lumen of a nasal cannula with circular cross-section is 1.5 mm (˜ 1/16 inches).

Long Nasal Prongs

While typical nasal prongs measure 10-12 mm in length, there can be the need for longer prongs during oxygen and NO therapy. Long prongs, i.e., prongs measuring 13 to 200 mm, are in fluid communication deep within the nasal cavity, reducing breath detection interference from the environment and/or entrainment of ambient gas. This can result in cleaner respiratory signals to analyze for breath detection. Longer prongs deliver NO, and/or oxygen and/or other drugs towards the mid to posterior nasal cavity or nasopharynx. As a patient inhales via the nose, the first gas that enters the patient airway is from the nasal cavity. By introducing medical gas to the mid to posterior of the nasal cavity, the medicinal gas can travel deeper and earlier into the lung when delivered without intentional delays. Long NO pulses that dose the entire inspiratory volume can also be delivered from this location. A further benefit to long nasal prongs is that they can extend beyond nasal valve, the smallest cross-sectional area region within the nasal passage, that might otherwise slow or obstruct medical gas pulse delivery. This decreases the back pressure to gas delivery and improves the right/left symmetry of delivery. An additional benefit to long prongs is the elimination of issues related to partial prong insertion, namely loss of breath detection signal and loss of medical gas to the environment. The actual length of long prongs can be tailored to a specific patient's anatomy by trimming their length.

In some embodiments of medicinal gas delivery systems with long nasal prongs, a medicinal gas pulse is delivered after exhalation but before inspiration occurs. This can be done without loss of medicinal gas because the medicinal gas will remain in the nasal cavity until inhalation occurs. In some embodiments, oxygen is delivered to the nasal cavity after exhalation and before inspiration to displace some or all of the carbon dioxide-rich gas within the nasal cavity. Various embodiments deliver NO in pulses while others deliver NO continuously through long prongs.

One potential drawback to long nasal prongs can be obstruction of the nares (the nasal valve is the narrowest part and could be blocked). In some embodiments, the lumens within a multi-lumen prong have different lengths. A lumen delivering a potentially toxic gas with very specific dose requirements (e.g., nitric oxide) is delivered through a long, slender lumen that reaches the mid to posterior nasal cavity, while a safe drug with less sensitive dosing requirements (e.g., oxygen) is delivered within the nares. This approach permits the nasal prong to taper along its length so that there is less obstruction within the nasal pathway.

For example, the nasal valve has a cross-sectional diameter of 5 mm, on average. This provides sufficient cross-sectional area for tubes to be passed through the nasal valve without significantly affecting the patient's ability to breathe through the nose. Nasogastric tubes, i.e., tubes routed through the nose to the stomach, are well tolerated by patients for months and available up to 6 mm in diameter. Thus, a thin-walled NO lumen measuring roughly 3 mm in diameter should be well tolerated.

Nasal cannulas on the market typically have two prongs to ensure drug delivery in the event that one of the nares is blocked. Utilizing a single long prong delivery tube that reaches beyond the nasal valve mitigates against this issue. In some embodiments, a single long prong delivery tube is utilized to deliver medical gas to a patient.

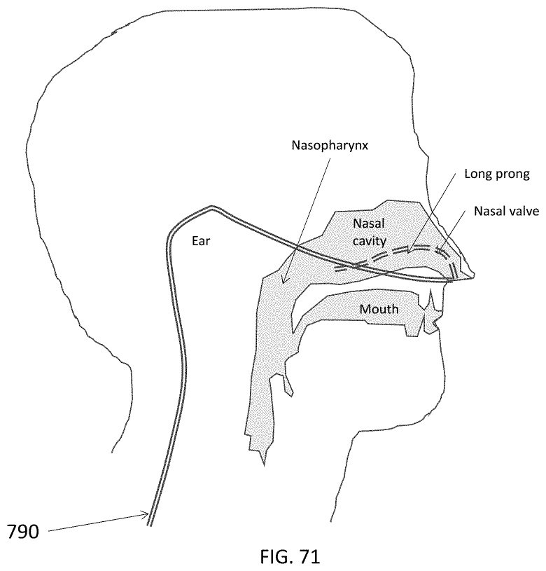

FIG. 71 depicts an exemplary delivery device positioned on the head of a patient. A single gas delivery lumen 790 travels up the side of the patient's neck and around their ear. The length of lumen eternal to the patient travels across the patient's cheek and into one nostril, shown in sold lines. The inserted portion of the lumen extends into the posterior nasal cavity, shown in dashed lines. In some embodiments, the inserted portion of the lumen is made from a lower durometer material and/or thinner wall to minimize tissue irritation. A single, low diameter lumen can be more aesthetically pleasing than a traditional nasal cannula with tubes over both cheeks, a hub below the nose and dual, large diameter prongs.

In some embodiments, a tool is supplied with the long-pronged nasal cannula to aid in cannula insertion. The tool consists of a slender rod that engages the proximal end of the long prong. As the tool is inserted into the nose, it pulls the long prong with it. Once the long prong is fully inserted, the tool is withdrawn and disconnects from the prong. In some embodiment, the end of the rod is inserted into a pocket in the prong material for simple insertion and removal.

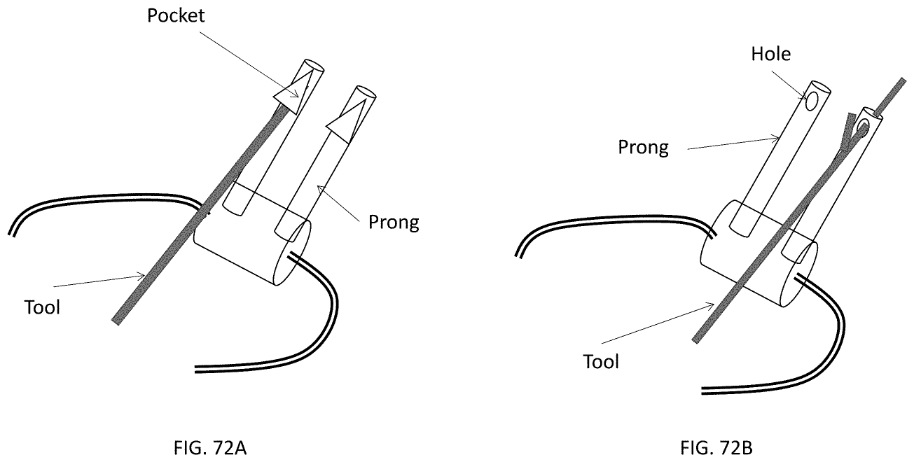

FIG. 72A depicts an embodiment of a long prong placement tool. The prong includes a pocket at the proximal end. A slender rod is inserted into the pocked. As the rod is inserted into the nose, the prong is pushed into position. The rod is then withdrawn, leaving the prong in place. In some embodiments, there are two parallel rods that place both prongs into their corresponding nostrils at the same time.

FIG. 72B depicts an embodiment of a long prong placement tool. The prong features a hole on the side. The tool features a blunt barb on the side. This can also simply be an increase in diameter beyond the diameter of the hole in the prong. The proximal end of the rod is inserted through the hole in the prong. Then, the tool is inserted into the nose of the patient. After fully inserting the prong, the rod is withdrawn, leaving the prong in place. In some embodiments, both prongs are placed at the same time.

Mouth Breathing Detection

In some embodiments, a pulmonary drug delivery device can identify the patient breathing mode (mouth vs. nasal) by differences in the acoustic sound of inhalation. The signal from a microphone can be processed to detect the difference in sound between mouth and nasal breathing. In some embodiments, this difference is customized for each patient.

When a patient inhales through their mouth, nasal cavity flow is decreased but typically non-zero. In some embodiments, a NO device will alarm after a breath has not been detected for a period of time (e.g., 45 seconds). In some embodiments, a NO delivery device will enter an asynchronous pulsing mode when breaths have not been detected for a period of time. The asynchronous pulsing mode, as the name implies, is not synchronized with the breath but increases the chance that some NO will be delivered to the patient.

Cannula Material Selection

NO delivery systems are generated from materials that are chemically inert to NO/NO2, such as silicone or polyvinyl chloride (PVC). In some embodiments, the NO lumen material includes an additive to make the material opaque or colored in order to mask discoloration associated with NO2 staining. This can help blind a patient in a clinical study so that the patient does not know whether or not they are receiving NO. It can also keep a delivery device looking new for a longer service life thereby reducing system operating cost and minimizing burdening the patient to replace disposable components.

Breath Detection Lumen

In some embodiments, breath detection is performed by measuring pressure fluctuations at the patient through an air-filled column with a pressure sensor within the NO generation/delivery device controller. In some embodiments, the breath detection lumen is a dedicated lumen. In some embodiments, breath detection occurs within the NO delivery lumen. As the NO lumen diameter decreases or is filled with scrubbing material and/or filters, the pressure signal can diminish, making breath detection more challenging. In some embodiments, a cannula includes a dedicated breath detection lumen that is parallel to the NO delivery lumen. In some embodiments, the breath detection and NO delivery lumen intersect soon after the NO lumen filter/flow restriction. In some embodiments, the lumens remain separated to a point closer to the patient.

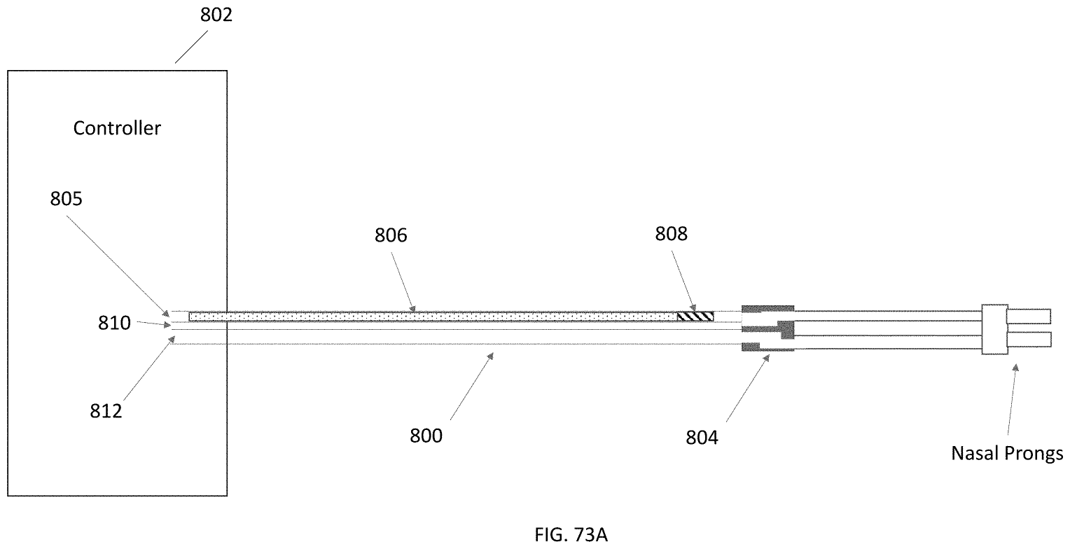

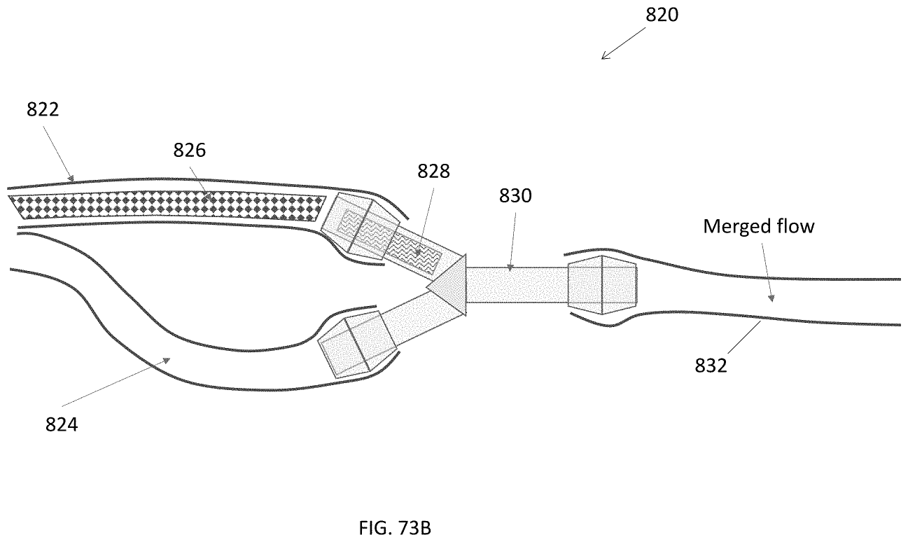

FIG. 73A depicts an exemplary cannula 800 with three lumens between the controller 802 and a junction point 804 along the length of the tubing. A NO delivery lumen 805 includes scrubbing material 806 and a filter 808 to remove particulate. A breath detection lumen 810 extends from the controller to the junction point as well. At the junction point, the breath detection and NO delivery lumens intersect, and a single lumen extends the rest of the distance to the patient. A third lumen, for example an auxiliary lumen 812, can be utilized for delivery of an additional gas (e.g., oxygen) and extends uninterrupted from the controller to the patient. In some embodiments, the auxiliary lumen is utilized to pull gas from the patient to the controller. In some embodiments, gas is sampled from patient exhalation for the measurement of exhaled NO. Since NO levels in exhaled gas are suppressed during inhaled NO treatment, measurement of exhaled NO levels (e.g., Fractional exhaled NO, FENO) can be an indication of NO treatment effect. Some NO generation and/or delivery systems respond to an absence of treatment effect, as indicated by a FENO measurement that is at baseline (pre-treatment levels) with an alarm. In other embodiments, systems respond by increasing the NO dose within a range of clinically acceptable levels. This same concept could apply to other types of delivery tubes, including tubing that delivers gas flow to a mask.

FIG. 73B depicts an exemplary embodiment of a delivery device 820 for merging a NO lumen 822 and a breath detect lumen 824. The NO lumen contains scrubber material 826 within it. In some embodiments, the scrubber material is one or more of a loose media, packed media, a coating, a co-extrusion, or a filament insert. The scrubber material may be pure or be compounded with another material for improved material properties, such as stiffness, dust generation, toughness, permeability, moldability, extrudability and other properties. A filter 828 is pressed into the end of a Y-fitting 830. The NO delivery lumen is attached to the outside of the Y lumen with a barb (shown). Other means of attachment such as adhesive bond, thermal bond, solvent bond, barb fitting, hose clamp and other means may also be used. The breath detect lumen connects to another leg of the Y-fitting so that the NO and breath detect paths merge. The merged lumens 832 traverse to the patient delivery location (e.g., nasal prongs, face mask, Scoop catheter, ET tube, etc.).

Mixing Element

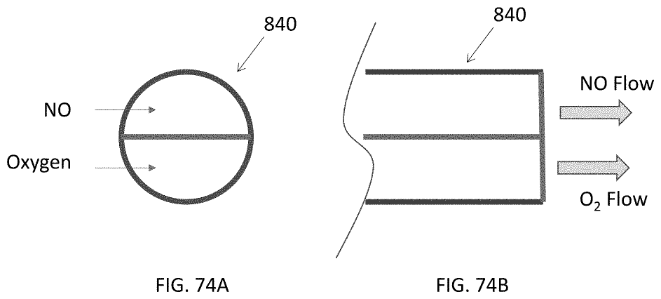

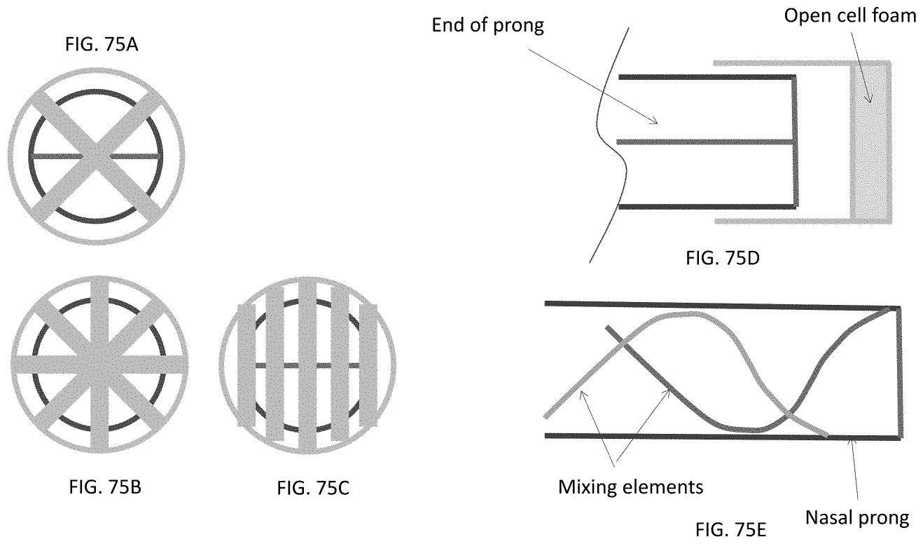

Two medicinal gases can be delivered to the nasal cavity and/or mouth through a delivery device 840 having dual-lumen prongs or split prongs, as shown in FIGS. 74A and 74B, which illustrates a cross-sectional view of the dual-lumen cannula and a side cross-sectional view of the dual-lumen cannula. When two medicinal gases are delivered concomitantly through a dual-lumen prong, the two flows interact with each other and with entrained ambient air. In some embodiments, a nasal or mouth prong for gas delivery includes a mixing element to mix injected NO gas with one or more of entrained air from inhalation, injected O₂, and other injected therapeutic gases. FIG. 75A-75 depicts various mixing element designs within and/or affixed to the end of a gas delivery prong. FIGS. 75A-75C show various cap designs that can provide a level of flow restriction and mixing/turbulence. Caps can be bonded to the end of the multi-lumen prong extrusion. FIG. 75D depicts a cap with an open cell foam that creates mixing of the gas streams as the flow through it. FIG. 75E depicts a nasal prong with static mixing elements within the lumen to mix one or more gases before they exit the prong. This approach can provide decreased NO concentration to reduce oxidation rate as well ensuring even distribution of NO within the lung.

Concomitant Oxygen Delivery

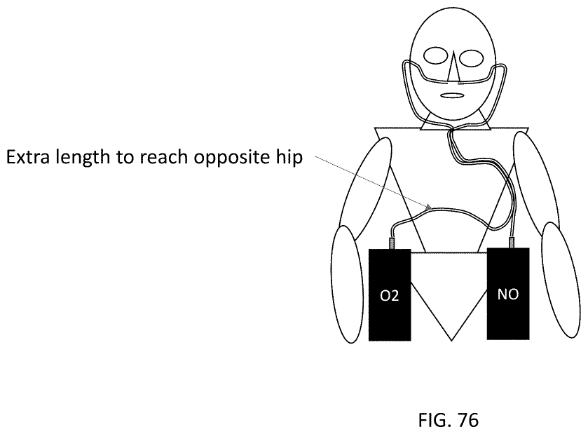

A nasal cannula can deliver nitric oxide and oxygen independently, sourced from separate devices. In some embodiments, the NO and oxygen lumens are routed from the nose separately over opposite ears of the patient prior to meeting in a 2-or more lumen extrusion. The extrusion traverses to a first device (NO or O₂) where the appropriate lumen is connected, then the remaining lumen traverses to the other device. FIG. 76 depicts an exemplary embodiment of a nasal cannula the routes to the NO device first. In some embodiments, one lumen is longer than the other to facilitate lumen handling. In some embodiments, the O₂ lumen is longer than the NO lumen and routes independently to the O₂ device. In some embodiments, the user routes the longer lumen around their back to minimize interference with daily activities.

Proximal Scrubber

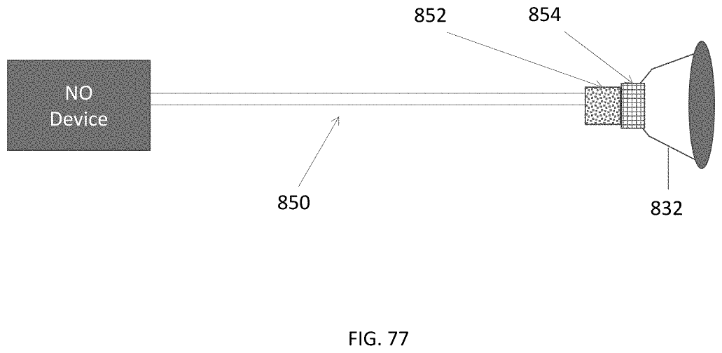

In some embodiments, for example in an ambulatory device, a proximal scrubber and/or filter is located at or near the patient end of the delivery device. This scrubber and filter can remove NO₂ that has formed during the transit from NO generation device to patient. The proximal scrubber can present a challenge to the user since it has some bulk and is typically hanging from the delivery device. In some embodiments, the proximal scrubber is placed at the base of the patient's next, like a pendant. In some embodiments, the proximal scrubber is located behind the patient's ear like a hearing aid. This approach allows for a more discrete location and is closer to the point of injection into the patient which helps minimize the amount of additional NO₂ that forms post proximal scrubber. As mentioned, the locations for lumen intersections identified in FIGS. 11A, 11B and 11C also serve as potential locations for a proximal scrubber. In another embodiment depicted in FIG. 77, an exemplary embodiment of a delivery device 850 is shown that includes a proximal scrubber 852 and/or particulate filter 854 as part of a mask 856.

Cannulas that Scrub NO₂: Filament

In some embodiments, a cannula includes a filament of NO₂-scrubbing material within the NO delivery lumen. Filaments can be an extrusion, cut from a sheet, or other means to create a slender, long structure with high surface area for scrubbing while still allowing gas to pass freely through the cannula. In some embodiments, the filament has side cuts for additional surface area and gas mixing.

In some embodiments, a filter is located downstream of the filament to collect particles of scrubbing material that can be released due to cannula motion and gas flow. In some embodiments, the filament is inserted into an existing tube or cannula at the time of manufacture. In some embodiments, a tube is over-extruded around a filament. In some embodiments, the filament is co-extruded with the outer tube walls. Scrubbing materials or often not biocompatible with skin, so a jacket material is often utilized to prevent skin-scrubbing material contact.

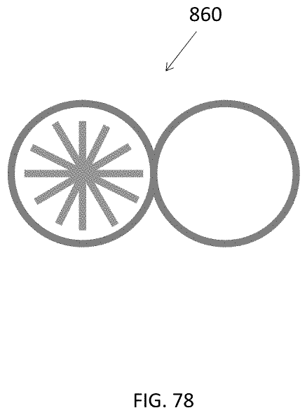

FIG. 78 depicts an exemplary delivery system 860 that utilizes a NO₂ scrubbing material splined filament that slides into the NO delivery lumen of a pre-existing delivery device.

Cannulas that Scrub NO₂: Coating & Compounding

Scrubbing materials require gas contact for NO₂ capture. A larger surface area of scrubbing material results in one or more of a greater ability to scrub and longer scrubber service life. Scrubber material can be added to the internal surfaces of a lumen via coating. In some embodiments, scrubber material is compounded with another material (e.g., polymer) and is extruded into a structure that resides within the tube of a delivery system. In some embodiments, a gas-tight jacket of material suitable for skin contact is over-extruded over the scrubbing lumen(s). In some embodiments, the scrubbing lumen(s) and outer layers are extruded at the same time (co-extrusion).

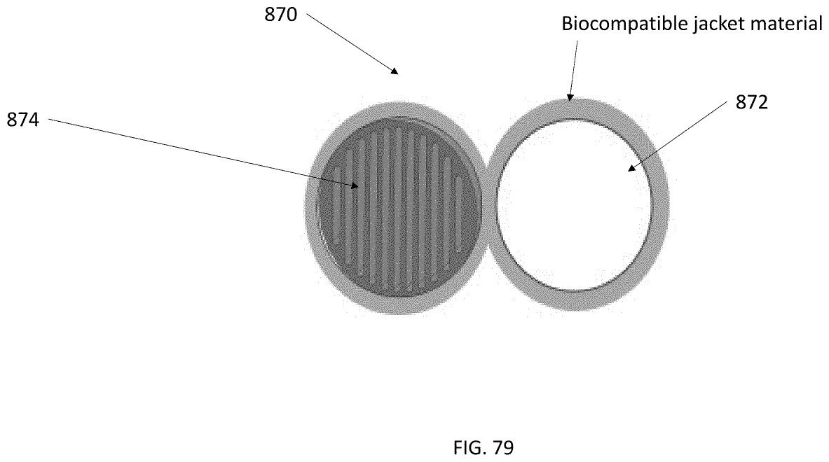

FIG. 79 depicts a cross-sectional view of a multi-lumen NO and oxygen delivery device 870. A lumen 872 can be used for oxygen delivery. A multi-lumen structure 874 with high surface area can be used as NO2-scrubbing lumens. Each of the scrubbing lumens are in fluid communication with each other at the ends of the delivery device. There is no harm if the lumens intersect along the length of the device due to manufacturing variation. In some embodiments, not shown, the width of the slot-shaped lumens varies to achieve similar flow restriction for all of the lumens. This ensures equal flow rates of NO-containing gas through each of the lumens so that they scrub and wear evenly.

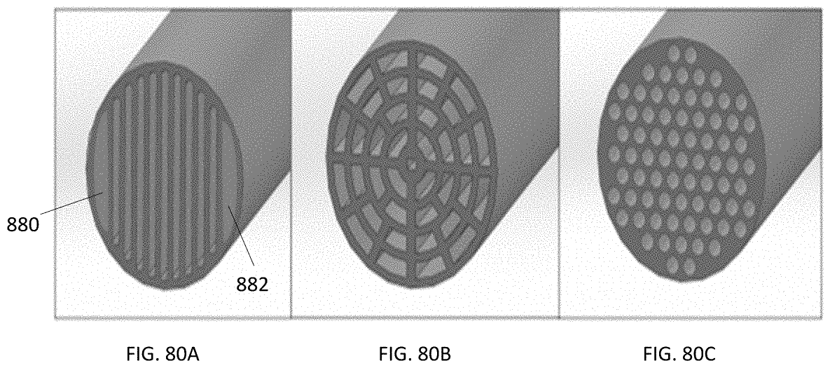

FIGS. 80A, 80B, and 80C depict various embodiments of high-surface area designs for the NO₂ scrubbing extrusion. FIG. 80A depicts an exemplary high surface area design consisting of parallel slits. The outer slits 880, 882 can be wider to increase cross-sectional area to more evenly equalize the mass flow rate through each of the slits to ensure even recruitment of scrubbing material and minimize flow restriction. In some embodiments, the width of each of the slits is different to achieve the same cross-sectional area and/or flow restriction for each slit. FIG. 80B depicts a high surface area extrusion design consisting of multiple rings and spokes creating multiple lumens through the extrusion. In some embodiments, the count and thickness of the rings and spokes are varied to achieve equivalent cross-sectional area and/or flow restriction for each of the lumens for even wear of scrubbing material. FIG. 80C depicts another embodiment of a high surface area extrusion for scrubbing with multiple equivalent lumens. This design allows for the cross-sectional area and flow restriction between the different lumens to be equivalent.

It should be noted that the objective of the high-surface area designs is to promote gas to scrubber material interaction. It is not critical that the lumens within the extrusion remain independent throughout the length of the delivery device. This important point allows for extrusions with less tolerance and thinner walls between lumens that may not be entirely continuous. Another aspect of high-surface area extrusions is the surface finish. When extrusion operations are run at or near the glass-transition temperature of a polymer, the surface are of the extrusion can be rough as the polymer is partially melted as it extrudes resulting in a rough surface finish. This rough surface finish can be a feature for further increasing surface area and gas/scrubber interaction.

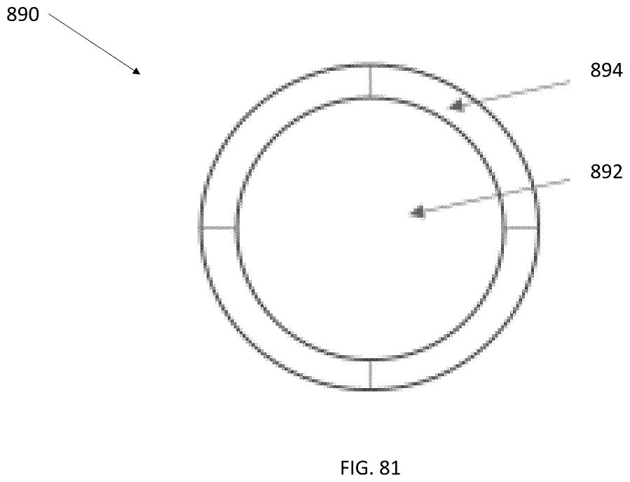

FIG. 81 depicts an exemplary embodiment of a delivery device 890 with an oxygen delivery lumen 892 in the center and multiple NO delivery lumens 894 around the periphery that can include scrubber material. This design allows for symmetrical flexural stiffness and high surface area for scrubbing due to placement of the scrubber lumens at the outer diameter. In some embodiments, one of the outer lumens is used for breath detection while the remainder of the outer lumens are utilized for NO delivery.

Scrubber Design

Color Indicator

In some embodiments, the controller has an optical sensor that can detect a change in color in the scrubber material. Soda lime, for example, can include an ingredient (e.g., ethyl violet) that changes color as the pH of the material becomes lower due to formation of nitric acid and carbonic acid within the moisture content of the scrubber. In another embodiment, a pH-sensing paper (e.g., litmus paper) is packaged between scrubber media and a scrubber cartridge wall so that it is visible to an optical sensor. The color change begins at the upstream end of the scrubber and travels along the flow path through the scrubber as the scrubber material is exhausted. In some embodiments, travel of the leading edge of the soda lime discoloration is characterized with respect to scrubber NO₂ efficacy. In some embodiments, an optical sensor is located adjacent to the scrubber and can detect the color change. When the color change travels sufficiently far along the length of a scrubber, the optical sensor can detect the color change, thereby triggering an alarm to replace the scrubber.

Packing

Carbon dioxide scrubbing material is commonly used in anesthesia systems. In that application, loose scrubber media (e.g., soda lime) is placed in a container with respiratory gas passing from the bottom to the top. Problems arise in this application due to non-uniform settling of the scrubber media and resulting channeling of gases. Channeling is the result of there being one or more low-resistance pathways through the bed of scrubber material. These low resistance paths handle a disproportionate amount of the gas flow resulting in inferior scrubbing and shorter scrubber life overall. One approach to achieving more uniform flow across a bed of scrubber media is to compact the scrubber media. This approach decreases the space between scrubber particles, making the gas flow path more tortuous. It also enables packing of more scrubber media into a given volume which can in turn provide more and longer scrubbing. In one embodiment, scrubber granules are compacted 15% by volume. In some embodiments, additional scrubber media is added to the volume and also compacted after the first compaction. Compaction of the scrubber media also reduces scrubber to scrubber variance.

Combination Humidifier/NO Generation Device

In some embodiments, a NO generator is integrated into a humidifier. For example, a NO generation device generates NO and introduces it to a patient inspiratory flow as the flow passes through a breathing circuit humidifier. Reactant gas for generating NO can come from a variety of sources, including ambient air, house compressed air, compressed gas cylinders, and the patient inspiratory stream.

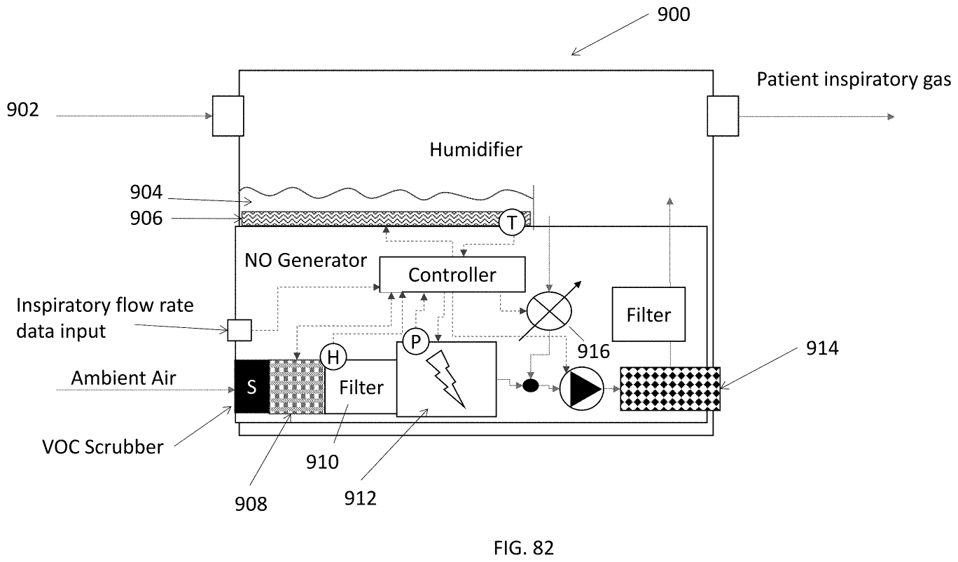

FIG. 82 depicts an exemplary embodiment of a combination NO generator and humidification device 900. The humidifier operates by receiving inspiratory gas 902 which then passes over heated water 904 using a heater 906 to increase the water content of the inspiratory gas. Ambient air enters the NO generator and passes through a humidity management stage 908 to dry the reactant gas and a filter 910 to remove particulates and/or VOCs. The humidity management stage may be active (e.g., variable temperature and flow rate gas flowing over Nafion tubing), or passive (e.g., gas flowing over desiccant material). The reactant gas then passes through a plasma chamber 912 where electrical discharges formed by one or more electrodes or microwave antennas generate NO and NO₂ to form a product gas containing NO. The product gas passes through a scrubber 914 (e.g., a removable scrubber) to remove NO₂ and one or more filters to remove particulates and/or VOCs before introduction to the patient inspiratory flow. In the embodiment shown, the product gas is optionally diluted with humidified patient inspiratory gas a varying amount to maintain moisture within the scrubber material (e.g., soda lime), thereby prolonging the scrubber service life. In some embodiments, it is desirable to maintain a constant flow rate through the plasma chamber. Thus, pump speed is increased when humidified air is blended with product gas. In some embodiments, a variable flow controller 916 (e.g., proportional valve) is utilized to vary the level of humid gas blending with the product gas. In some embodiments, a binary valve is utilized to control humid gas flow through the NO generator. The NO production level set within the NO generator can be a constant for the service life of the system. In some embodiments, the NO production level is variable based on a user setting or calculated based on a variety of inputs, including inspiratory flow rate, inspiratory gas mixture, patient condition, patient respiratory rate, patient dose, and other factors. In some embodiments, the inspiratory flow rate is communicated to the NO-generating humidifier by wired or wireless means so that NO can be introduced to the inspiratory stream in a proportional manner to maintain a constant inhaled concentration.

The system depicted in FIG. 82 is controlled by a software-controlled circuit that receives sensor information (e.g., water temperature, inspiratory flow rate, reactant gas pressure, reactant gas temperature, reactant gas humidity, product gas concentration, etc.) and controls the device operation by varying one or more of water temperature, active humidity management, pump flow rate, valve position, and plasma chamber activity.

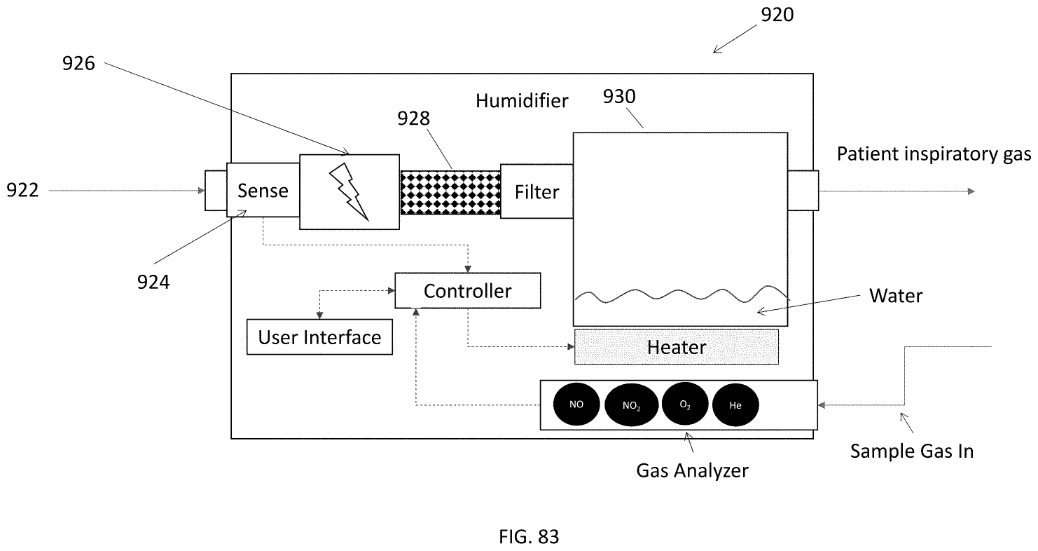

FIG. 83 depicts another exemplary embodiment of a combination NO generator and humidifier 920. In this example, patient inspiratory gas 922 serves as the reactant gas. In some embodiments, the system measures properties of the incoming reactant gas including one or more sensors 924, such as one or more sensors to measure humidity, temperature, oxygen level, pressure, and/or flow rate. In some embodiments, one or more properties of the reactant gas is provided to the NO generator from an external treatment device such as a ventilator. Inspiratory gas passes through the plasma chamber 926, propelled by an external source of pressure/flow. Plasma activity within the plasma chamber is varied based on the target product gas concentration and the reactant gas parameters mentioned above to produce a product gas. In the example shown, the target product gas concentration is equal to the target inhaled concentration since all of the inspiratory gas stream is dosed. The product gas passes through a scrubber 928 and enters a chamber 930 where warmed water elevates humidity levels prior to exiting the device and continuing to flow to the patient. In some embodiments, product gas is humidified before scrubber to ensure that the scrubber material does not dry out.

The water within a NO generation heater can become acidic over time due to the solubility of NO2 in water. Some embodiments include a means to measure the pH of the water. Some embodiments generate an alarm to notify a user that the water needs to be replaced when the pH reaches a threshold. In some embodiments, the system can automatically replace acidic water with fresh water when the pH reaches a threshold.

Some embodiments of a combination NO generator/humidifier include one or more gas concentration sensors to measure one or more of oxygen, nitric oxide, nitrogen dioxide, helium to measure concentrations in one or more of the reactant gas, product gas, and/or inspired gas. In some embodiments, these measurements are made from gas samples collected within the enclosure of the device or sourced externally as shown in FIG. 83 from another location (e.g., a T fitting closer to the patient). In some cases, sample gas is dried using one or more of a water trap (with or without cooling), Nafion tubing and desiccant prior to exposure to the gas sensors to prolong the viability of the sensors. One of the benefits of combining a NO generator with a humidifier is that the humidifier is typically located in close proximity to the patient. Proximity to the patient reduces the transit time of NO gas to the patient, in turn reducing the amount of time that the ratio of NO to NO2 within the inspired gas can be altered from NO oxidation. In some embodiments, analysis of externally sourced gas is not required because the device is sufficiently close to the patient that the gas mixture within the device and inhaled by the patient are either effectively the same or known to be different within a predictable and tolerable range.

Although linear architectures are depicted in the exemplary humidifier figures, it should be understood that any NO generation architecture with the requisite sensing components could be integrated into a humidifier. For example, a recirculation architecture for NO generation and delivery could be incorporated into a humidifier.

Breath Detection

In some embodiments, sensitivity of breath detection is turned up during a window of time that a breath is likely. For example, after the end of exhalation has been detected.

In some embodiments, breath detection sensitivity is increased during night/sleeping hours. In some embodiments, breath detection sensitivity is increased when connection of a 7-foot cannula is detected.

In some embodiments, a NO pulse is introduced to the cannula before a breath is detected. After breath is detected, additional NO and/or purge gas pushes the NO pulse the remainder of the distance to the patient. In some embodiments, the NO pulse is introduced, or staged within the cannula based on the detection of the end of the prior breath.

In some embodiments, a NO system relies on breath detection for slow breathing and breath prediction for fast breathing. This is advantageous because 1) slow breathing is more random and fast breathing is more periodic, 2) slow breathing involves longer inspirations which are less sensitive to delays associated with detecting and delivering NO, and 3) predicting breaths at fast rates enables a system to generate NO and start sending NO down the cannula early so that all of the NO has been delivered early in the breath.

When a pressure measurement is utilized to capture the breath detection signal, the polarity and magnitude of the signal can vary with treatment type. For example, the pressure measured through a nasal cannula in a patient that is spontaneously breathing will decrease as the patient inspires. In contrast, the pressure signal in the inspiratory limb of a ventilator will increase when inhalation begins. In some embodiments, a NO delivery system is capable of detecting respiratory events in a pressure signal for a variety of treatments. In some embodiments, the NO delivery device requires user selection of the breath detection method. In other embodiments, the NO delivery device automatically detects the type of treatment being administered based on one or more of the timing, polarity, shape, frequency content, and magnitude of the pressure signal.

In some embodiments, accelerometer data from the controller is used as an input to the breath detection signal to filter out motion artifact. This is done by utilizing the accelerometer data to identify patient motion. For example, a large deceleration event can be evidence of a patient landing a foot on the ground as they ambulate. The same deceleration can create motion artifact in the cannula pressure signal as it moves in response to the deceleration and patient motion. In some embodiments, the controller can use the timing and magnitude of acceleration events as inputs to the breath detection algorithm to minimize false positives. In some embodiments, the controller can sense that the patient is sedentary based on accelerometer data and increase the sensitivity of the breath detection algorithm to improve breath detection timing and accuracy. In some embodiments, accelerations sensed by the accelerometer can indicate that a patient has transitioned from a sedentary state to an active state. In this case, patient oxygen consumption and respiratory rate are expected to increase. In some embodiments, a NO generation system senses the increase in patient activity level and increases the NO dose delivered in anticipation of increased oxygen demand. For example, a NO generation device may increase the dose from 2 mg/hr to 6 mg/hr when patient activity beyond a specific threshold is detected by the treatment controller.

In some embodiments, accelerometer data can be used to detect events that might physically damage the device. For example, if it has been dropped (very high acceleration), the system will run diagnostics and then flag the unit so when the patient comes into a clinic, it can be inspected for damage or pre-emptively replaced.

EMG Breath Detection

In some embodiments, one or more electromyogram (EMG) measurements of the diaphragm are utilized for breath detection. The diaphragm initiates a breath by contracting to enlarge the pleural cavity, expand the lungs and pull in air. Thus, detecting inspiration at the diaphragm provides an early signal that a breath will occur.

Earlier inspiration information provides more time for analysis of breath data prior to generating a trigger signal for more reliable inspiration detection. Diaphragm EMG breath detection can also be less prone to interference from environmental factors, talking, delivery device interface, and other factors. EMG breath detection is also more reliable in detecting shallow breathing. Another benefit of EMG breath detection is that breaths are detected independent of whether the patient is breathing through their mouth or nose. In some embodiments, an EMG measurement device is located on an adhesive patch that is applied to the torso of a patient at the height of the diaphragm. In some embodiments, the patch includes a means of wireless communication between the patch and NO device (e.g., Bluetooth). In other embodiments, an acoustic or ultrasound signal is generated by the patch when an inspiration is detected and received by the NO device.

In some embodiments, the EMG device broadcasts the EMG signal to an external device (e.g., gas delivery system) that performs further analysis. In some embodiments, the EMG device processes the EMG data and only delivers inspiration trigger information. Other information that various embodiments of an EMG breath detection device may communicate include one or more of EMG signal strength, battery status, wireless signal strength, error codes, serial number, calibration information, expiration date, and elapsed time in-situ (for timely replacement). An EMG patch may receive various types of information from a gas delivery system as well, including patient factors (fat percentile, ideal body weight, disease type, disease state, EMG sensor location), and treatment factors (target gas pulse delay, drug dose prescription, subset of breaths to dose, etc.). Some or all of these factors may serve as inputs to how the EMG device operates. For example, the EMG device may increase its sensitivity based on the size of a patient (e.g., muscles are further from the skin in obese patients). In other scenarios, the EMG sensor detects respirations, waits for a period corresponding to a planned delay, then delivers a breath detect signal to the NO delivery device.

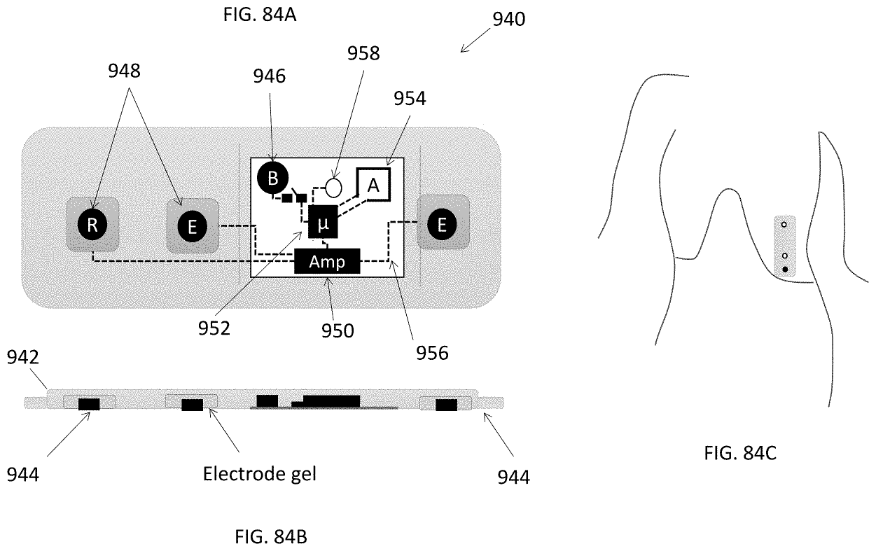

FIGS. 84A, 84B, and 84C depicts an exemplary embodiment of an EMG breath detection device 940. The device, as shown in a side view in FIG. 84B, includes a flexible substrate material 942 with adhesive 644 on an adhesive side. The device includes a circuit (shown in the top view shown in FIG. 84A) powered by a battery 946 that includes two or more electrodes 948, an amplifier 950, a processor 952, and an antenna 954. In some embodiments, the electronic components are mounted to on a flex circuit 956. In some embodiments, the electrodes are surface electrodes. In some embodiments, the electrodes are needle electrodes. The electrodes are in contact with electrolyte gel. One electrode serves as a reference electrode for the one or more other electrodes. A protective layer (e.g., wax paper) covers the adhesive and gel during transport and storage.

In some embodiments, an EMG patch includes a light indicator 958 (e.g., LED) that communicates the status of the device and/or battery. In some embodiments, the EMG patch communicates status, error messages, battery voltage and other information wirelessly to the NO device.

The protective layer is removed prior to placement on the skin, as shown in FIG. 84C. In some embodiments, the EMG device turns on when the protective layer is removed from the adhesive. This is beneficial by reducing use steps. In some embodiments, the protective layer includes a magnet that opens a reed switch. When the protective layer is removed, the reed switch closes thereby closing the power circuit and activating the EMG device.

In some applications, the EMG device is placed on the chest to measure parasternal intracostal muscle activity. In one embodiment, the EMG sensing electrodes are located at the 6^{th} and 8^{th} intercostal space along the anterior axillary line.

In some embodiments, the EMG sensor is adhered to a patient's skin. In some embodiments, the EMG sensor is part of a band or garment. In one embodiment, the EMG device (or other worn sensor) is wirelessly charged from a NO device that is located nearby while in use. This eliminates the need for a patient to replace batteries on the EMG device, or frequently replace EMG devices.

Bioimpedance Breath Detection

In some embodiments of a NO delivery system, patient respiratory activity is monitored by measuring chest bioimpedance. Bioimpedance offers similar benefits to EMG with earlier inhalation information. This approach to breath detection detects changes in the impedance of the chest as the diaphragm moves and the lung volume changes. In some embodiments, two electrodes are placed, one each, on the left and right sides of the chest. In some embodiments, three are more electrodes are utilized to provide a reference measurement in addition to a dynamic measurement. Low current (e.g., 1 μA) is sent to a first electrode. In the two-electrode embodiment, the voltage at the second electrode is measured and the impedance (voltage divided by the known current) is calculated. In the three-electrode embodiment, voltage is measured at the second electrode as well as the third electrode. The voltages measured are compared (e.g., calculating a ratio) and the calculated value is tracked over time. Variation in the chest impedance measurement can be correlated to various stages of the respiratory cycle, enabling a NO delivery system to detect inspiration.

Chest Band Breath Detection

In some embodiments of a NO delivery system, patient respiratory activity is monitored by measuring changes in the shape of the chest wall. In some embodiments, this is done with a or more sensors that measure the shape of the chest. In some embodiments, the sensors are in a band that encircles the chest and/or abdomen. In some embodiments, the sensors do not fully encircle the patient. In some embodiments, the device is held to the patient with a stretchable portion (e.g., elastic). In other embodiments, the device is held to the patient with adhesive like a band-aid. Various types of strain and/or displacement sensors can be utilized. In some embodiments, the sensing portion completely encircles the patient chest. In some embodiments, the sensing portion covers a portion of the chest wall (e.g., one side). The band is typically in a transverse plane (horizontal in a standing person). In some embodiments, the band is placed at the elevation of the chest. In some embodiments, the band is placed at the elevation of the abdomen. In some embodiment, the band presses a balloon against the skin of the patient. The pressure within the balloon is measured and varies with respiration. The band is utilized to detect changes in the shape of the patient due to respiration. This approach can be beneficial to NO delivery because chest wall changes occur early in inspiration, enabling a system to detect breath early and with high confidence. Chest band devices communicate with gas delivery devices by either wired or wireless means. In some embodiments, these devices have the processing capability to identify inspirations and send a trigger signal. In some embodiments, they stream data to another device (e.g. NO delivery device, cell phone, etc.) that processes the data for breath detection.

A NO generation device will not know the volume of a current inspiratory event, however, prior inspiratory events can be used to predict the timing of the next inspiratory event. In some embodiments, the system can predict the timing of the next inspiratory event in order to dose the early parts of inspiration. A NO generation device can detect other respiratory events, such as the time of peak inspiratory flow (via max vacuum pressure), time of end of inspiration (via pressure returning to atmospheric), beginning of exhalation (pressure going positive), and end of exhalation (pressure returning to atmospheric) as other inputs into estimating the timing of the next inspiration.

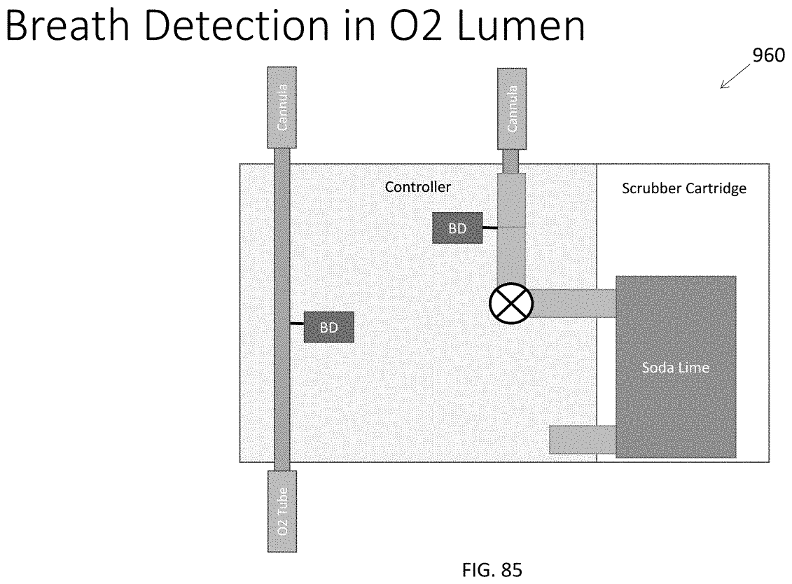

FIG. 85 depicts an embodiment of a NO generator with an oxygen pass-through. This feature enables the NO generation device 960 to monitor the patient's use of the oxygen lumen for detection of inspiratory events. The oxygen lumen is typically unobstructed, whereas, in some embodiments, the NO lumen may have scrubbing material and filters that diminish signal strength. Thus, it can be beneficial to detect breath through the oxygen lumen. In the embodiment depicted, a secondary breath detection sensor is utilized in the NO delivery path. In some embodiments, a NO generation system uses both sensors as a redundant means to detect breath. Breath detection can be achieved through any number of means, including but not limited to pressure, flow, strain of the oxygen lumen wall, microphone, and temperature.

Activity in the oxygen lumen can create pressure artifacts within the NO delivery lumen, affecting the signal used for detection of breath. Typically, these artifacts occur at the terminal end of the lumen where the flow of the NO lumen and the O₂ lumen merge. Artifact is also possible from swelling of the oxygen lumen due to pressure imparting a pressure on the NO lumen. This type of interference between lumens becomes problematic when the O₂ delivery is not synchronized with the NO delivery. One way this happens is if pneumatic coupling between the delivery device and patient is minimal and the O₂ generator fails to detect breaths properly. Many oxygen concentrators on the market respond to an absence of detected breaths by generating periodic O₂ pulses that are asynchronous with actual patient breathing. These asynchronous O₂ pulses can present a challenge to breath detection through the NO lumen. Depending on the construction of the lumen, the anatomy of the nose and degree of nasal prong insertion, flow out of the O₂ lumen can cause a measurable pressure change in the NO lumen that might be confused with a breath by the breath detect method, thus causing NO pulse delivery to not occur or to occur at the wrong time. Unlike O₂ generation, some applications of NO generation require the pulse to be synchronized with breathing to avoid vasodilation of unhealthy parts of the lung, conserve battery power, and minimize introduction of NO/NO₂ to the ambient environment (e.g. NO gas delivered late in the breath is delivered to the airway and exhaled). To avoid generating NO in response to asynchronous O₂ pulses that do not correspond to actual breaths, measurements of one or more of pressure and flow within the O₂ lumen can be used by the breath detect method to compensate the pressure readings within the NO lumen and provide more reliable breath detection. Asynchronous O2 delivery can be detected by a NO delivery system based on one or more of the following techniques: 1) Detection of O2 delivery at a frequency known to be used by the O2 delivery system for n breaths, 2) Detection of O2 delivery during patient exhalation, 3) Communication from the O2 delivery device that it is in asynchronous mode, 4) detection of incomplete breath signals (i.e., O2 pulses produce either a positive or a negative pressure in the nasal cavity. These events are associated with inhalation (negative) and exhalation (positive) pressure. When an NO delivery system detects one type of event without the other, asynchronous O2 delivery may be the reason. In some embodiments, a NO generation system alarms when asynchronous O2 delivery is detected to alert the user that there is a problem with breath detection.

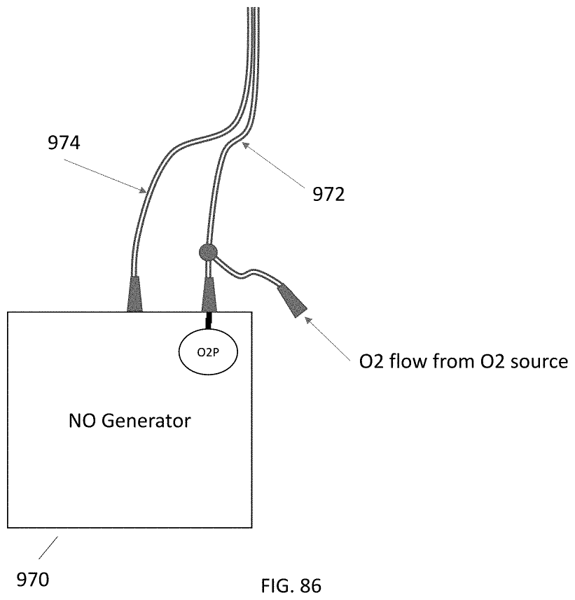

FIG. 86 depicts an exemplary embodiment of a NO generation system 970 that uses an oxygen delivery lumen 972 for breath detection. A dual-lumen delivery device connects the controller with a NO lumen 974 and the O₂ lumen 972. The NO lumen is utilized to deliver NO to the patient and optionally detect breath. The O₂ lumen provides fluid communication from a pressure sensor within the NO generator to the patient. The O₂ lumen bifurcates between the NO device and the patient with a connector for receiving oxygen from an oxygen source.

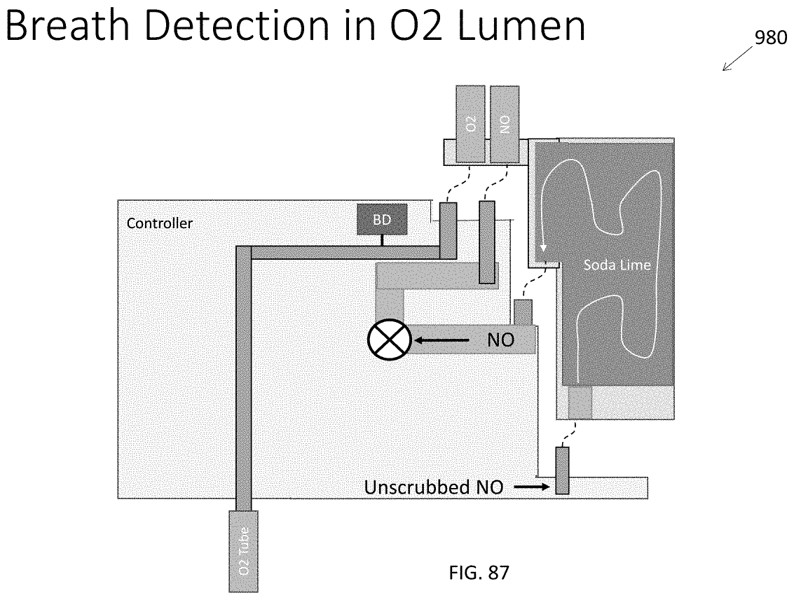

FIG. 87 depicts another embodiment of a NO generator 980 with oxygen through-flow. A pressure sensor within the oxygen lumen is utilized to detect one or more of breath, obstruction, kink, presence/absence of cannula. The oxygen lumen sensor measurements can be utilized to determine the frequency of oxygen device use. Thus, it can monitor O2 compliance with a pressure and/or flow signal within the O2 lumen. The scrubber cartridge in this design is installed from above. When pressed down into the system, four independent gas connections are established for unscrubbed product gas in, scrubbed product gas out, product/bypass gas in and oxygen in. The delivery device (e.g., cannula) is attached to the scrubber cartridge to facilitate installation and replacement of the cannula and scrubber cartridge at the same time. In some embodiments, the delivery device is permanently attached to the scrubber cartridge and is replaced at the same time. Dashed lines FIG. 87 depict pneumatic connections that are established when the cartridge is fully inserted.

Environmental Compensation

A portable NO generator is expected to be capable of operating in a range of environmental conditions. For example, outdoor air conditions can be humid, with high levels of water within the air. When air is compressed, the relative humidity increases and can reach levels where condensation can occur. Condensation within a NO generator can be determinantal. Liquid water can fill voids intended to serve as dead space. NO₂, a water-soluble molecule, can enter liquid water within a system, forming nitric acid and corroding internal components. Another risk is that liquid water/acid exits the device and is delivered to the patient.

As an example, air at 40 deg C. and 95% humidity requires roughly 50% of the water removed to prevent condensation at 10 psi. It follows that a NO generation device operating at elevated pressures, requires some level of water removal from reactant gas before it is pressurized by a pump.

In some embodiments, reactant gas is dried prior to the pump. This prevents condensation within the system. In some embodiments, reactant gas is dried completely to at or near 0% RH. In some embodiments, reactant gas is sufficiently dried to prevent condensation without removing all of the humidity from the gas. In some embodiments, reactant gas humidity is controlled to non-zero level (e.g., 15% RH).

Further benefits can be had by drying the reactant gas before the plasma chamber. Drying reactant gas completely eliminates risk of hydrogen-containing gas species in product gas, simplifies plasma control, decreases wear on electrodes, in addition to preventing condensation within the system. Depending on the type and quantity of scrubber material, dried reactant/product gas passing through the scrubber can dry it out prematurely, reducing NO₂ sequestration. In some embodiments, product gas is hydrated post-plasma chamber, and pre-scrubber to protect the scrubber from drying out. In some embodiments, product gas is hydrated with hydrating beads (e.g., water loaded silica gel) after the plasma chamber and before the product gas scrubber.

Water content in the reactant gas (i.e., humidity) can affect NO production more than 40%, requiring compensation in NO production. For example, a treatment controller can increase the plasma duty cycle to counteract the reduction in NO production associated with elevated humidity. There are benefits to drying reactant gas before the plasma chamber as well to avoid humidity effects on the NO generation. Holding the humidity level constant, even when the humidity is not zero, provides benefits in simplifying the plasma control algorithm. In one exemplary embodiment, a NO generation system treatment controller is connected (wired or wirelessly) to a humidity sensor that measures the water content in reactant gas entering the plasma chamber. In some embodiments, absolute humidity is measured. In other embodiments, absolute humidity is calculated from measurements of temperature, pressure and relative humidity of the reactant gas. In some embodiments, an ambient humidity measurement is used in addition to and/or instead of a reactant gas humidity measurement. The treatment controller includes a relationship between NO production and reactant gas humidity (e.g., look-up table, equation, etc.) stored in its memory. The system measures the humidity of the reactant gas entering the plasma chamber, references the compensation parameters in memory, determines a NO production correction factor, and alters the NO production level accordingly to compensate for the expected change in production. For example, if it is expected for there to be a 20% decrease in NO production based on the humidity level, the NO generation system will set the plasma parameters to produce 20% more NO. It should be noted that this compensation effect may not be the only compensation effect utilized when selecting the plasma parameters. A NO generation system may also compensate for reactant gas temperature, reactant gas pressure, electrode age/wear, delivery system type/size, anticipated NO loss, scrubber type, scrubber age and other parameters.

There are many ways humidity can be removed from reactant gas. In some embodiments, reactant gas passes over a desiccant (e.g., molecular sieve, silica gel, montmorillonite clay, etc.). Desiccant can be packaged as granules, sheets, a coating or compounded into the polymer that makes the walls, tubes or baffles of a design. In some embodiments, reactant gas passes through humidity exchange membrane tubing (e.g., Nafion) with a temperature, pressure, and or humidity gradient across the membrane that drives water out of the gas into the surrounding space.

In some embodiments, the controller actively controls the humidity level of the reactant and/or purge gas based on feedback from a gas humidity sensor. In some embodiments, the controller actively controls a heater that increases the reactant gas temperature, thereby lowering the relative humidity, to prevent condensation within the system. In some embodiments, the reactant gas heating is based on closed loop control from a reactant gas temperature sensor. The controller monitors the system for humidity, pressure and temperature conditions that could result in condensation within the system. In some embodiments, the peak pressure within the system is fixed and only the temperature and humidity vary. When reactant gas conditions at the sensor indicate that relative humidity levels are approaching 100%, the controller can mitigate using one or more methods listed below. In some embodiments, the controller only dehumidifies reactant gas when the relative humidity exceeds a threshold (e.g., 90%), above which condensation would occur at the peak pressure. In some embodiments, the relative humidity of reactant gas is managed at all times of operation. In such an embodiment, a pair of proportional valves or similar adjustable orifices control the relative flow restrictions of the dry and ambient (humid) gas inlets. Based on a humidity sensor reading of the blended gas, the coordinated control of these valves allows the humidity to be set at any level between fully dry and the ambient humidity. In some embodiments, humidity of incoming gas is actively controlled by adjusting the blend of desiccated dry air with non-desiccated ambient air to achieve a non-condensing humidity level. In some embodiments, one or more of temperature, pressure, and humidity are varied to drive water out of reactant gas flowing through a humidity exchange membrane tube.

Desiccant types and mesh sizes can affect humidity control. Mesh size affects flow restriction, as well as the surface area exposed to the flow. More surface area improves dehumidification. A downstream humidity sensor can be used to detect desiccant exhaustion. In some embodiments, silica is used as a desiccant. This material expands as it absorbs water and requires room for expansion within a NO generator design. In some embodiments, more than one desiccant material is utilized. Molecular sieve does not expand appreciably so it can be contained within a more rigid housing.

In some applications, desiccant is in the form of sheets. The sheets can be flat or have a topology. Having a topology facilitates packing of layers of sheet material in stacks or in a spiral while maintaining a pathway for glasses to flow through the stack/spiral. Sheet desiccant material can provide a benefit in decreased flow restriction, reduced product variation, and improved flow and dead volume consistency.

When granular desiccant material is used, features are required to prevent desiccant from migrating. This can be accomplished by including baffles and/or screens before and/or after the desiccant zone within a NO generation system. Given that an ambulatory NO generation system is portable, it can experience accelerations from multiple directions and be set down on any face. It follows that granular desiccant (and soda lime material for that matter) will settle to the lowest point possible when not packed. Thus, baffles and screens need to function from many angles. Desiccant materials can erode when they move relative to each other, forming dust that can clog filters and internal passageways within a device. In some embodiments, acceleration and erosion of desiccant material is mitigated by packing desiccant granules along with open-cell foam particles. The open cell foam particles fill up the remaining space in the desiccant chamber while still permitting gas to pass through and can compress to accommodate any volume changes in desiccant material.

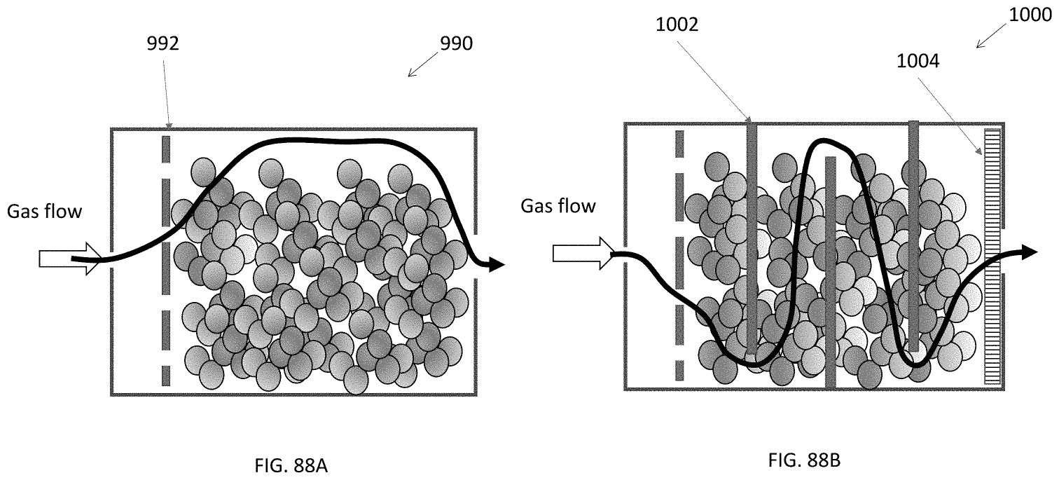

FIG. 88A depicts an embodiment of a granular desiccant chamber 990 that at least partially desiccates gas. Gas passes through an initial perforated baffle 992 that has pore sizes that are small enough to prevent desiccant migration. Granules have settled to the bottom of the chamber, creating a pathway for gas to pass above the desiccant. At faster flow rates, the gas will be partially dried. It can be important to make sure that desiccant does not migrate out of the chamber through the exit point on the right side of the chamber and potentially clog the gas flow path, which will at least partially depend on the size of the exit point.

FIG. 88B depicts another embodiment of a desiccant chamber 1000 with solid, non-perforated baffles 1002 that force gas flow to pass through the desiccant material. Even though the desiccant has settled the same amount as in FIG. 88A, the gas is forced through the desiccant rather than circumventing it. The geometry shown can be extrapolated to three dimensions, allowing the desiccant to be positioned in any orientation without concern of flow bypass. The baffles also effectively lengthen the desiccant flow path, thereby increasing the gas contact time with the desiccant media. A filter 1004 at the exit of the chamber prevents desiccant migration and collects any particles that might be shed from the desiccant material. In some embodiments, a helical flow path is filled with desiccant to ensure that gas passes through desiccant regardless of orientation of the helix.

In architectures with a bypass pneumatic pathway, humidity management of gas flowing through the bypass channel and gas flowing through the plasma chamber can vary. In some embodiments, purge gas is dehumidified less than reactant gas for one or more reasons including that it is pressurized less within the system and there are no chemical reactions occurring in purge gas that water could affect. This approach can be beneficial because less desiccant is required for the system overall, reducing cost, mass and size of a NO generation device. In some embodiments, purge gas is not dried. In some embodiments, purge gas is dried to 0% RH. In some embodiments, purge gas is actively dried to a controlled level that prevents condensation. In some embodiments, the purge gas is made by a constant blend ratio of ambient air and dried air. The humidity in the purge line may vary, but will remain below condensing levels at all times.

In some embodiments, a NO generation device detects environmental moisture with an ambient humidity sensor and based on this reading either dries or does not dry incoming gas. This approach can preserve desiccant for when it is needed based on ambient conditions, extending desiccant life. In the case of active drying methods requiring power (fans, heaters, etc.), this approach preserves energy and/or battery life.

In some embodiments, humidity of gas within the NO generation device is increased. Reasons for doing this can include prevention of drying out a scrubber or driving gas humidity to a non-zero target level. One way of doing this is with desiccant beads that are designed for a specific humidity level and will absorb or release water to drive humidity to the target. In some embodiments, desiccant used for adding humidity can be replenished by adding water. This approach can have benefits in preventing drying out of the scrubber.

Some types of desiccant change volume significantly as they absorb water. In some embodiments, desiccant beads are placed in a balloon or compliant tube to avoid air spaces (minimal void space), maintain density, and allow for expansion due to water absorption.

In some embodiments, desiccant beads are loaded to a particular humidity at the time of manufacturer. The loading level is selected so that the beads can pull down excessively high humidity and pull up excessively low humidity. In some embodiments, the target humidity of the desiccant beads are the same as that of soda lime (15-20%). In some embodiments, the water content in the beads is different than that of soda lime and it is necessary to prevent water transfer between the two material during storage before use. For example, desiccant beads designed to dry to 0% would pull water content from soda lime during storage without a partition between the two materials.

In some embodiments, the desiccant pathway and scrubber pathway through a cartridge are covered with an adhesive film or foil during storage. In some embodiments, the film is removed by the user before connecting the cartridge to a NO generation device. In some embodiments, the film is pierced by elements of the system when the cartridge is inserted to established fluid communication between the controller and the cartridge. In some embodiments, a NO generation system can detect piercing the film (e.g., optically, measured force) and generate an alarm if the film was not detected. In some embodiments, the system will only permit NO generation if the film was detected upon cartridge insertion.

Desiccant can be packaged with the scrubber, the battery, the delivery device or on its own. Similarly, the VOC scrubber, reactant gas particle filter, NO2 scrubber, and final particle filter can be installed individually or packaged in common assemblies.

Once a cartridge has been removed from its packaging, it should be immediately installed into the NO device. In some embodiments, when a gas conditioning cartridge is removed from its packaging, it should be installed immediately in a NO generation device to begin use. There is a risk that a user could take too long to install the cartridge resulting in contamination of the various materials and/or interaction between the various materials. In some instances, it is possible that the packaging of the cartridge has failed and one or more sensitive materials in the cartridge have been exposed to air for a period of time and have been altered. In some embodiments, there is a marker on the cartridge that changes color after air exposure for a certain amount of time. In one embodiment, the marker is in the form of an adhesive label with chemistry on it that darkens over the course of 15 minutes. A NO generator that detects a dark color on the air sensor label during cartridge installation can reject that cartridge as not being a fresh example. Example chemistries are similar to that of apple and avocado that turn brown when exposed to air. In one embodiment, the sensor material is printed in a word or symbol indicating “Do not use” to alert the user that the cartridge has been contaminated and should be discarded.

In some embodiments, a NO generation system battery charger dries out desiccant beads. The battery can be located in the same housing as the beads so that the battery charger dries out beads too. In some embodiments, the battery charger could heat the desiccant to dry it out so that it can be reused.

Gas Conditioning Cartridge Design

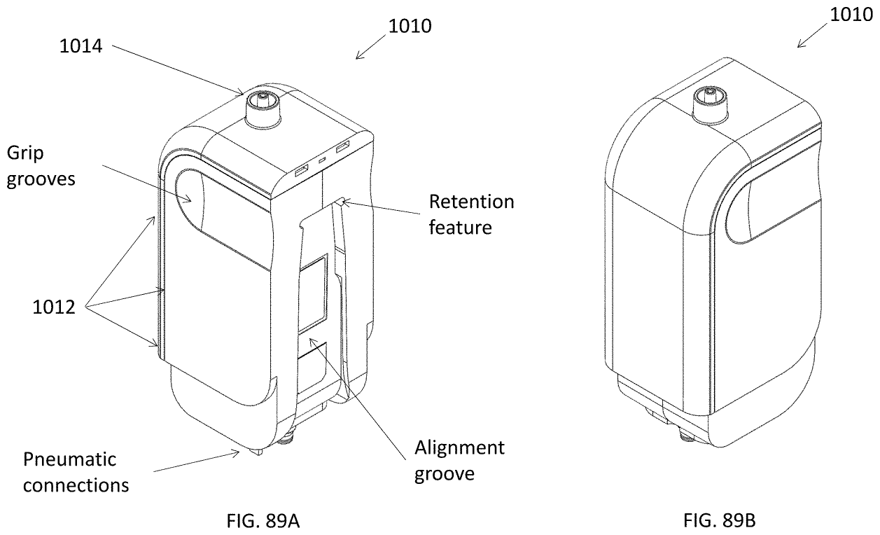

FIGS. 89A and 89B depict an embodiment of a gas conditioning cartridge (GCC) 1010. The external surfaces are smooth and easily cleaned. Ambient air enters the cartridge through an air inlet gap 1012 between the cap component and main body. This perimeter inlet permits air ingress into the system when it is resting with any side on a flat surface. The GCC engages with the controller with a dove-tail groove. Divots at the top of the groove engage with detents or button-actuated pins that retain the GCC in place once fully inserted. A pneumatic connection 1014 delivers NO into a delivery device. The connection consists of a wall around a nipple. The wall protects the nipple from impact and also restricts the outer diameter of the mating connector. This prevents the oxygen connector from being connected in the wrong place. Pneumatic connections to the rest of the system are at the bottom of the GCC.

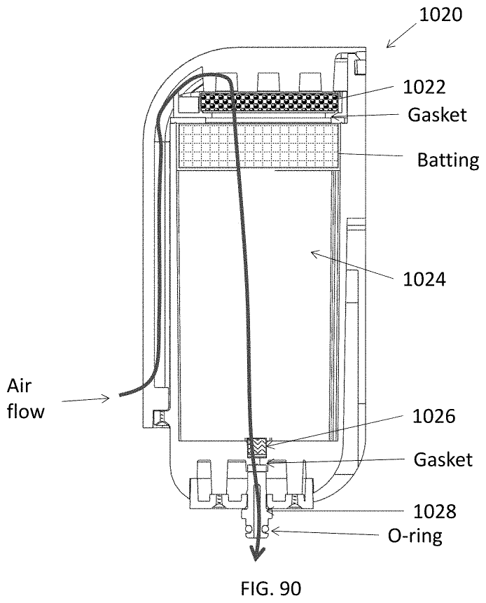

FIG. 90 illustrates an exemplary embodiment of a cross-section of a gas conditioning cartridge 1020. Air is pulled in through a perimeter intake along three sides of the GCC, allowing flow independent of device orientation. Air is first pulled through a VOC filter 1022 (activated carbon in this case), then passes through a volume of molecular sieve 1024. An inert open cell material (e.g., polyethylene batting, polypropylene foam) maintains light compression on the sieve material and prevents sieve migration. The air then passes through a particulate filter 1026 before entering the durable instrument via a pneumatic fitting 1028.

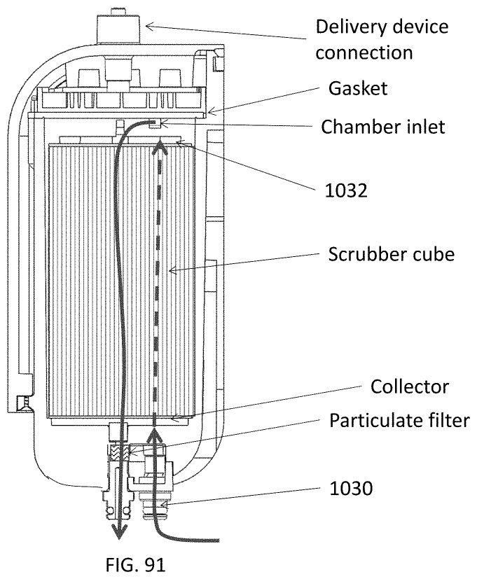

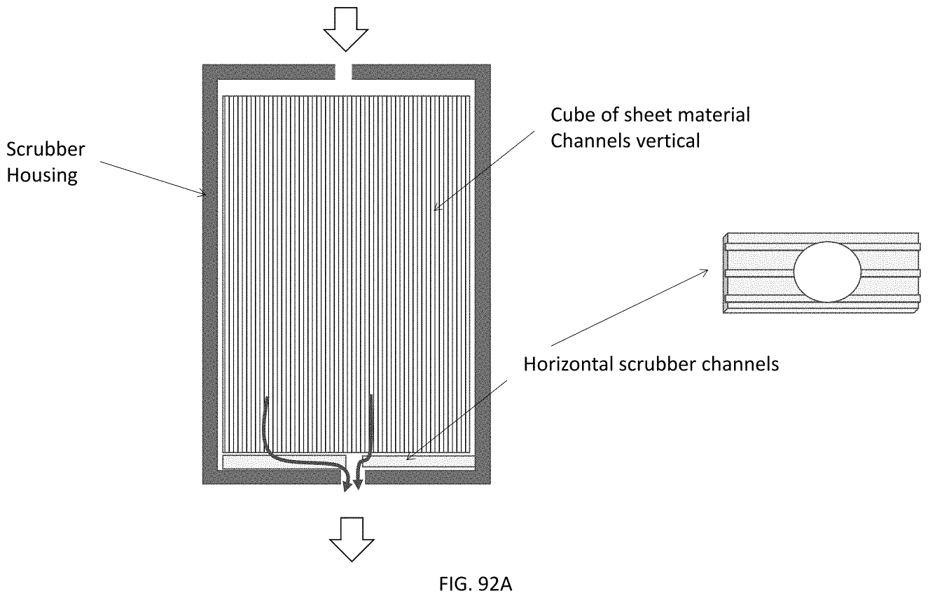

FIG. 91 presents a cross section of the GCC in the region of the NO2 scrubber. NO Product gas enters the GCC through a pneumatic fitting 1030, and then travels via an internal pathway (denoted with dashed line) to the distal end of the scrubber chamber. The gas is then introduced to the chamber, which is filled with a stack of soda lime sheets with ridges. As gas accumulates within the scrubber chamber, the pressure increases, and nitrogen dioxide is removed. A scrubber retainer 1032 constrains the scrubber and ensures its position does not shift with device orientation. When breaths are detected, a valve in the main device opens to permit product gas to leave the scrubber chamber. Before leaving the GCC, the gas passes through a particulate filter. This geometry was designed to minimize the dead volume downstream of the soda lime material since any gas downstream of the scrubber sheets is no longer actively scrubbed for NO2.

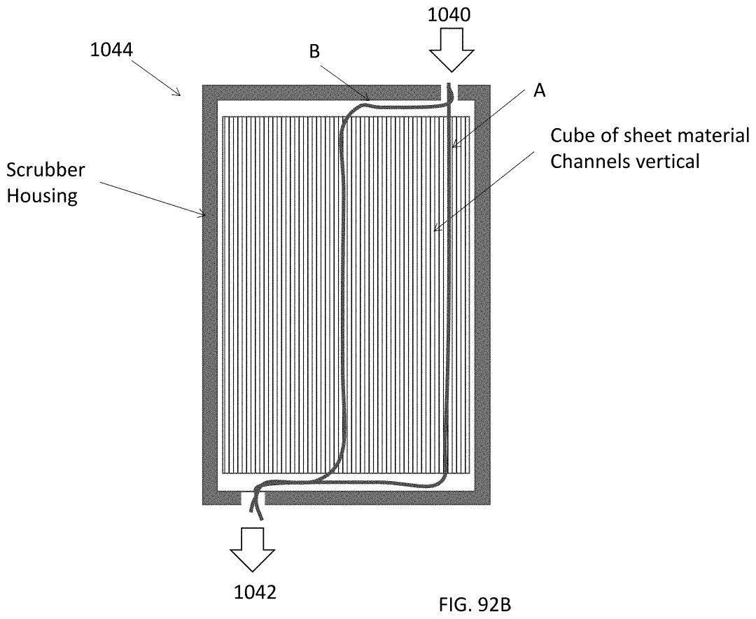

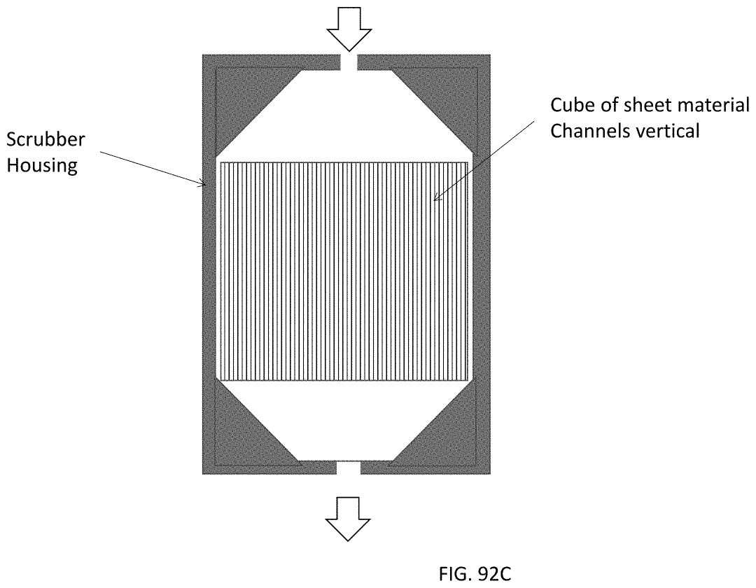

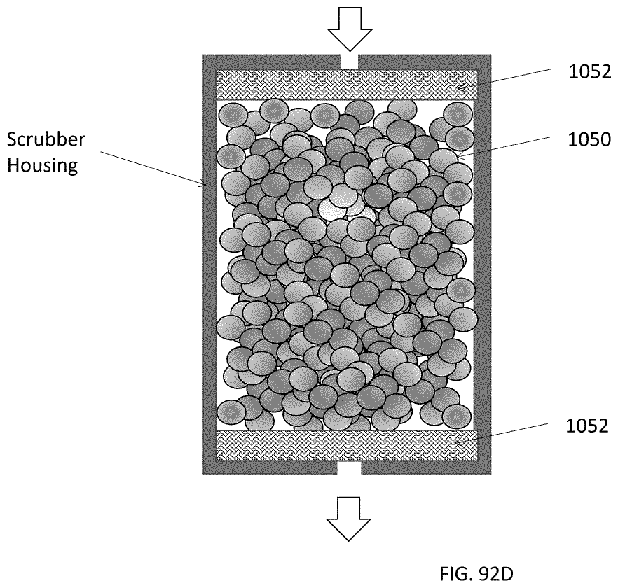

An air gap between the scrubber material and the chamber wall is provided at the bottom of the design to permit gas from each of the channels within the sheet material to travel to the exit. It is important to permit easy gas flow through this area so that all channels within the scrubber have similar gas flow to ensure maximal scrubbing and scrubber longevity. In some embodiments, additional scrubber sheet material is placed in the air gap to further reduce the post-scrubber dead volume, as shown in FIG. 92A.

FIG. 92B depicts a scrubber housing filled with scrubber material (sheet material in this case). The product gas inlet 1040 and product gas outlet 1042 are on opposite ends and sides of the chamber to promote even flow through the scrubber 1044. Each flow path has a similar amount of left-to-right path length as well as top-to-bottom path length to prevent the gas flow from taking a short-cut. This similarity in flow path is depicted by comparing the path length of path A and path B in FIG. 92B.

FIG. 92C depicts an exemplary scrubber chamber that has tapered or conical entry and/or exit geometry to evenly recruit channels within the scrubber.

FIG. 92D depicts an exemplary scrubber chamber that utilizes granular scrubber material 1050 (e.g., soda lime). The material is held in place by open cell material 1052 (e.g., foam, filter, textile, etc.). The open cell material provides gentle compression on the scrubber granules to prevent migration and relative motion which prevents clogging and dust generation, respectively. The open cell material also ensures fluid communication with the entire cross-section of the scrubber to improve uniformity of gas flow through the chamber. The chamber dimensions, scrubber granule size, and granule quantity affect the quantity of dead volume in the scrubber, the scrubber service life and level of NO2 scrubbing.

Striking the correct balance of scrubber surface area, dead volume and flow restriction is important to achieving sufficient NO2 removal while satisfying pulse delivery timing requirements. Flow restriction and surface area have an inverse relationship and would be potential tradeoffs, depending on the available dead volume and operating pressure. Higher levels of surface area provide greater levels of scrubbing. The dead volume within the scrubber is determined by the maximum pulse volume to be delivered to the patient and the operating pressure, however larger scrubber dead volumes are acceptable. A larger dead volume results in shallower pressure deviations as pulses are delivered to the patient, improving a NO delivery system's tolerance to changes in breath rate. Flow restriction is minimized. Effective designs can be achieved with grooved sheet material and appropriately-sized granular soda lime. In one exemplary design, the dead volume is 38 ml with only 12 ml of dead volume within the scrubber.

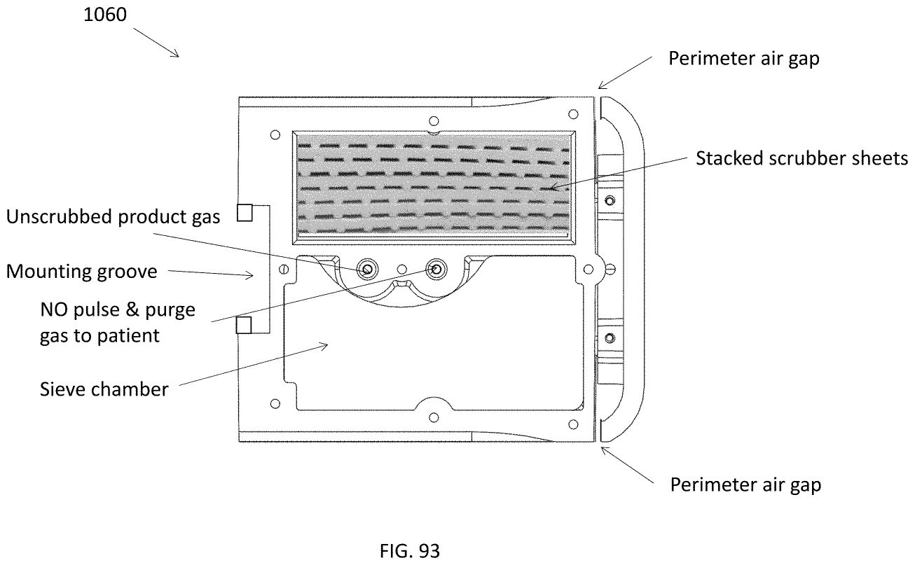

FIG. 93 depicts a horizontal cross section of an embodiment of a GCC 1060. The scrubber chamber is filled with sheet scrubber material. The sheets are banded together with polymer sheets or stapled together to form a cuboid shape (i.e., rectangular prism) for ease of insertion to the molded GCC housing. Molded-in paths route unscrubbed product gas to the top of the scrubber chamber and route final product gas and purge gas through the GCC to the outlet at the top of the cartridge. The cavity on the bottom houses a molecular sieve for drying the reactant gas. Perimeter inlets are shown on the right side of the image for letting air into the cartridge.

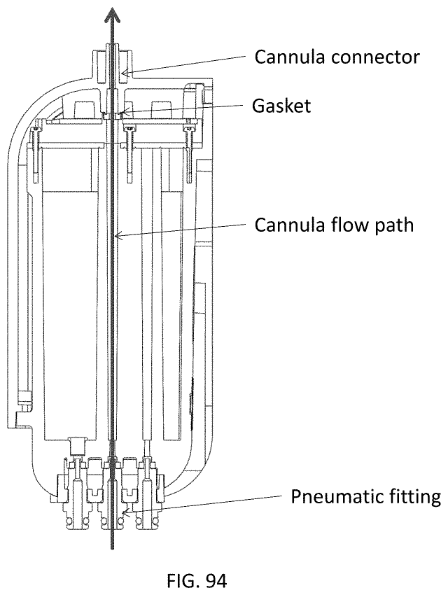

FIG. 94 depicts a cross section of the GCC at the location of the scrubbed product gas and purge gas delivery path. Gas enters the GCC through a pneumatic fitting at the bottom of the cartridge and travels via an internal path to the distal end of the GCC. After passing through a gasket, the gas leaves the GCC via a custom connector fitting. Other fittings shown at the bottom of the GCC are for other purposes described above.

Accessories

In some embodiments, an ambulatory wearable satchel can provide drying to reactant gas and insulate the patient from surface temperatures. In some embodiments, the satchel includes a pocket that houses desiccant material and the NO generator sources reactant gas through that pocket.

SPO₂ and Methemoglobin Sensor

When NO combines with hemoglobin, a molecule called “methemoglobin” is formed. Methemoglobin is unable to bind with oxygen which can lead to decreased oxygen uptake in a patient. In some embodiments, a sensor can be provided that measures one or more of SpO₂ and methemoglobin and can be used for feedback for NO therapy. SPO₂ and methemoglobin are measured noninvasively, using an optical method. In some embodiments, the sensor is placed on a user's ear, or foot or finger. In some embodiments, the NO generator varies the NO dose as a function of SPO₂ and/or methemoglobin. For example, when SPO₂ is high, the patient is well oxygenated and is not requiring a large dose of NO. A NO generator can decrease the dose automatically in response to high SpO₂ levels. When SPO₂ is less than a threshold (e.g., 90%), the NO dose is increased (e.g., 10%) up to a limit. Increasing the dose in a patient is generally harmless unless methemoglobin levels rise. By monitoring methemoglobin levels, a NO generator can decrease the NO dose in response to methemoglobin levels exceeding a threshold instantaneously, or for a particular length of time. For reference, the panic threshold for methemoglobin is about 10%, typically 5% is the threshold used during high concentration NO therapy (>150 ppm). In monitoring, the methemoglobin warning threshold is about 2%.

In some embodiments, SpO₂ measurement input is used for closed loop control to expedite the weaning of the patient from the NO therapy. The NO delivery device decreases the dose according to a schedule. If the dose is decreased faster than the patient can handle, their SpO₂ levels decrease and the NO delivery device either slows or reverses the weaning process until SpO₂ levels recover. In some embodiments, a NO delivery system weans a patient by decreasing the NO dose by 1 mg/hr every 30 minutes. In one scenario, a patient has an SpO2 of 95% at the beginning of the weaning process. In one embodiment of a weaning process, a NO delivery device monitors the patient SpO2 throughout the weaning process. In some embodiments, if the SpO2 level decreases 1 point, the NO device holds the current NO level for longer than the scheduled 30 minutes (e.g., 1 hour). If the SpO2 decreases 2 points or more, the NO device returns to the previous NO setting. In some embodiments, the thresholds, durations and increments/decrements of NO in an automated weaning process are user-defined. These settings can be stored in device memory for future use. In some embodiments, a NO device stores multiple weaning programs so that the user can select which weaning program that want to deploy. In some embodiments, there are weaning programs assigned to specific patient indications. In some embodiments, when the NO device is unable to wean a patient because of SpO2 response, the NO device does one or more of alarm, notify a physician (wired, wirelessly, visually, audibly), abort weaning, postpone weaning until SpO2 values recover to the initial value, and postpone weaning until SpO2 values stabilize.

In some embodiments, a NO delivery system monitors methemoglobin levels in the patient's blood either directly or through an external device. External devices can be connected with wires or wirelessly. Every patient has their own rate of methemoglobin clearance rate and their own rate of NO uptake. In some embodiments, a NO delivery device delivers as much NO as a patient can tolerate, as is often the case in treating pulmonary and airway infections. In this application, a NO delivery device manages the methemoglobin at or just below the patient's maximum clearance rate by varying the NO dose. This is often done with a PID controller using NO dose to control methemoglobin levels. This automated process can provide a huge improvement in patient care due to its ability to make treatment adjustments real time without clinician intervention. It also reduces the labor requirements for weaning when compared to manual weaning methods, which are the standard of care today. In some embodiments, SpO2 and/or Methemoglobin are measured continuously. In some embodiments, they are measured intermittently (e.g., for 5 seconds every 30 seconds). This reduces the computational burden of monitoring the patient. In some embodiments, the monitoring frequency is variable. For example, as the SpO2 level decreases, the frequency of monitoring is increased. In some embodiments, a NO delivery system generates an alarm (aubible, tactile or visual) to the user in the event of unacceptable SpO₂ and/or Methemoglobin levels.

Delivery Device with Optical Transmission

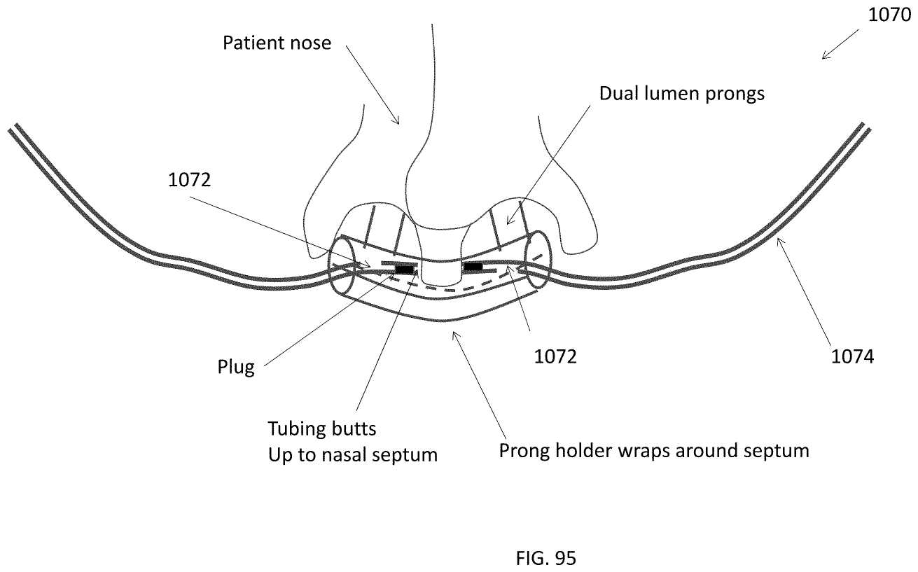

FIG. 95 depicts an exemplary gas delivery cannula 1070 that utilizes the cannula tubing as light pipes to send and receive optical information. In some embodiments, one or more of SpO2 and MetHb are measured optically through the cannula. The tubing extends from the device to the patient with the tubing terminating orthogonal with respect to and pressed against the nasal septum. A tubing skive 1072 cut in the side of the tubing permits delivered gas to exit the lumen. The nasal prong housing orients the tubing with respect to the nasal septum. In some embodiments, the nasal prong housing lightly clips on the nasal septum to maintain good optical contact between the tubing the septum. In the embodiment depicted, a first lumen sends light through the septum, where it is received by a second lumen. In some embodiments, the two lumens delivery the same gas. In other embodiments, the two lumens serve different purposes (e.g., NO and O2 delivery).

In some embodiments, silicone tubing is utilized for its NO chemical resistance, biocompatibility and optical translucence (i.e., nearly 100% transmission with light frequencies from ˜350 nm to ˜1600 nm light). In some embodiments, the cannula tubing is covered by an exterior coating 1074, such as an opaque material, to prevent loss of light through the tubing wall, particularly in regions of tubing flexion. In some embodiments, the tubing has a reflective coating that reflects light back into the tubing as it exits the wall. In some embodiments, optical fiber is integrated into the tubing to conduct light signals from the NO controller to the patient nasal septum and back.

Optical measurements can be measured either continuously or intermittently. In some embodiments, the SpO2 and/or MetHb is measured every 5 minutes, for example. In a typical application of NO, the MetHb>5% is considered an important threshold. The time required to reach this threshold depends on the NO dose, patient genetics (e.g., MetHb clearance rate) and the underlying patient condition. The threshold may be met within a few hours of treatment in some cases, whereas the threshold is never met in other cases. In some embodiments, the device will check at the beginning of the therapy for the baseline MetHb level and set the interval for checking MetHb and adjusting NO dose according to the baseline MetHb level. In some embodiments, this interval is in the range of every 1 to 20 minutes. In some embodiments, MetHb checks and NO adjustments are done continuously.

Every joint (bonded connection) in the delivery system is a potential source of light loss. In some embodiments, joints are minimized by extending cannula tubing to terminate on one side of the septum and the other tube terminates on the other side of the septum.

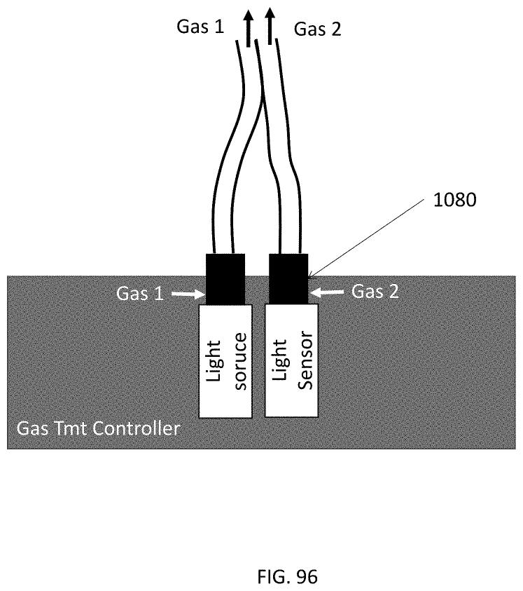

FIG. 96 depicts an exemplary connection of an optical measurement/gas delivery device with the gas source. The tubing butts against an optical coupling 1080 for good optical contact. In some embodiments, an index-matching material (e.g., oil) is utilized to improve light transmission from the controller to the delivery device and back. Light is emitted in the target frequency (e.g., 660 nm and 940 nm for SpO₂ measurement) into one of the delivery device lumens. Light is received by a sensor that monitors a second lumen of the delivery device. A similar approach can be utilized to quantify levels of carboxyhemoglobin, methemoglobin, hemoglobin and total oxygen levels when the appropriate light frequencies are utilized. In some embodiments, these measurements are for patient monitoring purposes. Data can be recorded and/or transmitted and alarm thresholds can be set for each parameter. In some embodiments, some parameters serve as input to the NO delivery control algorithm. For example, when methemoglobin levels increase beyond a threshold, NO dose can be automatically decreased to prevent methemoglobinemia and the possible need to cease NO delivery altogether. Oxygen levels can be tracked to understand the well-being of the patient and the effect of NO on the patient's need for oxygen.

In some embodiments, a delivery device with optical properties can be used such that the disposable component is inexpensive and simple with no electronics within it. The patient can also be monitored non-invasively. In addition, there are no additional use steps to connecting and donning an optical delivery device. The benefits of an optical deliver device are many, including feedback to the system to titrate minimally effective dose (typically based on SpO₂). as well as protection against the potential for NO overdose resulting in high MetHb levels.

Firefighter Applications

Nitric oxide is a product of combustion in fires. As a result, nitric oxide inhalation and methemoglobin levels are a concern for firefighters. In one application, a SPO₂ and methemoglobin sensor are used to monitor firemen such that a central person can monitor firemen and tell them when to get away from the fire. In some embodiments, the sensor has wireless communication capability. In some embodiments, the sensor electrically connects with or is incorporated in another patient monitoring system worn by the firefighter.

Safety

NO₂ Limits

In some embodiments, NO₂ thresholds are based on an allowable mass of NO₂ per unit time (e.g., mg/hr). This provides a benefit over concentration-based safety limits because it is independent of the breath volume. For example, the safety threshold for NO₂ inhalation can be 83 μg/hour. In some embodiments, the NO2 threshold is an absolute limit, while other systems utilize a moving average to trigger an alarm and brief excursions above the limit are acceptable. For example, an NO2 alarm is triggered by exceeding the 83 ug/hr threshold for more than 10 minutes. NO delivery systems can also have a threshold for NO2 delivery that results in immediate alarm and/or treatment cessation.

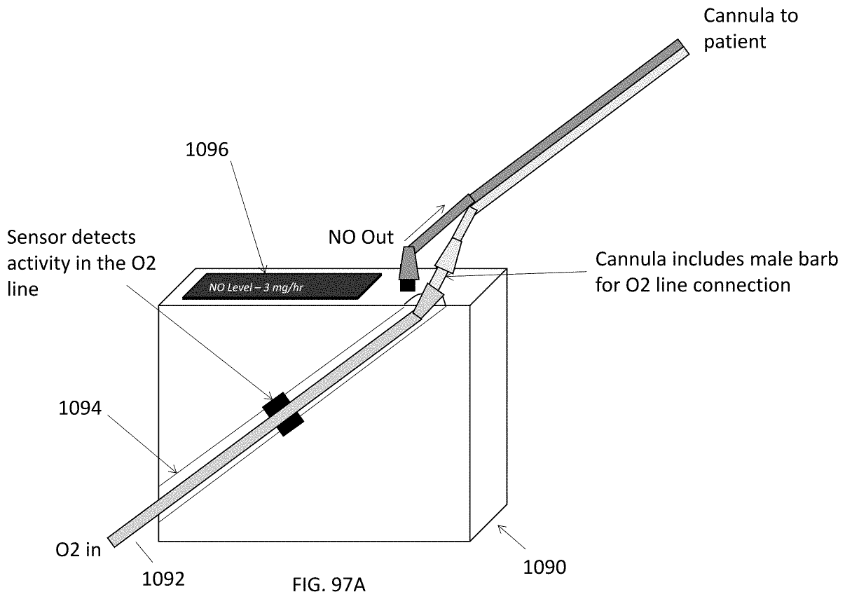

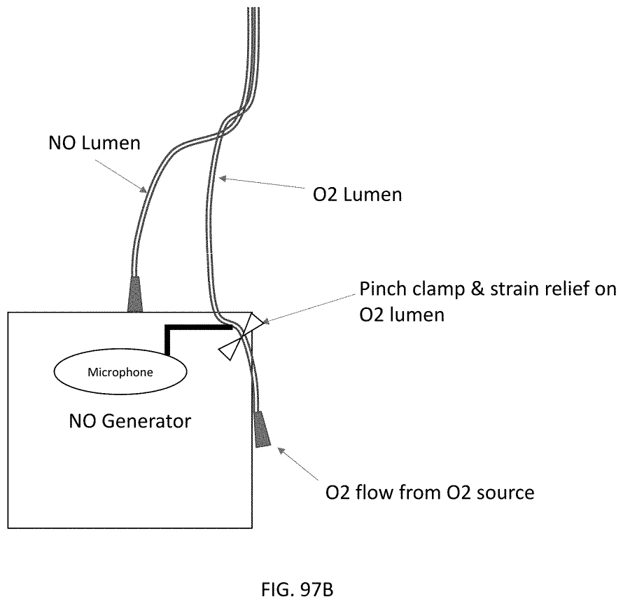

FIG. 97A depicts an exemplary embodiment of a NO generation device 1090 for use with concomitant oxygen delivery. The NO generation device includes a housing with a connection to a delivery device. The connection between delivery device and NO generator includes one or more lumens for NO delivery and optionally NO return, and breath detection. A separate lumen for oxygen delivery is also included in the delivery device. In some embodiments, the delivery device is a nasal cannula. In some embodiments, the delivery device is a face mask.

As shown, the oxygen lumen 1092 inserts into a groove 1094 within the enclosure of the NO device for protection of the tube and/or monitoring of activity within the oxygen tube. A groove can also be useful in managing the O2 line so that it doesn't interfere with the user's view of the user interface 1096. In some embodiments, the oxygen lumen connects to an extension tube, as shown. In some embodiments, the oxygen-carrying lumen in the delivery system is the same length as the NO lumen, as shown. In some embodiments, the oxygen lumen is a different length than the NO lumen. In some embodiments, the oxygen lumen is longer than the NO lumen so that it extends around the patient to reach a portable oxygen concentrator on the contralateral side of the patient. The NO lumen is typically shorter in length and smaller in diameter than the oxygen lumen because NO is affected by transit time and oxygen is not.

Oxygen activity can be monitored by the NO generator non-invasively with a transducer located on the exterior of the oxygen lumen. The transducer transforms vibrations and/or mechanical strain in the oxygen tubing into electrical signals that can be used to monitor oxygen activity. This can be an important feature in monitoring patient behavior to ensure compliance with prescribed treatment and also to ensure that equipment is functioning properly. Example transducers include a microphone, strain gauge, pressure sensor, load cell, capacitive sensor, and others. In some embodiments, the oxygen information collected is qualitative, such as binary information regarding time of use of oxygen. Some embodiments are calibrated for the hoop strain of the oxygen tubing, for example, and provide more quantitative information regarding oxygen flow rate. In some embodiments, the O2 lumen is partially pinched to create a flow restriction in the O2 lumen, as shown in FIG. 97B. FIG. 97B depicts an exemplary embodiment of a NO delivery device that operates simultaneously with an oxygen delivery device. The oxygen lumen is inserted into a feature within the NO device that pinches the NO lumen without fully occluding it. The pinched region of the oxygen lumen generates turbulence in the oxygen flow downstream of the pinch. A microphone in the NO device listens for sounds in the region downstream of the pinch to detect oxygen flow within the oxygen lumen. In some embodiments, the microphone is calibrated to quantify oxygen flow rates. In some embodiments, the microphone signal serves as an input to a NO pulse control algorithm for determining when to release NO to the patient. In some embodiments, an optical sensor detects installation of the oxygen lumen into the groove. The light beam of the optical sensor is broken when the tube is inserted. In some embodiments, the source and detector are on opposite sides of the groove. In some embodiments, light from the source reflects on the opposite side of the groove and is detected by a sensor that is adjacent to the source.

In some embodiments, an oxygen delivery device (i.e., oxygen concentrator) communicates directly with a NO delivery device via wired or wireless means to convey one or more of O2 concentration, O2 flow rate, breath detection trigger signal, remaining battery life, calculated respiratory rate, and other information. In some embodiments, a NO generation system alters the flow rate of NO to prevent flow rates that exceed patient comfort levels, based on the indicated O2 flow rate. Similar dose can be delivered to the patient by increasing the NO concentration. In another embodiment, a NO delivery device uses the breath detection signal from the oxygen delivery device as an input into determining when to deliver NO. In some embodiments, a NO device utilizes the respiratory rate information from an O2 delivery device to determine NO pulse parameters (e.g., delay, duration, dose).

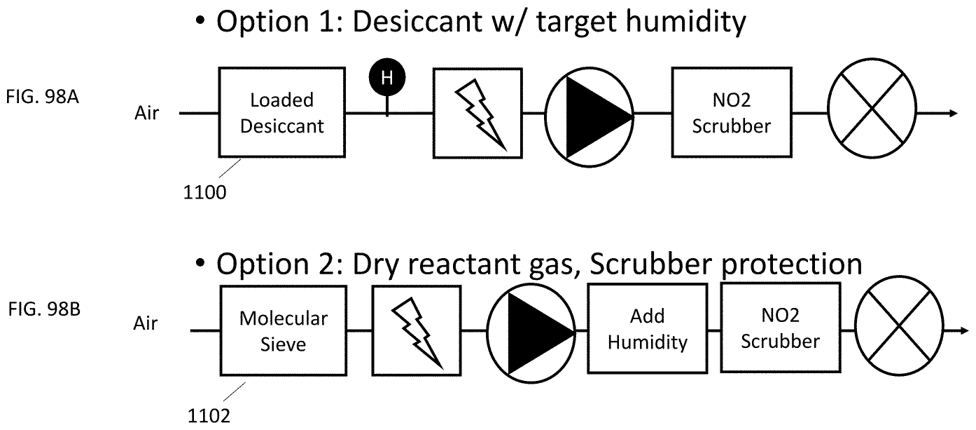

FIG. 98A depicts an exemplary NO generator with a reactant gas preconditioning stage. The preconditioning stage consists of desiccant material 1100 that can alter the humidity of the reactant gas. In some embodiments, the desiccant stage eliminates all humidity in the reactant gas, making the humidity sensor shown optional. In some embodiments, the desiccant material is capable of driving humidity towards a target value. Reaching the target humidity value is not essential in all embodiments. For example, the ambient humidity range for a product may be wider than the range of reactant gas humidity that will not condense within the system. In such a case, a desiccant stage only needs to pull out sufficient water in ultra-humid gas to prevent condensation while pulling up humidity in ultra-dry reactant gas to protect other components of the system, such as gas sensors and scrubbers. In cases of variable reactant gas humidity, the controller can compensate for the final humidity value, as indicated by a humidity sensor, by adjusting the plasma parameters to achieve the target level of NO production. Blending humid and dry reactant gas results in lower amounts of desiccant required for a given amount of service life. This can provide increases in the service life of disposables and the overall size and weight of a device.

FIG. 98B depicts a NO generation device with a desiccant stage that dries reactant gas to extremely low humidity levels. This can be done with molecular sieve materials 1102 (shown), clay, and silica materials, requiring varying levels of desiccant material. NO production control within the plasma chamber is simplified in this embodiment because compensation for production variation stemming from humidity variation is not required. For applications utilizing a scrubber material that requires moisture to function (e.g., soda lime), a humidification stage post-plasma chamber can be utilized to introduce moisture back into the product gas as a way to protect the scrubber from drying out. In some embodiments, the amount of moist scrubber material can be increased to ensure that sufficient inherent moisture is present in the scrubber material to withstand the drying effects of product gas over the expected service life of the scrubber. One advantage of this approach is that little to no moisture enters the plasma chamber, effectively eliminating the potential for forming compounds containing hydrogen in the product gas. These acidic compounds can present a risk when inhaled and also can be corrosive to system internal components. Operating a NO generation system with dry reactant gas can prolong electrode service life, lower the breakdown voltage within the plasma chamber and simplify NO production controls. Lowering the breakdown voltage allows for a simplified design effort to achieve appropriate creepage and clearance within the device to avoid uncontrolled arching.

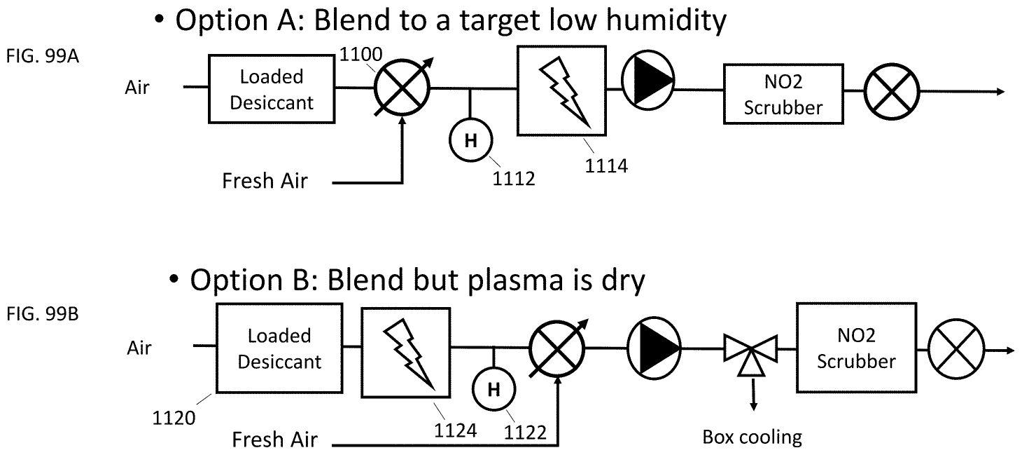

FIG. 99A depicts a NO generation system that blends a mixture of desiccated reactant gas and ambient gas to a target humidity level with a 3-way valve. Blending can be done with a valve 1110, such as proportional valves or binary valves, used with PWM. This approach offers a benefit of reducing the amount of desiccant required since desiccation is only required when ambient conditions are sufficiently humid as to present a condensation risk. In some embodiments, the system blends desiccated and ambient gas to a non-condensing level (e.g., 50% RH). In some embodiments, the system targets a lower humidity level to decrease variation of NO production within the plasma chamber, simplify plasma control and reduce potential for production of hydrogen-containing compounds. A humidity sensor 1112 is shown for closed-loop control of reactant gas humidity as it enters the plasma chamber 1114. In some embodiments, the humidity sensor is located after the plasma chamber.

FIG. 99B depicts an exemplary embodiment whereby all reactant gas flows through a desiccant stage 1120 prior to the plasma chamber. A humidity sensor 1122 downstream of the plasma chamber 1124 detects exhaustion of the desiccant material by sensing an increase in reactant gas humidity levels. When humidity levels exceed a threshold, a NO generation device can alert the user to replace the desiccant. A separate gas pathway provides gas flow into the system for other purposes, such as purging a delivery device and/or device cooling. Purge gas can be a blend of desiccated gas and ambient gas or pure ambient gas, depending on the ambient humidity. In some embodiments, a humidity sensor is located after the blending location either in addition to or instead of the humidity sensor shown. By measuring the humidity of the blended gas, a NO generation system can mix the two gases to achieve a non-condensing humidity level while minimizing desiccant use. The system shown includes a 3-way valve that selects between directing gas towards the patient (right) and out of the system. The system can generate NO and deliver it into the delivery device, then turn off the plasma and continue to push reactant gas down the delivery device to purge the system of NO. Then, the system can direct gas out of the system to cool the device enclosure and repeat the process again.

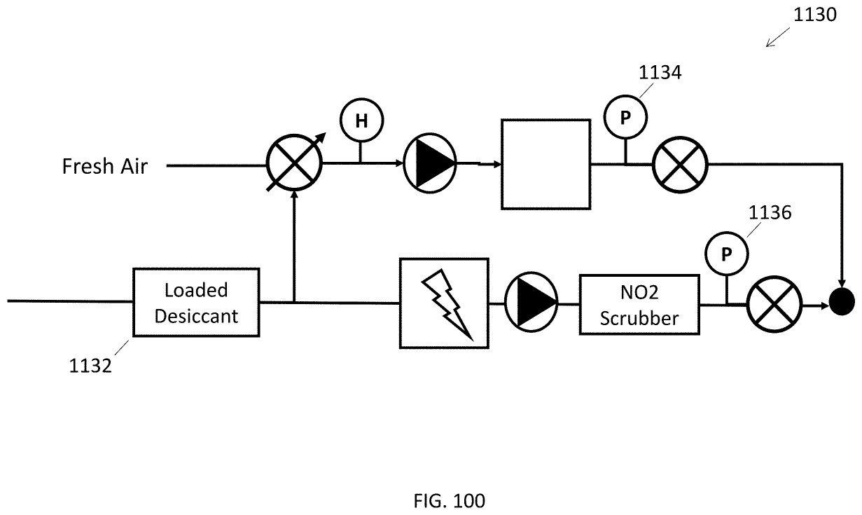

FIG. 100 depicts an exemplary bypass architecture system 1130 that desiccates all of the reactant gas entering the plasma chamber using a loaded desiccant 1132 and can also blend purge gas in the bypass channel. A flow controller, depicted as a 3-way proportional valve, can variably blend the purge gas to a non-condensing level at the pressure required in the bypass reservoir based on feedback from a humidity sensor. Pressure sensors 1134, 1136 in fluid communication with the bypass reservoir and the scrubber provide information to the system controller. The pressure measurements are utilized for one or more of pressure alarms, pump control feedback, calculation of pulse flow rate. Examples of pressure alarms are presented in the table below:

| Situation | Pressure signal | System response |
|---|---|---|
| Pump failure | Pressure does not rise | Alarm |
| Gas inlet blocked | Pressure does not rise | Alarm |
| Delivery device kinked/ | Pressure decreases too | Alarm |
| obstructed | slowly | |

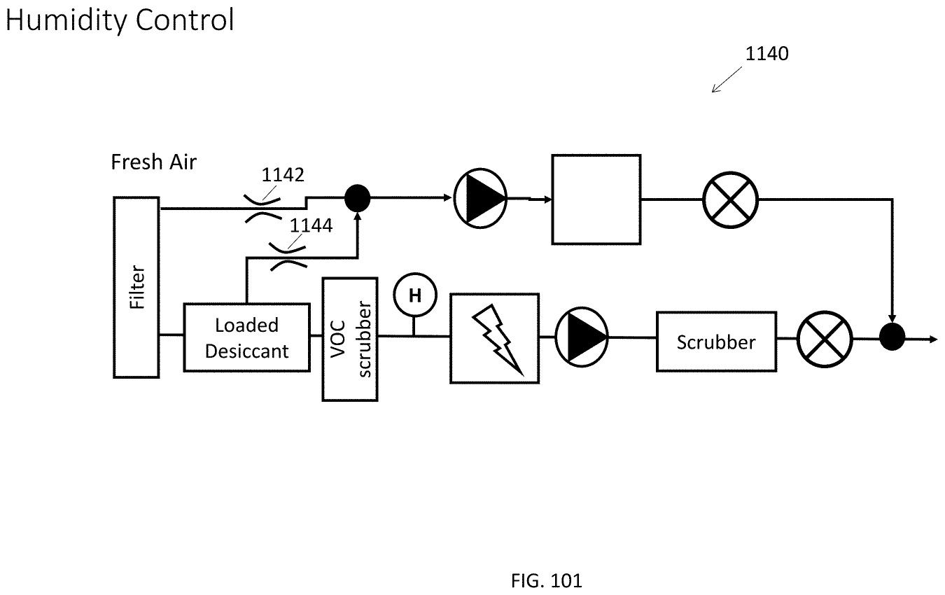

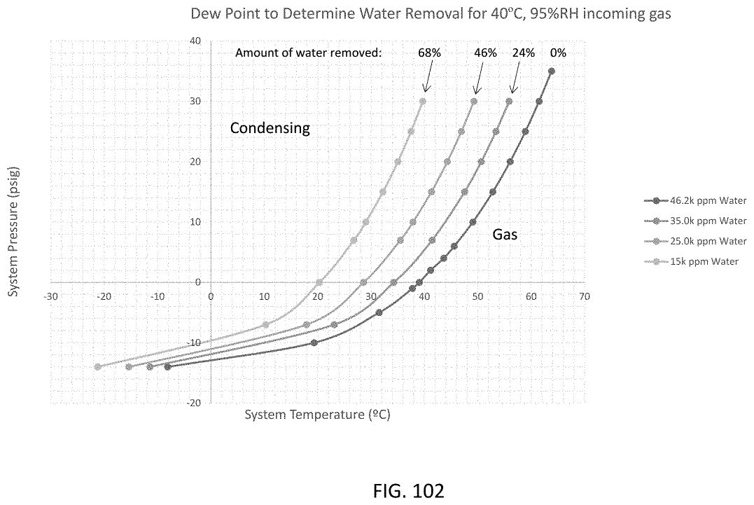

FIG. 101 depicts an exemplary bypass architecture system 1140 with a fixed blending ratio for purge gas provided by critical or fixed orifices 1142, 1144 that define a mix ratio. The mix ratio is the same for all environmental conditions, resulting in some variation in purge gas humidity. The mix ratio is selected to ensure that purge gas does not condense at worst case ambient humidity levels and purge gas pressures. Use of fixed orifices simplifies the overall complexity of the system and reduces mass. FIG. 102 an exemplary graph representing the dew point for gases of varying humidity as a function of pressure and humidity for a specific water content of gas. In this case, the water content of ambient air at 95% RH and 40 deg C. is used, but similar plots can be generated for other environmental conditions. Each line represents the pressure temperature relationship of the dew point for a specific amount of water removal. As an example, a NO generation device operates in these conditions (95% RH and 40 deg C.), utilizing a flow rate of 250 ml/min for purge gas and the gas is pressurized to 10 psig in the purge gas reservoir. By looking at the 40 deg C. on the X axis and 10 psig on the Y axis, FIG. 102 indicates that nearly 46% of the water content in the ambient air needs to be removed to prevent condensation at 10 psi. For the purpose of having a modest safety factor, a target of 50% of the water will be removed. To remove 50% of the water content of the ambient air, 50% of the incoming purge gas flow rate can be dried to 0% RH. As this applies to FIG. 101, it suggests that the flow restriction of the desiccant pathway and the non-desiccant pathway should be equal so that the pump sources a 50/50 mix to prevent condensation. Since the 50/50 mix was selected for the worst-case ambient conditions, all other ambient conditions will have less water content and thus be further to the right on the plot, away from the dew point line that the system was designed for. In other words, the system will continue to remove 100% of the humidity in 50% of the incoming air and can be certain that condensation will not occur within the system.

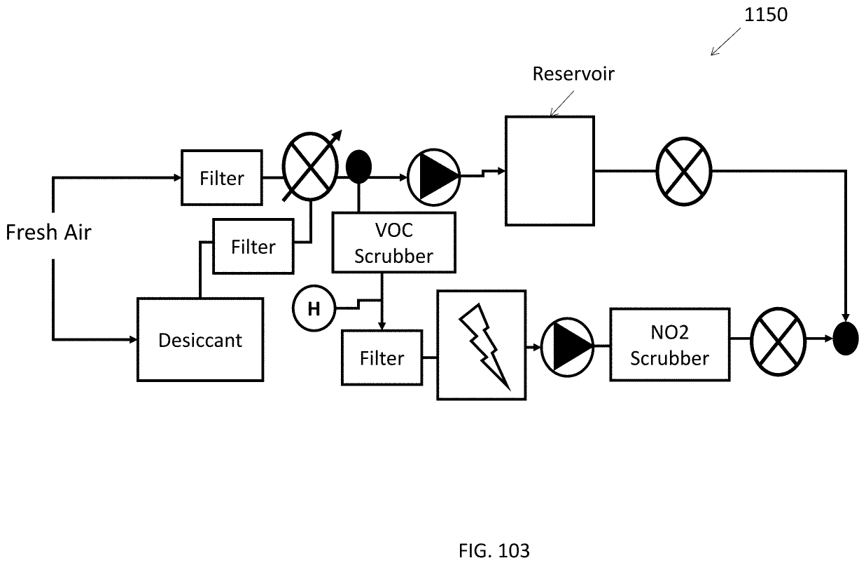

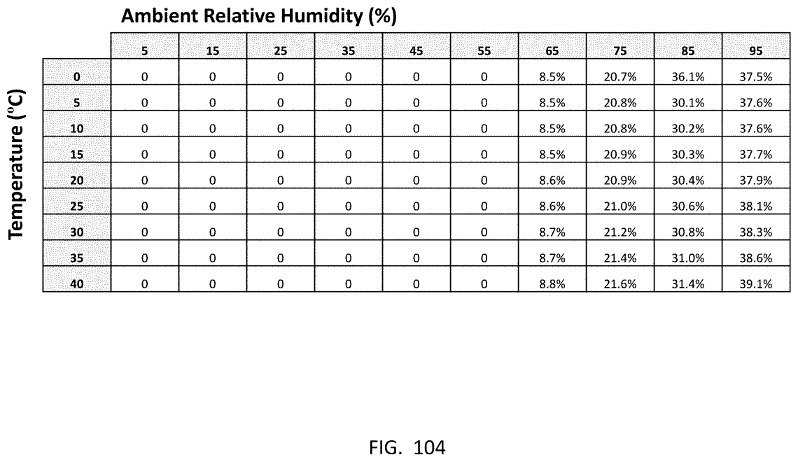

FIG. 103 depicts an exemplary bypass architecture design 1150 with a variable blending stage at the inlet. In some embodiments, all gas is blended to the same level to ensure no condensation at the system operating pressures. The ability to variably blend the incoming gas enables a system to dry the minimum amount of incoming gas to prevent condensation within the system, thereby prolonging the service life of the desiccant. FIG. 104 depicts an exemplary look up table that a NO generation system and/or delivery system that operates at 10 psi max internal pressure can use to prevent condensation within the system. The system measures the ambient temperature and humidity and then looks up the amount of water to remove from the incoming gas (such as ambient air) in the table. The % water to remove is equivalent to the fraction of incoming gas to be sourced from the dry, desiccant path. In another embodiment referring to the look up table of FIG. 104, a simplified 10 psi system could simply completely desiccate 39% of the incoming gas and blend it with the remaining 61% of incoming gas to prevent condensation in all cases. In some embodiments, the mix of ambient and desiccated gas is varied for reactant gas entering the plasma chamber and purge gas. Direction of flow and gas humidity are controlled by a flow controller at the blending point and pump activity in each flow path. For example, when the system fills the bypass reservoir, the plasma chamber path pump is stopped and vice-versa. A VOC scrubber (e.g., filter comprised of activated carbon, potassium permanganate, etc. filter) is depicted on the plasma chamber pathway. In some embodiments, the VOC scrubber is located at the inlet of the device for both gas pathways. Locating the VOC scrubber only in the plasma chamber path reduces the amount of VOC scrubber required. This is acceptable because purge gas consists of ambient gas that the patient is already breathing, hence there is no safety reason to clean that air up further. It is beneficial to scrub gas entering the plasma chamber, however, to ensure that there are no VOCs entering the plasma chamber as this could create additional compounds in the product gas and potentially alter the device NO dose levels due to VOC combustion. An optional filter after the VOC scrubber collects any particulate released from the VOC scrubber.

Pulsed NO Delivery with Other Respiratory Device Applications

A pulsed NO device can be used with a ventilator or any respiratory device. Alternatively, NO delivery systems can provide continuous NO to an inspiratory flow stream. In some embodiments, the amount of NO delivered to the inspiratory flow stream is a constant level. Providing a constant NO concentration and flow rate to a dynamic inspiratory flow results in varying NO concentration within the inspiratory limb. The degree to which NO concentration varies within a patient's inspired volume of gas depends on several factors, including but not limited to:
Inspiratory limb length. A longer inspiratory limb provides volume for mixing of NO and inspiratory gas,
Presence/absence of accessory devices such as a humidifier. Accessory devices add volume for NO mixing,
The fraction of volume delivered that is bias flow. In cases where the patient has a low tidal volume (e.g., neonate), the volume of gas breathed by the patient is much less than the volume of gas provided by bias flow. If the NO generator introduces NO at levels to dose the bias flow, the low volume inspirations will not affect the delivered dose very much.
The amplitude of the inspiratory flow path flow rate.

To address the limitations of constant concentration and flow rate, some embodiments deliver an amount of NO proportional to inspiratory flow rate, for example. Some embodiments of NO delivery systems include a gas analysis capability to measure the NO and NO2 levels at the patient to quantify the affects of all of the treatment variables on delivered dose. Some embodiments use the NO and/or NO2 measurements to compensate NO delivery and/or production in order to achieve the target delivered dose.

The complexity of generating a constant concentration in a dynamic inspiratory flow can be avoided by introducing pulsed NO at the patient to achieve equivalent NO dosing at the patient. In this case, a NO generation device senses inspiration from either the patient, the inspiratory flow or from another active treatment device (e.g. ventilator). In some embodiments, the amount of NO provided in the NO pulse is a function at least in part of the magnitude of the inspiration based on inspiratory flow rate data.

In some embodiments, a NO generation and/or delivery device can deliver either a continuous flow of NO or a pulsed flow of NO, depending on the patient treatment conditions. In patient treatments where the patient tidal volume is small (e.g., neonates) or when the respiratory cycle is rapid (e.g., high frequency ventilation), breath detection can be challenging, and a NO generation device can dose continuously (i.e., provide a continuous stream of NO) to adequately dose the patient. Pulsing NO limits the amount of production that is required of the NO device because gas that does not enter the patient is not dosed with NO, enabling the NO device to be smaller, lighter and longer-lasting (i.e. electrode life, battery life). In some embodiments, when inspirations are detected (e.g., if the breath signal exceeds a threshold indicates breaths of a certain magnitude regardless of frequency, or if breaths are detected at a consistent frequency), a NO delivery system transitions from continuous NO delivery to pulsed NO delivery. A NO delivery device can default to bias flow/continuous delivery and switch to pulsed mode when breaths are detected. In another embodiment, the NO delivery device can be switched between a pulsed mode and continuous mode by a user. Continuous NO delivery can either be a constant flow of NO or a flow in proportion to the ventilator (or any respiratory device) flow. In some embodiments, NO pulse timing is based on a time schedule, rather than synchronized with patient respiration. For example, a patient receiving high frequency ventilation (e.g., 15 Hz) could receive NO every other second so that 50% of their breaths are dosed. In some embodiments, NO product gas output is pulse-width-modulated (PWM) and adjusted based on a physiologic measurement (e.g., SpO2).

Pulsed NO delivery offers the following benefits over continuous NO generation and or delivery. 1) Decreased need for and reliance on gas sensing capability at the patient because the transit time from device to patient is so rapid, the delivery device is known (i.e., standard cannula or delivery tubes used) and the NO is not exposed to high levels of Oxygen until it is being delivered to the patient. 2) Electrodes last longer because less NO is being generated overall. 3) Negligible dead volume is added to an existing treatment setup when NO is added. This decreases the potential for the existing set-up to require alteration and recalibration which can result in treatment interruption. 4) Negligible gas volume is added to the inspiratory flow due to the potential for high pulse concentration resulting in less oxygen dilution in the inspired gas (e.g. 15 ml pulse added to a 500 ml tidal volume). 5) Enables smaller, more portable NO generation devices. In some embodiments, pulsed NO generation devices are in the form of modules. More than one module can be linked together to provide redundancy in NO delivery, as needed.

In some embodiments, a pulsed NO delivery device utilizes purge gas with a non-atmospheric level of oxygen (i.e., >21%). This can make-up for any reduction in oxygen content within the inspired gas due to dilution from the NO pulse. In some embodiments, the NO delivery device prolongs the purge pulse to not only purge the delivery device of NO but also deliver all of the gas volume required for a breath. In doing so, the NO generation device serves as a ventilator providing all of the inspiratory gas that a patient requires in addition to inhaled NO.

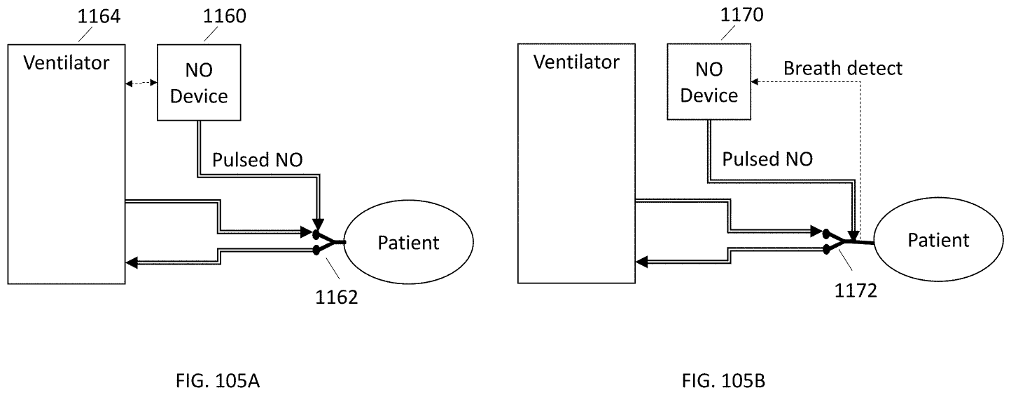

FIG. 105A depicts an exemplary NO device 1160 connected to a patient end of the inspiratory limb 1162. NO gas is kept separate from inspiratory flow until the point of injection. This can prevent exposure of NO to high oxygen levels that can occur in the inspiratory limb. In some embodiments, the NO lumen is filled with NO-containing gas that is injected in a first-in, first-out fashion. In some embodiments, the NO lumen is flushed with non-NO containing gas between pulses to prevent NO oxidation between breaths. Some embodiments inject the NO into the patient Wye connector, as shown. Other embodiments inject NO into an endotracheal tube to ensure that the NO will enter the patient and not be swept away in bias flow.

The system shown in FIG. 105A provides additional benefits in that less NO can be generated overall. NO can be introduced only as the patient inhales so that the balance of gas circulating within the ventilation circuit is not dosed. This can be particularly beneficial in anesthesia circuits where gas in the inspiratory circuit is recycled to conserve anesthesia.

The NO pulse generated can vary in concentration, duration, flow profile and timing to dose specific regions/depths of the respiratory tract and lung to varying degrees. When NO gas is introduced to inspiratory limb flow at the inspiratory side of the Wye connector, it is unclear whether or not all of the inspiratory flow will enter the patient. Thus, some embodiments will select NO pulse parameters that accurately dose the entire inspiratory flow for the duration of the inspiratory event. Given the short distance between Wye connector inlet and ET tube connection, some Wye connectors include a mixing element to ensure a homogeneous gas mixture prior to flow entering the ET tube.

Owing to the rapid uptake of NO by a patient, little to no NO exits the patient upon exhalation. This effect in concert with the lower overall NO generation of a pulsed approach results in less NO and NO₂ entering the environment of the patient and caregivers.

Owing to the rapid delivery of NO to the patient in the system depicted in FIG. 105A, it can be unnecessary to measure NO and NO₂ concentration at the patient. In some embodiments, the NO and/or NO₂ are measured within the NO generation and/or delivery device before the pulse is delivered through the delivery lumen. This can be acceptable because the transit time is on the order of tens of milliseconds which is very little time for additional NO oxidation. Thus, a measurement of NO and/or NO₂ within the NO generator is very much representative of the concentration of NO and NO₂ inhaled by the patient. In some embodiments, the NO generation system receives an inspiratory flow rate measurement from a sensor or external device (e.g., ventilator) and can calculate an intra-pulse concentration of NO per breath as a function of inspiratory flow rate, NO flow rate and NO concentration. In some embodiments where the quantity of NO gas that actually enters the patient is known, the patient dose is defined in units of mass per unit time (e.g., mg/hr) and all that is tracked is the mass of NO delivered to the inspiratory gas pathway. NO and NO₂ gas concentration measurement within the NO generation system may be direct, using one or more sensors (optical, chemiluminescent, electrochemical, etc.).

FIG. 105A depicts an exemplary embodiment of a NO generation system with two-way communication with an external device. External devices include but are not limited to ventilators, anesthesia machines, CPAP machines, BiPAP machines, high frequency ventilators, oxygen concentrators, ECMO machines, automated CPR machines, and patient monitors. The external device, a ventilator 1164 in this example, can provide information about the treatment (e.g., inspiratory flow rate, inspiratory pressure, breath timing, inspiratory gas oxygen content, a breath trigger signal) and information about the patient (e.g., inspiratory oxygen concentration, SpO2, Methemoglobin levels). In some embodiments, the external system supplies inputs to the NO generation system, such as a source of reactant gas, electrical power, internet access, WiFi access, GSM access and the like. The external device may also provide a user interface, back up battery, power supply, alarm system and other features for the NO generation system. In some embodiments, the external device controls the NO generation device. In some embodiments, the NO generation device is controlled through the user interface of the external device. For example, in one embodiment, a ventilator user interface includes a NO button to turn the NO generator ON/OFF and a knob to adjust inhaled concentration of NO. In some embodiments, the NO generation device has no user interface and relies solely on user inputs through an external user interface. In another embodiment, the user interface for the NO generation device is from an external device (e.g., cell phone, tablet computer, laptop computer, etc.).

FIG. 105B depicts an exemplary NO generation system 1170 operating independently of a concomitant therapy. A gas-filled lumen is shown that communicates pressure signals to the NO generation device for detection of patient breath patterns (inspiration, exhalation, etc.). In other embodiments (not shown), a breath detection sensor is located at the inspiratory flow and measured information is communicated back to the NO controller with wired or wireless communication. The breath detection sensor can measure pressure and/or flow with one or more of the following methods: a temperature sensor, a pressure sensor, a flow sensor, a strain sensor, a potentiometer, a LVDT, an optical encoder, a strain sensor, a capnography sensor, chest band, chest impedance and other types of sensors.

The system depicted in FIG. 105B delivers NO gas directly to the ET tube 1172. This approach is advantageous because all of the NO delivered during patient inspiration enters the patient. Thus, the NO delivery system does not need to estimate for uninspired gas that passes through the Wye connector during patient inspiration without being inspired and overproduce NO, accordingly.

Breath detection signals can vary with treatment type when NO is delivered to the ET tube. For example, a ventilated patient inhales when the ventilator sends a positive pressure to the patient, requiring a positive slope to detect breath. When a patient is being weaned from a ventilator, the ventilator is programmed to respond to a dip in pressure (negative slope in pressure) indicating that the patient is initiating a breath. In some embodiments, a NO delivery device receives input from a user or external device as to what type of treatment is being administered. In other embodiments, the NO device uses more sophisticated algorithms to detect breath despite varying types of breath signals. As an example, some embodiments measure pressure through a lumen connected to the ET tube and detect a breath by analyzing pressure data for a positive pressure event that has faster onset (pressure rate of change with respect to time) than patient exhalation and thus must be an inspiratory event from a ventilator. Other ET NO delivery systems utilize an alternative means for breath detection that is not affected by the patient treatment, such as measuring inspiratory flow rate, chest impedance or chest shape change (AKA a chest band).

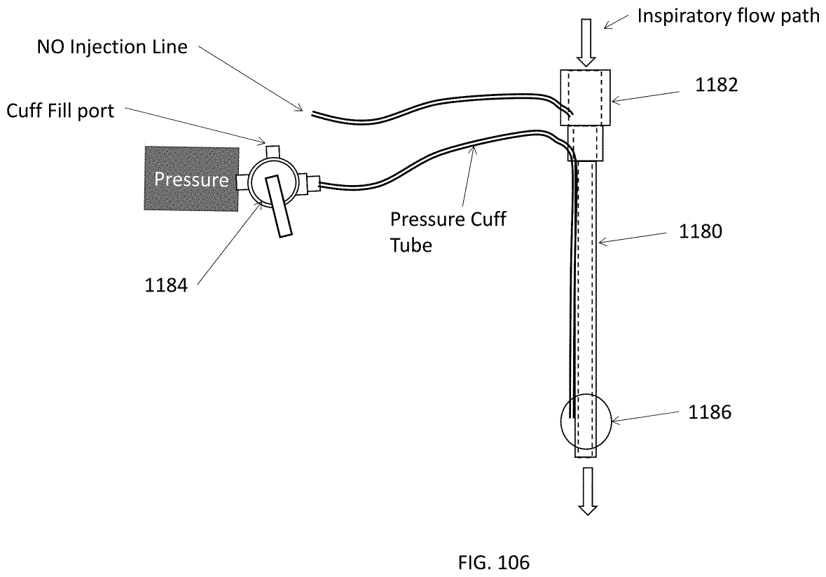

FIG. 106 depicts an exemplary ET tube for NO delivery. NO enters the inspiratory air flow of the airway tube 1180 at the distal end of the flow path, below the connector 1182. A pressure transducer connects to the cuff fill lumen for detection of the respiration cycle. As the patient exhales, the pressure in the cuff increases. As the patient inhales, the pressure in the cuff decreases. A stopcock 1184 enables the user to fill the cuff 1186 and connect the pressure sensor to the cuff. Similarly, the pressure signal from a cuff on a urinary catheter can be utilized to detect the respiratory cycle for breath detection.

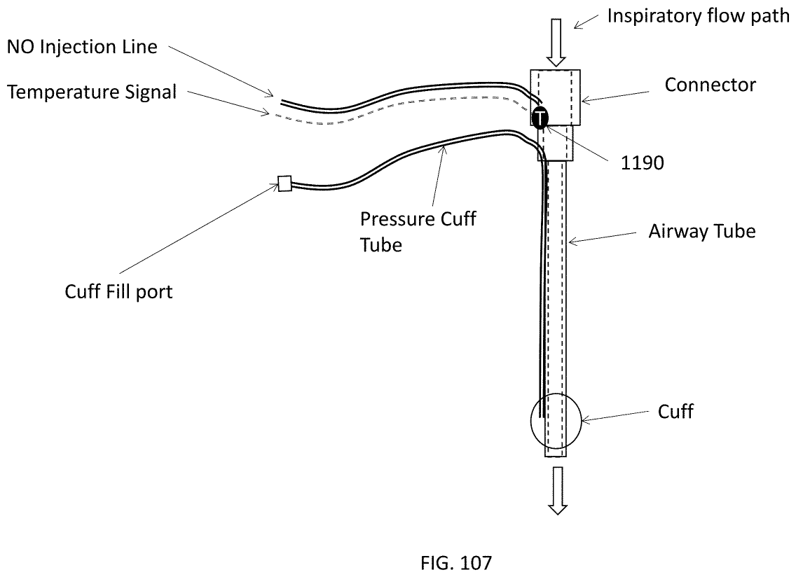

FIG. 107 depicts an exemplary ET tube for NO delivery with a fast temperature sensor 1190 in the wall for breath detection. Inspiration is detected by a decrease in temperature as cooler ambient air enters the patient. Other embodiments include a flow sensor (e.g., delta pressure) in the ET tube for inspiration detection.

In some embodiments, flow rate through the ET tube is measured for breath detection. This can be done using a delta-pressure across a flow restriction or a hot wire approach. In some embodiments, a partially-obstructive flapper valve within the ET tube inspired gas pathway is utilized to detect respiratory events. The flapper moves toward the patient during inhalation and away from the patient during exhalation. The position of the flapper with respect to neutral can be detected in multiple ways including but not limited to using optics, strain gages, or displacement transducers.

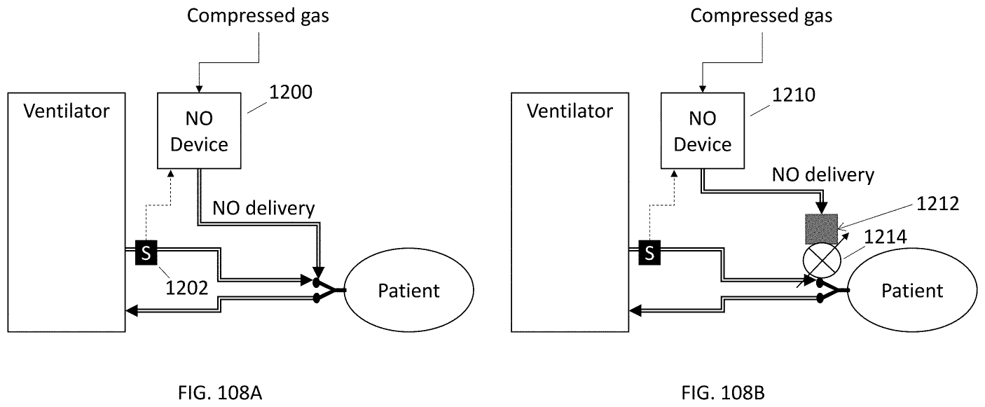

FIG. 108A depicts an embodiment of a NO generation device connected to a ventilation circuit. An inspiratory flow sensor 1202, labeled “S”, informs the NO generation device 1200 of the breathing pattern for accurate flow dosing. Flow dosing can be pulsed or continuous. The NO generator in this example, receives a source of reactant gas from an external source of compressed gas containing nitrogen and oxygen. In some embodiments, the NO delivery line includes a capability to scrub the gas for NO₂. The NO₂ scrubbing capability can come from one or more of a NO₂-scrubbing coating, a NO₂-scrubbing co-extrusion, a NO₂-scrubbing insert, or a physical scrubber in series with the NO delivery lumen. In some embodiments, the scrubber is located after the NO is injected into the inspiratory stream. In some embodiments, scrubber chemistry includes one or more of soda lime, TEMPO, and ascorbic acid.

FIG. 108B depicts an embodiment of an NO generation device 1210 having a pressurized scrubber 1212 located at the patient Wye or ET fitting. A flow controller 1214 releases pressurized NO from the scrubber, as required. This approach allows for the NO to be scrubbed immediately before delivery and NO is located at the NO injector. By being at the injector, there is minimal to no delay in NO delivery, enabling a NO delivery system to deliver NO faster without the transit time that occurs with a purged delivery device. Purging of the NO line is not required between breaths. In order to know the concentration of NO at the injector, the NO device can calculate the quantity of NO lost to oxidation and interaction with system components (e.g., scrubber) based on factors including one or more of residence time, temperature, pressure, scrubber type, and scrubber age.

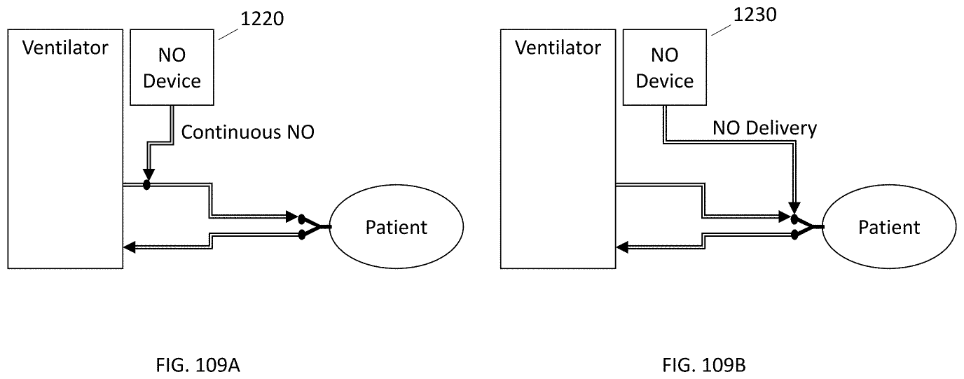

FIGS. 109A and 109B illustrates exemplary embodiments of NO generation systems that demonstrate that NO can be introduced at various locations within the inspiratory limb. When NO from an NO device 1220 is introduced far from the patient, as in FIG. 109A, NO delivery is often continuous to ensure that the concentration of all gas within the inspiratory limb is consistent. Constant concentration of all gas is important since it is unclear which subset of the inspiratory limb gases will be inhaled. The gas molecules passing by the NO injector during an inspiratory event are not the gas molecules that the patient is inhaling at that instant. Whether or not the gas molecules dosed during one inspiratory event are inhaled by a subsequent inspiratory event depends on the breath rate, inspiratory limb volume (function of length, diameter and quantity of additional devices in series, (e.g., humidifier), and inspiratory flow rate. In some instances, the patient breathing and inspiratory limb set-up results in the patient inhaling gas that is out of phase with the gas that is dosed during an inspiratory event.

This issue gets simpler when the NO is introduced at or near the patient. This is because the volume of gas between the injection location and the patient goes to near-zero and it is possible to be certain that the injected NO will be inhaled by the same breath that it was injected for. Injection near the patient also provides the patient with fresher NO that was not injected at an earlier time and transferred through the inspiratory limb with variable amounts of oxygen. This faster delivery of NO to the patient decreases the potential for NO2 formation. When NO from an NO device 1230 is introduced close to the patient, as shown in FIG. 109B, NO can be introduced intermittently. In other words, the NO generator does not need to deliver NO to the inspiratory limb when the patient is not inhaling.

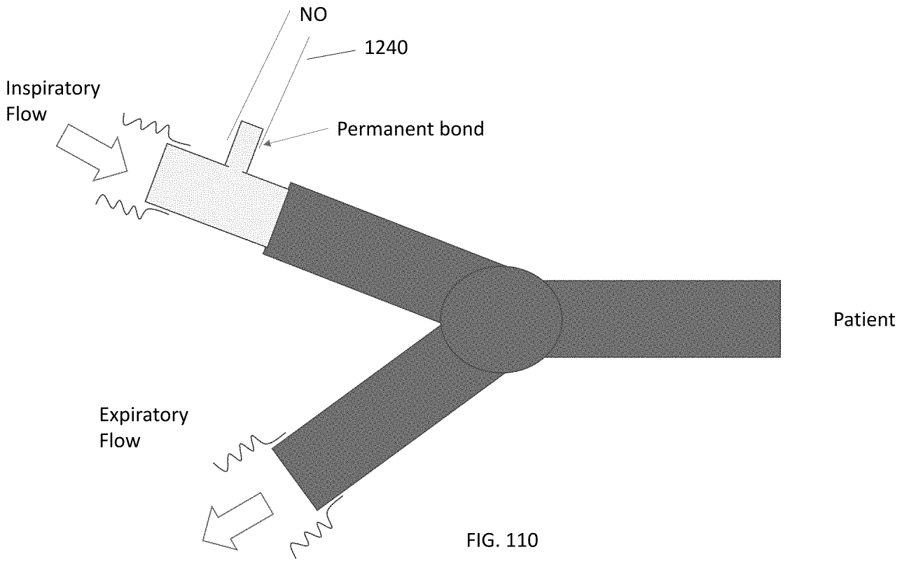

FIG. 110 depicts an exemplary NO injector design that interfaces with a typical patient Y-fitting and ventilator tubing. In some embodiments, an NO delivery tube 1240 is permanently bonded to the T-fitting to reduce use steps and prevent use error. This can provide a simple means to introduce NO to the inspiratory flow while minimizing the amount of weight hanging from the ET tube. In some embodiments, pressure-based breath detection occurs through the NO delivery lumen. In some embodiments (not shown), wires for a sensor at or near the inspiratory limb pass through the NO delivery lumen. In some embodiments, the NO is delivered through a fitting in the Y-fitting instead of having an additional T-fitting. This reduces parts count and pneumatic interfaces which can introduce leaks. In some embodiments, mixing elements (static and/or dynamic, not shown) within the Y fitting blend the NO with the inspiratory limb gas before it reaches the intersection point in the Y fitting.

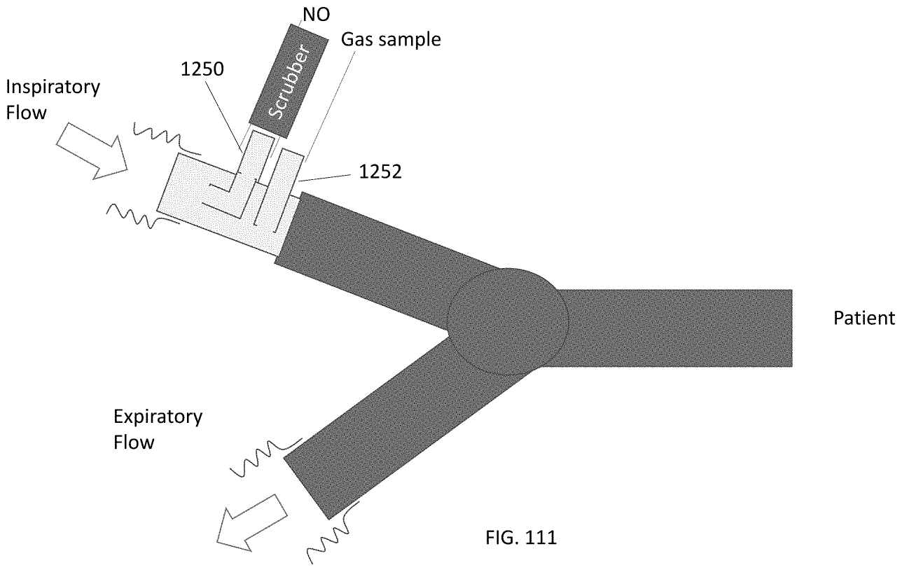

FIG. 111 depicts an exemplary NO injection design that includes a gas sampling port. A dual lumen extrusion is connected to a T-fitting. One lumen 1250 carries NO-containing gas to the inspiratory flow. The other lumen 1252 is used to provide a gas sample of the inspiratory flow to one or more gas sensors within the NO generator for analysis. NO is introduced to the inspiratory flow in the retrograde direction to improve mixing with the inspiratory flow. In some embodiments, NO is introduced to the inspiratory flow through a shower-head design that disperses the NO into the inspiratory flow rapidly to reduce the NO oxidation rate and provide even dosing within the lung. In some embodiments (not shown), a mixing element downstream of the NO injector disperses the NO throughout the inspiratory gas. The gas sampling port is located downstream of the NO injection location. In some embodiments, the gas sampling port is downstream of the mixing elements as well to ensure a homogeneous gas mixture for analysis.

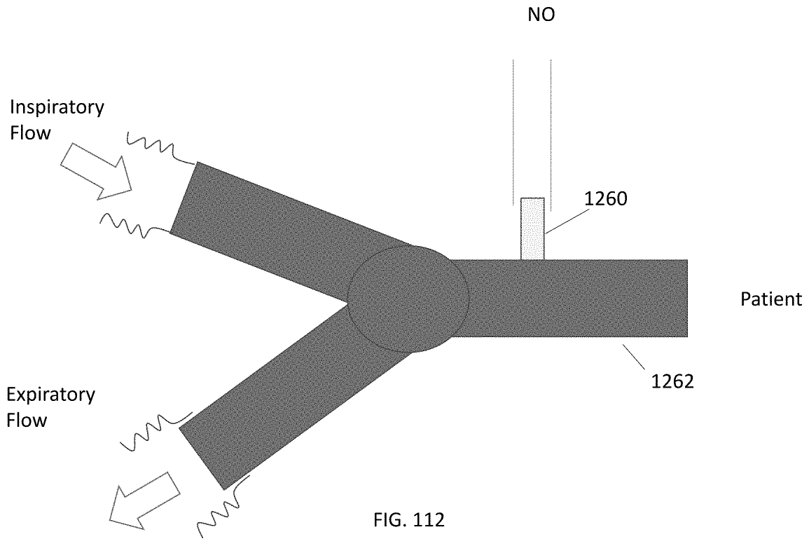

FIG. 112 depicts an exemplary embodiment of an NO injection design where the NO is introduced through an NO lumen 1260 to the patient leg 1262 of the Wye fitting. Similarly, the NO can be introduced to the ET tube. This approach provides a benefit in that NO introduced as the patient inhales is guaranteed to go into the patient. When NO is introduced before the patient Wye, there is a risk that some of the NO will go to the exhaust leg of the Wye fitting without going to the patient first.

In some embodiments, as described, the NO is mixed into the inspiratory stream prior to sampling and measurement. In some embodiments, the system is calibrated to account for a lack of mixing of NO prior to gas sampling. In some applications, the inspiratory gas will be at warmed temperature and high humidity. This can present a challenge for a gas measurement system due to condensation of the water content of the gas as it cools to ambient temperature. In some embodiments, the sampled gas passes through a water trap prior to entering the NO generation system. In one embodiment, the conduit of sampled gas provides a gas communication path that is used for breath detection. Depending on the type of gas sample pump technology, breath detection can happen when the gas sample pump is on. In all cases, the gas sample lumen can be used for breath detection when the gas sample pump is off. In some embodiments, a pressure measurement is made within the water trap to detect inspiratory events.

To recap, NO can also be delivered near a patient through an ET Tube, scoop catheter, face mask, nasal cannula, mouth cannula and other means. NO delivery through each of these means can be either continuous or pulsed.

In some embodiments, the NO generator is near the patient with a short delivery tube (e.g., 0.5 m). This provides faster NO delivery to the patient and improved breath detection signals. In other embodiments, the NO generator is located further from the patient, and a longer delivery tube is utilized (e.g., 2 m). Being further from the patient provides a benefit of there being less clutter near the patient so that it is easier to address patient needs. In one embodiment, a ventilator tubing set includes an independent lumen for transporting NO from the NO device to a point closer to the patient prior to NO mixing with inspiratory gas. In one example, the NO lumen and inspiratory gas lumen intersect at the Wye-fitting connection. In another example, the NO lumen is connected to the ventilator tubing but has a separate proximal fitting for connection to a Wye fitting, ET tube, T-fitting, mask, or other component near the patient.

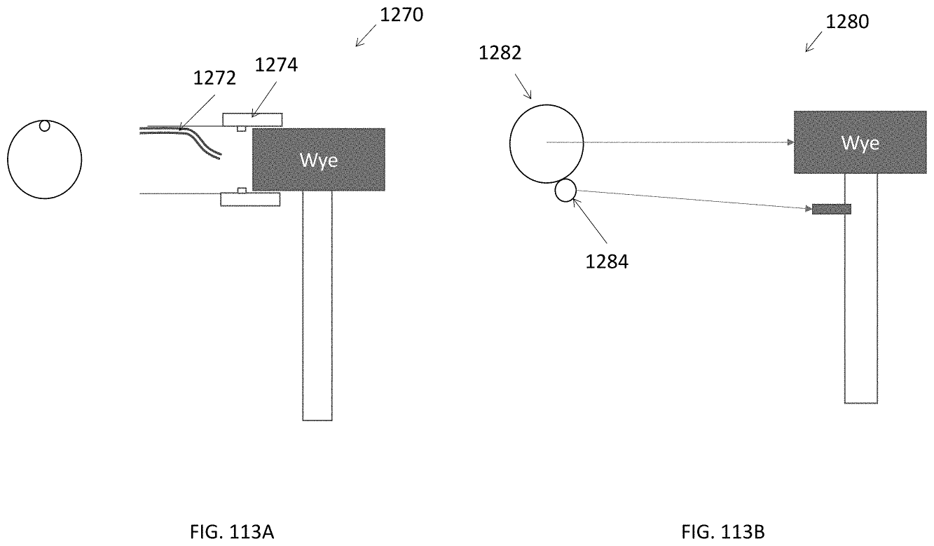

FIG. 113A depicts an embodiment of a dual-lumen inspiratory line 1270 with a dedicated lumen 1272 for NO delivery. In some embodiments, the NO lumen is also utilized for breath detection as well. In some embodiments, the dual lumen extrusion is bonded to a fitting 1274 that connects the tube to the next component in the system (e.g., Wye fitting, mask, etc.). In some embodiments, at the proximal (i.e., patient) end of the tube, the NO lumen separates from the inner wall of the inspiratory lumen, as shown. This can introduce the NO to the more central portion of the flow of inspiratory gas for improved mixing.

FIG. 113B depicts an embodiment of a dual lumen extrusion 1280 with one lumen 1282 flowing inspiratory gas and the other lumen 1284 delivering NO. At the proximal end of the line, the NO lumen and inspiratory lumen are separated and connected to the ET tube and Wye fitting, respectively. This approach of combining tubes mitigates against entanglement and clutter near the patient.

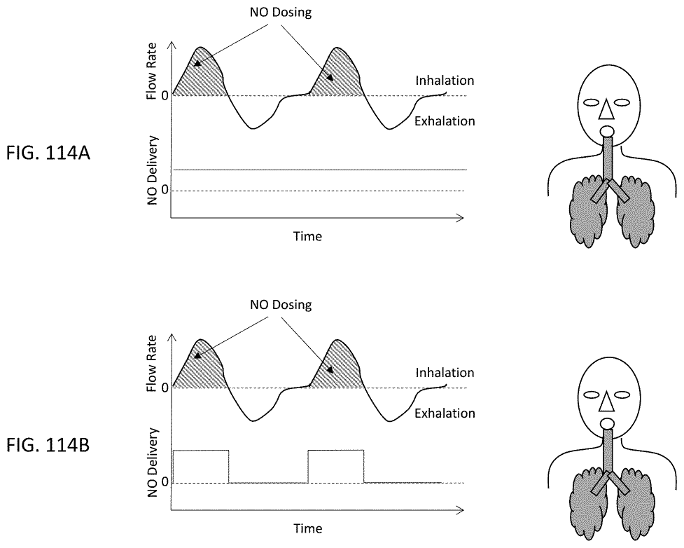

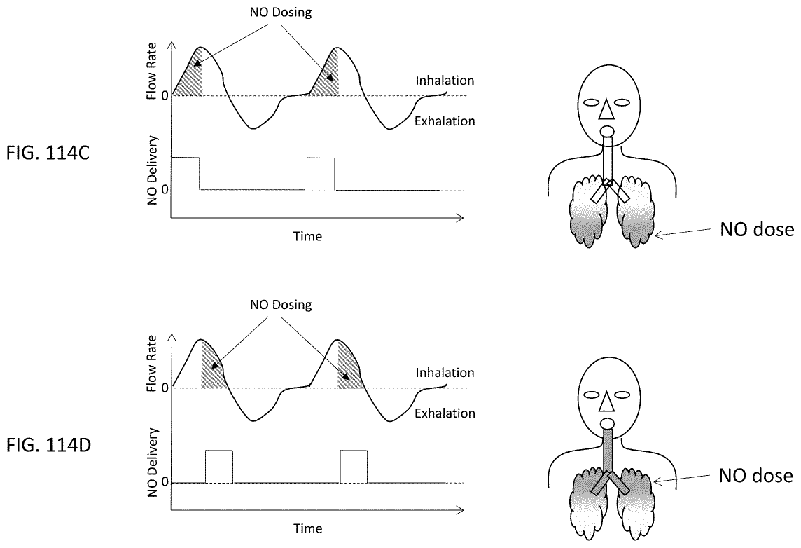

FIGS. 114A-114D depict exemplary graphs of the effect of dosing various portions of the inspired volume of gas. It should be noted that these approaches apply to any type of NO source, including tanks, electric NO generators, liquid NO generators, and NO generation from solids. FIG. 114A depicts an exemplary graph showing flow rate and NO delivery over time using a NO system that delivers NO to an inspiratory limb continuously. This approach doses all of the gas within the inspiratory limb to some extent. When the quantity of NO molecules added is proportional to the inspiratory flow rate, the concentration within the inspiratory limb is constant. When NO is introduced far from the patient, proportional dosing improves the accuracy of the inhaled dose because the concentration of NO at the patient is well controlled and well-blended with inspiratory gas. The image on the right side shows the dosing to the upper airway and entire lung indicated by shading. This approach generates more NO than is necessary because all gas flowing through the inspiratory circuit is dosed.

FIG. 114B depicts an exemplary graph showing flow rate and NO delivery over time where only the volume of inspiratory gas that is inhaled is dosed. This approach requires either a known volume of inspiratory limb between the NO injection point and the patient or NO injection proximal to the patient. The right side of the image shows that when only the inspired gas is dosed, NO is still delivered to the entire upper airway and lung. This approach introduces less NO to the inspiratory circuit overall. When tanks of NO are used, the tanks last longer when less NO is delivered. When electrically generated NO is used, this approach conserves electrodes, scrubbers and electricity. When solid and liquid materials that derive NO are used, they too are conserved when only the inhaled subset of inspiratory gas is dosed.

FIG. 114C depicts an exemplary graph showing flow rate and NO delivery over time in which NO is introduced to the first half of the breath. This can be achieved when the volume of inspiratory limb between NO injector and patient is known, albeit with some mixing and dilution along the leading and trailing edges of the NO pulse. Dosing an initial portion of the breath can also be achieved by dosing close to the patient. Dosing close to the patient is achieved in any number of ways, including face mask, nasal cannula, T-fitting upstream of the patient Y fitting, ET tube, Scoop catheter, and other methods. Dosing of a first portion of the breath delivers NO to the more compliant parts of the lung which in healthy individuals corresponds to the basal regions but in diseased lung can be in other anatomical regions where it mixes with existing gas within the airways (anatomical dead space) and alveolar regions (alveolar volume). There is superior ventilation/perfusion matching and oxygenation with targeting NO delivery to more compliant healthier lung in several pulmonary diseases. In some embodiments, this is accomplished by NO delivery early or mid-inspiration. In some embodiments, this approach is applied to patients with pulmonary arterial hypertension (PAH), chronic obstructive pulmonary disease (COPD), and interstitial lung disease (ILD).

FIG. 114D depicts an exemplary graph showing flow rate and NO delivery over time in which NO is delivered to the latter part of the inspired volume. Depending on the condition of the patient, it can be desirable to treat specific regions of the airway and/or lung and not other regions. One example is treatment of an upper airway infection while minimizing exposure of the deeper lung to NO. In this case, the NO pulse would be introduced late in the inspiratory event so that the NO only enters the patient to the depth of the airways. The ability to tailor the location of the NO bolus within an inspiratory volume is a way to deliver NO to specific regions of the airway and lung aiming for better patient oxygenation, targeted treatment and reduced environmental contamination. In some embodiments, the latter portion of the breath is dosed by detecting inspiration and delaying the NO delivery. In another embodiment, the later portion of the breath is dosed by triggering NO delivery off the peak inspiratory flow. When the upper airway is treated, the patient will exhale some level of NO and NO₂. When a face mask is used, the exhaled gas can pass through a NOx scrubber to remove NO and NO₂ from exhaled gas prior to introduction to the environment. In some embodiments, upper airway dosing is utilized to treat bacterial, viral, or fungal infections of the upper airway.

In some embodiments, a patient receives CPAP treatment via an oral mask or a nasal mask/pillow. A NO delivery device is connected to the inspiratory/expiratory CPAP limb (mask and or tubing) with a gas lumen. The NO delivery device detects inspiratory events in one or more ways including but not limited to receiving trigger signals from the CPAP device, measuring flow or the inspiratory flow, a thermistor, or by other means, e.g., sensing chest expansion, chest impedance, and measuring pressure of the inspiratory flow. In some embodiments, a NO delivery lumen connects to the mask of a CPAP system. As the patient inhales, a transient dip in pressure within the mask occurs, signaling the beginning of inhalation. The NO device delivers NO to the mask as the patient inhales. After delivering NO, some embodiments of the NO device purge the NO lumen with a gas that does not contain NO, typically air. As the patient exhales, exhaled gases pass through a one-way valve in the mask and through a NOx scrubber that removes NO and NO₂. The NOx scrubber is comprised of one or more materials such as soda lime, calcium hydroxide, potassium hydroxide, sodium hydroxide, TEMPO, potassium permanganate, ascorbic acid, activated carbon, and other materials.

In some embodiments, a NO delivery device can be coupled with a blower for high dose NO treatment. This type of treatment is typically deployed to treat respiratory infections. The blower provides pressurized inspiratory gas that opens the lung to maximize exposure of the lung tissue to NO. The inspiratory gas can be sourced from house air, a cylinder, or ambient air. NO delivery can be continuous, dosing all of the inspired gas, or intermittent to dose a subset of the inspiratory gas.

Manual Respiration

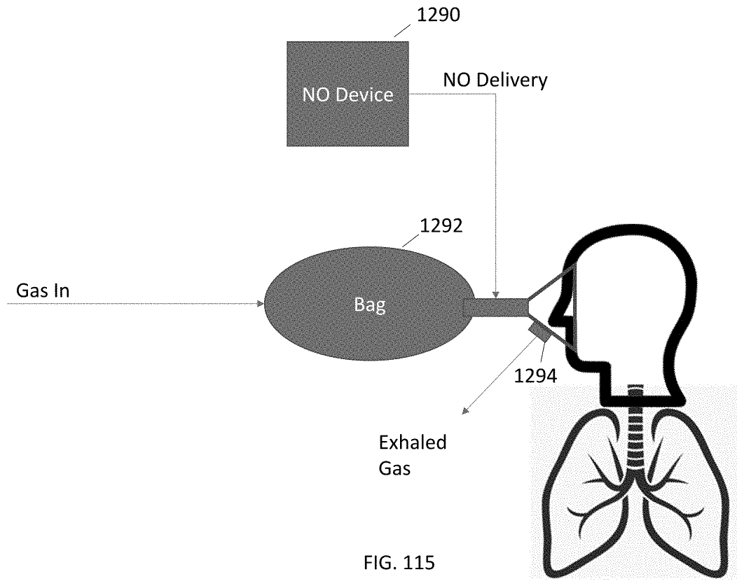

Manual respiration of a patient is common in the field and in the hospital. “Bagging,” as it is commonly referred as, involves connecting a bladder to a gas source. The gas source is typically air with varying amounts of oxygen up to 100% oxygen. The standard of care for delivering NO during manual respiration is to introduce NO to the source gas upstream of the bag. NO mixes with the additional gas and transfers through tubing and into the bag. As the patient inspires, the bag is squeezed in the hands of the user. NO and other gas within the bag passes through a mask interface (typically) and into the patient. When a properly sealing mask is used, 100% of the gas sourced for inspiration comes from the bag. FIG. 115 depicts an embodiment of a NO generation and/or delivery device 1290 in use with a bag 1292. Exhaled gases exit through a valve 1294 in the bag/mask assembly as shown.

Nitrogen dioxide (NO₂) accumulation between breaths is a concern. At slow breath rates, appreciable amounts of NO₂ can accumulate within the bag. Furthermore, if manual respiration with NO is paused for any amount of time, common practice is to fully squeeze the bag two to three times to purge aged gas from the bag prior to resuming manual respiration. This is usually done by holding the bag and mask away from the patient, squeezing the bag several times and then placing the mask back over the patient's nose and mouth.

FIG. 115 depicts an alternative way to introduce NO to a bag circuit. The bag is filled by a source gas. NO is introduced to the inspiratory flow after the bag during inspiration. This eliminates nitrogen dioxide build-up associated with NO aging within the bag. The NO generator can detect the inspiratory event with measurements from several kinds of sensors, including, but not limited to, pressure, flow, sound, acceleration, displacement, strain, thermal, optical, and other means. For example, the NO generator can detect an increase in pressure within the mask through either the NO delivery lumen or a dedicated breath detection lumen indicative of the bag being squeezed and onset of an inspiration. In some embodiments, inspiration is detected by observing a deviation in the flow rate and or pressure of gas to the bag that accompanies the bag being squeezed. Other means of breath detection include, but are not limited to, pressure within the bag/mask, flow rate within the gas tubing/bag/mask assembly (e.g., delta-pressure or hot wire flow sensor), microphones that measure sound levels, strain sensors within the bag, and other approaches.

Once inspiration has been detected, the NO generation device releases a pulse into the inspiratory pathway within tens of milliseconds, thereby dosing the current inspiration. This approach of introducing NO after the bag eliminates the risk associated with aging NO within the bag and enables more rapid resumption of bagging after a pause.

This same approach could be used with a tank-based NO delivery system as well using a flow controller to control NO flow into the inspiratory gas pathway. In some embodiments, the flow controller is located at the NO device to release high-pressure NO/N₂ pulses and a passive check-valve (one-way valve) is located at the mask or inspiratory gas path to prevent NO exiting the delivery tube between breaths. In another embodiment, the delivery tube contains high pressure NO/N₂ gas that is controlled by a flow controller located at or near the bag/mask.

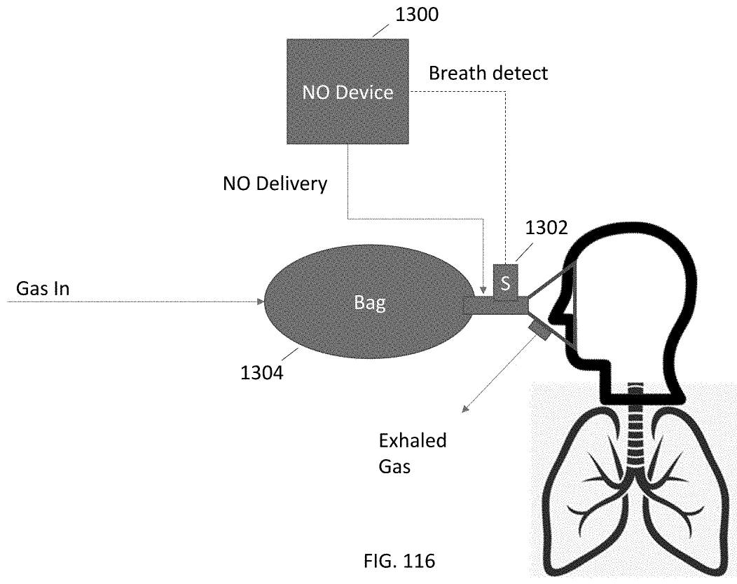

FIG. 116 depicts an embodiment of a NO generation device 1300 that utilizes a remote sensor 1230 located in the bag/mask assembly 1304 that is utilized to detect an inspiratory event. A dashed line back to the controller indicates the sensor signal being transmitted to the NO generator. Sensor data can be wired or wireless. In the case of wireless transmission, the sensor would include a battery as well.

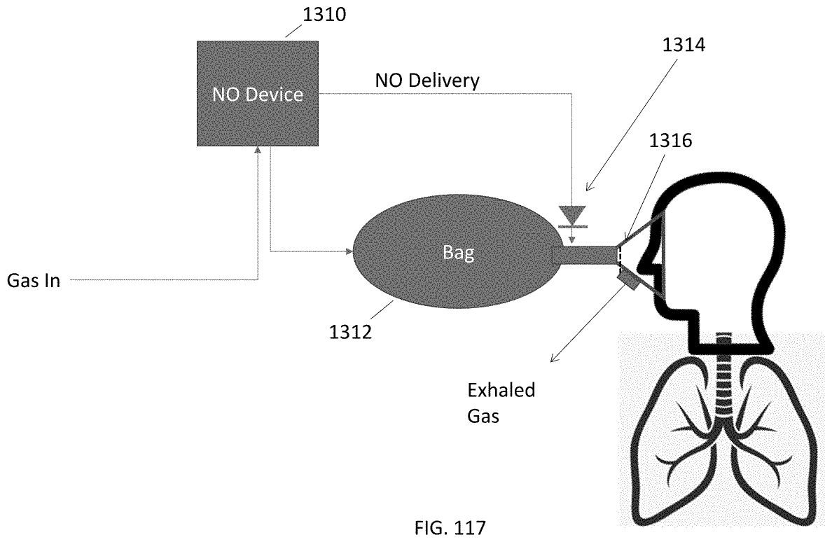

FIG. 117 depicts an embodiment of an NO device 1310 whereby the inspiratory gas flows through the NO device. Within the NO device, one or more of pressure, flow, velocity, strain, temperature, or other parameters are measured to detect breath. Upon breath detection, the NO device delivers a NO pulse to the bag/mask assembly 1312 downstream of the bag. In some embodiments, the NO delivery is continuous instead of pulsed. A check valve 1314 within the NO delivery line prevents mixing of NO and inspiratory gas between pulses for non-pulsed applications. In some embodiments, the NO delivery is proportional to the flow within the inspiratory gas pathway. Pulsed NO can be advantageous in the areas of battery life, electrode life, and scrubber life. When NO is pulsed at the onset of inspiration, almost all NO is absorbed by the patient, resulting in very little NO and/or NO₂ exhaled. This can be advantageous since excess NO and NO₂ that do not enter the patient are exhaled into the environment and can present a risk to care givers and others in the air space of the patient. In some embodiments, shown in FIG. 117, the exhalation pathway of the mask includes a NOx scrubber to remove NO and NO₂ before introduction into the environment. An additional feature depicted in FIG. 117 is a filter 1316 in the inlet gas path of the mask to remove particulates in the gas from electrodes, scrubber materials and other parts of the system. Placing the filter in this location can be advantageous because it is close to the patient, eliminating the potential sources of particulate, and is in a location of large-cross-sectional area which can reduce flow restriction for the generation device.

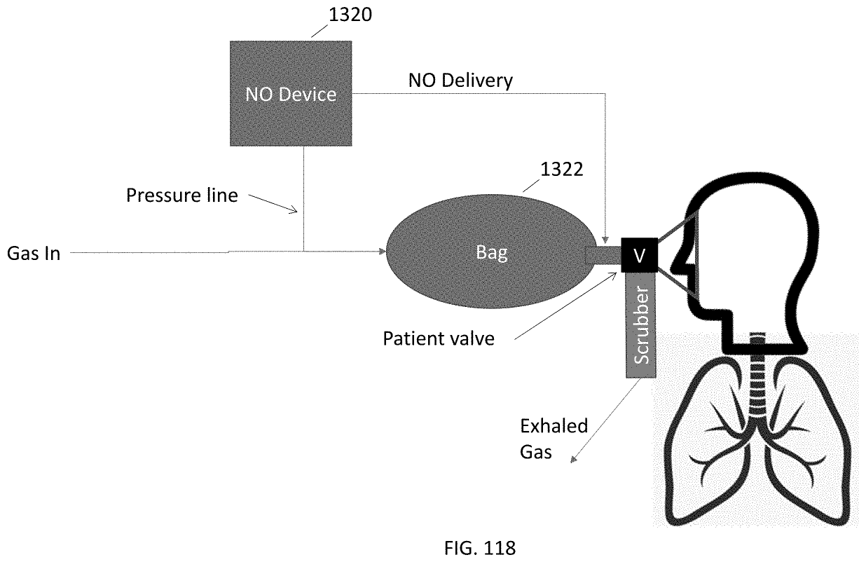

FIG. 118 depicts an embodiment of an NO device used with a manual resuscitation system. The NO device 1320 receives a pressure signal from the bag gas source line. When the bag 1322 is squeezed, flow into the bag ceases, causing an increase in the pressure within the line. NO is delivered to the system after the bag. The patient receives pressurized bag gas and NO through a nasal mask. The pressure of bag gas delivery closes the patient exhalation valve as it is delivered. After the bolus of bag gas and NO are delivered, the pressure within the mask decreases and the patient exhalation valve permits exhaled gas to exit the mask through a NOx scrubber and into the environment. In some embodiments, the NOx scrubber includes a filter to protect care givers from potentially air-borne contaminants from the patient. This configuration prevents NO2 from forming in the bag, which allows for less NO waste.

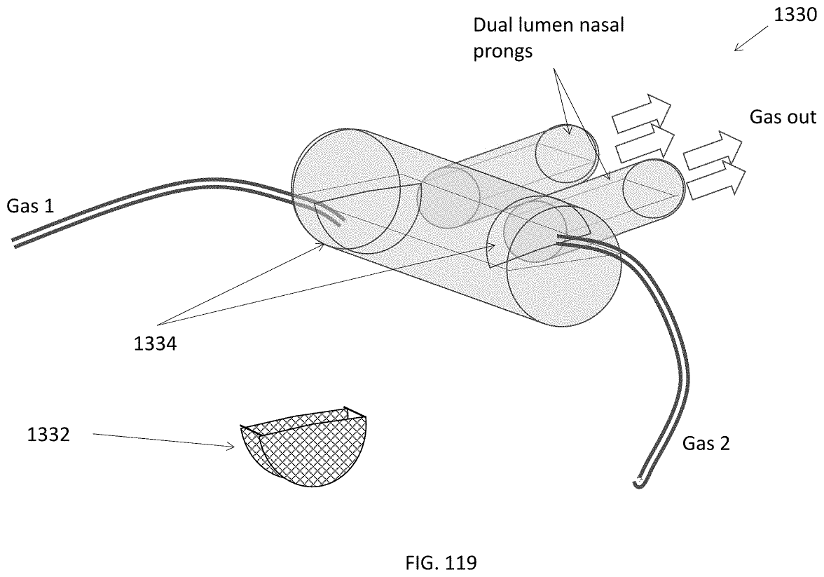

FIG. 119 depicts an exemplary embodiment of a dual-lumen cannula with dual-lumen prongs and gas filtration. The dual-lumen cannula 1330 includes particle filter elements 1332 located in the prong housing at filter locations 1334 for one or more of the gas flow paths. By placing the filter in the prong housing instead of either the gas lumen extrusion or individual prongs, a larger filter with larger cross-sectional area can be used, providing greater filter life and less flow restriction. In some embodiments, scrubbing material is also located within the prong housing to minimize bulk of the cannula and scrub gas as late as possible.

Electrode Design

Nitric oxide generation systems that utilize electrical discharge between two or more electrodes to generate a plasma can encounter wear and drifts in performance over time. In some embodiments, multiple electrodes are energized simultaneously to form an electrode array. When energized, electrical break down occurs between one pair of electrodes at a time. Each time that high voltage is applied to the electrode array, a different pair of electrodes can fire. As individual electrodes wear, the gap increases, requiring more energy to break down. Within an electrode array, electrical breakdown typically occurs at the shortest gap available. This behavior evens out the wear between electrode gaps over time, thereby prolonging the service life of the electrode array assembly.

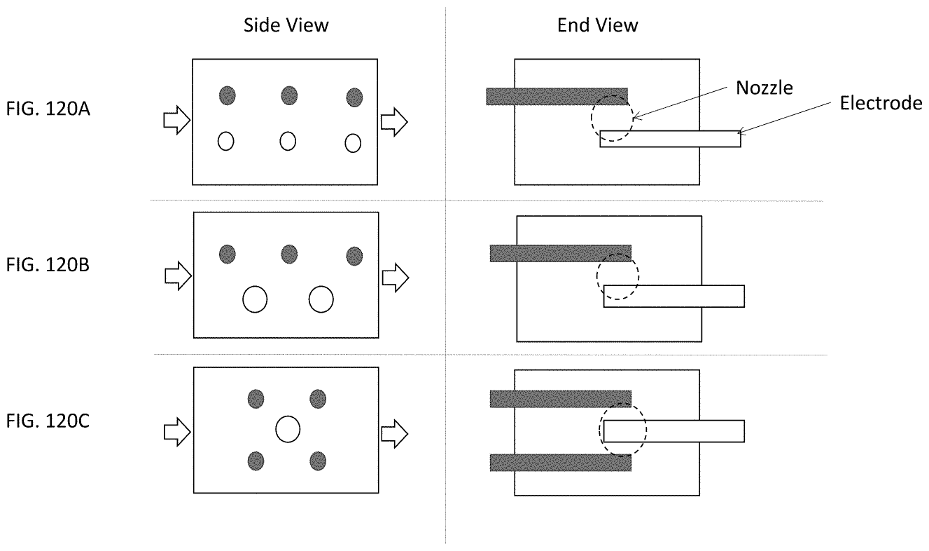

FIG. 120A depicts an exemplary embodiment of an electrode array consisting of three pairs of parallel electrodes forming three gaps. Dark colored electrodes extend from one wall of a plasma chamber and light-colored electrodes extend from an opposite wall. Dark electrodes are connected to one polarity and light-colored electrodes are connected to the other polarity. The right image demonstrates the reactant gas nozzle depicted as a dashed line circle and how it aligns with the electrode gaps.

FIG. 120B depicts an exemplary embodiment of an electrode array with 5 electrodes forming 4 gaps. The diameter of the light-colored electrodes is larger in some embodiments to provide more material for wear since they arc in two locations around the periphery. FIG. 120C depicts an exemplary embodiment of an electrode array with 5 electrodes forming 4 gaps. The diameter of the center electrode is larger in some embodiments because it includes 4 arcing locations and will tend to be hot. The thicker diameter includes more material to wear and provides better thermal conductivity for heat management.

All patents, patent applications, and published references cited herein are hereby incorporated by reference in their entirety. It will be appreciated that several of the above-disclosed and other features and functions, or alternatives thereof, may be desirably combined into many other different systems or application. Various alternatives, modifications, variations, or improvements therein may be subsequently made by those skilled in the art.

1. A nitric oxide generation system, comprising:
a plasma chamber configured to ionize a reactant gas including nitrogen and oxygen to form a product gas that includes nitric oxide (NO);
a scrubber downstream from the plasma chamber and having a volume at least partially containing NO₂ scrubbing material;
a flow controller downstream of the scrubber, the flow controller configured to control the flow of product gas from the scrubber to a delivery device; and
a pump configured to convey the product gas from the plasma chamber into the scrubber, the pump configured to pressurize the product gas in the scrubber when the flow controller is positioned to restrict the flow of product gas from the scrubber;
wherein the pressurized product gas accumulates within the scrubber and is at least partially scrubbed of NO₂ prior to passage from the scrubber through the flow controller.

2. The system of claim 1, wherein a reactant gas flow rate through the plasma chamber is continuous.

3. The system of claim 2, wherein the reactant gas flow rate through the plasma chamber is a constant value.

4. The system of claim 1, wherein a reactant gas flow rate through the plasma chamber is intermittent.

5. The system of claim 1, wherein a pressure within the plasma chamber is at or below atmospheric pressure.

6. The system of claim 1, further comprising a pressure sensor to measure the pressure in the scrubber.

7. The system of claim 6, further comprising a controller configured to regulate an amount of NO in the product gas by modulating a plasma in the plasma chamber, the controller utilizing a pressure measurement in the scrubber to determine a flow rate of the product gas out of the scrubber.

8. The system of claim 1, wherein the product gas is delivered intermittently.

9. The system of claim 8, wherein a product gas delivery flow rate varies pulse to pulse.

10. The system of claim 8, wherein a product gas delivery flow rate varies within a pulse.

11. The system of claim 1, wherein a mass of the product gas in the scrubber is at least a mass of a single NO pulse.

12. The system of claim 1, wherein the volume between the scrubber and the flow controller is less than 5 ml.

13. The system of claim 1, wherein the volume between the scrubber and the flow controller is less than 10 ml.

14. The system of claim 1, further comprising a parallel flow path that includes a pressurized non-NOx containing gas.

15. The system of claim 14, wherein the pressurized reactant gas is utilized to push an NO pulse to a patient and purge at least a portion of at least one of a pneumatic pathway within the system and the delivery device of NO and NO₂.

16. The system of claim 1, wherein the product gas is configured to accumulate such that an increase in an oxidation due to the pressure in the scrubber is more than offset by an improvement in scrubbing due to one or more of an increase in a residence time and the pressure in the scrubber.

17. The system of claim 1, further comprising a controller configured to calculate an estimated amount of NO loss within the system due to at least one of oxidation of NO and interaction between the product gas and components of the system.

18. The system of claim 17, wherein the controller is configured to control the plasma chamber to overproduce NO in anticipation of the estimated amount of NO loss calculated by the controller.

19. The system of claim 1, wherein a product gas flow rate entering the scrubber is different than from product gas flow rate exiting the scrubber.

20. The system of claim 1, wherein a mass of gas between the pump and the flow controller, including the scrubber, is greater than a mass of a pulse of gas to be delivered to a delivery device.

21. A nitric oxide generation system, comprising:
a plasma chamber configured to ionize a reactant gas including nitrogen and oxygen to form a product gas that includes nitric oxide (NO);
a scrubber downstream having a volume at least partially containing NO₂ scrubbing material;
a flow controller downstream of the scrubber, the flow controller configured to control the flow of product gas from the scrubber to a delivery device;
a pump configured to push the product gas from the plasma chamber into the scrubber, the pump configured to pressurize the product gas in the scrubber when the flow controller is positioned to restrict the flow of product gas from the scrubber; and
a controller configured to regulate an amount of NO in the product gas by the plasma chamber, the controller utilizing a pressure measurement in the scrubber to determine a mass flow rate of the product gas out of the scrubber,
wherein the pressurized product gas accumulates within the scrubber and is at least partially scrubbed of NO₂ prior to passage from the scrubber through the flow controller, and
wherein a mass of gas in the scrubber and pneumatic connections between the pump and the flow controller is greater than a mass of a pulse of gas to be delivered to a delivery device.

22. The system of claim 21, wherein a reactant gas flow rate through the plasma chamber is continuous.

23. The system of claim 22, wherein the reactant gas flow rate through the plasma chamber is a constant value.

24. The system of claim 21, wherein a reactant gas flow rate through the plasma chamber is intermittent.

25. The system of claim 21, wherein a pressure within the plasma chamber is at or below atmospheric pressure.

26. The system of claim 21, further comprising a pressure sensor to measure the pressure in the scrubber.

27. The system of claim 26, further comprising a controller configured to regulate the amount of NO in the product gas by modulating a plasma in the plasma chamber, the controller utilizing a pressure measurement in the scrubber to determine a flow rate of the product gas out of the scrubber.

28. The system of claim 21, wherein the product gas is delivered intermittently.

29. The system of claim 28, wherein a product gas delivery flow rate varies pulse to pulse.

30. The system of claim 28, wherein a product gas delivery flow rate varies within a pulse.

31. The system of claim 21, wherein a mass of the product gas in the scrubber is at least a mass of a single NO pulse.
